(19) United States
(12) Patent Application Publication
(10) Pub. No.: US 2025/0101382 A1
(43) Pub. Date: Mar. 27, 2025

(54) GENETICALLY MODIFIED CELLS AND COMPOSITIONS AND USES THEREOF

(71) Applicant: Sana Biotechnology, Inc., Seattle, WA (US)

(72) Inventors: Charles E. MURRY, Seattle, WA (US); Danny EL-NACHEF, Seattle, WA (US)

(73) Assignee: Sana Biotechnology, Inc., Seattle, WA (US)

(21) Appl. No.: 18/845,742

(22) PCT Filed: Mar. 10, 2023
(86) PCT No.: PCT/US2023/064189

§ 371 Date: Sep. 10, 2024

Related U.S. Application Data
(60) Provisional application No. 63/319,261, filed on Mar. 11, 2022.

(51) Int. Cl. C12N 5/077; A61K 35/34; A61P 9/06; C12N 9/22; C12N 15/11; C12N 15/90

(52) U.S. Cl. CPC C12N 5/0657; A61K 35/34; A61P 9/06; C12N 9/22; C12N 15/11; C12N 15/907; C12N 2310/20; C12N 2506/02; C12N 2510/00

(57) ABSTRACT

Provided are engineered cells containing one or more modifications, such as genetic modifications, for use in cardiac cell therapies. In some embodiments, the one or more modifications attenuate or prevent engraftment arrhythmia associated with a cardiac cell therapy.

### CROSS-REFERENCE TO RELATED APPLICATIONS

This application claims priority to U.S. Provisional Patent Application No. 63/319,261, filed Mar. 11, 2022, entitled “Genetically Modified Cells and Compositions and Uses Thereof,” the contents of which is hereby incorporated by reference in its entirety for all purposes.

### INCORPORATION BY REFERENCE OF SEQUENCE LISTING

The present application is being filed along with a Sequence Listing in electronic format. The Sequence Listing is provided as a file entitled 186152006840SeqList.xml, created Mar. 10, 2023, which is 53,994 bytes in size. The information in the electronic format of the Sequence Listing is incorporated by reference in its entirety.

### FIELD

The present disclosure relates in some aspects to engineered cells containing one or more modifications, such as genetic modifications, and compositions and uses thereof, such as for cardiac cell therapies. In some embodiments, the one or more modifications attenuate or prevent engraftment arrhythmia associated with a cardiac cell therapy.

### SUMMARY

Various strategies are available for treating heart conditions and diseases. Primary cardiomyocytes, as well as differentiated cardiomyocytes derived from pluripotent cells, have been investigated for use in treating heart conditions and diseases, including myocardial infarction (MI). The propensity for such cells to cause engraftment arrhythmia (EA) following engraftment in a subject reduces the potential efficacy of such treatments. While pharmacological agents and genetic engineering have been investigated for use in attenuating and preventing EA, they can have unwanted side effects, do not sufficiently reduce or eliminate EA, or both. Thus, there remains a need for improved methods of engineering cells, including for therapeutic use in cardiac cell therapies. Provided herein are embodiments that meet such needs.

Provided herein is an engineered cell comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the one or more modifications reduce expression of one or more of CACNA1G, HCN4, and SLC8A1, relative to a cell of the same cell type that does not comprise the one or more modifications. In some embodiments, the one or more modifications increase expression of one or more of KCNJ2, TRDN, SRL, HRC, and CASQ2, relative to a cell of the same cell type that does not comprise the one or more modifications. In some embodiments, the one or more modifications (a) reduce expression of one or more of CACNA1G, HCN4, and SLC8A1; and (b) increase expression of one or more of KCNJ2, TRDN, SRL, HRC, and CASQ2, relative to a cell of the same cell type that does not comprise the one or more modifications.

In some embodiments, the engineered cell comprises one or more modifications that reduce expression of CACNA1G. In some embodiments, the engineered cell comprises one or more modifications that reduce expression of HCN4 and/or SLC8A1. In some embodiments, the engineered cell comprises one or more modifications that reduce expression of HCN4. In some embodiments, the engineered cell comprises one or more modifications that reduce expression of SLC8A1. In some embodiments, the engineered cell comprises one or more modifications that reduce expression of HCN4 and SLC8A1. In some embodiments, the engineered cell comprises one or more modifications that increase expression of KCNJ2. In some of any of such embodiments, the engineered cell comprises one or more modifications that increase expression of TRDN. In some of any of such embodiments, the engineered cell comprises one or more modifications that increase expression of SRL. In some of any of such embodiments, the engineered cell comprises one or more modifications that increase expression of HRC. In some of any of such embodiments, the engineered cell comprises one or more modifications that increase expression of CASQ2. In some embodiments, the engineered cell comprises one or more modifications that (a) reduce expression of CACNA1G, HCN4, and SLC8A1; and (b) increase expression of KCNJ2.

In some embodiments, the engineered cell is a pluripotent stem cell (PSC). In some embodiments, the PSC is an induced pluripotent stem cell (iPSC). In some embodiments, the PSC is an embryonic stem cell (ESC). In some embodiments, the engineered cell is an engineered therapeutic cell, such as a primary cardiac cell or a cardiomyocyte differentiated from a PSC. In some embodiments, the engineered cell is a primary cardiac cell. In some embodiments, the engineered cell is a cardiomyocyte or a precursor thereof. In some embodiments, the engineered cell is a cardiomyocyte. In some embodiments, the engineered cell is a primary cardiomyocyte. In some embodiments, the cardiomyocyte or a precursor thereof has been differentiated from a pluripotent stem cell (PSC) in vitro. In some embodiments, the in vitro differentiation of the cardiomyocyte or a precursor thereof from a PSC comprises differentiation in suspension culture.

In some of any of such embodiments, the engineered cell comprises one or more modifications that: (a) inactivate or disrupt one or more alleles of: (i) one or more major histocompatibility complex (MHC) class I molecules or one or more molecules that regulate expression of the one or more MHC class I molecules; and/or (ii) one or more MHC class II molecules or one or more molecules that regulate expression of the one or more MHC class II molecules; and/or (b) increase expression of one or more tolerogenic factors in the engineered cell, relative to a cell of the same cell type that does not comprise the one or more modifications. In some embodiments, the one or more modifications in (a) reduce expression of one or more major histocompatibility complex (MHC) human leukocyte antigen (HLA) class I molecules and/or MHC HLA class II molecules, relative to a cell of the same cell type that does not comprise the one or more modifications.

In some embodiments, the engineered cell comprises one or more modifications that (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell of the same cell type that does not comprise the one or more modifications. In some embodiments, the engineered cell comprises one or more modifications that increase expression of one or more tolerogenic factors, relative to a cell of the same cell type that does not comprise the one or more modifications. In some embodiments, the engineered cell comprises one or more modifications that reduce expression of one or more major histocompatibility complex (MHC) human leukocyte antigen (HLA) class I molecules and/or MHC HLA class II molecules, relative to a cell of the same cell type that does not comprise the one or more modifications. In some embodiments, the engineered cell comprises one or more modifications that reduce expression of one or more major histocompatibility complex (MHC) human leukocyte antigen (HLA) class I molecules, relative to a cell of the same cell type that does not comprise the one or more modifications. In some embodiments, the engineered cell comprises one or more modifications that reduce expression of MHC HLA class II molecules, relative to a cell of the same cell type that does not comprise the one or more modifications. In some embodiments, the engineered cell comprises one or more modifications that reduce expression of one or more major histocompatibility complex (MHC) human leukocyte antigen (HLA) class I and MHC HLA class II molecules, relative to a cell of the same cell type that does not comprise the one or more modifications. In some embodiments, the engineered cell comprises one or more modifications that (i) increase expression of one or more tolerogenic factors; and (ii) reduce expression of one or more major histocompatibility complex (MHC) human leukocyte antigen (HLA) class I molecules and/or MHC HLA class II molecules, relative to a cell of the same cell type that does not comprise the one or more modifications. In some embodiments, the engineered cell comprises one or more modifications that (i) increase expression of one or more tolerogenic factors; and (ii) reduce expression of one or more major histocompatibility complex (MHC) human leukocyte antigen (HLA) class I and MHC HLA class II molecules, relative to a cell of the same cell type that does not comprise the one or more modifications.

In some of any of such embodiments, the one or more MHC class I molecules is one or more human leukocyte antigen (HLA) class I molecules. In some of any of such embodiments, the one or more MHC HLA class I molecules is selected from the group consisting of HLA-A, HLA-B, and HLA-C. In some of any of such embodiments, the one or more molecules that regulate expression of the one or more MHC class I molecules is/are selected from the group consisting of B-2 microglobulin (B2M) gene and/or the transporter 1, ATP binding cassette subfamily B member (TAP1). In some of any of such embodiments, the one or more molecules that regulate expression of the one or more MHC class I molecules regulate cell surface protein expression of the one or more MHC class I molecules. In some of any of such embodiments, the one or more modifications in (a)(i) reduce cell surface trafficking of the one or more MHC HLA class I molecules.

In some embodiments, MHC HLA class I molecules are selected from the group consisting of HLA-A, HLA-B, HLA-C, and a combination thereof. In some embodiments, the MHC HLA class I molecules is HLA-A. In some embodiments, the MHC HLA class I molecules are HLA-A and HLA-B. In some embodiments, the MHC HLA class I molecules is HLA-B. In some embodiments, the MHC HLA class I molecules are HLA-B and HLA-C. In some embodiments, the MHC HLA class I molecules is HLA-C. In some embodiments, the MHC HLA class I molecules are HLA-A and HLA-C. In some embodiments, the MHC HLA class I molecules are HLA-A, HLA-B, and HLA-C.

In some of any of such embodiments, the one or more molecules that regulate cell surface protein expression of the one or more MHC class I molecules is B2M. In some embodiments, the one or more modifications comprise a modification that regulates cell surface protein expression of the one or more MHC class I molecules and the modification inactivates or disrupts one or more alleles of B2M. In some embodiments, cell surface trafficking of the one or more MHC class I molecules is reduced in the modified cell relative to the cell of the same cell type that does not comprise the one or more modifications.

In some embodiments, the one or more modifications in (ii) reduce expression of one or more MHC HLA class I molecules. In some embodiments, the one or more modifications in (ii) reduce expression of MHC HLA class I molecules HLA-A, HLA-B, and HLA-C. In some embodiments, the one or more modifications in (ii) reduce protein expression of one or more MHC HLA class I molecules. In some embodiments, the one or more modifications that reduce protein expression reduce expression of an HLA-A protein, an HLA-B protein, or HLA-C protein. In some embodiments, the one or more modifications that reduce protein expression reduce expression of an HLA-A protein. In some embodiments, the one or more modifications that reduce protein expression reduce expression of an HLA-B protein. In some embodiments, the one or more modifications that reduce protein expression reduce expression of an HLA-C protein. In some embodiments, a gene encoding an HLA-A protein, an HLA-B protein, or an HLA-C protein, respectively, is knocked out. In some embodiments, a gene encoding an HLA-A protein is knocked out. In some embodiments, a gene encoding an HLA-B protein is knocked out. In some embodiments, a gene encoding an HLA-C protein is knocked out.

In some embodiments, the engineered cell comprises one or more modifications that reduce cell surface expression of one or more MHC HLA class I molecules. In some embodiments, the engineered cell comprises one or more modifications that reduce a function of one or more MHC HLA class I molecules. In some embodiments, the function is antigen presentation.

In some of any of such embodiments, the one or more modifications in (a) inactivates or disrupts one or more alleles of B2M, NLRC5, or TAP1. In some embodiments, the one or more modifications in (a) inactivates or disrupts one or more alleles of B2M. In some embodiments, the modification that inactivates or disrupts one or more alleles of B2M reduces mRNA expression of the B2M gene. In some embodiments, the modification that inactivates or disrupts one or more alleles of B2M reduces protein expression of B2M. In some embodiments, the modification that inactivates or disrupts one or more alleles of B2M comprises: inactivation or disruption of one allele of the B2M gene; inactivation or disruption of both alleles of the B2M gene; or inactivation or disruption of all B2M coding alleles in the cell. In some embodiments, the inactivation or disruption comprises an indel in the B2M gene. In some embodiments, the inactivation or disruption comprises a frameshift mutation or a deletion of a contiguous stretch of genomic DNA of the B2M gene.

In some embodiments, the one or more modifications in (ii) reduce expression of the B-2 microglobulin (B2M) gene and/or the transporter 1, ATP binding cassette subfamily B member (TAP1) gene. In some embodiments, the one or more modifications in (ii) reduce expression of the B-2 microglobulin (B2M) gene. In some embodiments, the one or more modifications in (ii) reduce expression of the transporter 1, ATP binding cassette subfamily B member (TAP1) gene. In some embodiments, the one or more modifications in (ii) reduce expression of the B-2 microglobulin (B2M) gene and the transporter 1, ATP binding cassette subfamily B member (TAP1) gene. In some embodiments, the one or more modifications that reduce expression in (ii) reduce expression of the B2M gene.

In some embodiments, the one or more modifications that reduce expression reduces mRNA expression of the gene. In some embodiments, the one or more modifications that reduce expression reduces protein expression of a protein encoded by the gene. In some embodiments, the one or more modifications that reduce expression comprises inactivation or disruption of one allele of the gene. In some embodiments, the one or more modifications that reduce expression comprises inactivation or disruption of both alleles of the gene. In some embodiments, the one or more modifications that reduce expression comprises inactivation or disruption of all coding sequences of the gene in the cell. In some embodiments, the inactivation or disruption comprises an indel in one allele of the gene. In some embodiments, the inactivation or disruption comprises an indel in both alleles of the gene. In some embodiments, the one or more modifications that reduce expression comprises a frameshift mutation or a deletion of a contiguous stretch of genomic DNA of the gene. In some embodiments, the gene is knocked out.

In some embodiments, the one or more modifications that reduce expression reduces mRNA expression of the B2M gene. In some embodiments, the one or more modifications that reduce expression reduces protein expression of a protein encoded by the B2M gene. In some embodiments, the one or more modifications that reduce expression comprises inactivation or disruption of one allele of the B2M gene. In some embodiments, the one or more modifications that reduce expression comprises inactivation or disruption of both alleles of the B2M gene. In some embodiments, the one or more modifications that reduce expression comprises inactivation or disruption of all coding sequences of the B2M gene in the cell. In some embodiments, the inactivation or disruption comprises an indel in one allele of the B2M gene. In some embodiments, the inactivation or disruption comprises an indel in both alleles of the B2M gene. In some embodiments, the one or more modifications that reduce expression comprises a frameshift mutation or a deletion of a contiguous stretch of genomic DNA of the B2M gene. In some embodiments, the B2M gene is knocked out.

In some embodiments, the one or more modifications that reduce expression reduces mRNA expression of the TAP1 gene. In some embodiments, the one or more modifications that reduce expression reduces protein expression of a protein encoded by the TAP1 gene. In some embodiments, the one or more modifications that reduce expression comprises inactivation or disruption of one allele of the TAP1 gene. In some embodiments, the one or more modifications that reduce expression comprises inactivation or disruption of both alleles of the TAP1 gene. In some embodiments, the one or more modifications that reduce expression comprises inactivation or disruption of all coding sequences of the TAP1 gene in the cell. In some embodiments, the inactivation or disruption comprises an indel in one allele of the TAP1 gene. In some embodiments, the inactivation or disruption comprises an indel in both alleles of the TAP1 gene. In some embodiments, the one or more modifications that reduce expression comprises a frameshift mutation or a deletion of a contiguous stretch of genomic DNA of the TAP1 gene. In some embodiments, the TAP1 gene is knocked out.

In some embodiments, the one or more modifications that reduce expression of one or more MHC HLA class I molecules is generated by nuclease-mediated gene editing. In some embodiments, the nuclease-mediated gene editing is mediated by a zinc finger nuclease (ZFN), a TAL-effector nuclease (TALEN), or a CRISPR-Cas system that targets the gene. In some embodiments, the nuclease-mediated gene editing uses a CRISPR-Cas system comprising a CRISPR-Cas nuclease and a guide RNA (gRNA) having a targeting domain that is complementary to at least one target site within the gene. In some embodiments, the CRISPR-Cas system is a ribonucleoprotein (RNP) complex comprising the gRNA and a Cas protein.

In some embodiments, the one or more modifications in (ii) reduce expression of MHC HLA class I and class II molecules. In some embodiments, the one or more modifications in (ii) reduce expression of MHC HLA class II molecules HLA-DP, HLA-DQ, and/or HLA-DR. In some embodiments, the one or more modifications in (ii) reduce expression of HLA-DP. In some embodiments, the one or more modifications in (ii) reduce expression of HLA-DQ. In some embodiments, the one or more modifications in (ii) reduce expression of HLA-DR. In some embodiments, the one or more modifications in (ii) reduce expression of HLA-DP and HLA-DQ. In some embodiments, the one or more modifications in (ii) reduce expression of HLA-DP and HLA-DR. In some embodiments, the one or more modifications in (ii) reduce expression of HLA-DQ and HLA-DR. In some embodiments, the one or more modifications in (ii) reduce expression of MHC HLA class II molecules HLA-DP, HLA-DQ, and HLA-DR.

In some embodiments, the one or more modifications in (ii) reduce protein expression of one or more MHC class II molecules. In some embodiments, the one or more modifications that reduce protein expression reduce expression of an HLA-DP protein, an HLA-DQ protein, or an HLA-DR protein. In some embodiments, a gene encoding an HLA-DP protein, an HLA-DQ protein, or an HLA-DR protein, respectively, is knocked out. In some embodiments, a gene encoding an HLA-DP protein is knocked out. In some embodiments, a gene encoding an HLA-DQ protein is knocked out. In some embodiments, a gene encoding an HLA-DR protein is knocked out.

In some embodiments, the engineered cell comprises one or more modifications that reduce cell surface expression of one or more MHC HLA class II molecules. In some embodiments, the one or more MHC class II molecules is one or more human leukocyte antigen (HLA) class II molecules. In some embodiments, the one or more modifications in (a) reduce cell surface trafficking of the one or more MHC class II molecules. In some embodiments, the one or more modifications in (a) reduce a function of the one or more MHC class II molecules, optionally wherein the function is antigen presentation.

In some of any of such embodiments, the one or more molecules that regulate expression of the one or more MHC class II molecules is/are selected from the group consisting of CIITA and CD74. In some of any of such embodiments, the modification is a modification that regulates expression of the one or more MHC class II molecules, and the modification inactivates or disrupts one or more alleles of CIITA. In some of any of such embodiments, the modification that inactivates or disrupts one or more alleles of CIITA reduces mRNA expression of the CIITA gene. In some of any of such embodiments, the modification that inactivates or disrupts one or more alleles of CIITA reduces protein expression of CIITA. In some of any of such embodiments, the modification that inactivates or disrupts one or more alleles of CIITA comprises: inactivation or disruption of one allele of the CIITA gene; inactivation or disruption of both alleles of the CIITA gene; or inactivation or disruption of all CIITA coding alleles in the cell. In some of any of such embodiments, the inactivation or disruption comprises an indel in the CIITA gene. In some of any of such embodiments, the inactivation or disruption is a frameshift mutation or a deletion of a contiguous stretch of genomic DNA of the CIITA gene.

In some of any of such embodiments, expression of HLA-A, HLA-B, HLA-C, HLA-DP, HLA-DQ, and HLA-DR are reduced in the engineered cell.

In some embodiments, the engineered cell comprises one or more modifications that reduce a function of one or more MHC HLA class II molecules. In some embodiments, the function is antigen presentation.

In some embodiments, the one or more modifications in (ii) reduce expression of the CD74 gene. In some embodiments, the one or more modifications that reduce expression reduce mRNA expression of the CD74 gene. In some embodiments, the one or more modifications that reduce expression reduces expression of a CD74 protein. In some embodiments, the one or more modifications that reduce expression comprises inactivation or disruption of one allele of the CD74 gene. In some embodiments, the one or more modifications that reduce expression comprises inactivation or disruption of both alleles of the CD74 gene. In some embodiments, the one or more modifications that reduce expression comprises inactivation or disruption of all CD74 coding sequences in the cell. In some embodiments, the inactivation or disruption comprises an indel in one allele of the CD74 gene. In some embodiments, the inactivation or disruption comprises an indel in both alleles of the CD74 gene. In some embodiments, the one or more modifications that reduce expression comprises a frameshift mutation or a deletion of a contiguous stretch of genomic DNA of the CD74 gene. In some embodiments, the CD74 gene is knocked out.

In some embodiments, the one or more modifications that reduce expression of one or more MHC HLA class II molecules is generated by nuclease-mediated gene editing. In some embodiments, the nuclease-mediated gene editing is mediated by a zinc finger nuclease (ZFN), a TAL-effector nuclease (TALEN), or a CRISPR-Cas system that targets the CD74 gene. In some embodiments, the nuclease-mediated gene editing uses a CRISPR-Cas system comprising a CRISPR-Cas nuclease and a guide RNA (gRNA) having a targeting domain that is complementary to at least one target site within the CD74 gene. In some embodiments, the CRISPR-Cas system is a ribonucleoprotein (RNP) complex comprising the gRNA and a Cas protein.

In some of any of such embodiments, expression of HLA-A, HLA-B, HLA-C, HLA-DP, HLA-DQ, and HLA-DR are reduced in the modified cell.

In some embodiments, the one or more modifications in (ii) reduce expression of the CIITA gene. In some embodiments, the one or more modifications that reduce expression reduce mRNA expression of the CIITA gene. In some embodiments, the one or more modifications that reduce expression reduces expression of a CIITA protein. In some embodiments, the one or more modifications that reduce expression comprises inactivation or disruption of one allele of the CIITA gene. In some embodiments, the one or more modifications that reduce expression comprises inactivation or disruption of both alleles of the CIITA gene. In some embodiments, the one or more modifications that reduce expression comprises inactivation or disruption of all CIITA coding sequences in the cell. In some embodiments, the inactivation or disruption comprises an indel in one allele of the CIITA gene. In some embodiments, the inactivation or disruption comprises an indel in both alleles of the CIITA gene. In some embodiments, the one or more modifications that reduce expression comprises a frameshift mutation or a deletion of a contiguous stretch of genomic DNA of the CIITA gene. In some embodiments, the CIITA gene is knocked out.

In some embodiments, the one or more modifications that reduce expression of one or more MHC HLA class II molecules is generated by nuclease-mediated gene editing. In some embodiments, the nuclease-mediated gene editing is mediated by a zinc finger nuclease (ZFN), a TAL-effector nuclease (TALEN), or a CRISPR-Cas system that targets the CIITA gene. In some embodiments, the nuclease-mediated gene editing uses a CRISPR-Cas system comprising a CRISPR-Cas nuclease and a guide RNA (gRNA) having a targeting domain that is complementary to at least one target site within the CIITA gene. In some embodiments, the CRISPR-Cas system is a ribonucleoprotein (RNP) complex comprising the gRNA and a Cas protein.

In some embodiments, the one or more tolerogenic factors are one or more of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the one or more tolerogenic factors are one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, the one or more tolerogenic factors in (i) are selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof. In some embodiments, the one or more tolerogenic factors in (i) are selected from the group consisting of CD47, PD-L1, HLA-E, HLA-G, CCL21, FASL, SERPINB9, CD200, MFGE8, and any combination thereof. In some embodiments, the one or more tolerogenic factors comprises CD47. In some embodiments, the one or more tolerogenic factors is CD47. In some embodiments, the one or more tolerogenic factors comprise CD47, and the one or more modifications that increases expression of CD47 comprise an exogenous polynucleotide encoding the CD47 protein. In some embodiments, the one or more modifications that increases expression of CD47 comprise an exogenous polynucleotide encoding the CD47 protein. In some embodiments, the exogenous polynucleotide encoding the CD47 protein is integrated into the genome of the engineered cell. In some embodiments, the exogenous polynucleotide encoding the CD47 protein encodes an amino acid sequence having at least 85% sequence identity to the amino acid sequence set forth in SEQ ID NO: 2. In some embodiments, the exogenous polynucleotide encoding the CD47 protein encodes the amino acid sequence set forth in SEQ ID NO:2.

In some embodiments, the exogenous polynucleotide is integrated by non-targeted insertion into the genome of the engineered cell. In some embodiments, the exogenous polynucleotide is integrated by introduction of the exogenous polynucleotide into the cell using a lentiviral vector. In some embodiments, the exogenous polynucleotide is integrated by targeted insertion into a target genomic locus of the engineered cell. In some embodiments, the target genomic locus is a safe harbor locus, a B2M gene locus or a CIITA gene locus. In some embodiments, the target genomic locus is a safe harbor locus, a B2M gene locus, a CIITA gene locus, a CACNA1G locus, a HCN4 locus, or a SLC8A1 locus. In some embodiments, the target genomic locus is selected from the group consisting of: a CCR5 gene locus, a CXCR4 gene locus, a PPP1R12C (also known as AAVS1) gene locus, an albumin gene locus, a SHS231 locus, a CLYBL gene locus, and a ROSA26 gene locus.

In some embodiments, the one or more modifications that reduce expression in (a) comprise reduced surface protein expression; and/or the one or more modifications that increase expression in (b) comprise increased surface protein expression. In some embodiments, the one or more modifications that reduce expression in (a) comprise reduced surface protein expression. In some embodiments, the one or more modifications that increase expression in (b) reduce expression in comprise increased surface protein expression. In some embodiments, the one or more modifications that reduce expression in (a) comprise reduced surface protein expression; and the one or more modifications that increase expression in (b) comprise increased surface protein expression.

Provided herein is an engineered cell comprising one or more modifications that: (a) reduce expression of CACNA1G, HCN4, and SLC8A1, one or more MHC HLA class I molecules, and/or one or more MHC HLA class II molecules; and (b) increase expression of KCNJ2 and CD47, relative to a cell of the same cell type that does not comprise the one or more modifications. In some embodiments, the one or more modifications reduce expression of CACNA1G, HCN4, and SLC8A1, relative to a cell of the same cell type that does not comprise the one or more modifications. In some embodiments, the one or more modifications reduce expression of one or more of MHC HLA class I molecules, and/or one or more of MHC HLA class II molecules, relative to a cell of the same cell type that does not comprise the one or more modifications. In some embodiments, the one or more modifications increase expression of KCNJ2 and CD47, relative to a cell of the same cell type that does not comprise the one or more modifications.

Also provided herein is an engineered cell comprising one or more modifications that: (a) inactivate or disrupt one or more alleles of CACNA1G, HCN4, and SLC8A1, one or more MHC HLA class I molecules, and/or one or more MHC HLA class II molecules; and (b) increase expression of KCNJ2 and CD47, relative to a cell of the same cell type that does not comprise the one or more modifications. In some embodiments, the one or more modifications of (a) reduce expression of CACNA1G, HCN4, and SLC8A1, one or more MHC HLA class I molecules, and/or one or more MHC HLA class II molecules, relative to a cell of the same cell type that does not comprise the one or more modifications.

Also provided herein is an engineered cell comprising one or more modifications that: (a) reduce expression of CACNA1G, HCN4, and SLC8A1, one or more MHC HLA class I molecules, and one or more MHC HLA class II molecules; and (b) increase expression of KCNJ2 and CD47, relative to a cell of the same cell type that does not comprise the one or more modifications. In some embodiments, the one or more modifications that reduce expression of one or more MHC HLA class I molecules and/or one or more MHC class II molecules reduce expression of B2M gene and CD74. In some embodiments, the one or more modifications that reduce expression of one or more MHC HLA class I molecules and/or one or more MHC class II molecules reduce expression of B2M and CIITA.

Also provided herein is an engineered cell comprising one or more modifications that: (a) inactivate or disrupt one or more alleles of CACNA1G, HCN4, and SLC8A1, one or more MHC HLA class I molecules, and one or more MHC HLA class II molecules; and (b) increase expression of KCNJ2 and CD47, relative to a cell of the same cell type that does not comprise the one or more modifications. In some embodiments, the one or more modifications of (a) reduce expression of CACNA1G, HCN4, and SLC8A1, one or more MHC HLA class I molecules, and one or more MHC HLA class II molecules, relative to a cell of the same cell type that does not comprise the one or more modifications.

Also provided herein is an engineered cell comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.

Also provided herein is an engineered cell comprising one or more modifications that (a) inactivate or disrupt one or more alleles of CACNA1G, HCN4, SLC8A1, B2M, and CIITA; and (b) increase expression of KCNJ2 and CD47, relative to a cell of the same cell type that does not comprise the one or more modifications. In some embodiments, the one or more modifications of (a) reduce expression of CACNA1G, HCN4, SLC8A1, B2M, and CIITA, relative to a cell of the same cell type that does not comprise the one or more modifications.

Also provided herein is an engineered primary human cell comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.

In some embodiments, the engineered cell is an engineered therapeutic cell, such as a primary cardiac cell or a cardiomyocyte that has been differentiated from a PSC.

Also provided herein is an engineered induced pluripotent stem cell (iPSC) or embryonic stem cell (ESC) comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.

Also provided herein is an engineered cardiomyocyte that has been differentiated in vitro from any of the engineered cell provided herein.

Also provided herein is an engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cardiomyocyte that does not comprise the one or more modifications.

Also provided herein is an engineered cardiomyocyte that has been differentiated in vitro from a pluripotent stem cell (PSC), wherein the engineered cardiomyocyte comprises one or more modifications that (a) inactivate or disrupt one or more alleles of: (i) CACNA1G, HCN4, and SLC8A1; (ii) MHC HLA class I molecules and one or more MHC HLA class II molecules; (b) increase expression of one or more of KCNJ2, TRDN, SRL, HRC, CASQ2, and CD47; or (d) a combination thereof; or (c) a combination thereof, relative to a cardiomyocyte differentiated in vitro from a PSC that does not comprise the one or more modifications. In some embodiments, the one or more modifications of (a) reduce expression of one or more of CACNA1G, HCN4, and SLC8A1; and reduce expression of one or more of MHC HLA class I molecules and one or more MHC HLA class II molecules, relative to a cardiomyocyte that does not comprise the one or more modifications.

Also provided herein is an engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cardiomyocyte that does not comprise the one or more modifications. In some embodiments, the one or more modifications that reduce expression of one or more MHC HLA class I molecules and one or more MHC class II molecules reduce expression of B2M gene and CIITA. In some embodiments, the one or more modifications that reduce expression of one or more MHC HLA class I molecules and one or more MHC class II molecules reduce expression of B2M and CD74.

Also provided herein is an engineered cardiomyocyte that has been differentiated in vitro from a pluripotent stem cell (PSC), wherein the engineered cardiomyocyte comprises one or more modifications that: (a) inactivate or disrupt one or more alleles of: (i) CACNA1G, HCN4, and SLC8A1; (ii) B2M, TAP1, and CIITA; (b) increase expression of one or more of KCNJ2, TRDN, SRL, HRC, CASQ2, and CD47; or (c) a combination thereof, relative to a cardiomyocyte differentiated in vitro from a PSC that does not comprise the one or more modifications.

Also provided herein is an engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) reduce expression of one or more of B2M, TAP1, and CIITA; (c) increase expression of one or more of KCNJ2, TRDN, SRL, HRC, CASQ2, and CD47; or (d) a combination thereof, relative to a cardiomyocyte that does not comprise the one or more modifications.

In some embodiments, the engineered cardiomyocyte is an engineered therapeutic cell, such as for use in a cardiac cell therapy.

In some of any of such embodiments, the reduced expression and/or increased expression is relative to a primary cardiomyocyte that does not comprise the one or more modifications.

In some embodiments, the engineered cell comprises one or more modifications that reduce expression of CACNA1G. In some embodiments, the engineered cell comprises one or more modifications that reduce expression of HCN4 and/or SLC8A1 . . . . In some embodiments, the engineered cell comprises one or more modifications that reduce expression of HCN4. In some embodiments, the engineered cell comprises one or more modifications that reduce expression of SLC8A1. In some embodiments, the engineered cell comprises one or more modifications that reduce expression of HCN4 and SLC8A1. In some embodiments, the engineered cell comprises one or more modifications that increase expression of KCNJ2. In some embodiments, the engineered cell comprises one or more modifications that increase expression of TRDN. In some embodiments, the engineered cell comprises one or more modifications that increase expression of SRL. In some embodiments, the engineered cell comprises one or more modifications that increase expression of HRC. In some embodiments, the engineered cell comprises one or more modifications that increase expression of CASQ2.

In some embodiments, the engineered cell comprises one or more modifications that (a) reduce expression of CACNA1G, HCN4, and SLC8A1; and (b) increase expression of KCNJ2.

In some embodiments, the engineered cardiomyocyte comprises one or more modifications that reduce expression of CACNA1G. In some embodiments, the engineered cardiomyocyte comprises one or more modifications that reduce expression of HCN4 and/or SLC8A1. In some embodiments, the engineered cardiomyocyte comprises one or more modifications that reduce expression of HCN4. In some embodiments, the engineered cardiomyocyte comprises one or more modifications that reduce expression of SLC8A1. In some embodiments, the engineered cardiomyocyte comprises one or more modifications that reduce expression of HCN4 and SLC8A1.

In some embodiments, the engineered cardiomyocyte comprises one or more modifications that increase expression of KCNJ2. In some embodiments, the engineered cardiomyocyte comprises one or more modifications that (a) reduce expression of CACNA1G, HCN4, and SLC8A1; and (b) increase expression of KCNJ2.

In some of any of such embodiments, the engineered cell or cardiomyocyte comprises one or more modifications that: (a) inactivate or disrupt one or more alleles of: (i) one or more major histocompatibility complex (MHC) class I molecules or one or more molecules that regulate expression of the one or more MHC class I molecules; and/or (ii) one or more MHC class II molecules or one or more molecules that regulate expression of the one or more MHC class II molecules; and/or (b) increase expression of one or more tolerogenic factors in the engineered cell or cardiomyocyte, relative to a cell of the same cell type that does not comprise the one or more modifications. In some embodiments, the one or more modifications (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.

In some embodiments, the engineered cell comprises one or more modifications that: (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications. In some embodiments, the engineered cell comprises one or more modifications that increase expression of one or more tolerogenic factors, relative to a cell or cardiomyocyte that does not comprise the one or more modifications. In some embodiments, the engineered cell comprises one or more modifications that reduce expression of one or more major histocompatibility complex (MHC) human leukocyte antigen (HLA) class I molecules and/or MHC HLA class II molecules, relative to a cell or cardiomyocyte that does not comprise the one or more modifications. In some embodiments, the engineered cell comprises one or more modifications that: (i) increase expression of one or more tolerogenic factors; and (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.

In some embodiments, the engineered cardiomyocyte comprises one or more modifications that: (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications. In some embodiments, the engineered cardiomyocyte comprises one or more modifications that increase expression of one or more tolerogenic factors, relative to a cell or cardiomyocyte that does not comprise the one or more modifications. In some embodiments, the engineered cardiomyocyte comprises one or more modifications that reduce expression of one or more major histocompatibility complex (MHC) human leukocyte antigen (HLA) class I molecules and/or MHC HLA class II molecules, relative to a cell or cardiomyocyte that does not comprise the one or more modifications. In some embodiments, the engineered cardiomyocyte comprises one or more modifications that: (i) increase expression of one or more tolerogenic factors; and (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.

In some embodiments, MHC HLA class I molecules are selected from the group consisting of HLA-A, HLA-B, HLA-C, and a combination thereof. In some embodiments, the one or more modifications reduce expression of one or more MHC HLA class I molecules. In some embodiments, the one or more modifications reduce expression of MHC HLA class I molecules HLA-A, HLA-B, and HLA-C. In some embodiments, the one or more modifications reduce protein expression of one or more MHC HLA class I molecules. In some embodiments, the one or more modifications that reduce protein expression reduce expression of an HLA-A protein, an HLA-B protein, or HLA-C protein. In some embodiments, a gene encoding an HLA-A protein, an HLA-B protein, or an HLA-C protein, respectively, is knocked out.

In some embodiments, the engineered cell comprises one or more modifications that reduce cell surface expression of one or more MHC HLA class I molecules. In some embodiments, the engineered cell comprises one or more modifications that reduce a function of one or more MHC HLA class I molecules. In some embodiments, the function is antigen presentation. In some embodiments, the one or more modifications that reduce expression reduce expression of the B2M gene. In some embodiments, the one or more modifications reduce expression of MHC HLA class I and class II molecules. In some embodiments, the one or more modifications reduce expression of MHC HLA class II molecules HLA-DP, HLA-DQ, or HLA-DR. In some embodiments, the one or more modifications reduce protein expression of one or more MHC class II molecules. In some embodiments, the one or more modifications that reduce protein expression reduce expression of an HLA-DP protein, an HLA-DQ protein, or an HLA-DR protein. In some embodiments, a gene encoding an HLA-DP protein, an HLA-DQ protein, or an HLA-DR protein, respectively, is knocked out.

In some embodiments, the engineered cell or cardiomyocyte comprises one or more modifications that reduce cell surface expression of one or more MHC HLA class II molecules. In some embodiments, the engineered cell or cardiomyocyte comprises one or more modifications that reduce a function of one or more MHC HLA class II molecules. In some embodiments, the function is antigen presentation.

In some embodiments, the one or more modifications reduce expression of the CIITA gene. In some embodiments, the one or more modifications reduce expression of the CD74 gene. In some embodiments, the one or more tolerogenic factors comprise DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, the one or more tolerogenic factors comprises CD47. In some embodiments, the one or more tolerogenic factors comprise CD47, and the one or more modifications that increases expression of CD47 comprise an exogenous polynucleotide encoding the CD47 protein. In some embodiments, the one or more modifications that increases expression of CD47 comprise an exogenous polynucleotide encoding the CD47 protein.

In some embodiments, the phenotype of the engineered cell comprises B2Mindel/indel; CIITAindel/indel; and CD47tg. In some embodiments, the phenotype of the engineered cardiomyocyte comprises B2Mindel/indel; CIITAindel/indel; and CD47tg.

In some of any of such embodiments, the engineered cell or cardiomyocyte further comprises a modification for expression of an exogenous safety switch. In some of any of such embodiments, the safety switch is a system wherein upon activation, cells downregulate expression of the one or more tolerogenic factors and/or upregulate expression of one or more immune signaling molecules thereby marking the engineered cell or cardiomyocyte for elimination by the host immune system. In some of any of such embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some of any of such embodiments, the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some of any of such embodiments, the safety switch is a suicide gene. In some embodiments, the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

In some of any of such embodiments, the safety switch and the one or more tolerogenic factors are expressed from a bicistronic cassette integrated into the genome of the engineered cell or cardiomyocyte. In some of any of such embodiments, the bicistronic cassette is integrated at a non-target locus in the genome of the engineered cell or cardiomyocyte. In some of any of such embodiments, the bicistronic cassette is integrated into a target genomic locus of the engineered cell or cardiomyocyte.

In some of any of such embodiments, the engineered cell or cardiomyocyte comprises an exogenous polynucleotide encoding a safety switch. In some embodiments, the safety switch is a system wherein upon activation, cells downregulate expression of the one or more tolerogenic factors and/or upregulate expression of one or more immune signaling molecules thereby marking the cell for elimination by the host immune system. In some of any of such embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some of any of such embodiments, the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some of any of such embodiments, the safety switch is a suicide gene. In some embodiments, the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

In some of any of such embodiments, the safety switch and genes associated with the safety switch are expressed from a bicistronic cassette integrated into the genome of the engineered cell or cardiomyocyte. In some of any of such embodiments, the safety switch and the one or more tolerogenic factors are expressed from a bicistronic cassette integrated into the genome of the engineered cell or cardiomyocyte. In some of any of such embodiments, the bicistronic cassette is integrated by non-targeted insertion into the genome of the engineered cell or cardiomyocyte. In some of any of such embodiments, the bicistronic cassette is integrated by targeted insertion into a target genomic locus of the engineered cell or cardiomyocyte. In some of any of such embodiments, the one or more tolerogenic factors is CD47.

In some of any of such embodiments, the inactivation or disruption is by one or more gene edits. In some of any of such embodiments, the cell comprises a genome editing complex. In some of any of such embodiments, the one or more gene edits are made by a genome editing complex. In some of any of such embodiments, the genome editing complex comprises a genome targeting entity and a genome modifying entity. In some of any of such embodiments, the genome targeting entity localizes the genome editing complex to the one or more alleles that are inactivated or disrupted, optionally wherein the genome targeting entity is a nucleic acid-guided targeting entity. In some of any of such embodiments, the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

In some of any of such embodiments, the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

In some of any of such embodiments, the genome modifying entity cleaves, deaminates, nicks, polymerizes, interrogates, integrates, cuts, unwinds, breaks, alters, methylates, demethylates, or otherwise destabilizes the target locus. In some of any of such embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

In some of any of such embodiments, the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof. In some of any of such embodiments, the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof. In some of any of such embodiments, the genome targeting entity and the genome modifying entity are different domains of a single polypeptide. In some of any of such embodiments, the genome editing entity and genome modifying entity are two different polypeptides that are operably linked together. In some of any of such embodiments, the genome editing entity and genome modifying entity are two different polypeptides that are not linked together.

In some of any of such embodiments, the genome editing complex comprises a guide nucleic acid having a targeting domain that is complementary to at least one target locus, optionally wherein the guide nucleic acid is a guide RNA (gRNA). In some of any of such embodiments, the one or more modifications are made by the genome editing complex. In some of any of such embodiments, the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

In some of any of such embodiments, the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

In some of any of such embodiments, the modifications made by the genome editing complex are made using a guide RNA (gRNA) having a targeting domain that is complementary to at least one target site. In some of any of such embodiments, the genome editing complex is an RNA-guided nuclease. In some of any of such embodiments, the RNA-guided nuclease comprises a Cas nuclease and a guide RNA (CRISPR-Cas combination). In some of any of such embodiments, the CRISPR-Cas combination is a ribonucleoprotein (RNP) complex comprising the gRNA and the Cas nuclease. In some of any of such embodiments, the Cas nuclease is a Type II or Type V Cas protein. In some of any of such embodiments, the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

In some of any of such embodiments, the engineered cell or cardiomyocyte has been differentiated from a pluripotent stem cell (PSC) in vitro. In some of any of such embodiments, the in vitro differentiation of the engineered cell or cardiomyocyte from a PSC comprises differentiation in suspension culture. In some of any of such embodiments, differentiation of the cardiomyocyte from the PSC comprises differentiation in suspension culture. In some of any of such embodiments, one or more modifications that reduce expression and/or the one or more modifications that increase expression is carried out prior to the differentiation. In some of any of such embodiments, one or more modifications that reduce expression and/or the one or more modifications that increase expression is carried out subsequent to the differentiation. In some of any of such embodiments, one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.

In some embodiments, the engineered cell is human. In some embodiments, the engineered cardiomyocyte is human.

Provided herein is a composition comprising a plurality of any of the engineered cells provided herein.

Also provided herein is a composition comprising a plurality of any of the engineered cardiomyocytes provided herein.

In some embodiments, the composition is a therapeutic composition, such as a cardiac cell therapy. In some embodiments, the engineered cells of the therapeutic composition are cardiomyocytes differentiated from PSCs or primary cardiac cells.

In some embodiments, the composition comprises between about 5×108 and 1×1010 engineered cardiomyocytes, inclusive of each. In some embodiments, the composition comprises between about 1×109 and about 5×109 engineered cardiomyocytes, inclusive of each. In some embodiments, the composition comprises a pharmaceutically acceptable carrier.

In some of any of such embodiments, at least at or about 50%, at least at or about 60%, at least at or about 70%, at least at or about 80%, or at least at or about 90% of the cells in the population are reduced for expression of one or more MHC class I molecules and/or for expression of B2M. In some of any of such embodiments, at least at or about 50%, at least at or about 60%, at least at or about 70%, at least at or about 80%, or at least at or about 90% of the cells in the population are reduced for expression of one or more MHC class II molecules and/or for expression of CIITA. In some of any of such embodiments, at least at or about 50%, at least at or about 60%, at least at or about 70%, at least at or about 80%, or at least at or about 90% of the cells in the population comprise inactivation or disruption of one or more alleles of: one or more MHC class I molecules and/or B2M. In some of any of such embodiments, at least at or about 50%, at least at or about 60%, at least at or about 70%, at least at or about 80%, or at least at or about 90% of the cells in the population comprise inactivation or disruption of one or more alleles of: one or more MHC class II molecules and/or CIITA. In some of any of such embodiments, at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications. In some of any of such embodiments, at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications. In some of any of such embodiments, at least at or about 50%, at least at or about 60%, at least at or about 70%, at least at or about 80%, or at least at or about 90% of the cells in the population expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any of such embodiments, the inactivation or disruption is by one or more gene edits.

In some of any of such embodiments, the cells of the plurality of the engineered cardiomyocytes comprise a genome editing complex. In some of any of such embodiments, the one or more gene edits are made by a genome editing complex. In some of any of such embodiments, the genome editing complex comprises a genome targeting entity and a genome modifying entity. In some of any of such embodiments, the genome targeting entity localizes the genome editing complex to the one or more alleles that are inactivated or disrupted, optionally wherein the genome targeting entity is a nucleic acid-guided targeting entity. In some of any of such embodiments, the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

In some of any of such embodiments, the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

In some of any of such embodiments, the genome modifying entity cleaves, deaminates, nicks, polymerizes, interrogates, integrates, cuts, unwinds, breaks, alters, methylates, demethylates, or otherwise destabilizes the target locus. In some of any of such embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some of any of such embodiments, the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

In some of any of such embodiments, the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

In some of any of such embodiments, the genome targeting entity and the genome modifying entity are different domains of a single polypeptide. In some of any of such embodiments, the genome editing entity and genome modifying entity are two different polypeptides that are operably linked together. In some of any of such embodiments, the genome editing entity and genome modifying entity are two different polypeptides that are not linked together. In some of any of such embodiments, the genome editing complex comprises a guide nucleic acid having a targeting domain that is complementary to at least one target locus, optionally wherein the guide nucleic acid is a guide RNA (gRNA).

In some of any of such embodiments, the one or more modifications are made by the genome editing complex. In some of any of such embodiments, the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

In some of any of such embodiments, the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

In some of any of such embodiments, the modifications made by the genome editing complex are made using a guide RNA (gRNA) having a targeting domain that is complementary to at least one target site. In some of any of such embodiments, the genome editing complex is an RNA-guided nuclease. In some of any of such embodiments, the RNA-guided nuclease comprises a Cas nuclease and a guide RNA (CRISPR-Cas combination). In some of any of such embodiments, the CRISPR-Cas combination is a ribonucleoprotein (RNP) complex comprising the gRNA and the Cas nuclease. In some of any of such embodiments, the Cas nuclease is a Type II or Type V Cas protein. In some of any of such embodiments, the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

In some of any of such embodiments, the composition comprises a pharmaceutically acceptable excipient. In some of any of such embodiments, the composition comprises a cryoprotectant.

Also provided herein is a method of producing an engineered cell, the method comprising: (a) reducing expression of one or more of CACNA1G, HCN4, and SLC8A1; (b) increasing expression of one or more of KCNJ2, TRDN, SRL, HRC, and CASQ2; or (c) a combination thereof, in the cell. In some embodiments, the method comprises reducing expression of one or more of CACNA1G, HCN4, and SLC8A1. In some embodiments, the method comprises increasing expression of one or more of KCNJ2, TRDN, SRL, HRC, and CASQ2. In some embodiments, the method comprises (a) reducing expression of one or more of CACNA1G, HCN4, and SLC8A1; and (b) increasing expression of one or more of KCNJ2, TRDN, SRL, HRC, and CASQ2.

In some embodiments, the method comprises reducing expression of CACNA1G in the cell. In some embodiments, the method comprises reducing expression of HCN4 and/or SLC8A1 in the cell. In some embodiments, the method comprises reducing expression of HCN4 in the cell. In some embodiments, the method comprises reducing expression of SLC8A1 in the cell. In some embodiments, the method comprises reducing expression of HCN4 and SLC8A1 in the cell. In some embodiments, the method comprises increasing expression of KCNJ2 in the cell. In some of any of such embodiments, the method comprises increasing expression of TRDN in the cell. In some of any of such embodiments, the method comprises increasing expression of SRL in the cell. In some of any of such embodiments, the method comprises increasing expression of HRC in the cell. In some of any of such embodiments, the method comprises increasing expression of CASQ2 in the cell. In some embodiments, the method comprises: (a) reducing expression of CACNA1G, HCN4, and SLC8A1; and (b) increasing expression of KCNJ2, in the cell.

In some embodiments, the engineered cell is a pluripotent stem cell (PSC). In some embodiments, the PSC is an induced pluripotent stem cell (iPSC). In some embodiments, the PSC is an embryonic stem cell (ESC). In some embodiments, the engineered cell is a primary cardiac cell. In some embodiments, the engineered cell is a cardiomyocyte or a precursor thereof. In some embodiments, the engineered cell is a cardiomyocyte. In some embodiments, the engineered cell is a primary cardiomyocyte. In some embodiments, the cardiomyocyte or a precursor thereof has been differentiated from a pluripotent stem cell (PSC) in vitro. In some embodiments, the in vitro differentiation of the cardiomyocyte or a precursor thereof from a PSC comprises differentiation in suspension culture. In some embodiments, the method further comprises differentiating the PSC into a cardiomyocyte. In some embodiments, differentiation of the cardiomyocyte from the PSC comprises differentiation in suspension culture.

In some of any of such embodiments, the reducing expression and/or the increasing expression is carried out prior to the differentiation. In some of any of such embodiments, the reducing expression and/or the increasing expression is carried out subsequent to the differentiation. In some of any of such embodiments, part of the reducing expression and/or the increasing expression is carried out prior to the differentiation; and part of the reducing expression and/or the increasing expression is carried out subsequent to the differentiation. In some of any of such embodiments, one or more modifications that reduce expression and/or the one or more modifications that increase expression is carried out prior to the differentiation. In some of any of such embodiments, one or more modifications that reduce expression and/or the one or more modifications that increase expression is carried out subsequent to the differentiation. In some of any of such embodiments, one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.

In some embodiments, the engineered cell is an engineered therapeutic cell, such as a primary cardiac cell or a cardiomyocyte that has been differentiated from a PSC.

In some embodiments, the engineered cell comprises one or more modifications that: (a) inactivate or disrupt one or more alleles of: (i) one or more major histocompatibility complex (MHC) class I molecules or one or more molecules that regulate expression of the one or more MHC class I molecules; and/or (ii) one or more MHC class II molecules or one or more molecules that regulate expression of the one or more MHC class II molecules; and/or (b) increase expression of one or more tolerogenic factors in the engineered cell or cardiomyocyte, relative to a cell of the same cell type that does not comprise the one or more modifications. In some embodiments, the one or more modifications (i) increase expression of one or more tolerogenic factors; and/or (ii) reduce expression of one or more major histocompatibility complex (MHC) class I molecules and/or MHC class II, relative to a cell of the same cell type that does not comprise the one or more modifications. In some of any of such embodiments, the one or more molecules that regulate cell surface protein expression of the one or more MHC class I molecules is B2M. In some of any of such embodiments, the one or more modifications comprise a modification that regulates cell surface protein expression of the one or more MHC class I molecules and the modification inactivates or disrupts one or more alleles of B2M. In some of any of such embodiments, cell surface trafficking of the one or more MHC class I molecules is reduced in the modified cell relative to the cell of the same cell type that does not comprise the one or more modifications.

In some embodiments, the engineered cell comprises one or more modifications that: (i) increase expression of one or more tolerogenic factors; and/or (ii) reduce expression of one or more major histocompatibility complex (MHC) class I molecules and/or MHC class II, relative to a cell of the same cell type that does not comprise the one or more modifications. In some embodiments, the engineered cell comprises one or more modifications that increase expression of one or more tolerogenic factors, relative to a cell of the same cell type that does not comprise the one or more modifications. In some embodiments, the engineered cell comprises one or more modifications that reduce expression of one or more major histocompatibility complex (MHC) class I molecules and/or MHC class II, relative to a cell of the same cell type that does not comprise the one or more modifications. In some embodiments, the engineered cell comprises one or more modifications that: (i) increase expression of one or more tolerogenic factors; and (ii) reduce expression of one or more major histocompatibility complex (MHC) class I molecules and/or MHC class II, relative to a cell of the same cell type that does not comprise the one or more modifications.

In some embodiments, MHC HLA class I molecules are selected from the group consisting of HLA-A, HLA-B, HLA-C, and a combination thereof. In some embodiments, the one or more modifications in (ii) reduce expression of one or more MHC HLA class I molecules. In some embodiments, the one or more modifications in (ii) reduce expression of MHC HLA class I molecules HLA-A, HLA-B, and HLA-C. In some embodiments, the one or more modifications in (ii) reduce protein expression of one or more MHC HLA class I molecules. In some embodiments, the one or more modifications that reduce protein expression reduce expression of an HLA-A protein, an HLA-B protein, or HLA-C protein. In some embodiments, a gene encoding an HLA-A protein, an HLA-B protein, or an HLA-C protein, respectively, is knocked out.

In some of any of such embodiments, the one or more modifications in (a) inactivates or disrupts one or more alleles of B2M, NLRC5, or TAP1. In some embodiments, the one or more modifications in (a) inactivates or disrupts one or more alleles of B2M. In some of any of such embodiments, the modification that inactivates or disrupts one or more alleles of B2M reduces mRNA expression of the B2M gene. In some of any of such embodiments, the modification that inactivates or disrupts one or more alleles of B2M reduces protein expression of B2M. In some of any of such embodiments, the modification that inactivates or disrupts one or more alleles of B2M comprises: inactivation or disruption of one allele of the B2M gene; inactivation or disruption of both alleles of the B2M gene; or inactivation or disruption of all B2M coding alleles in the cell.

In some embodiments, the engineered cell comprises one or more modifications that reduce cell surface expression of one or more MHC HLA class I molecules. In some embodiments, the engineered cell comprises one or more modifications that reduce a function of one or more MHC HLA class I molecules. In some embodiments, the function is antigen presentation. In some embodiments, the one or more modifications that reduce expression in (ii) reduce expression of the B2M gene.

In some embodiments, the one or more modifications in (ii) reduce expression of MHC HLA class I and class II molecules.

In some embodiments, the one or more modifications in (ii) reduce expression of MHC HLA class II molecules HLA-DP, HLA-DQ, or HLA-DR. In some embodiments, the one or more modifications in (ii) reduce protein expression of one or more MHC class II molecules. In some embodiments, the one or more modifications that reduce protein expression reduce expression of an HLA-DP protein, an HLA-DQ protein, or an HLA-DR protein. In some embodiments, a gene encoding an HLA-DP protein, an HLA-DQ protein, or an HLA-DR protein, respectively, is knocked out. In some embodiments, the engineered cell comprises one or more modifications that reduce cell surface expression of one or more MHC HLA class II molecules. In some embodiments, the engineered cell comprises one or more modifications that reduce a function of one or more MHC HLA class II molecules. In some embodiments, the function is antigen presentation. In some embodiments, the one or more modifications in (ii) reduce expression of the CIITA gene. In some embodiments, the one or more modifications in (ii) reduce expression of the CD74 gene. In some embodiments, the one or more tolerogenic factors comprise one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, wherein the one or more tolerogenic factors comprises CD47. In some embodiments, the one or more modifications that increases expression of CD47 comprise an exogenous polynucleotide encoding the CD47 protein.

In some embodiments, the phenotype of the engineered cell comprises B2Mindel/indel; CIITAindel/indel; and CD47tg.

In some of any of such embodiments, the reducing in (a) is by one or more gene edits. In some of any of such embodiments, the inactivating or disrupting of the one or more alleles is by one or more gene edits. In some of any of such embodiments, the cell comprises a genome editing complex. In some of any of such embodiments, the one or more gene edits are made by a genome editing complex. In some of any of such embodiments, the genome editing complex comprises a genome targeting entity and a genome modifying entity. In some of any of such embodiments, the genome targeting entity localizes the genome editing complex to the one or more alleles that are inactivated or disrupted, optionally wherein the genome targeting entity is a nucleic acid-guided targeting entity.

In some of any of such embodiments, the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

In some of any of such embodiments, the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

In some of any of such embodiments, the genome modifying entity cleaves, deaminates, nicks, polymerizes, interrogates, integrates, cuts, unwinds, breaks, alters, methylates, demethylates, or otherwise destabilizes the target locus. In some of any of such embodiments, the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof. In some of any of such embodiments, the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

In some of any of such embodiments, the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

In some of any of such embodiments, the genome targeting entity and the genome modifying entity are different domains of a single polypeptide. In some of any of such embodiments, the genome editing entity and genome modifying entity are two different polypeptides that are operably linked together. In some of any of such embodiments, the genome editing entity and genome modifying entity are two different polypeptides that are not linked together. In some of any of such embodiments, the genome editing complex comprises a guide nucleic acid having a targeting domain that is complementary to at least one target locus, optionally wherein the guide nucleic acid is a guide RNA (gRNA).

In some of any of such embodiments, the one or more modifications are made by the genome editing complex. In some of any of such embodiments, the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

In some of any of such embodiments, the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

In some of any of such embodiments, the modifications made by the genome editing complex are made using a guide RNA (gRNA) having a targeting domain that is complementary to at least one target site. In some of any of such embodiments, the genome editing complex is an RNA-guided nuclease. In some of any of such embodiments, the RNA-guided nuclease comprises a Cas nuclease and a guide RNA (CRISPR-Cas combination). In some of any of such embodiments, the CRISPR-Cas combination is a ribonucleoprotein (RNP) complex comprising the gRNA and the Cas nuclease. In some of any of such embodiments, the Cas nuclease is a Type II or Type V Cas protein.

In some of any of such embodiments, the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

Provided herein is a cardiac cell therapy comprising a plurality of cardiomyocytes produced by any of the methods provided herein. Also provided herein is a cardiac cell therapy comprising a plurality of primary cardiac cells produced by any of the methods provided herein.

Provided herein is a method of treatment comprising administering any of the cardiac cell therapies provided herein to a subject.

Also provided herein is a method of treatment comprising administering a cardiac cell therapy comprising a plurality of any of the cardiomyocytes provided herein to a subject. Also provided herein is a method of treatment comprising administering a cardiac cell therapy comprising a plurality of any of the primary cardiac cells provided herein to a subject.

Also provided herein is a method of treatment comprising administering a cardiac cell therapy to a subject, wherein the cardiac cell therapy comprises engineered cardiac cells comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to cardiomyocytes that do not comprise the one or more modifications. In some embodiments, the cardiac cell therapy comprises engineered cardiomyocytes comprising one or more modifications that reduce expression of one or more of CACNA1G, HCN4, and SLC8A1. In some embodiments, the cardiac cell therapy comprises engineered cardiomyocytes comprising one or more modifications that increase expression of one or more of KCNJ2, TRDN, SRL, HRC, and CASQ2. In some embodiments, the cardiac cell therapy comprises engineered cardiomyocytes comprising one or more modifications that: (a) reduce expression of one or more of CACNA1G, HCN4, and SLC8A1; and (b) increase expression of one or more of KCNJ2, TRDN, SRL, HRC, and CASQ2. In some embodiments, the cardiac cells are cardiomyocytes. In some embodiments, the cardiac cells are cardiomyocytes differentiated from PSCs. In some embodiments, the cardiac cells are primary cardiac cells.

In some embodiments, the engineered cardiomyocytes comprise one or more modifications that reduce expression of CACNA1G. In some embodiments, the engineered cardiomyocytes comprise one or more modifications that reduce expression of HCN4 and/or SLC8A1. In some embodiments, the engineered cardiomyocytes comprise one or more modifications that reduce expression of HCN4. In some embodiments, the engineered cardiomyocytes comprise one or more modifications that reduce expression of SLC8A1. In some embodiments, the engineered cardiomyocytes comprise one or more modifications that reduce expression of HCN4 and SLC8A1. In some embodiments, the engineered cardiomyocytes comprise one or more modifications that increase expression of KCNJ2. In some embodiments, the engineered cardiomyocytes comprise one or more modifications that increase expression of TRDN. In some embodiments, the engineered cardiomyocytes comprise one or more modifications that increase expression of SRL. In some embodiments, the engineered cardiomyocytes comprise one or more modifications that increase expression of HRC. In some embodiments, the engineered cardiomyocytes comprise one or more modifications that increase expression of CASQ2. In some embodiments, the engineered cardiomyocytes comprise one or more modifications that (a) reduce expression of CACNA1G, HCN4, and SLC8A1; and (b) increase expression of KCNJ2.

In some embodiments, the cardiac cell therapy is administered as a suspension of cardiomyocytes or as an engineered tissue graft comprising cardiomyocytes and a matrix. In some embodiments, administration of the cardiac cell therapy comprises delivery into a subject's heart tissue. In some embodiments, delivery into a subject's heart tissue is by intravenous injection, intraarterial injection, intracoronary injection, intramuscular injection, intraperitoneal injection, intramyocardial injection, trans-endocardial injection, trans-epicardial injection, and/or infusion. In some embodiments, administration of the cardiac cell therapy to the subject results in less engraftment arrhythmia (EA) in the subject, relative to a cardiac cell therapy comprising cardiomyocytes not having the one or more modifications In some embodiments, administration of the cardiac cell therapy to the subject does not cause engraftment arrhythmia (EA) in the subject.

In some embodiments, the cardiac cell therapy comprises between about 5×10⁸ and 1×10¹⁰ engineered cardiomyocytes, inclusive of each. In some embodiments, the cardiac cell therapy comprises between about 1×10⁹ and about 5×10⁹ engineered cardiomyocytes, inclusive of each. In some embodiments, the cardiac cell therapy comprises a pharmaceutically acceptable carrier.

In some embodiments, the subject has a heart disease or condition. In some embodiments, the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart disease or condition is myocardial infarction (MI).

In some of any of such embodiments, the method further comprises administering one or more immunosuppressive agents to the subject. In some of any of such embodiments, the subject has been administered one or more immunosuppressive agents. In some of any of such embodiments, the one or more immunosuppressive agents are a small molecule or an antibody. In some of any of such embodiments, the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-a), and an immunosuppressive antibody. In some of any of such embodiments, the one or more immunosuppressive agents comprise cyclosporine. In some of any of such embodiments, the one or more immunosuppressive agents comprise mycophenolate mofetil. In some of any of such embodiments, the one or more immunosuppressive agents comprise a corticosteroid In some of any of such embodiments, the one or more immunosuppressive agents comprise cyclophosphamide. In some of any of such embodiments, the one or more immunosuppressive agents comprise rapamycin. In some of any of such embodiments, the one or more immunosuppressive agents comprise tacrolimus (FK-506). In some of any of such embodiments, the one or more immunosuppressive agents comprise anti-thymocyte globulin.

In some of any of such embodiments, the one or more immunosuppressive agents are one or more immunomodulatory agents. In some of any of such embodiments, the one or more immunomodulatory agents are a small molecule or an antibody. In some of any of such embodiments, the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands. In some of any of such embodiments, the one or more immunosuppressive agents are or have been administered to the subject prior to administration of the cardiac cell therapy.

In some of any of such embodiments, the one or more immunosuppressive agents are or have been administered to the subject at least 1, 2, 3, 4, 5, 6, 7, 8, 9, 10, 11, 12, 13, or 14 days prior to administration of the cardiac cell therapy. In some of any of such embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the cardiac cell therapy. In some of any of such embodiments, the one or more immunosuppressive agents are or have been administered to the subject at least 1, 2, 3, 4, 5, 6, 7, 8, 9, 10, 11, 12, 13, or 14 days after administration of the cardiac cell therapy. In some of any of such embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the cardiac cell therapy.

In some of any of such embodiments, the one or more immunosuppressive agents are or have been administered to the subject on the same day as the first administration of the cardiac cell therapy. In some of any of such embodiments, the one or more immunosuppressive agents are or have been administered to the subject after administration of the cardiac cell therapy. In some of any of such embodiments, the one or more immunosuppressive agents are or have been administered to the subject after administration of a first and/or second administration of the cardiac cell therapy. In some of any of such embodiments, the one or more immunosuppressive agents are or have been administered to the subject prior to administration of a first and/or second administration of the cardiac cell therapy.

In some of any of such embodiments, the one or more immunosuppressive agents are or have been administered to the subject at least 1, 2, 3, 4, 5, 6, 7, 8, 9, 10, 11, 12, 13, or 14 days prior to administration of a first and/or second administration of the cardiac cell therapy. In some of any of such embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the cardiac cell therapy. In some of any of such embodiments, the one or more immunosuppressive agents are or have been administered to the subject at least 1, 2, 3, 4, 5, 6, 7, 8, 9, 10, 11, 12, 13, or 14 days after administration of a first and/or second administration of the cardiac cell therapy. In some of any of such embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the cardiac cell therapy.

In some of any of such embodiments, the one or more immunosuppressive agents are administered at a lower dosage compared to the dosage of one or more immunosuppressive agents administered to reduce immune rejection of immunogenic cells that do not comprise the modifications of the cardiac cell therapy.

In some of any of such embodiments, the engineered cardiomyocyte is capable of controlled killing of the engineered cardiomyocyte.

In some of any of such embodiments, the engineered cardiomyocyte comprises a safety switch. In some embodiments, the safety switch induces controlled cell death in the presence of a drug or prodrug, or upon activation by a selective exogenous compound. In some of any of such embodiments, the safety switch is a system wherein upon activation, cells downregulate expression of the one or more tolerogenic factors and/or upregulate expression of one or more immune signaling molecules thereby marking the cell for elimination by the host immune system. In some of any of such embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some of any of such embodiments, the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some of any of such embodiments, the safety switch is an inducible protein capable of inducing apoptosis of the engineered cardiomyocyte. In some embodiments, the inducible protein capable of inducing apoptosis of the engineered cardiomyocyte is a caspase protein. In some embodiments, the caspase protein is caspase 9.

In some of any of such embodiments, the safety switch is a suicide gene. In some embodiments, the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

In some of any of such embodiments, the safety switch is activated to induce controlled cell death after the administration of the one or more immunosuppressive agents to the subject. In some of any of such embodiments, the safety switch is activated to induce controlled cell death prior to the administration of the one or more immunosuppressive agents to the subject. In some of any of such embodiments, the safety switch is activated to induce controlled cell death after the administration of the cardiac cell therapy to the subject. In some of any of such embodiments, the safety switch is activated to induce controlled cell death in the event of cytotoxicity or other negative consequences to the subject.

In some of any of such embodiments, the method comprises administering an agent that allows for depletion of an engineered cardiomyocyte of the plurality of cardiomyocytes. In some embodiments, the agent that allows for depletion of the engineered cardiomyocyte is an antibody that recognizes a protein expressed on the surface of the engineered cardiomyocyte. In some of any of such embodiments, the antibody is selected from the group consisting of an antibody that recognizes CCR4, CD16, CD19, CD20, CD30, EGFR, GD2, HER1, HER2, MUC1, PSMA, and RQR8. In some embodiments, the antibody is selected from the group consisting of mogamulizumab, AFM13, MOR208, obinutuzumab, ublituximab, ocaratuzumab, rituximab, rituximab-RIIb, tomuzotuximab, RO5083945 (GA201), cetuximab, Hul4.18K322A, Hul4.18-IL2, Hu3F8, dinituximab, c.60C3-Rllc, and biosimilars thereof.

In some of any of such embodiments, the method comprises administering an agent that recognizes the one or more tolerogenic factors on the surface of the engineered cardiomyocyte. In some of any of such embodiments, the engineered cardiomyocyte is engineered to express the one or more tolerogenic factors. In some of any of such embodiments, the one or more tolerogenic factors is CD47.

In some of any of such embodiments, the method further comprises administering one or more additional therapeutic agents to the subject. In some of any of such embodiments, the subject has been administered one or more additional therapeutic agents.

In some of any of such embodiments, the method further comprises monitoring the therapeutic efficacy of the method. In some of any of such embodiments, the method further comprises monitoring the prophylactic efficacy of the method. In some of any of such embodiments, the method is repeated until a desired suppression of one or more disease symptoms occurs.

In some of any of such embodiments, the engineered cardiomyocytes comprise one or more modifications that: (a) inactivate or disrupt one or more alleles of: (i) one or more major histocompatibility complex (MHC) class I molecules or one or more molecules that regulate expression of the one or more MHC class I molecules; and/or (ii) one or more MHC class II molecules or one or more molecules that regulate expression of the one or more MHC class II molecules; and/or (b) increase expression of one or more tolerogenic factors in the engineered cell, relative to a cell of the same cell type that does not comprise the one or more modifications. In some embodiments, the one or more modifications in (a) increase expression of one or more tolerogenic factors, relative to cardiomyocytes that do not comprise the one or more modifications that make the engineered cardiomyocytes hypoimmunogenic.

In some of any of such embodiments, the one or more MHC class I molecules is one or more human leukocyte antigen (HLA) class I molecules. In some of any of such embodiments, the one or more MHC HLA class I molecules is selected from the group consisting of HLA-A, HLA-B, and HLA-C. In some of any of such embodiments, the one or more molecules that regulate expression of the one or more MHC class I molecules is/are selected from the group consisting of B-2 microglobulin (B2M) gene and/or the transporter 1, ATP binding cassette subfamily B member (TAP1). In some of any of such embodiments, the one or more molecules that regulate expression of the one or more MHC class I molecules regulate cell surface protein expression of the one or more MHC class I molecules. In some of any of such embodiments, the one or more modifications in (a) reduce cell surface trafficking of the one or more MHC HLA class I molecules.

In some embodiments, the engineered cardiomyocytes comprise one or more modifications that: (i) increase expression of one or more tolerogenic factors; and/or (ii) reduce expression of one or more major histocompatibility complex (MHC) class I molecules and/or MHC class II, relative to cardiomyocytes that do not comprise the one or more modifications that make the engineered cardiomyocytes hypoimmunogenic. In some embodiments, MHC HLA class I molecules are selected from the group consisting of HLA-A, HLA-B, HLA-C, and a combination thereof. In some embodiments, the one or more modifications in (ii) reduce expression of one or more MHC HLA class I molecules. In some embodiments, the one or more modifications in (ii) reduce expression of MHC HLA class I molecules HLA-A, HLA-B, and HLA-C. In some embodiments, the one or more modifications in (ii) reduce protein expression of one or more MHC HLA class I molecules. In some embodiments, the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out. In some embodiments, the engineered cardiomyocytes comprise one or more modifications that reduce cell surface expression of one or more MHC HLA class I molecules. In some embodiments, the engineered cardiomyocytes comprise one or more modifications that reduce a function of one or more MHC HLA class I molecules, optionally wherein the function is antigen presentation. In some embodiments, the one or more modifications in (ii) that reduce expression reduce expression of the B2M gene.

In some of any of such embodiments, the one or more molecules that regulate cell surface protein expression of the one or more MHC class I molecules is B2M. In some of any of such embodiments, the one or more modifications comprise a modification that regulates cell surface protein expression of the one or more MHC class I molecules and the modification inactivates or disrupts one or more alleles of B2M. In some of any of such embodiments, cell surface trafficking of the one or more MHC class I molecules is reduced in the modified cell relative to the cell of the same cell type that does not comprise the one or more modifications.

In some of any of such embodiments, the one or more modifications in (a) inactivates or disrupts one or more alleles of B2M, NLRC5, or TAP1. In some of any of such embodiments, the one or more modifications in (a) inactivates or disrupts one or more alleles of B2M. In some of any of such embodiments, the modification that inactivates or disrupts one or more alleles of B2M reduces mRNA expression of the B2M gene. In some of any of such embodiments, the modification that inactivates or disrupts one or more alleles of B2M reduces protein expression of B2M. In some of any of such embodiments, the modification that inactivates or disrupts one or more alleles of B2M comprises: inactivation or disruption of one allele of the B2M gene; inactivation or disruption of both alleles of the B2M gene; or inactivation or disruption of all B2M coding alleles in the cell. In some of any of such embodiments, the inactivation or disruption comprises an indel in the B2M gene. In some of any of such embodiments, the inactivation or disruption comprises a frameshift mutation or a deletion of a contiguous stretch of genomic DNA of the B2M gene.

In some embodiments, the one or more modifications in (ii) reduce expression of MHC HLA class I and class II molecules. In some embodiments, the one or more modifications in (ii) reduce expression of MHC HLA class II molecules HLA-DP, HLA-DQ, or HLA-DR. In some embodiments, the one or more modifications in (ii) reduce protein expression of one or more MHC class II molecules. In some embodiments, the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out. In some embodiments, the engineered cardiomyocytes comprise one or more modifications that reduce cell surface expression of one or more MHC HLA class II molecules. In some embodiments, the engineered cardiomyocytes comprise one or more modifications that reduce a function of one or more MHC HLA class II molecules. In some embodiments, the function is antigen presentation. In some embodiments, the one or more modifications in (ii) reduce expression of the CIITA gene. In some embodiments, the one or more modifications in (ii) reduce expression of the CD74 gene.

In some embodiments, the one or more tolerogenic factors comprise one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, the one or more tolerogenic factors comprises CD47. In some embodiments, the one or more tolerogenic factors comprise CD47, and the one or more modifications that increases expression of CD47 comprise an exogenous polynucleotide encoding the CD47 protein. In some embodiments, the one or more modifications that increases expression of CD47 comprise an exogenous polynucleotide encoding the CD47 protein.

In some embodiments, the phenotype of the engineered cardiomyocytes comprises B2Mindel/indel; CIITAindel/indel; and CD47tg.

In some of any of such embodiments, the cardiomyocytes are autologous to the subject. In some of any of such embodiments, the cardiomyocytes are allogeneic to the subject. In some of any of such embodiments, the subject is a human.

### BRIEF DESCRIPTION OF THE DRAWINGS

FIG. 1A shows Uniform Manifold Approximation and Projection (UMAP) plots representative of gene expression across all time points (Days 9-31 of differentiation), as analyzed by single cell RNAseq.

FIG. 1B shows the data from FIG. 1A analyzed at each of days 9, 18, 21, 21-25, and 31.

FIG. 2A shows spatial quantification of CACNA1G, CACNA1H, and CACNA1I expression at onset of engraftment arrhythmia (EA; left), mid-EA (middle), and post-EA (right).

FIG. 2B shows the sequenced mRNA transcripts from FIG. 2A mapped to the region of tissue from which they were expressed.

### DETAILED DESCRIPTION

Provided herein, in some aspects, are compositions and methods for reducing engraftment arrhythmia (EA) associated with the transplant of cardiac cells or tissue into a subject, including for the treatment of a cardiac disease or condition. To overcome the problem of transplanted cardiac cells or tissue (i.e. grafts) resulting in EA in a subject, disclosed herein is an engineered therapeutic cell, or a population or pharmaceutical composition thereof, which has the ability to reduce or prevent EA when grafted into a subject. In aspects of the engineered therapeutic cells provided herein, EA is reduced or prevented in the subject following transplant of the engineered cells into the subject. The engineered therapeutic cells described herein may be derived from cells including, but not limited to, pluripotent stem cells (PSCs), such as embryonic stem cells (ESCs) or induced pluripotent stem cells (iPSCs), cardiomyocytes differentiated from PSCs, and primary cardiac cells (e.g., primary cardiomyocytes). In some embodiments, the engineered therapeutic cells are cardiac cells (e.g., cardiomyocytes) that have been differentiated from PSCs, such as ESCs or iPSCs. In some embodiments, the engineered therapeutic cells are primary cardiac cells (e.g., primary cardiomyocytes).

Also provided herein are engineered cells that contain one or modifications that reduce expression of one or more targets relative to a cell of the same cell type not having the one or more modifications. In some embodiments the engineered cells are PSCs, such as ESCs or iPSCs.

In some embodiments, the cells are engineered to have one or more modifications that yield constitutive reduced expression of one or more targets relative to a cell of the same cell type not having the one or more modifications. In some embodiments, the cells are engineered to have one or more modifications that yield regulatable reduced expression of one or more targets relative to a cell of the same cell type not having the one or more modifications. In some embodiments, following engineering, the engineered cells are differentiated into cardiac cells (e.g., cardiomyocytes), i.e. therapeutic engineering cells), such for administration to a subject).

In some embodiments, the engineered cells contain one or modifications that increase expression of one or more targets relative to a cell of the same cell type not having the one or more modifications. In some embodiments, the cells are engineered to have one or more modifications that yield constitutive increased expression of one or more targets relative to a cell of the same cell type not having the one or more modifications. In some embodiments, the cells are engineered to have one or more modifications that yield regulatable increased expression of one or more targets relative to a cell of the same cell type not having the one or more modifications. In some embodiments, following engineering, the engineered cells are differentiated into cardiac cells (e.g., cardiomyocytes), i.e. therapeutic engineering cells, such for administration to a subject).

In some embodiments, the engineered cells provided herein contain one or more modifications (e.g., genetic modifications) that result in decreased expression (e.g., reduced or eliminated expression) of one or more of CACNA1G, CACNA1H, HCN4, and SLC8A1; increased expression (e.g., overexpression or increased expression) of one or more of KCNJ2, TRDN, SRL, HRC, and CASQ2; or any combination thereof. In provided aspects, the altered expression is relative to a cell of the same cell type that does not contain the one or more modifications, such as a wild-type or unmodified cell of the same cell type or a cell that otherwise is the same but that lacks the one or more modifications herein to alter expression of one or more of CACNA1G, CACNA1H, HCN4, SLC8A1, KCNJ2, TRDN, SRL, HRC, and CASQ2.

In some embodiments, the engineered cells provided herein also contain one or more modifications (e.g., genetic modifications) that result in increased expression (e.g., overexpression or increased expression) of one or more tolerogenic factors (e.g., CD47); and/or decreased expression (e.g., reduced or eliminated expression) of MHC HLA class I molecules and/or MHC HLA class II molecules. In some embodiments, the modifications present in the engineered cell provide for increased (e.g. increased or overexpressed) cell surface expression of the one of more tolerogenic factors; and/or decreased (e.g. reduced or eliminated) cell surface expression of MHC HLA class I molecules and/or MHC HLA class II molecules, such as an increase or overexpression of the one or more tolerogenic factors on the cell surface and reduced or eliminated expression of MHC HLA class I molecules and/or MHC HLA class II molecules on the cell surface. In provided aspects, the altered expression is relative to a cell of the same cell type that does not contain the one or more modifications, such as a wild-type or unmodified cell of the same cell type or a cell that otherwise is the same but that lacks the one or more modifications herein to alter expression of the one or more tolerogenic factors and/or MHC HLA class I molecules and/or MHC HLA class II molecules.

Thus, in some embodiments, the engineered cells provided herein contain one or more modifications that result in altered expression of: one or more of CACNA1G, CACNA1H, HCN4, SLC8A1, KCNJ2, TRDN, SRL, HRC, and CASQ2; one or more tolerogenic factors; one or more MHC HLA class I molecules and/or MHC HLA class II molecules; or any combination thereof. In some aspects, the engineered cells provided herein contain one or more modifications that result in altered expression of: one or more of CACNA1G, CACNA1H, HCN4, SLC8A1, KCNJ2, TRDN, SRL, HRC, and CASQ2; one or more tolerogenic factors; one or more of MHC HLA-A, HLA-B, HLA-C and/or MHC HLA-DP, HLA-DQ, HLA-DR; or any combination thereof. In some aspects, the engineered cells provided herein contain one or more modifications that result in altered expression of: one or more of CACNA1G, CACNA1H, HCN4, SLC8A1, KCNJ2, TRDN, SRL, HRC, and CASQ2; one or more tolerogenic factors; one or more of B2M, TAP1, CIITA, and CD74 or any combination thereof. In some aspects, the engineered cells provided herein contain one or more modifications that result in altered expression of: one or more of CACNA1G, CACNA1H, HCN4, SLC8A1, KCNJ2, TRDN, SRL, HRC, and CASQ2; one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof); one or more of B2M, TAP1, CIITA, and CD74 or any combination thereof.

Exemplary methods to introduce modifications into a cell to alter expression are described herein. For instance, any of a variety of methods for overexpressing or increasing expression of a gene or protein may be used, such as by introduction or delivery of an exogenous polynucleotide encoding a protein (i.e. a transgene) or introduction of delivery of a fusion protein of a DNA-targeting domain and a transcriptional activator targeting a gene. Also, any of a variety of methods for reducing or eliminating expression of a gene or protein may be used, including non-gene editing methods such as by introduction or delivery of inhibitory nucleic acids (e.g. RNAi) or gene editing methods involving introduction or delivery of a targeted nuclease system (e.g. CRISPR/Cas). In some embodiments, the method for reducing or eliminating expression is via a nuclease-based gene editing technique.

In some embodiments, genome editing technologies utilizing rare-cutting endonucleases (e.g., the CRISPR/Cas, TALEN, zinc finger nuclease, meganuclease, and homing endonuclease systems) are used to reduce or eliminate expression of genes, including immune genes (e.g., by deleting genomic DNA of critical immune genes) in human cells. In some embodiments, the genome editing technology comprises use of nickases, base editing, prime editing, and gene writing.

In certain embodiments, genome editing technologies or other gene modulation technologies are used to: insert one or more of the KCNJ2, TRDN, SRL, HRC, and CASQ2 genes; reduce or eliminate expression of one or more of the CACNA1G, CACNA1H, HCN4, and SLC8A1 genes; or any combination thereof, thus producing engineered cells that can result in reduced or eliminated EA following engraftment in a subject.

Therefore, the engineered cells provided herein exhibit modulated expression (e.g., reduced or eliminated expression) of one or more of CACNA1G, CACNA1H, HCN4, and SLC8A1, and/or modulated expression (e.g., increased expression or overexpression) of one or more of KCNJ2, TRDN, SRL, HRC, and CASQ2. In some embodiments, the engineered cells provided herein exhibit modulated expression (e.g., reduced or eliminated expression) of CACNA1G, HCN4, and SLC8A1, and modulated expression (e.g., increased expression or overexpression) of KCNJ2.

In some embodiments, the engineered cells provided herein do not cause engraftment arrhythmia following engraftment in a subject.

In certain embodiments, genome editing technologies or other gene modulation technologies are used to also insert tolerance-inducing (tolerogenic) factors in the engineered cells, (e.g., CD47), thus producing engineered cells that can evade immune recognition upon engraftment in a recipient subject. Therefore, in some embodiments, the engineered cells provided herein exhibit modulated expression (e.g., reduced or eliminated expression) of one or more genes and factors that affect MHC class I molecule and/or MHC class II molecule, and modulated expression (e.g., increased expression or overexpression) of tolerogenic factors, such as CD47. In some embodiments, the engineered cells evade the recipient subject's immune system.

In some aspects, engineered cells provided herein are not subject to an innate immune cell rejection or an adaptive immune cell rejection (e.g., hypoimmunogenic cells). For example, in some embodiments, the engineered cells are not susceptible to NK cell-mediated lysis and macrophage engulfment. In some embodiments, the engineered cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

In some embodiments, the engineered cells provided herein are PSCs (e.g., ESCs or iPSCs) that are differentiated into cardiomyocytes, such as by a method comprising adherent or suspension culture. In some embodiments, the engineered cells are cardiomyocytes that have been previously differentiated from iPSCs, such as by a method comprising adherent or suspension culture. In some embodiments, the cardiomyocytes differentiated from PSCs are transplanted into a subject. In some embodiments, the engineered cells are primary cardiac cells (e.g., primary cardiomyocytes). In some embodiments, the engineered cells are primary cardiomyocytes. In some embodiments, the primary cardiomyocytes are transplanted into a subject.

The present disclosure is based, at least in part, on the inventors' findings and unique perspectives regarding the engineering of cells useful for administration as a cardiac cell therapy. In particular, observations described herein indicate that among CACNA1G, CACNA1H, and CACNA1I, CACNA1G (corresponding to the Cav3.1 T-type calcium channel) is the primary T-type calcium channel-encoding gene expressed in cells throughout the differentiation (e.g., non-adherent differentiation) of PSCs into cardiomyocytes. In addition, observations herein demonstrate that CACNA1G is the most highly expressed T-type calcium channel-encoding gene in human cardiomyocyte grafts during and after engraftment arrhythmia in transplanted subjects.

Thus, such engineering, including reducing or eliminating expression of CACNA1G may help avoid EA in a subject into which the engineered cells, or a population or composition thereof, are transplanted and engraft.

In some aspects, the engineered cell comprises one or more modifications that: (a) reduce expression of one or more of CACNA1G, CACNA1H, HCN4, and SLC8A1; (b) increase expression of one or more of KCNJ2, TRDN, SRL, HRC, and CASQ2; or (c) a combination thereof, relative to a cell of the same cell type that does not comprise the one or more modifications.

In some aspects, provided herein is a method of producing an engineered cell, the method comprising: (a) reducing expression of one or more of CACNA1G, CACNA1H, HCN4, and SLC8A1; (b) increasing expression of one or more of KCNJ2, TRDN, SRL, HRC, and CASQ2; or (c) a combination thereof, in the cell. In some embodiments, the method comprises reducing expression of CACNA1G in the cell. In some embodiments, the method comprises reducing expression of HCN4 and/or SLC8A1 in the cell. In some embodiments, the method comprises increasing expression of KCNJ2 in the cell. In some embodiments, the method comprises (a) reducing expression of CACNA1G, HCN4, and SLC8A1; and (b) increasing expression of KCNJ2, in the cell.

In some embodiments, the one or more modifications that increase expression comprise increased surface expression of a protein encoded by the one or more genes and/or the one or more modifications that reduce expression comprise reduced surface expression of a protein encoded by the one or more genes. In some embodiments, the engineered cell is selected from a PSC, such as an ESC or an iPSC, a cardiomyocyte differentiated from a PSC, and a primary cardiac cell (e.g., a primary cardiomyocyte).

In some aspects, provided herein is a population of engineered cells comprising a plurality of any of the engineered described herein. In some embodiments, at least 50%, 60%, 70%, 80%, 90%, 95%, 96%, 97%, 98%, 99%, 99.9%, or 99.99% of cells in the population comprise increased expression of KCNJ2 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of HCN4 and/or SLC8A1 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of CACNA1G relative to cells of the same cell type that are unaltered or unmodified.

In some aspects, the engineered cell further comprises one or more modifications that: (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell of the same cell type that does not comprise the one or more modifications.

In some aspects, any of the methods provided herein further comprise introducing in an engineered cell one or more modifications that: (i) increase the expression of one or more tolerogenic factors in the engineered cell; and/or (ii) reduce or eliminate the expression of MHC HLA class I molecules and/or MHC HLA class II molecules in the engineered cell, relatively to a cell of the same cell type that is not introduced with the one or more modifications.

In some embodiments, the one or more tolerogenic factors comprise one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, the one or more tolerogenic factors is or comprises CD47. In some embodiments, the one or more modifications that reduce expression of MHC HLA class I molecules is a modification that reduces expression of beta-2 microglobulin (B2M). In some embodiments, the one or more modifications that reduce expression of MHC HLA class I molecules is a modification that reduces expression of Transporter 1, ATP Binding Cassette Subfamily B Member (TAP1). In some embodiments, the one or more modifications that reduce expression of MHC HLA class II molecules is a modification that reduces expression of CIITA. In some embodiments, the one or more modifications that reduce expression of MHC HLA class II molecules is a modification that reduces expression of CD74. In some embodiments, the one or more modifications that reduce expression of MHC HLA class I and II molecules is a modification that reduces expression of B2M and CIITA. In some embodiments, the one or more modifications that reduce expression of MHC HLA class I and II molecules is a modification that reduces expression of B2M and CD74. In some embodiments, the one or more modifications that reduce expression of MHC HLA class I and II molecules is a modification that reduces expression of TAP1 and CIITA. In some embodiments, the one or more modifications that reduce expression of MHC HLA class I and II molecules is a modification that reduces expression of TAP1 and CD74.

In some embodiments, the one or more modifications that increase expression comprise increased surface expression, and/or the one or more modifications that reduce expression comprise reduced surface expression. In some embodiments, the engineered cell is selected from a PSC, such as an ESC or an iPSC, a cardiomyocyte differentiated from a PSC, and a primary cardiac cell (e.g., a primary cardiomyocyte).

In some aspects, provided herein is a population of engineered cells comprising a plurality of any of the engineered cells described herein. In some embodiments, at least 50%, 60%, 70%, 80%, 90%, 95%, 96%, 97%, 98%, 99%, 99.9%, or 99.99% of cells in the population comprise an exogenous polynucleotide encoding CD47. In some embodiments, at least 50%, 60%, 70%, 80%, 90%, 95%, 96%, 97%, 98%, 99%, 99.9%, or 99.99% of cells in the population comprise increased expression of CD47. In some embodiments, at least 50%, 60%, 70%, 80%, 90%, 95%, 96%, 97%, 98%, 99%, 99.9%, or 99.99% of cells in the population comprise reduced expression of B2M, TAP1, and/or CIITA relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of B2M relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of TAP1 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of CIITA relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of B2M and CIITA relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of TAP1 and CIITA relative to cells of the same cell type that are unaltered or unmodified.

In some aspects, provided herein is a population of engineered cells comprising a plurality of any of the engineered cells described herein. In some embodiments, at least 50%, 60%, 70%, 80%, 90%, 95%, 96%, 97%, 98%, 99%, 99.9%, or 99.99% of cells in the population comprise an exogenous polynucleotide encoding CD47. In some embodiments, at least 50%, 60%, 70%, 80%, 90%, 95%, 96%, 97%, 98%, 99%, 99.9%, or 99.99% of cells in the population comprise increased expression of CD47. In some embodiments, at least 50%, 60%, 70%, 80%, 90%, 95%, 96%, 97%, 98%, 99%, 99.9%, or 99.99% of cells in the population comprise reduced expression of B2M, TAP1, and/or CD74 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of B2M relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of TAP1 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of CD74 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of B2M and CD74 relative to cells of the same cell type that are unaltered or unmodified. In some embodiments, at least 50%, 60%, 70%, 80%, 90%, 95%, 96%, 97%, 98%, 99%, 99.9%, or 99.99% of cells in the population comprise reduced expression of TAP1 and CD74 relative to cells of the same cell type that are unaltered or unmodified.

In some embodiments, provided herein is a composition (e.g., a pharmaceutical composition any of the populations of engineered cells described herein). In some embodiments, the population of engineered cells comprises a population of engineered cells selected from the group consisting of engineered PSCs, including iPSCs and ESCs, cardiomyocytes differentiated from PSCs, and primary cardiac cells (e.g., primary cardiomyocytes).

Also provided herein is a method of treating a heart disease or condition (e.g. a myocardial infarction) in a subject comprising administering to the subject a population of any of the engineered cells described herein, any of the compositions of engineered cells described herein, or any of the pharmaceutical compositions of engineered cells described herein. In some embodiments, the engineered cells produced from any of the methods described herein result in reduced or eliminated EA following engraftment in the subject. In some embodiments, the population, composition, or pharmaceutical composition comprising the engineered cells is administered to an MHC-mismatched allogeneic subject. In some embodiments, the engineered cells produced from any one of the methods described herein evade immune rejection when repeatedly administered (e.g., transplanted or grafted) to MHC-mismatched allogeneic subject.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); and, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, “treatment” (and grammatical variations thereof such as “treat” or “treating”) refers to complete or partial amelioration or reduction of rejection of a cardiac cell therapy or a symptom, adverse effect or outcome, or phenotype associated therewith. Desirable effects of treatment include, but are not limited to, preventing occurrence or recurrence of rejection of a cardiac cell therapy, alleviation of symptoms, diminishment of any direct or indirect pathological consequences of the rejection, preventing rejection of a cardiac cell therapy, decreasing the rate of such rejection, amelioration or palliation of the rejection, and cessation of rejection or improved prognosis. The terms do not imply complete prevention or cure of rejection or complete elimination of any symptom or effect(s) on all symptoms or outcomes.

“Preventing,” as used herein, includes providing prophylaxis with respect to the occurrence or recurrence of rejection of a cardiac cell therapy in a subject that may be predisposed to the rejection but has not yet been diagnosed with the rejection. In some embodiments, the methods described herein prevent the onset of rejection of a cardiac cell therapy.

“Attenuating,” as used herein, includes reducing the severity of, reducing, and/or delaying the onset of rejection of a cardiac cell therapy. In some embodiments, the methods described herein attenuate rejection of a cardiac cell therapy.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, cells, or composition refers to an amount effective, at dosages and for periods of time necessary, to achieve a desired therapeutic result, such as for treatment of a disease, condition, or disorder (e.g. rejection of a cardiac cell therapy), and/or pharmacokinetic or pharmacodynamics effect of the treatment. The therapeutically effective amount may vary according to factors such as the disease or condition state, age, sex, and weight of the subject, and the populations of cells administered. In some embodiments, the provided methods involve administering the molecules, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease or condition (e.g. rejection of a cardiac cell therapy), the prophylactically effective amount will be less than the therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

The term “pluripotent cells” refers to cells that can self-renew and proliferate while remaining in an undifferentiated state and that can, under the proper conditions, be induced to differentiate into specialized cell types. The term “pluripotent cells,” as used herein, encompasses embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm, mesoderm, or ectoderm. The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al, Stem Cells 27 (11): 2667-74 (2009); Huangfu et al., Nature Biotechnol. 26 (7): 795 (2008); Woltjen et al, Nature 458 (7239): 766-770 (2009); and Zhou et al, Cell Stem Cell 8:381-384 (2009); each of which is incorporated by reference herein in their entirety.) As used herein, “hiPSCs” are human induced pluripotent stem cells, and “riPSCs” are rhesus induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: S SEA-3, S SEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the term “exogenous” with reference to a polypeptide or a polynucleotide is intended to mean that the referenced molecule is introduced into the cell of interest. The exogenous molecule, such as exogenous polynucleotide, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell, but can be introduced into a cell by one or more genetic, biochemical or other methods.

The term “endogenous” refers to a referenced molecule, such as a polynucleotide (e.g. gene), or polypeptide, that is present in a native or unmodified cell. For instance, the term when used in reference to expression of an endogenous gene refers to expression of a gene encoded by an endogenous nucleic acid contained within the cell and not exogenously introduced.

A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “increased expression”, “enhanced expression” or “overexpression” means any form of expression that is additional to the expression in an original or source cell that does not contain the modification for modulating a particular gene expression, for instance a wild-type expression level (which can be absence of expression or immeasurable expression as well). Reference herein to “increased expression,” “enhanced expression” or “overexpression” is taken to mean an increase in gene expression and/or, as far as referring to polypeptides, increased polypeptide levels and/or increased polypeptide activity, relative to the level in a cell that does not contain the modification, such as the original source cell prior to the engineering to introduce the modification, such as an unmodified cell or a wild-type cell. The increase in expression, polypeptide levels or polypeptide activity can be at least 5%, 10%, 20%, 30%, 40% or 50%, 60%, 70%, 80%, 85%, 90%, or 100% or even more. In some cases, the increase in expression, polypeptide levels or polypeptide activity can be at least 2-fold, 5-fold, 10-fold, 20-fold, 30-fold, 40-fold, 50-fold, 60-fold, 70-fold, 80-fold, 90-fold, 100-fold, 200-fold or more.

The “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “hypoimmunogenic cell,” herein is meant a cell that gives rise to a reduced immunological rejection response when transferred into an allogeneic host. In preferred embodiments, hypoimmunogenic cells do not give rise to an immune response. Thus, “hypo-immunogenic” or “hypoimmune” refers to a significantly reduced or eliminated immune response when compared to the immune response of a parental (i.e., wild-type” or “wt”) cell prior to immunoengineering as outlined herein.

Hypoimmunogenicity of a cell can be determined by evaluating the immunogenicity of the cell such as the cell's ability to elicit adaptive and innate immune responses. Such immune response can be measured using assays recognized by those skilled in the art.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically a tolerogenic factor is a factor that induces immunological tolerance to an engineered cell so that the engineered cell is not targeted, such as rejected, by the host immune system of a recipient. Hence, a tolerogenic factor may be a hypoimmunity factor. Examples of tolerogenic factors include immune cell inhibitory receptors (e.g. CD47), proteins that engage immune cell inhibitory receptors, checkpoint inhibitors and other molecules that reduce innate or adaptive immune recognition.

The terms “decrease,” “reduced,” “reduction,” and “decrease” are all used herein generally to mean a decrease by a statistically significant amount. However, for avoidance of doubt, “decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modification” refers to any change or alteration in a cell that impacts gene expression in the cell. In some embodiments, the modification is a genetic modification that directly changes the gene or regulatory elements thereof encoding a protein product in a cell, such as by gene editing, mutagenesis or by genetic engineering of an exogenous polynucleotide or transgene.

As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A CRISPR/Cas system of the present disclosure can be used to induce an indel of any length in a target polynucleotide sequence.

In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently. Those skilled in the art will readily appreciate how to use the CRISPR/Cas systems of the present disclosure to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

“Modulation” of gene expression refers to a change in the expression level of a gene. Modulation of expression can include, but is not limited to, gene activation and gene repression. Modulation may also be complete, i.e, wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

The term “package insert” is used to refer to instructions customarily included in commercial packages of therapeutic products, that contain information about the indications, usage, dosage, administration, combination therapy, contraindications and/or warnings concerning the use of such therapeutic products.

A “pharmaceutically acceptable carrier” refers to an ingredient in a pharmaceutical formulation, other than an active ingredient, which is nontoxic to a subject. A pharmaceutically acceptable carrier includes, but is not limited to, a cell culture medium, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, the term “and/or” includes any and all combinations of one or more of the associated listed items.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### II. ENGINEERED CELLS

Provided herein are cells that comprise one or more modifications that regulate the expression of one or more target polynucleotide sequences, such as regulates the expression of one or more of CACNA1G, CACNA1H, HCN4, SLC8A1, KCNJ2, TRDN, SRL, HRC, and CASQ2. One of skill in the art can appreciate that the one or more modifications that regulate expression of CACNA1G, CACNA1H, HCN4, SLC8A1, KCNJ2, TRDN, SRL, HRC, and CASQ2 can be introduced into a cell prior to, simultaneously with, and/or subsequent to any of the modifications introduced into any of the cells as described in Section III. Further, one of skill in the art can appreciate that the one or more modifications that regulate expression of CACNA1G, CACNA1H, HCN4, SLC8A1, KCNJ2, TRDN, SRL, HRC, and CASQ2 can be introduced into a PSC prior to, simultaneously with, and/or subsequent to differentiation, as described in Section VI. In some embodiments, engineered cells that further comprise one or more modifications that regulate the expression of CACNA1G, CACNA1H, HCN4, SLC8A1, KCNJ2, TRDN, SRL, HRC, and CASQ2 reduce or prevent engraftment arrhythmia following engraftment in a subject.

In some embodiments, the engineered cell comprises one or more modifications that: (a) inactivate or disrupt one or more alleles of: (i) one or more major histocompatibility complex (MHC) class I molecules or one or more molecules that regulate expression of the one or more MHC class I molecules; and/or (ii) one or more MHC class II molecules or one or more molecules that regulate expression of the one or more MHC class II molecules; and/or (b) increase expression of one or more tolerogenic factors in the engineered cell, relative to a cell of the same cell type that does not comprise the one or more modifications. In some embodiments, the one or more modifications that inactivate or disrupt the one or more alleles reduce expression of one or more major histocompatibility complex (MHC) human leukocyte antigen (HLA) class I molecules and/or MHC HLA class II molecules, relative to a cell of the same cell type that does not comprise the one or more modifications.

In some embodiments, the modulation of expression of one or more of CACNA1G, CACNA1H, HCN4, SLC8A1, KCNJ2, TRDN, SRL, HRC, and CASQ2 is relative to the amount of expression of said molecule(s) in a cell of the same cell type that does not comprise the one or more modifications. In some embodiments, the modulation of expression is relative to an unmodified or a wildtype cell. In some embodiments, the unmodified or wildtype cell is a cell of the same cell type as the provided cells (e.g., the engineered cells). In some embodiments, the unmodified cell or wildtype cell expresses one or more of CACNA1G, CACNA1H, HCN4, SLC8A1, KCNJ2, TRDN, SRL, HRC, and CASQ2. In some embodiments, the unmodified cell or wildtype cell does not express one or more of KCNJ2, TRDN, SRL, HRC, and CASQ2. In some embodiments, wherein the unmodified cell or wildtype cell does not express one or more of KCNJ2, TRDN, SRL, HRC, and CASQ2, the provided engineered cells include a modification to overexpress one or more of KCNJ2, TRDN, SRL, HRC, and CASQ2 or increase the expression of one or more of KCNJ2, TRDN, SRL, HRC, and CASQ2 from 0%. It is understood that if the cell prior to the engineering does not express a detectable amount of one or more of KCNJ2, TRDN, SRL, HRC, and CASQ2, then a modification that results in any detectable amount of an expression of one or more of KCNJ2, TRDN, SRL, HRC, and CASQ2 is an increase in the expression compared to the similar cell that does not contain the modifications.

In some embodiments, the provided cells (e.g., the engineered cells) include a modification to increase expression of one or more of KCNJ2, TRDN, SRL, HRC, and CASQ2. In some embodiments, the modification includes increased expression of KCNJ2. In some embodiments, the modification includes increased expression of TRDN. In some embodiments, the modification includes increased expression of SRL. In some embodiments, the modification includes increased expression of CASQ2. In some embodiments, the modification is or includes increased expression of KCNJ2, TRDN, SRL, HRC, and CASQ2.

In some embodiments, the provided cells (e.g., the engineered cells) include a modification to decrease expression of one or more of CACNA1G, CACNA1H, HCN4, and SLC8A1. In some embodiments, the provided cells (e.g., the engineered cells) include a modification to decrease expression of one or more of CACNA1G, HCN4, and SLC8A1. In some embodiments, the modification includes decreased expression of CACNA1G. In some embodiments, the modification includes decreased expression of CACNA1H. In some embodiments, the modification includes decreased expression of HCN4 In some embodiments, the modification includes decreased expression of SLC8A1. In some embodiments, the modification is or includes decreased expression of CACNA1G, CACNA1H, HCN4, and SLC8A1. In some embodiments, the modification is or includes decreased expression of CACNA1G, HCN4, and SLC8A1.

In some embodiments, the cells include one or more modifications, such as genetic modifications, that reduce expression of one or more of CACNA1G, CACNA1H, HCN4, and SLC8A1 and one or more modifications that increase expression of one or more of KCNJ2, TRDN, SRL, HRC, and CASQ2. In some embodiments, the cells include one or more modifications, such as genetic modifications, that reduce expression of one or more of CACNA1G, HCN4, and SLC8A1 and one or more modifications that increase expression of one or more of KCNJ2, TRDN, SRL, HRC, and CASQ2. In some embodiments, the cells include one or more modifications, such as genetic modifications, that reduce expression of one or more of CACNA1G, HCN4, and SLC8A1 and one or more modifications that increase expression of KCNJ2. In some embodiments, the cells include one or more modifications, such as genetic modifications, that reduce expression of CACNA1G, HCN4, and SLC8A1 and one or more modifications that increase expression of KCNJ2.

In some embodiments, the engineered cells provided herein comprise a “suicide gene” or “suicide switch”. A suicide gene or suicide switch can be incorporated to function as a “safety switch” that can cause the death of the cell, such as after the cell is administered to a subject and if they cells should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al, Mol. Therap. 20 (10): 1932-1943 (2012), Xu et al, Cell Res. 8:73-8 (1998), both incorporated herein by reference in their entirety).

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies.

In some embodiments, a safety switch can be incorporated into, such as introduced, into the cells provided herein to provide the ability to induce death or apoptosis of cells containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44 (11): 1013-1019 (2016) (for iCaspase9); Wang et al., Blood 18 (5): 1255-1263 (2001) (for huEGFR); U.S. Patent Application Publication No. 20180002397 (for HER1); and Philip et al., Blood124 (8): 1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting a cell with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin inducible caspase 9 variant is called rapaCasp9. See Stavrou et al., Mal. Ther. 26 (5): 1266-1276 (2018). Thus, iCasp9 can be used as a safety switch to achieve controlled killing of the host cells.

In some embodiments, the method further comprises introducing an expression vector comprising an inducible suicide switch into the cell.

In some embodiments, the engineered cell is derived from a source cell already comprising one or more of the desired modifications. In some embodiments, in view of the teachings provided herein one of ordinary skill in the art will readily appreciate how to assess what modifications are required to arrive at the desired final form of an engineered cell, and that not all reduced or increased levels of target components are achieved via active engineering. In some embodiments, the modifications of the engineered cell may be in any order, and not necessarily the order listed in the descriptive language provided herein.

Once altered, the presence of expression of any of the molecules described herein can be assayed using known techniques, such as Western blots, ELISA assays, FACS assays, flow cytometry, and the like.

### A. Targets Having Reduced Expression

In some embodiments, the provided engineered cells comprise one or more modifications (e.g. genetic modifications) of one or more target polynucleotide or protein sequences (also interchangeably referred to as a target gene) that decrease (e.g. reduce or eliminate) the expression of one or more of CACNA1G, CACNA1H, HCN4, and SLC8A1. In some embodiments, the one or more modifications (e.g. genetic modifications) of one or more target polynucleotide or protein sequences (also interchangeably referred to as a target gene) that decrease (e.g. reduce or eliminate) the expression of one or more of CACNA1G, CACNA1H, HCN4, and SLC8A1 render the cell less likely to cause engraftment arrhythmia following engraftment in a subject.

In some embodiments, the cell to be modified is a cell that has not previously been introduced with the one or more modifications. In some embodiments, a genetic editing system is used to modify one or more of CACNA1G, CACNA1H, HCN4, and SLC8A1.

In some embodiments, the provided engineered cells comprise one or more modifications (e.g. genetic modifications) of one or more target polynucleotide or protein sequences (also interchangeably referred to as a target gene) that decrease (e.g. reduce or eliminate) the expression of one or more of CACNA1G, HCN4, and SLC8A1. In some embodiments, the one or more modifications (e.g. genetic modifications) of one or more target polynucleotide or protein sequences (also interchangeably referred to as a target gene) that decrease (e.g. reduce or eliminate) the expression of one or more of CACNA1G, HCN4, and SLC8A1 render the cell less likely to cause engraftment arrhythmia following engraftment in a subject.

In some embodiments, the cell to be modified is a cell that has not previously been introduced with the one or more modifications. In some embodiments, a genetic editing system is used to modify one or more of CACNA1G, HCN4, and SLC8A1.

In certain embodiments, the genome of the cell has been altered to reduce or eliminate components required or involved in facilitating expression of one or more of Cav3.1, Cav3.2, HCN4, and SLC8A1 on the surface of the cell. In certain embodiments, the genome of the cell has been altered to reduce or eliminate components required or involved in facilitating expression of one or more of Cav3.1, HCN4, and SLC8A1 on the surface of the cell. For instance, in some embodiments, expression of CACNA1G, a T-type calcium channel, is reduced or eliminated in the cell, thereby reducing or eliminating the expression of Cav3.1 by the engineered cell. In some embodiments, expression of CACNA1H is reduced or eliminated in the cell, thereby reducing or eliminating the expression of Cav3.2 by the engineered cell. In some embodiments, expression of HCN4 is reduced or eliminated in the cell, thereby reducing or eliminating the expression of HCN4 protein by the engineered cell. In some embodiments, expression of SLC8A1 is reduced or eliminated in the cell, thereby reducing or eliminating the expression of SLC8A1 protein by the engineered cell. In some embodiments, expression of CACNA1G and HCN4 is reduced or eliminated in the cell, thereby reducing or eliminating the expression of Cav3.1 and the HCN4 protein by the engineered cell. In some embodiments, expression of CACNA1G and SLC8A1 is reduced or eliminated in the cell, thereby reducing or eliminating the expression of Cav3.1 and the SLC8A1 protein by the engineered cell. In some embodiments, expression of SLC8A1 and HCN4 is reduced or eliminated in the cell, thereby reducing or eliminating the expression of the SLC8A1 and the HCN4 protein by the engineered cell. In some embodiments, expression of CACNA1G, SLC8A1, and HCN4 is reduced or eliminated in the cell, thereby reducing or eliminating the expression of Cav3.1, the SLC8A1 protein, and the HCN4 protein by the engineered cell.

Thus, in some embodiments, expression can be reduced via a gene, and/or function thereof, RNA expression and function, protein expression and function, longevity, or a combination thereof.

In some embodiments, reduced expression of a target is such that expression in an engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a source cell (i.e. a cell of the same cell type) prior to being engineered to reduce expression of the target. In some embodiments, reduced expression of a target is such that expression in an engineered cell is reduced to a level that is about 60% or less (such as any of about 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a reference cell or a reference cell population (such as a cell or population of the same cell type). In some embodiments, reduced expression of a target is such that expression in an engineered cell is reduced to a level that is at or less than a measured level of expression. In some embodiments, the level of a target is assessed in an engineered cell, a reference cell, or reference cell population in a stimulated or non-stimulated state. In some embodiments, the level of a target is assessed in an engineered cell, a reference cell, or reference cell population in a stimulated state such that the target is expressed (or will be if it is a capability of the cell in response to the stimulus). In some embodiments, the stimulus represents an in vivo stimulus.

In some embodiments, the provided engineered cells comprises one or more modifications, such as genetic modifications of one or more of CACNA1G, CACNA1H, HCN4, and SLC8A1. In some embodiments, the provided engineered cells comprises one or more modifications, such as genetic modifications of one or more of CACNA1G, HCN4, and SLC8A1. In some embodiments, the provided engineered cells comprises one or more modifications of CACNA1G, HCN4, and SLC8A1.

In some embodiments, the cell to be modified is a cell of the same cell type as the engineered cell that has not previously been introduced with the one or more modifications. In some embodiments, a genetic editing system is used to modify one or more target polynucleotide sequences that regulate the expression of one or more of CACNA1G, CACNA1H, HCN4, and SLC8A1. In some embodiments, a genetic editing system is used to modify one or more target polynucleotide sequences that regulate the expression of one or more of CACNA1G, HCN4, and SLC8A1. For instance, in some embodiments, CACNA1G is reduced or eliminated in the cell, thereby reducing or eliminating the expression of Cav3.1 by the engineered cell. In some embodiments, CACNA1H is reduced or eliminated in the cell, thereby reducing or eliminating the expression of Cav3.2 by the engineered cell.

In some embodiments, any of the described modifications in the engineered cell that decrease (e.g. reduce or eliminate) expression of one or more target polynucleotide or protein in the engineered cell may be combined together with one or more modifications to overexpress a polynucleotide described in Section II.B (e.g., one or more of KCNJ2, TRDN, SRL, HRC, and CASQ2).

In some embodiments, any of the described modifications in the engineered cell that decrease (e.g. reduce or eliminate) expression of one or more target polynucleotide or protein in the engineered cell may be combined together with one or more modifications to overexpress a polynucleotide described in Section II.B (e.g., one or more of KCNJ2, TRDN, SRL, HRC, and CASQ2).

In some embodiments, any of the described modifications in the engineered cell that decrease (e.g. reduce or eliminate) expression of one or more target polynucleotide or protein in the engineered cell may be combined together with one or more modifications to overexpress a polynucleotide described in Section III.B (e.g., CD47).

In some embodiments, any of the described modifications in the engineered cell that decrease (e.g. reduce or eliminate) expression of one or more target polynucleotide or protein in the engineered cell may be combined together with one or more modifications to overexpress a polynucleotide described in Section II.B and III.B (e.g., one or more of CD47, KCNJ2, TRDN, SRL, HRC, and CASQ2).

In some embodiments, any of the described modifications in the engineered cell that decrease (e.g. reduce or eliminate) expression of one or more target polynucleotide or protein in the engineered cell may be combined together with one or more modifications decrease (e.g. reduce or eliminate) expression of a polynucleotide described in Section III.A (e.g., B2M, TIP1, CD74, and/or CIITA).

In certain aspects, the engineered cells disclosed herein do not express one or more of CACNA1G, CACNA1H, HCN4, and SLC8A1. For example, in certain aspects, the engineered cells disclosed herein have been modified such that the cells do not express, or exhibit reduced expression of, one or more of CACNA1G, CACNA1H, HCN4, and SLC8A1. In some embodiments, one or more of CACNA1G, CACNA1H, HCN4, and/or SLC8A1 may be “knocked-out” of a cell. A cell that has a knocked-out CACNA1G, CACNA1H, HCN4, and/or SLC8A1gene may exhibit reduced or eliminated expression of each knocked-out gene.

In certain aspects, the engineered cells disclosed herein do not express one or more of CACNA1G, HCN4, and SLC8A1. For example, in certain aspects, the engineered cells disclosed herein have been modified such that the cells do not express, or exhibit reduced expression of, one or more of CACNA1G, HCN4, and SLC8A1. In some embodiments, one or more of CACNA1G, HCN4, and/or SLC8A1 may be “knocked-out” of a cell. A cell that has a knocked-out CACNA1G, HCN4, and/or SLC8A1gene may exhibit reduced or eliminated expression of each knocked-out gene.

In certain embodiments, the expression of one or more of CACNA1G, CACNA1H, HCN4, and SLC8A1 is modulated by targeting and deleting a contiguous stretch of genomic DNA, thereby reducing or eliminating expression of a target gene selected from the group consisting of CACNA1G, CACNA1H, HCN4, and SLC8A1. In certain embodiments, the expression of one or more of CACNA1G, HCN4, and SLC8A1 is modulated by targeting and deleting a contiguous stretch of genomic DNA, thereby reducing or eliminating expression of a target gene selected from the group consisting of CACNA1G, HCN4, and SLC8A1. In certain embodiments, the expression of CACNA1G is modulated by targeting and deleting a contiguous stretch of genomic DNA, thereby reducing or eliminating expression of a CACNA1G. In certain embodiments, the expression of HCN4 is modulated by targeting and deleting a contiguous stretch of genomic DNA, thereby reducing or eliminating expression of a HCN4. In certain embodiments, the expression of SLC8A1 is modulated by targeting and deleting a contiguous stretch of genomic DNA, thereby reducing or eliminating expression of a SLC8A1.

Exemplary methods for reducing expression of one or more of CACNA1G, CACNA1H, HCN4, and SLC8A1 are described in sections below. In some embodiments, the targeted polynucleotide sequence is one or both of CACNA1G and CACNA1H. In some embodiments, the cell comprises a genetic editing modification to the CACNA1G gene. In some embodiments, the cell comprises a genetic editing modification to the CACNA1H gene. In some embodiments, the cell comprises a genetic editing modification to the HCN4 gene. In some embodiments, the cell comprises genetic editing modifications to the SLC8A1 gene. In some embodiments, the cell comprises genetic editing modifications to the HCN4 and SLC8A1 genes. In some embodiments, the cell comprises genetic editing modifications to the CACNA1G, HCN4, and SLC8A1 genes. In some embodiments, the cell comprises genetic editing modifications to the CACNA1G, CACNA1H, HCN4 and SLC8A1 genes.

In some embodiments, the modification that reduces one or more of CACNA1G, CACNA1H, HCN4, and SLC8A1 expression reduces one or more of CACNA1G, CACNA1H, HCN4, and/or SLC8A1mRNA expression. In some embodiments, the reduced mRNA expression of CACNA1G, CACNA1H, HCN4, and SLC8A1 is relative to an unmodified or wild-type cell of the same cell type that does not comprise the modification. In some embodiments, the modification that reduces one or more of CACNA1G, HCN4, and SLC8A1 expression reduces one or more of CACNA1G, HCN4, and/or SLC8A1mRNA expression. In some embodiments, the reduced mRNA expression of CACNA1G, HCN4, and SLC8A1 is relative to an unmodified or wild-type cell of the same cell type that does not comprise the modification.

In some embodiments, the mRNA expression of CACNA1G is reduced by more than about 5%, such as reduced by more than about any of 10%, 20%, 30%, 40%, 50%, 60%, 70%, 80%, 90%, or more. In some embodiments, the mRNA expression of CACNA1H is reduced by more than about 5%, such as reduced by more than about any of 10%, 20%, 30%, 40%, 50%, 60%, 70%, 80%, 90%, or more. In some embodiments, the mRNA expression of HCN4 is reduced by up to about 100%, such as reduced by up to about any of 90%, 80%, 70%, 60%, 50%, 40%, 30%, 20%, 10%, 5%, or less. In some embodiments, the mRNA expression of SLC8A1 is reduced by any of about 5%, 10%, 20%, 30%, 40%, 50%, 60%, 70%, 80%, 90%, or 100%. In some embodiments, the mRNA expression of each of CACNA1G, CACNA1H, HCN4, and SLC8A1 is reduced by any of about 5%, 10%, 20%, 30%, 40%, 50%, 60%, 70%, 80%, 90%, or 100%. In some embodiments, the mRNA expression of each of CACNA1G, HCN4, and SLC8A1 is reduced by any of about 5%, 10%, 20%, 30%, 40%, 50%, 60%, 70%, 80%, 90%, or 100%.

In some embodiments, the mRNA expression of CACNA1G, CACNA1H, HCN4, and/or SLC8A1 is eliminated (e.g., 0% expression of CACNA1G, CACNA1H, HCN4, and/or SLC8A1 mRNA). In some embodiments, the modification that reduces CACNA1G, CACNA1H, HCN4, and/or SLC8A1 mRNA expression eliminates CACNA1G, CACNA1H, HCN4, and/or SLC8A1 gene activity. In some embodiments, the mRNA expression of CACNA1G, CACNA1H, HCN4, and/or SLC8A1 is reduced by any of about 5%, 10%, 20%, 30%, 40%, 50%, 60%, 70%, 80%, 90%, or 100%. In some embodiments, the mRNA expression of CACNA1G, CACNA1H, HCN4, and/or SLC8A1 is eliminated (e.g., 0% expression of CACNA1G, CACNA1H, HCN4, and/or SLC8A1 mRNA). In some embodiments, the modification that reduces CACNA1G, CACNA1H, HCN4, and/or SLC8A1 mRNA expression eliminates CACNA1G, CACNA1H, HCN4, and/or SLC8A1 gene activity.

In some embodiments, the mRNA expression of CACNA1G, HCN4, and/or SLC8A1 is eliminated (e.g., 0% expression of CACNA1G, HCN4, and/or SLC8A1 mRNA). In some embodiments, the modification that reduces CACNA1G, HCN4, and/or SLC8A1 mRNA expression eliminates CACNA1G, HCN4, and/or SLC8A1 gene activity. In some embodiments, the mRNA expression of CACNA1G, HCN4, and/or SLC8A1 is reduced by any of about 5%, 10%, 20%, 30%, 40%, 50%, 60%, 70%, 80%, 90%, or 100%. In some embodiments, the mRNA expression of CACNA1G, HCN4, and/or SLC8A1 is eliminated (e.g., 0% expression of CACNA1G, HCN4, and/or SLC8A1 mRNA). In some embodiments, the modification that reduces CACNA1G, HCN4, and/or SLC8A1 mRNA expression eliminates CACNA1G, HCN4, and/or SLC8A1 gene activity.

In some embodiments, the modification that reduces CACNA1G, CACNA1H, HCN4, and/or SLC8A1 expression reduces Cav3.1, Cav3.2, HCN4, and/or SLC8A1 protein expression. In some embodiments, the reduced protein expression of Cav3.1, Cav3.2, HCN4, and/or SLC8A1 is relative to an unmodified or wild-type cell of the same cell type that does not comprise the modification. In some embodiments, the protein expression of Cav3.1, Cav3.2, HCN4, and/or SLC8A1 is reduced by more than about 5%, such as reduced by more than about any of 10%, 20%, 30%, 40%, 50%, 60%, 70%, 80%, 90%, or more. In some embodiments, the protein expression of Cav3.1, Cav3.2, HCN4, and/or SLC8A1 is reduced by up to about 100%, such as reduced by up to about any of 90%, 80%, 70%, 60%, 50%, 40%, 30%, 20%, 10%, 5%, or less. In some embodiments, the protein expression of Cav3.1, Cav3.2, HCN4, and/or SLC8A1 is reduced by any of about 5%, 10%, 20%, 30%, 40%, 50%, 60%, 70%, 80%, 90%, or 100%. In some embodiments, the protein expression of Cav3.1, Cav3.2, HCN4, and/or SLC8A1 is eliminated (e.g., 0% expression of Cav3.1, Cav3.2, HCN4, and/or SLC8A1 protein). In some embodiments, the modification that reduces Cav3.1, Cav3.2, HCN4, and/or SLC8A1 protein expression eliminates CACNA1G, CACNA1H, HCN4, and/or SLC8A1 gene activity.

In some embodiments, the modification that reduces CACNA1G, HCN4, and/or SLC8A1 expression reduces Cav3.1, HCN4, and/or SLC8A1 protein expression. In some embodiments, the reduced protein expression of Cav3.1, HCN4, and/or SLC8A1 is relative to an unmodified or wild-type cell of the same cell type that does not comprise the modification. In some embodiments, the protein expression of Cav3.1, HCN4, and/or SLC8A1 is reduced by more than about 5%, such as reduced by more than about any of 10%, 20%, 30%, 40%, 50%, 60%, 70%, 80%, 90%, or more. In some embodiments, the protein expression of Cav3.1, HCN4, and/or SLC8A1 is reduced by up to about 100%, such as reduced by up to about any of 90%, 80%, 70%, 60%, 50%, 40%, 30%, 20%, 10%, 5%, or less. In some embodiments, the protein expression of Cav3.1, HCN4, and/or SLC8A1 is reduced by any of about 5%, 10%, 20%, 30%, 40%, 50%, 60%, 70%, 80%, 90%, or 100%. In some embodiments, the protein expression of Cav3.1, HCN4, and/or SLC8A1 is eliminated (e.g., 0% expression of Cav3.1, HCN4, and/or SLC8A1 protein). In some embodiments, the modification that reduces Cav3.1, HCN4, and/or SLC8A1 protein expression eliminates CACNA1G, HCN4, and/or SLC8A1 gene activity.

In some embodiments, the modification that reduces CACNA1G, CACNA1H, HCN4, and/or SLC8A1 expression comprises inactivation or disruption of CACNA1G, CACNA1H, HCN4, and/or SLC8A1 gene. In some embodiments, the modification that reduces CACNA1G, CACNA1H, HCN4, and/or SLC8A1 expression comprises inactivation or disruption of one allele of the CACNA1G, CACNA1H, HCN4, and/or SLC8A1 gene. In some embodiments, the modification that reduces CACNA1G, CACNA1H, HCN4, and/or SLC8A1 expression comprises inactivation or disruption comprises inactivation or disruption of both alleles of the CACNA1G, CACNA1H, HCN4, and/or SLC8A1 gene.

In some embodiments, the modification that reduces CACNA1G, HCN4, and/or SLC8A1 expression comprises inactivation or disruption of CACNA1G, HCN4, and/or SLC8A1 gene. In some embodiments, the modification that reduces CACNA1G, HCN4, and/or SLC8A1 expression comprises inactivation or disruption of one allele of the CACNA1G, HCN4, and/or SLC8A1 gene. In some embodiments, the modification that reduces CACNA1G, HCN4, and/or SLC8A1 expression comprises inactivation or disruption comprises inactivation or disruption of both alleles of the CACNA1G, HCN4, and/or SLC8A1 gene.

In some embodiments, the modification that reduces CACNA1G expression comprises inactivation or disruption of CACNA1G gene. In some embodiments, the modification that reduces CACNA1G expression comprises inactivation or disruption of one allele of the CACNA1G gene. In some embodiments, the modification that reduces CACNA1G expression comprises inactivation or disruption comprises inactivation or disruption of both alleles of the CACNA1G gene.

In some embodiments, the modification that reduces CACNA1G, HCN4, and SLC8A1 expression comprises inactivation or disruption of the CACNA1G, HCN4, and SLC8A1 gene. In some embodiments, the modification that reduces CACNA1G, HCN4, and SLC8A1 expression comprises inactivation or disruption of one allele of the CACNA1G, HCN4, and SLC8A1 gene. In some embodiments, the modification that reduces CACNA1G, HCN4, and SLC8A1 expression comprises inactivation or disruption comprises inactivation or disruption of both alleles of the CACNA1G, HCN4, and SLC8A1 gene.

In some embodiments, the modification comprises inactivation or disruption of CACNA1G, CACNA1H, HCN4, and/or SLC8A1 coding sequences in the cell. In some embodiments, the modification comprises inactivation or disruption of all CACNA1G, CACNA1H, HCN4, and/or SLC8A1 coding sequences in the cell. In some embodiments, the inactivation or disruption comprises an indel in the CACNA1G, CACNA1H, HCN4, and/or SLC8A1 gene. In some embodiments, the modification is a frameshift mutation of genomic DNA of the CACNA1G, CACNA1H, HCN4, and/or SLC8A1 gene. In some embodiments, the modification is a deletion of genomic DNA of the CACNA1G, CACNA1H, HCN4, and/or SLC8A1 gene. In some embodiments, the modification is a deletion of a contiguous stretch of genomic DNA of the CACNA1G, CACNA1H, HCN4, and/or SLC8A1 gene. In some embodiments, the CACNA1G, CACNA1H, HCN4, and/or SLC8A1 gene is knocked out.

In some embodiments, the modification comprises inactivation or disruption of CACNA1G, HCN4, and/or SLC8A1 coding sequences in the cell. In some embodiments, the modification comprises inactivation or disruption of all CACNA1G, HCN4, and/or SLC8A1 coding sequences in the cell. In some embodiments, the inactivation or disruption comprises an indel in the CACNA1G, HCN4, and/or SLC8A1 gene. In some embodiments, the modification is a frameshift mutation of genomic DNA of the CACNA1G, HCN4, and/or SLC8A1 gene. In some embodiments, the modification is a deletion of genomic DNA of the CACNA1G, HCN4, and/or SLC8A1 gene. In some embodiments, the modification is a deletion of a contiguous stretch of genomic DNA of the CACNA1G, HCN4, and/or SLC8A1 gene. In some embodiments, the CACNA1G, HCN4, and/or SLC8A1 gene is knocked out.

In some embodiments, the modification comprises inactivation or disruption of CACNA1G, HCN4, and SLC8A1 coding sequences in the cell. In some embodiments, the modification comprises inactivation or disruption of all CACNA1G, HCN4, and SLC8A1 coding sequences in the cell. In some embodiments, the inactivation or disruption comprises an indel in the CACNA1G, HCN4, and SLC8A1 gene. In some embodiments, the modification is a frameshift mutation of genomic DNA of the CACNA1G, HCN4, and SLC8A1 gene. In some embodiments, the modification is a deletion of genomic DNA of the CACNA1G, HCN4, and SLC8A1 gene. In some embodiments, the modification is a deletion of a contiguous stretch of genomic DNA of the CACNA1G, HCN4, and SLC8A1 gene. In some embodiments, the CACNA1G, HCN4, and SLC8A1 gene is knocked out.

In some embodiments, the modification comprises inactivation or disruption of CACNA1G coding sequences in the cell. In some embodiments, the modification comprises inactivation or disruption of all CACNA1G coding sequences in the cell. In some embodiments, the inactivation or disruption comprises an indel in one allele of the CACNA1G gene. In some embodiments, the inactivation or disruption comprises an indel in both alleles of the CACNA1G gene. In some embodiments, the modification is a frameshift mutation of genomic DNA of the CACNA1G gene. In some embodiments, the modification is a deletion of genomic DNA of the CACNA1G gene. In some embodiments, the modification is a deletion of a contiguous stretch of genomic DNA of the CACNA1G gene. In some embodiments, the CACNA1G gene is knocked out.

In some embodiments, the engineered comprises reduced expression of one or more of CACNA1G, CACNA1H, HCN4, and SLC8A1, wherein reduced is as described herein, such as relative to prior to engineering to reduce expression of one or more of CACNA1G, CACNA1H, HCN4, and SLC8A1, a reference cell or a reference cell population, or a measured value.

In some embodiments, the engineered comprises reduced expression of one or more of CACNA1G, HCN4, and SLC8A1, wherein reduced is as described herein, such as relative to prior to engineering to reduce expression of one or more of CACNA1G, HCN4, and SLC8A1, a reference cell or a reference cell population, or a measured value.

In some embodiments, the engineered comprises reduced expression of CACNA1G, wherein reduced is as described herein, such as relative to prior to engineering to reduce expression of CACNA1G, a reference cell or a reference cell population, or a measured value.

In some embodiments, the engineered cell is engineered to reduce expression of one or more of CACNA1G, CACNA1H, HCN4, and SLC8A1. In some embodiments, expression of one or more of CACNA1G, CACNA1H, HCN4, and SLC8A1, on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more of CACNA1G, CACNA1H, HCN4, and SLC8A1 expression prior to being engineered to reduce expression. In some embodiments, expression of one or more of CACNA1G, CACNA1H, HCN4, and SLC8A1 on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, CACNA1H, HCN4, and SLC8A1 expression on a reference cell or a reference cell population. In some embodiments, there is no cell surface presentation of one or more of CACNA1G, CACNA1H, HCN4, and SLC8A1 on the engineered cell (including no detectable expression, including as measured using known techniques, e.g., flow cytometry). In some embodiments, the engineered cell exhibits reduced protein expression of one or more of CACNA1G, CACNA1H, HCN4, and SLC8A1. In some embodiments, protein expression of one or more of CACNA1G, CACNA1H, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, CACNA1H, HCN4, and SLC8A1 protein expression prior to being engineered to reduce protein expression. In some embodiments, protein expression of one or more of CACNA1G, CACNA1H, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, CACNA1H, HCN4, and SLC8A1 prior to being engineered to reduce protein expression of one or more of CACNA1G, CACNA1H, HCN4, and SLC8A1. In some embodiments, the engineered cell exhibits no protein expression of one or more of CACNA1G, CACNA1H, HCN4, and SLC8A1, including no detectable protein expression, including as measured using known techniques, e.g., western blot or mass spectrometry). In some embodiments, the engineered cell does not comprise one or more of CACNA1G, CACNA1H, HCN4, and SLC8A1 (including no detectable protein, including as measured using known techniques, e.g., western blot or mass spectrometry). In some embodiments, the engineered cell exhibits reduced mRNA expression encoding one or more of CACNA1G, CACNA1H, HCN4, and SLC8A1. In some embodiments, mRNA expression encoding one or more of CACNA1G, CACNA1H, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding one or more of CACNA1G, CACNA1H, HCN4, and SLC8A1 prior to being engineered to reduce mRNA expression. In some embodiments, mRNA expression encoding one or more of CACNA1G, CACNA1H, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding one or more of CACNA1G, CACNA1H, HCN4, and SLC8A1 (including no detectable mRNA expression, including as measured using known techniques, e.g., sequencing techniques or PCR). In some embodiments, the engineered cell does not comprise mRNA encoding one or more of CACNA1G, CACNA1H, HCN4, and SLC8Alf (including no detectable mRNA, including as measured using known techniques, e.g., sequencing techniques or PCR). In some embodiments, the engineered cell comprises a gene inactivation or disruption of one or more of CACNA1G, CACNA1H, HCN4, and SLC8A1. In some embodiments, the engineered cell comprises a gene inactivation or disruption of one or more of CACNA1G, CACNA1H, HCN4, and SLC8A1 in one allele. In some embodiments, the engineered cell comprises a gene inactivation or disruption of one or more of CACNA1G, CACNA1H, HCN4, and SLC8A1 in both alleles. In some embodiments, the engineered cell is a knockout of one or more of CACNA1G, CACNA1H, HCN4, and SLC8A1.

In some embodiments, the engineered cell is engineered to reduce expression of one or more of CACNA1G, HCN4, and SLC8A1. In some embodiments, expression of one or more of CACNA1G, HCN4, and SLC8A1, on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more of CACNA1G, HCN4, and SLC8A1 expression prior to being engineered to reduce expression. In some embodiments, expression of one or more of CACNA1G, HCN4, and SLC8A1 on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, HCN4, and SLC8A1 expression on a reference cell or a reference cell population. In some embodiments, there is no cell surface presentation of one or more of CACNA1G, HCN4, and SLC8A1on the engineered cell (including no detectable expression, including as measured using known techniques, e.g., flow cytometry). In some embodiments, the engineered cell exhibits reduced protein expression of one or more of CACNA1G, HCN4, and SLC8A1. In some embodiments, protein expression of one or more of CACNA1G, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, HCN4, and SLC8A1 protein expression prior to being engineered to reduce protein expression. In some embodiments, protein expression of one or more of CACNA1G, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of one or more of CACNA1G, HCN4, and SLC8A1 prior to being engineered to reduce protein expression of one or more of CACNA1G, HCN4, and SLC8A1. In some embodiments, the engineered cell exhibits no protein expression of one or more of CACNA1G, HCN4, and SLC8A1, including no detectable protein expression, including as measured using known techniques, e.g., western blot or mass spectrometry). In some embodiments, the engineered cell does not comprise one or more of CACNA1G, HCN4, and SLC8A1 (including no detectable protein, including as measured using known techniques, e.g., western blot or mass spectrometry). In some embodiments, the engineered cell exhibits reduced mRNA expression encoding one or more of CACNA1G, HCN4, and SLC8A1. In some embodiments, mRNA expression encoding one or more of CACNA1G, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding one or more of CACNA1G, HCN4, and SLC8A1 prior to being engineered to reduce mRNA expression. In some embodiments, mRNA expression encoding one or more of CACNA1G, HCN4, and SLC8A1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding one or more of CACNA1G, HCN4, and SLC8A1 (including no detectable mRNA expression, including as measured using known techniques, e.g., sequencing techniques or PCR). In some embodiments, the engineered cell does not comprise mRNA encoding one or more of CACNA1G, HCN4, and SLC8A1f (including no detectable mRNA, including as measured using known techniques, e.g., sequencing techniques or PCR). In some embodiments, the engineered cell comprises a gene inactivation or disruption of one or more of CACNA1G, HCN4, and SLC8A1. In some embodiments, the engineered cell comprises a gene inactivation or disruption of one or more of CACNA1G, HCN4, and SLC8A1 in one allele. In some embodiments, the engineered cell comprises a gene inactivation or disruption of one or more of CACNA1G, HCN4, and SLC8A1 in both alleles. In some embodiments, the engineered cell is a knockout of one or more of CACNA1G, HCN4, and SLC8A1.

In some embodiments, the engineered cell is engineered to reduce expression of Cav3.1. In some embodiments, Cav3.1 is human Cav3.1. In some embodiments, Cav3.1 is human Cav3.1 and is or comprises the amino acid sequence of SEQ ID NO: 3. In some embodiments, expression of Cav3.1 on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of Cav3.1 expression prior to being engineered to reduce expression of Cav3.). In some embodiments, expression of Cav3.1 on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of Cav3.1 expression on a reference cell or a reference cell population (such as an average amount of Cav3.1 expression). In some embodiments, there is no expression of Cav3.1 on the engineered cell (including no detectable expression, including as measured using known techniques, e.g., flow cytometry). In some embodiments, the engineered cell exhibits reduced protein expression of Cav3.1. In some embodiments, protein expression of Cav3.1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of Cav3.1 protein expression prior to being engineered to reduce protein expression of Cav3.1. In some embodiments, protein expression of Cav3.1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of Cav3.1 prior to being engineered to reduce protein expression of Cav3.1. In some embodiments, the engineered cell exhibits no protein expression of Cav3.1, (including no detectable protein expression, including as measured using known techniques, e.g., western blot or mass spectrometry). In some embodiments, the engineered cell does not comprise the Cav3.1 T-type calcium channel (including no detectable protein, including as measured using known techniques, e.g., western blot or mass spectrometry). In some embodiments, the engineered cell exhibits reduced mRNA expression encoding Cav3.1. In some embodiments, mRNA expression encoding Cav3.1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding Cav3.1 prior to being engineered to reduce mRNA expression of Cav3.1. In some embodiments, mRNA expression encoding Cav3.1 of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding Cav3.1 (including no detectable mRNA expression, including as measured using known techniques, e.g., sequencing techniques or PCR). In some embodiments, the engineered cell does not comprise mRNA encoding Cav3.1 (including no detectable mRNA, including as measured using known techniques, e.g., sequencing techniques or PCR). In some embodiments, the engineered cell comprises a gene inactivation or disruption of the CACNA1G gene. In some embodiments, the engineered cell comprises a gene inactivation or disruption of the CACNA1G gene in one allele. In some embodiments, the engineered cell comprises a gene inactivation or disruption of the CACNA1G gene in both alleles. In some embodiments, the engineered cell is a CACNA1G knockout.

### B. Targets Having Increased Expression

In some embodiments, the provided engineered cells are genetically modified or engineered, such as by introduction of one or more modifications into a cell to overexpress a desired polynucleotide in the cell. In some embodiments, the engineered cell to be modified or engineered is an unmodified cell or non-engineered cell that has not previously been introduced with the one or more modifications. In some embodiments, the engineered cells provided herein are genetically modified to include one or more exogenous polynucleotides encoding an exogenous protein (also interchangeably used with the term “transgene”). As described, in some embodiments, the cells are modified to increase expression of more genes or encoded proteins that may contribute to the reduction or prevent of engraftment arrhythmia. In some embodiments, expression of a target gene (e.g., one or more of KCNJ2, TRDN, SRL, HRC, and CASQ2) is increased by expression of a fusion protein or a protein complex containing (1) a site-specific binding domain specific for the endogenous gene and (2) a transcriptional activator.

The one or more polynucleotides, e.g. exogenous polynucleotides, may be expressed (e.g. overexpressed) in the engineered cell together with one or more genetic modifications to reduce expression of a target polynucleotide described in Section II.A above, such as one or more of CACNA1G, CACNA1H, HCN4, and SLC8A1. The one or more polynucleotides, e.g. exogenous polynucleotides, may be expressed (e.g. overexpressed) in the engineered cell together with one or more genetic modifications to reduce expression of a target polynucleotide described in Section III.A, such as one or more of B2M, CIITA, CD74, and TAP1. The one or more polynucleotides, e.g. exogenous polynucleotides, may be expressed (e.g. overexpressed) in the engineered cell together with one or more genetic modifications to increase expression of a target polynucleotide described in Section III.B, such as CD47. In some embodiments, the provided engineered cells do not cause engraftment arrhythmia following engraftment of the cells in a subject.

In some embodiments, the engineered cell includes 1, 2, 3, 4, 5, 6, 7, 8, 9, 10 or more different overexpressed polynucleotides. In some embodiments, the engineered cell includes 1, 2, 3, 4, 5, 6, 7, 8, 9, 10 or more different overexpressed polynucleotides. In some embodiments, the overexpressed polynucleotide is an exogenous polynucleotide. In some embodiments, the engineered cell includes 1, 2, 3, 4, 5, 6, 7, 8, 9, 10 or more different exogenous polynucleotides. In some embodiments, the engineered cell includes 1, 2, 3, 4, 5, 6, 7, 8, 9, 10 or more different exogenous polynucleotides. In some embodiments, the overexpressed polynucleotide is an exogenous polynucleotide that is inserted or integrated into one or more genomic loci of the engineered cell.

In some embodiments, expression of a polynucleotide is increased, i.e. the polynucleotide is overexpressed, using a fusion protein containing a DNA-targeting domain and a transcriptional activator. Targeted methods of increasing expression using transactivator domains are known to a skilled artisan.

In some embodiments, engineered cell contains one or more exogenous polynucleotides in which the one or more exogenous polynucleotides are inserted or integrated into a genomic locus of the cell by non-targeted insertion methods, such as by transduction with a lentiviral vector. In some embodiments, the lentiviral vector comprises a piggyBac transposon. During transposition, the piggyback transposon recognizes transposon-specific inverted terminal repeats (ITRs) in a lentiviral vector, to allow for the efficient movement and integration of the vector contents into TTAA chromosomal sites. In some embodiments, the one or more exogenous polynucleotides are inserted or integrated into the genome of the cell by targeted insertion methods, such as by using homology directed repair (HDR). Any suitable method can be used to insert the exogenous polynucleotide into the genomic locus of the engineered cell by HDR including the gene editing methods described herein (e.g., a CRISPR/Cas system). In some embodiments, the one or more exogenous polynucleotides are inserted into one or more genomic locus, such as any genomic locus described herein (e.g. Table 4). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g. Table 4). In some embodiments, two or more exogenous polynucleotides are inserted into a different genomic loci, such as two or more genomic loci as described herein (e.g., Table 4).

Exemplary polynucleotides or overexpression, and methods for overexpressing the same, are described in the following subsections.

### C. Cells

In some embodiments, the present disclosure provides a cell (e.g., a PSC, a cardiomyocyte derived from a PSC, or a primary cardiac cell), or population thereof, that has been engineered (or modified) in which the genome of the cell has been modified such that expression of one or more gene as described herein is reduced or deleted or in which a gene or polynucleotide is overexpressed or increased in expression. In some embodiments, the engineered cell comprises one or more modifications that (a) reduce expression of one or more of CACNA1G, CACNA1H, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the engineered cell comprises one or more modifications that (a) reduce expression of one or more of CACNA1G, CACNA1H, HCN4, and SLC8A1; (b) increase expression of KCNJ2; or (c) a combination thereof, relative to a cell of the same cell type that does not comprise the one or more modifications. In some embodiments, the engineered cell comprises one or more modifications that (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the engineered cell comprises one or more modifications that (a) reduce expression of one or more of CACNA1G, HCN4, and SLC8A1; (b) increase expression of one or more of KCNJ2; or (c) a combination thereof, relative to a cell of the same cell type that does not comprise the one or more modifications.

In some embodiments, the cells that are engineered or modified as provided herein are pluripotent stem cells (PSCs). In some embodiments, the cells that are engineered or modified as provided herein are embryonic stem cells (ESCs). In some embodiments, the cells that are engineered or modified as provided herein are induced pluripotent stem cells (iPSCs).

In some embodiments, the cells that are engineered or modified as provided herein are cardiomyocytes or a precursor thereof. In some embodiments, the cardiomyocytes or precursors thereof are differentiated from PSCs. In some embodiments, the cells that are engineered or modified as provided herein are cardiomyocytes differentiated from PSCs. In some embodiments, the cells that are engineered or modified as provided herein are primary cardiac cells. In some embodiments, the cells that are engineered or modified as provided herein are primary epicardial cells. In some embodiments, populations of engineered cells are provided as a therapeutic cardiac cell therapy, including for a subject having a heart disease or condition. In some embodiments, the engineered cells of the cardiac cell therapy are cardiomyocytes differentiated from PSCs or primary cardiac cells having any of the one or more modifications described in Sections II and III.

The cell may be a vertebrate cell, for example, a mammalian cell, such as a human cell or a mouse cell. Preferably, the cell is amenable to modification. Preferably, the cell has or is believed to have therapeutic value, such that the cell may be used to treat a disease, disorder, defect or injury in a subject in need of treatment for same.

In some embodiments, the cell is a PSC that is engineered to contain modifications (e.g., genetic modifications) described herein. In some embodiments, the engineered PSC comprises one or more modifications that: (a) reduce expression of one or more of CACNA1G, CACNA1H, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the engineered PSC comprises one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the engineered PSC comprises one or more modifications that: (a) reduce expression of one or more of CACNA1G, CACNA1H, HCN4, and SLC8A1; (b) increase expression of KCNJ2; or (c) a combination thereof, relative to a cell of the same cell type that does not comprise the one or more modifications. In some embodiments, the engineered PSC comprises one or more modifications that: (a) reduce expression of one or more of CACNA1G, HCN4, and SLC8A1; (b) increase expression of KCNJ2; or (c) a combination thereof, relative to a cell of the same cell type that does not comprise the one or more modifications.

In some embodiments, the cell is a cardiomyocyte differentiated from a PSC that is engineered to contain modifications (e.g. genetic modifications) described herein. In some embodiments, the cardiomyocyte differentiated from a PSC comprises one or more modifications that: (a) reduce expression of one or more of CACNA1G, CACNA1H, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the cardiomyocyte differentiated from a PSC comprises one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the cardiomyocyte differentiated from a PSC comprises one or more modifications that: (a) reduce expression of one or more of CACNA1G, CACNA1H, HCN4, and SLC8A1; (b) increase expression of KCNJ2; or (c) a combination thereof, relative to a cell of the same cell type that does not comprise the one or more modifications. In some embodiments, the cardiomyocyte differentiated from a PSC comprises one or more modifications that: (a) reduce expression of one or more of CACNA1G, HCN4, and SLC8A1; (b) increase expression of KCNJ2; or (c) a combination thereof, relative to a cell of the same cell type that does not comprise the one or more modifications.

In some embodiments, the cell is a primary cardiac cell that is engineered to contain modifications (e.g. genetic modifications) described herein. In some embodiments, the primary cardiac cell comprises one or more modifications that: (a) reduce expression of one or more of CACNA1G, CACNA1H HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the cell is a primary cardiac cell that is engineered to contain modifications (e.g. genetic modifications) described herein. In some embodiments, the primary cardiac cell comprises one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications. In some embodiments, the cell is a primary cardiac cell that is engineered to contain modifications (e.g. genetic modifications) described herein. In some embodiments, the primary cardiac cell comprises one or more modifications that: (a) reduce expression of one or more of CACNA1G, CACNA1H HCN4, and SLC8A1; (b) increase expression of KCNJ2; or (c) a combination thereof, relative to a cell of the same cell type that does not comprise the one or more modifications. In some embodiments, the cell is a primary cardiac cell that is engineered to contain modifications (e.g. genetic modifications) described herein. In some embodiments, the primary cardiac cell comprises one or more modifications that: (a) reduce expression of one or more of CACNA1G, HCN4, and SLC8A1; (b) increase expression of KCNJ2; or (c) a combination thereof, relative to a cell of the same cell type that does not comprise the one or more modifications.

In some embodiments, the cell is further engineered to be hypoimmunogenic as described in Section III, and can be used to treat a variety of indications with cell therapy, including any as described herein. In some embodiments, the engineered cell can be used to treat a heart disease or condition. In some embodiments, the heart disease or condition is myocardial infarction (MI).

Thus, in some embodiments, the engineered therapeutic cells (e.g., engineered primary cardiac cells or engineered cardiomyocytes differentiated from iPSCs) are also engineered to be hypoimmunogenic by any of the methods described in Section III.

In some embodiments, the engineered cell comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the engineered cell comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the engineered cell comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the engineered cell comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

In some embodiments, the engineered primary cardiac cell is an engineered therapeutic cell, such as for use in a cardiac cell therapy.

In some embodiments, the cardiomyocyte differentiated from a PSC comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the cardiomyocyte differentiated from a PSC comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the cardiomyocyte differentiated from a PSC comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the cardiomyocyte differentiated from a PSC comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

In some embodiments, the primary cardiac cell comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the primary cardiac cell comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the primary cardiac cell comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the primary cardiac cell comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

In some embodiments, the cells that are engineered or modified as provided herein are cells from a healthy subject, such as a subject that is not known or suspected of having a particular disease or condition to be treated. For instance, if primary cells are isolated or obtained from a donor subject, such as for treating a heart disease or condition, the donor subject is a healthy subject if the subject is not known or suspected of suffering from a heart disease or condition or another disease or condition.

For therapeutic application, cells prepared according to the disclosed methods can typically be supplied in the form of a pharmaceutical composition comprising an isotonic excipient, and are prepared under conditions that are sufficiently sterile for human administration. The cells can be packaged in a device or container suitable for distribution or clinical.

Provided herein are engineered cardiac cell types for subsequent transplantation or engraftment into subjects. In some embodiments, a cardiac cell is a cardiomyocyte. In some embodiments, the cardiomyocyte is a contractile cell. In some embodiments, the cardiomyocyte is a conducting cell. In some embodiments, a cardiac cell is a cardiac fibroblast. In some embodiments, a cardiac cell is a primary cardiac cell. In some embodiments, a cardiac cell is a primary epicardial cell. In some embodiments, a cardiac cell is a primary cardiomyocyte. In some embodiments, a cardiac cell is a primary cardiac fibroblast.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a heart disease or condition in a subject. The methods described herein can be used to treat, ameliorate, prevent or slow the progression of a number of cardiac diseases or their symptoms, such as those resulting in pathological damage to the structure and/or function of the heart. The terms “heart disease,” “heart condition,” “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the population of engineered cells are maintained in culture, in some cases expanded, prior to administration. In certain embodiments, the population of engineered cells are cryopreserved prior to administration.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arrhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

### D. Exemplary Embodiments of Engineered Cells

In some embodiments, the engineered cells and populations thereof are engineered PSCs, cardiomyocytes differentiated therefrom, or primary cardiac cells. In some embodiments, cardiomyocytes differentiated therefrom, or primary cardiac cells that have been engineered are for use in a cardiac cell therapy, such as for treating a subject having a heart disease or condition.

In some embodiments, the engineered cell is a human cell or an animal cell. In some embodiments, the engineered cell is a cell isolated from a subject to whom the engineered cells will be administered. In some embodiments, the engineered cell is a cell isolated from a donor subject (e.g., a healthy donor subject not suspected of having a disease or condition at the time the donor sample is obtained from the individual donor subject). In some embodiments, the engineered cell is selected from a pluripotent stem cell (e.g., an embryonic stem cell or an induced pluripotent stem cell), a cardiomyocyte differentiated therefrom, or a primary cardiac cell (e.g., a primary cardiomyocyte). In some embodiments, the engineered cell is selected from embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, or a transdifferentiated version of any of the foregoing cells. In some embodiments, the engineered cell is a PSC. In some embodiments, the PSC is an ESC. In some embodiments, the PSC is an iPSC. In some embodiments, the engineered cell is a cardiomyocyte differentiated from a PSC, including by any of the methods described in Section VI. In some embodiments, the engineered cell is a primary cardiac cell. In some embodiments, the primary cardiac cell is a primary cardiomyocyte. In some embodiments, the engineered cell is a progenitor cell. In some embodiments, the engineered cell is a partially reprogrammed somatic cell. In some embodiments, the engineered cell is a somatic cell which has been partially reprogrammed to an intermediate state between an induced pluripotent stem cell and the somatic cell from which it was derived. In some embodiments, the engineered cell is a multipotent cell. In some embodiments, the engineered cell is a totipotent cell. In some embodiments, the engineered cell is a transdifferentiated version of an embryonic stem cell, induced-pluripotent stem cell, progenitor cell, partially reprogrammed somatic cell, multipotent cell, or totipotent cell.

In some embodiments, the engineered cells and populations thereof exhibit reduced expression of one or more of CACNA1G, CACNA1H, HCN4, and SLC8A1. In some embodiments, the engineered cells and populations thereof exhibit increased expression of one or more of KCNJ2, TRDN, SRL, HRC, and CASQ2.

In some embodiments, the engineered cells and populations thereof exhibit reduced expression of one or more of CACNA1G, CACNA1H, HCN4, and SLC8A1, and increased expression of one or more of KCNJ2, TRDN, SRL, HRC, and CASQ2.

In some embodiments, the engineered cells and populations thereof exhibit reduced expression of CACNA1G. In some embodiments, the engineered cells and populations thereof exhibit reduced expression of CACNA1G and increased expression of one or more molecules of KCNJ2, TRDN, SRL, HRC, and CASQ2. In some embodiments, the engineered cells and populations thereof exhibit reduced expression of CACNA1G and increased expression of KCNJ2. In some embodiments, the engineered cells and populations thereof exhibit reduced expression of CACNA1G, HCN4, and SLC8A1, and increased expression of one or more molecules of KCNJ2, TRDN, SRL, HRC, and CASQ2. In some embodiments, the engineered cells and populations thereof exhibit reduced expression of CACNA1G, HCN4, and SLC8A1, and increased expression of one or more molecules of KCNJ2.

One skilled in the art will appreciate that levels of expression such as increased (e.g., overexpression) or reduced expression of a gene, protein or molecule can be referenced or compared to a comparable cell. In some embodiments, an engineered cell having increased expression of KCNJ2 refers to a modified cell having a higher level of KCNJ2 protein compared to an unmodified cell. In some embodiments, an engineered cell having increased expression of TRDN refers to a modified cell having a higher level of triadin protein compared to an unmodified cell. In some embodiments, an engineered cell having increased expression of SRL refers to a modified cell having a higher level of sarcalumenin protein compared to an unmodified cell. In some embodiments, an engineered cell having increased expression of HRC refers to a modified cell having a higher level of HRC protein compared to an unmodified cell. In some embodiments, an engineered cell having increased expression of CASQ2 refers to a modified cell having a higher level of calsequestrin-2 protein compared to an unmodified cell. In some embodiments, an engineered cell having reduced expression of CACNA1G refers to a modified cell having a lower level of Cav3.1 protein compared to an unmodified cell. In some embodiments, an engineered cell having reduced expression of CACNA1H refers to a modified cell having a lower level of Cav3.2 protein compared to an unmodified cell. In some embodiments, an engineered cell having reduced expression of HCN4 refers to a modified cell having a lower level of HCN4 protein compared to an unmodified cell. In some embodiments, an engineered cell having reduced expression of SLC8A1 refers to a modified cell having a lower level of SLC8A1 protein compared to an unmodified cell.

In some embodiments, the engineered cell is further engineered to be hypoimmunogenic by any of the method described in Section III.

Thus, in some embodiments, the engineered cells and populations thereof exhibit reduced expression of one or more of CACNA1G, CACNA1H, HCN4, SLC8A1, B2M, and CIITA. In some embodiments, the engineered cells and populations thereof exhibit reduced expression of one or more of CACNA1G, CACNA1H, HCN4, SLC8A1, B2M, and CD74. In some embodiments, the engineered cells and populations thereof exhibit increased expression of one or more of KCNJ2, TRDN, SRL, HRC, CASQ2, and CD47. In some embodiments, the engineered cells and populations thereof exhibit reduced expression of one or more of CACNA1G, HCN4, SLC8A1, B2M, and CIITA. In some embodiments, the engineered cells and populations thereof exhibit reduced expression of one or more of CACNA1G, HCN4, SLC8A1, B2M, and CD74. In some embodiments, the engineered cells and populations thereof exhibit increased expression of one or more of KCNJ2 and CD47.

In some embodiments, the engineered cells and populations thereof exhibit reduced expression of one or more of B2M, CIITA, CACNA1G, CACNA1H, HCN4, and SLC8A1, and increased expression of one or more of CD47, KCNJ2, TRDN, SRL, HRC, and CASQ2. In some embodiments, the engineered cells and populations thereof exhibit reduced expression of one or more of B2M, CD74, CACNA1G, CACNA1H, HCN4, and SLC8A1, and increased expression of one or more of CD47, KCNJ2, TRDN, SRL, HRC, and CASQ2. In some embodiments, the engineered cells and populations thereof exhibit reduced expression of one or more of B2M, CIITA, CACNA1G, HCN4, and SLC8A1, and increased expression of one or more of CD47, KCNJ2, TRDN, SRL, HRC, and CASQ2. In some embodiments, the engineered cells and populations thereof exhibit reduced expression of one or more of B2M, CD74, CACNA1G, HCN4, and SLC8A1, and increased expression of one or more of CD47, KCNJ2, TRDN, SRL, HRC, and CASQ2. In some embodiments, the engineered cells and populations thereof exhibit reduced expression of one or more of B2M, CIITA, CACNA1G, CACNA1H, HCN4, and SLC8A1, and increased expression of one or more of KCNJ2 and CD47. In some embodiments, the engineered cells and populations thereof exhibit reduced expression of one or more of B2M, CID74, CACNA1G, CACNA1H, HCN4, and SLC8A1, and increased expression of one or more of KCNJ2 and CD47. In some embodiments, the engineered cells and populations thereof exhibit reduced expression of B2M, CIITA, CACNA1G, HCN4, and SLC8A1, and increased expression of KCNJ2 and CD47. In some embodiments, the engineered cells and populations thereof exhibit reduced expression of B2M, CD74, CACNA1G, HCN4, and SLC8A1, and increased expression of KCNJ2 and CD47.

### III. HYPOIMMUNOGENIC ENGINEERING OF CELLS

Provided herein are cells that comprise one or more modifications that regulate the expression of one or more target polynucleotide sequences, such as regulates the expression of MHC HLA class I molecules, MHC HLA class II molecules, or MHC HLA class I and MHC HLA class II molecules. One of skill in the art can appreciate that the one or more modifications that regulate expression of MCH class I molecules and/or class II molecules can be introduced into a cell prior to, simultaneously with, and/or subsequent to any of the modifications introduced into any of the cells as described in Section II. One of skill in the art can also appreciate that the one or more modifications that regulate expression of MCH class I molecules and/or class II molecules can be introduced into a PSC prior to, simultaneously with, and/or subsequent to differentiation, as described in Section VI. In some embodiments, the cells are engineered cells produced by any of the methods as described in Section II. In some embodiments, engineered cells that further comprise one or more modifications that regulate the expression of one or more target polynucleotide sequences, such as regulates the expression of MHC HLA class I molecules, MHC HLA class II molecules, or MHC HLA class I and MHC HLA class II molecules, are hypoimmunogenic.

In some embodiments, the provided cells (e.g., the engineered cells) also include one or more modifications to modulate (e.g., increase) expression of one or more tolerogenic factors. In some embodiments, the modulation of expression of the tolerogenic factor (e.g., increased expression), and the modulation of expression of the MHC HLA class I molecules and/or MHC HLA class II molecules (e.g., reduced or eliminated expression) is relative to the amount of expression of said molecule(s) in a cell of the same cell type that does not comprise the one or more modifications. In some embodiments, the modulation of expression is relative to an unmodified or a wildtype cell. In some embodiments, the unmodified or wildtype cell is a cell of the same cell type as the provided cells (e.g., the engineered cells). In some embodiments, the unmodified cell or wildtype cell expresses the one or more tolerogenic factors, the MHC HLA class I molecules, and/or the MHC HLA class II molecules. In some embodiments, the unmodified cell or wildtype cell does not express the one or more tolerogenic factors, the MHC HLA class I molecules, and/or the MHC HLA class II molecules. In some embodiments, wherein the unmodified cell or wildtype cell does not express the one or more tolerogenic factors is used to generate the engineered hypoimmune cell, the provided engineered cells include a modification to overexpress the one or more tolerogenic factors or increase the expression of the one or more tolerogenic factors from 0%. It is understood that if the cell prior to the hypoimmunogenic engineering does not express a detectable amount of the one or more tolerogenic factors, then a modification that results in any detectable amount of an expression of the one or more tolerogenic factors is an increase in the expression compared to the similar cell that does not contain the modifications.

In some embodiments, modulation of expression of the one or more tolerogenic factors (e.g., increased expression), and the modulation of expression of the MHC HLA class I molecules and/or MHC HLA class II molecules (e.g., reduced or eliminated expression) is relative to the amount of expression of said molecule(s) in a cell of the same cell type that does comprise not the modification(s). In some embodiments wherein a cell of the same cell type that does not express the one or more tolerogenic factors is used to generate the engineered hypoimmune cell, the provided engineered hypoimmune cells include a modification to overexpress the one or more tolerogenic factors or increase the expression of the one or more tolerogenic factors from 0%. It is understood that if the cell prior to the hypoimmune engineering does not express a detectable amount of the one or more tolerogenic factors, then a modification that results in any detectable amount of an expression of the one or more tolerogenic factors is an increase in the expression compared to the similar cell that does not contain the modifications.

In some embodiments, the provided cells (e.g., the engineered cells) include a modification to increase expression of one or more tolerogenic factors. One of skill in the art can appreciate that the one or more modifications that increase expression of one or more tolerogenic factors can be introduced into a cell prior to, simultaneously with, and/or subsequent to any of the modifications introduced into any of the cells as described in Section II. One of skill in the art can also appreciate that the one or more modifications that increase expression of one or more tolerogenic factors can be introduced into a PSC prior to, simultaneously with, and/or subsequent to differentiation, as described in Section VI.

In some embodiments, the one or more tolerogenic factors comprise one or more of DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, and H2-M3 (including any combination thereof). In some embodiments, the tolerogenic factor is one or more of DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). In some embodiments, the modification to increase expression of the one or more tolerogenic factors is or includes increased expression of CD47. In some embodiments, the modification to increase expression of the one or more tolerogenic factors is or includes increased expression of PD-L1. In some embodiments, the modification to increase expression of the one or more tolerogenic factors is or includes increased expression of HLA-E. In some embodiments, the modification to increase expression of the one or more tolerogenic factors is or includes increased expression of HLA-G. In some embodiments, the modification to increase expression of the one or more tolerogenic factors is or includes increased expression of CCL21, PD-L1, FasL, Serpinb9, H2-M3 (HLA-G), CD47, CD200, and Mfge8.

In some embodiments, the cells include one or more modifications, such as genetic modifications, that reduce expression of MHC HLA class I molecules and one or more modifications that increase expression of CD47. In other words, the engineered hypoimmune cells comprise exogenous CD47 protein and exhibit reduced or silenced surface expression of one or more MHC HLA class I molecules. In some embodiments, the cells include one or more genetic modifications that reduce expression of MHC HLA class II molecules and a modification that increases expression of CD47. In some instances, the engineered hypoimmune cells comprise exogenous CD47 protein and exhibit reduced or silenced surface expression of one or more MHC HLA class II molecules. In some embodiments, the engineered hypoimmune cells include one or more genetic modifications that reduce or eliminate expression of MHC HLA class I molecules, one or more genetic modifications that reduce or eliminate expression of MHC HLA class II molecules, and a modification that increases expression of CD47. In some embodiments, the engineered hypoimmune cells comprise exogenous CD47 protein, exhibit reduced or silenced surface expression of one or more MHC HLA class I molecules and exhibit reduced or lack surface expression of one or more MHC HLA class II molecules. In some embodiments, the phenotype of the cells is B2M^{indel/indel}, CIITA^{indel/indel}, CD47^{tg} cells.

In some embodiments, populations of engineered cells are provided as a therapeutic cardiac cell therapy, including for a subject having a heart disease or condition. In some embodiments, the engineered cells of the cardiac cell therapy are cardiomyocytes differentiated from PSCs or primary cardiac cells having any of the one or more modifications described in Sections II and III.

In some embodiments, the population of engineered hypoimmune cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

In some embodiments, the engineered hypoimmune cells provided herein comprise a “suicide gene” or “suicide switch”. A suicide gene or suicide switch can be incorporated to function as a “safety switch” that can cause the death of the cell, such as after the cell is administered to a subject and if they cells should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al, Mol. Therap. 20 (10): 1932-1943 (2012), Xu et al, Cell Res. 8:73-8 (1998), both incorporated herein by reference in their entirety).

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies, including those using one or more tolerogenic factors.

In some embodiments, a safety switch can be incorporated into, such as introduced, into the cells provided herein to provide the ability to induce death or apoptosis of cells containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44 (11): 1013-1019 (2016) (for iCaspase9); Wang et al., Blood 18 (5): 1255-1263 (2001) (for huEGFR); U.S. Patent Application Publication No. 20180002397 (for HER1); and Philip et al., Blood124 (8): 1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting a cell with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin inducible caspase 9 variant is called rapaCasp9. See Stavrou et al., Mal. Ther. 26 (5): 1266-1276 (2018). Thus, iCasp9 can be used as a safety switch to achieve controlled killing of the host cells.

In some embodiments, the safety switch may be an exogenously administered agent that recognizes one or more tolerogenic factors on the surface of the engineered hypoimmune cell. In some embodiments, the exogenously administered agent is an antibody directed against or specific to a tolerogenic factor, e.g. an anti-CD47 antibody. In some embodiments, the exogenously administered agent is a CD47/SIRPa binding inhibitor, such as an antibody or peptide. Exemplary antibodies include but are not limited to AO-176 (Arch); IBI188 (letaplimab) (Innovent) STI-6643 (Sorrento); and Zai: ZL-1201. See also PCT/US2021/013735 and PCT/US2021/054326, each incorporated herein by reference in their entirety. By recognizing and blocking a tolerogenic factor on the engineered cell, an exogenously administered antibody may block the immune inhibitory functions of the tolerogenic factor, thereby re-sensitizing the immune system to the engineered cells. For instance, for an engineered cell that overexpresses CD47, an exogenously administered anti-CD47 antibody may be administered to the subject, resulting in masking of CD47 on the engineered cell and triggering of an immune response to the engineered cell.

In some embodiments, the method further comprises introducing an expression vector comprising an inducible suicide switch into the cell.

In some embodiments, the engineered hypoimmune cell is derived from a source cell already comprising one or more of the desired modifications. In some embodiments, in view of the teachings provided herein one of ordinary skill in the art will readily appreciate how to assess what modifications are required to arrive at the desired final form of an engineered hypoimmune cell, and that not all reduced or increased levels of target components are achieved via active engineering. In some embodiments, the modifications of the engineered hypoimmune cell may be in any order, and not necessarily the order listed in the descriptive language provided herein.

Once altered, the presence of expression of any of the molecules described herein can be assayed using known techniques, such as Western blots, ELISA assays, FACS assays, flow cytometry, and the like.

### A. Targets Having Reduced Expression

In some embodiments, the provided engineered cells comprise one or more modifications (e.g. genetic modifications) of one or more target polynucleotide or protein sequences (also interchangeably referred to as a target gene) that regulate (e.g. reduce or eliminate) the expression of MHC HLA class I molecules, MHC HLA class II molecules, or MHC HLA class I and MHC HLA class II molecules. In some embodiments, the one or more modifications (e.g. genetic modifications) of one or more target polynucleotide or protein sequences (also interchangeably referred to as a target gene) that regulate (e.g. reduce or eliminate) the expression of MHC HLA class I molecules, MHC HLA class II molecules, or MHC HLA class I and MHC HLA class II molecules make the cell hypoimmune. In some embodiments, the cell to be modified is a cell that has not previously been introduced with the one or more modifications to make the cell hypoimmune, including any of the engineered cells described in Section II. In some embodiments, a genetic editing system is used to modify one or more target polynucleotide sequences that regulate (e.g. reduce or eliminate) the expression of MHC HLA class I molecules, MHC HLA class II molecules, or MHC HLA class I and MHC HLA class II molecules.

In certain embodiments, the genome of the cell has been altered to reduce or delete components required or involved in facilitating HLA expression, such as expression of MHC HLA class I molecules and/or MHC HLA class II molecules on the surface of the cell. For instance, in some embodiments, expression of beta-2-microgloublin (B2M), a component of MHC class I molecules, is reduced or eliminated in the cell, thereby reducing or eliminating the cell surface expression of MHC HLA class I molecules by the engineered cell. In some embodiments, expression of Transporter 1, ATP Binding Cassette Subfamily B Member (TAP1) is reduced or eliminated in the cell, thereby reducing or eliminating the expression of one or more MHC HLA class I molecules by the engineered cell. In some embodiments, expression of TAP1 is reduced or eliminated in the cell, thereby reducing or eliminating the cell surface expression of MHC HLA class I molecules by the engineered cell. Thus, in some embodiments, expression can be reduced via a gene, and/or function thereof, RNA expression and function, protein expression and function, localization (such as cell surface expression), longevity, or a combination thereof.

In some embodiments, an MHC in humans is also called a human leukocyte antigen. For instance, a human MHC class I is also known as an HLA class I and a human MHC class II is also known as an HLA class II. Thus, reference to MHC is intended to include the corresponding human HLA molecules, unless stated otherwise.

In some embodiments, reduced expression of a target is such that expression in an engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a source cell prior to being engineered to reduce expression of the target. In some embodiments, reduced expression of a target is such that expression in an engineered cell is reduced to a level that is about 60% or less (such as any of about 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a reference cell or a reference cell population (such as a cell or population of the same cell type or a cell having reduced or eliminated immunogenic response). In some embodiments, reduced expression of a target is such that expression in an engineered cell is reduced to a level that is at or less than a measured level of expression (such as a level known to exhibit reduced or eliminated immunogenic response due to the presence of the target). In some embodiments, the level of a target is assessed in an engineered cell, a reference cell, or reference cell population in a stimulated or non-stimulated state. In some embodiments, the level of a target is assessed in an engineered cell, a reference cell, or reference cell population in a stimulated state such that the target is expressed (or will be if it is a capability of the cell in response to the stimulus). In some embodiments, the stimulus represents an in vivo stimulus.

In some embodiments, the provided engineered cells comprises one or more modifications, such as genetic modifications, of one or more target polynucleotide sequences (also interchangeably referred to as a target gene) that regulate (e.g., reduce or eliminate) the expression of either MHC class I molecules, MHC class II molecules, or MHC class I molecule and MHC class II molecules. In some embodiments, an MHC in humans is also called a human leukocyte antigen. For instance, a human MHC class I molecule is also known as an HLA class I and a human MHC class II molecule is also known as an HLA class II molecule. In some embodiments, the cell to be modified is a cell that has not previously been introduced with the one or more modifications to make the cell hypoimmune, including any of the engineered cells described in Section II. In some embodiments, a genetic editing system is used to modify one or more target polynucleotide sequences that regulate the expression of either MHC class I molecules, MHC class II molecules, or MHC class I molecule and MHC class II molecules. In certain embodiments, the genome of the cell has been altered to reduce or delete components require or involved in facilitating HLA expression, such as expression of MHC class I molecule and/or MHC class II molecules on the surface of the cell. For instance, in some embodiments, B2M, a component of MHC class I molecules, is reduced or eliminated in the cell, thereby reducing or eliminating the cell surface expression of MHC HLA class I molecules by the engineered cell. In some embodiments, TAP1 is reduced or eliminated in the cell, thereby reducing or eliminating the expression of MHC class I molecules by the engineered cell. In some embodiments, TAP1 is reduced or eliminated in the cell, thereby reducing or eliminating the cell surface expression of MHC class I molecules by the engineered cell.

In some embodiments, any of the described modifications in the engineered hypoimmune cell that regulate (e.g. reduce or eliminate) expression of one or more target polynucleotide or protein in the engineered cell may be combined together with one or more modifications to overexpress a polynucleotide (e.g. a tolerogenic factor, such as CD47) described in Section IV.B.3.

In some embodiments, reduction of MHC class I molecule and/or MHC class II molecule expression can be accomplished, for example, by one or more of the following: (1) targeting the polymorphic HLA alleles (HLA-A, HLA-B, HLA-C) and MHC class II molecule genes directly (HLA-DP, HLA-DQ, HLA-DR); (2) removal of B2M, which will reduce surface trafficking of all MHC class I molecules; (3) removal of TAP1, which will disrupt the expression of HLA-A, -B, and -C genes; (4) deletion of one or more components of the MHC enhanceosomes, such as LRC5, RFX-5, RFXANK, RFXAP, IRFI, NF-Y (including NFY-A, NFY-B, NFY-C), CD74, and CIITA that are critical for HLA expression; and/or (5) removal of B2M, which will reduce surface trafficking of all MHC class I molecules.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of TAP1, NLRC5, CIITA, CD74, RFX5, RFXAP, RFXANK, NFY-A, NFY-B, NFY-C and/or IRF-1), blocking surface trafficking of MHC class I molecules (e.g., knocking out expression of B2M), and/or targeting with HLA-Razor (see, e.g., WO2016183041).

Beta-2-microglobulin (B2-microglobulin) is a component of MHC (major histocompatibility complex) class I molecules. B2M associates with MHC class I molecules HLA-A, HLA-B and HLA-C at the cell surface under normal conditions. Thus, in some embodiments, decreasing expression of, or knocking out, B2M interferes with surface trafficking of MHC class I molecules, such that surface expression of MHC class I molecules (HLA-A, HLA-B and HLA-C) is reduced or decreased. Thus, in some embodiments, reducing expression of MHC class I molecules is achieved by reducing surface trafficking of MHC class I molecules (HLA-A, HLA-B and HLA-C), such as by reducing expression of, or knocking out, B2M.

In some embodiments, decreasing or eliminating expression of B2M decreases the expression of MHC class I molecules by reducing surface trafficking of HLA-A, HLA-B, and HLA-C.

TAP1 is a transcriptional regulator. When TAP1 is disrupted, expression of MHC class I genes (HLA-A, HLA-B, and HLA-C) is similarly disrupted. Thus, in some embodiments, decreasing expression of, or knocking out, TAP1 interferes with expression of MHC class I genes, such that expression of MHC class I molecules (HLA-A, HLA-B and HLA-C) is reduced or decreased by virtue of reduced expression of the genes encoding the same. Thus, in some embodiments, reducing expression of MHC class I molecules is achieved by reducing expression of MHC class I encoding genes, such as by reducing expression of, or knocking out, TAP1.

In some embodiments, decreasing or eliminating expression of TAP1 decreases the expression of genes encoding MHC class I molecules by reducing expression of such genes.

CIITA is a master regulator of MHC class II gene expression. When CIITA is disrupted, expression of MHC class I genes (HLA-A, HLA-B, and HLA-C) is similarly disrupted. Thus, in some embodiments, decreasing expression of, or knocking out, CIITA interferes with expression of MHC class II genes, such that expression of MHC class II molecules is reduced or decreased by virtue of reduced expression of the genes encoding the same. Thus, in some embodiments, reducing expression of MHC class II molecules is achieved by reducing expression of MHC class II encoding genes, such as by reducing expression of, or knocking out, CIITA.

In some embodiments, decreasing or eliminating expression of CIITA decreases the expression of genes encoding MHC class II molecules by reducing expression of such genes.

In certain aspects, the engineered hypoimmune cells disclosed herein do not express one or more human leukocyte antigens (e.g., HLA-A, HLA-B and/or HLA-C) corresponding to MHC class I molecule and/or MHC class II molecule and are thus characterized as being hypoimmunogenic. For example, in certain aspects, the engineered hypoimmune cells disclosed herein have been modified such that the cells do not express, or exhibit reduced expression of, one or more of the following MHC class I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, the expression of MHC class I molecules and/or MHC class II molecules is modulated by targeting and deleting a contiguous stretch of genomic DNA, thereby reducing or eliminating expression of a target gene selected from the group consisting of B2M, TAP1, CIITA, CD74, and NLRC5.

In some embodiments, the provided engineered cells comprise a modification, such as a genetic modification, of one or more target polynucleotide sequence that regulate MHC class I. Exemplary methods for reducing expression of MHC class I molecule are described in sections below. In some embodiments, the targeted polynucleotide sequence is one or both of B2M and NLRC5. In some embodiments, the cell comprises a genetic editing modification to the B2M gene. In some embodiments, the cell comprises a genetic editing modification to the NLRC5 gene. In some embodiments, the cell comprises a genetic editing modification to the TAP1 gene. In some embodiments, the cell comprises genetic editing modifications to the B2M and CIITA genes. In some embodiments, the cell comprises genetic editing modifications to the TAP1 and CIITA genes. In some embodiments, the cell comprises genetic editing modifications to the B2M and CD74 genes. In some embodiments, the cell comprises genetic editing modifications to the TAP1 and CD74 genes.

In some embodiments, the provided engineered hypoimmune cells comprise a modification, such as a genetic modification, of one or more target polynucleotide sequence that regulate MHC class II molecule. Exemplary methods for reducing expression of MHC class II molecule are described in sections below.

In some embodiments, the cell comprises a genetic editing modification to the CIITA gene.

In some embodiments, the provided engineered hypoimmune cells comprise one or more modifications, such as genetic modifications, of one or more target polynucleotide sequence that regulate MHC class I molecules and MHC class II molecules. Exemplary methods for reducing expression of MHC class I molecules and MHC class II molecules are described in sections below. In some embodiments, the cell comprises genetic editing modifications to the B2M and NLRC5 genes. In some embodiments, the cell comprises genetic editing modifications to the CIITA and NLRC5 genes. In particular embodiments, the cell comprises genetic editing modifications to the B2M, CIITA and NLRC5 genes. In some embodiments, the cell comprises genetic editing modifications to the TAP1 and NLRC5 genes. In some embodiments, the cell comprises genetic editing modifications to the CIITA and NLRC5 genes. In particular embodiments, the cell comprises genetic editing modifications to the TAP1, CIITA and NLRC5 genes.

In some embodiments, the modification that reduces B2M, TAP1, CIITA and/or NLRC5 expression reduces B2M, TAP1, CIITA and/or NLRC5 mRNA expression. In some embodiments, the reduced mRNA expression of B2M, TAP1, CIITA and/or NLRC5 is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of TAP1 is reduced by more than about 5%, such as reduced by more than about any of 10%, 20%, 30%, 40%, 50%, 60%, 70%, 80%, 90%, or more. In some embodiments, the mRNA expression of B2M, TAP1, CIITA and/or NLRC5 is reduced by up to about 100%, such as reduced by up to about any of 90%, 80%, 70%, 60%, 50%, 40%, 30%, 20%, 10%, 5%, or less. In some embodiments, the mRNA expression of B2M, CIITA and/or NLRC5 is reduced by any of about 5%, 10%, 20%, 30%, 40%, 50%, 60%, 70%, 80%, 90%, or 100%. In some embodiments, the mRNA expression of B2M, CIITA and/or NLRC5 is eliminated (e.g., 0% expression of B2M, CIITA and/or NLRC5 mRNA). In some embodiments, the modification that reduces B2M, CIITA and/or NLRC5 mRNA expression eliminates B2M, CIITA and/or NLRC5 gene activity. In some embodiments, the mRNA expression of TAP1, CIITA and/or NLRC5 is reduced by any of about 5%, 10%, 20%, 30%, 40%, 50%, 60%, 70%, 80%, 90%, or 100%. In some embodiments, the mRNA expression of TAP1, CIITA and/or NLRC5 is eliminated (e.g., 0% expression of TAP1, CIITA and/or NLRC5 mRNA). In some embodiments, the modification that reduces TAP1, CIITA and/or NLRC5 mRNA expression eliminates TAP1, CIITA and/or NLRC5 gene activity.

In some embodiments, the modification that reduces B2M, TAP1, CIITA and/or NLRC5 expression reduces B2M, TAP1, CIITA and/or NLRC5 protein expression. In some embodiments, the reduced protein expression of B2M, TAP1, CIITA and/or NLRC5 is relative to an unmodified or wild-type cell of the same cell type that does not comprise the modification. In some embodiments, the protein expression of B2M, TAP1, CIITA and/or NLRC5 is reduced by more than about 5%, such as reduced by more than about any of 10%, 20%, 30%, 40%, 50%, 60%, 70%, 80%, 90%, or more. In some embodiments, the protein expression of B2M, TAP1, CIITA and/or NLRC5 is reduced by up to about 100%, such as reduced by up to about any of 90%, 80%, 70%, 60%, 50%, 40%, 30%, 20%, 10%, 5%, or less. In some embodiments, the protein expression of B2M, TAP1, CIITA and/or NLRC5 is reduced by any of about 5%, 10%, 20%, 30%, 40%, 50%, 60%, 70%, 80%, 90%, or 100%. In some embodiments, the protein expression of B2M, TAP1, CIITA and/or NLRC5 is eliminated (e.g., 0% expression of B2M, CIITA and/or NLRC5 protein). In some embodiments, the modification that reduces B2M, TAP1, CIITA and/or NLRC5 protein expression eliminates B2M, CIITA and/or NLRC5 gene activity.

In some embodiments, the modification that reduces B2M, TAP1, CIITA and/or NLRC5 expression comprises inactivation or disruption of the B2M, TAP1, CIITA and/or NLRC5 gene. In some embodiments, the modification that reduces B2M, TAP1, CIITA and/or NLRC5 expression comprises inactivation or disruption of one allele of the B2M, TAP1, CIITA and/or NLRC5 gene. In some embodiments, the modification that reduces B2M, TAP1, CIITA and/or NLRC5 expression comprises inactivation or disruption comprises inactivation or disruption of both alleles of the B2M, TAP1, CIITA and/or NLRC5 gene.

In some embodiments, the modification comprises inactivation or disruption of one or more B2M, TAP1, CIITA and/or NLRC5 coding sequences in the cell. In some embodiments, the modification comprises inactivation or disruption of all B2M, TAP1, CIITA and/or NLRC5 coding sequences in the cell. In some embodiments, the inactivation or disruption comprises an indel in the B2M, TAP1, CIITA and/or NLRC5 gene. In some embodiments, the modification is a frameshift mutation of genomic DNA of the B2M, TAP1, CIITA and/or NLRC5 gene. In some embodiments, the modification is a deletion of genomic DNA of the B2M, TAP1, CIITA and/or NLRC5 gene. In some embodiments, the modification is a deletion of a contiguous stretch of genomic DNA of the B2M, TAP1, CIITA and/or NLRC5 gene. In some embodiments, the B2M, TAP1, CIITA and/or NLRC5 gene is knocked out.

In some embodiments, the cell comprises a genetic editing modification to the CD74 gene.

In some embodiments, the provided engineered hypoimmune cells comprise one or more modifications, such as genetic modifications, of one or more target polynucleotide sequence that regulate MHC class I molecules and MHC class II molecules. Exemplary methods for reducing expression of MHC class I molecules and MHC class II molecules are described in sections below. In some embodiments, the cell comprises genetic editing modifications to the B2M and NLRC5 genes. In some embodiments, the cell comprises genetic editing modifications to the CD74 and NLRC5 genes. In particular embodiments, the cell comprises genetic editing modifications to the B2M, CD74 and NLRC5 genes. In some embodiments, the cell comprises genetic editing modifications to the TAP1 and NLRC5 genes. In some embodiments, the cell comprises genetic editing modifications to the CD74 and NLRC5 genes. In particular embodiments, the cell comprises genetic editing modifications to the TAP1, CD74 and NLRC5 genes.

In some embodiments, the modification that reduces B2M, TAP1, CD74 and/or NLRC5 expression reduces B2M, TAP1, CD74 and/or NLRC5 mRNA expression. In some embodiments, the reduced mRNA expression of B2M, TAP1, CD74 and/or NLRC5 is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of TAP1 is reduced by more than about 5%, such as reduced by more than about any of 10%, 20%, 30%, 40%, 50%, 60%, 70%, 80%, 90%, or more. In some embodiments, the mRNA expression of B2M, TAP1, CD74 and/or NLRC5 is reduced by up to about 100%, such as reduced by up to about any of 90%, 80%, 70%, 60%, 50%, 40%, 30%, 20%, 10%, 5%, or less. In some embodiments, the mRNA expression of B2M, CD74 and/or NLRC5 is reduced by any of about 5%, 10%, 20%, 30%, 40%, 50%, 60%, 70%, 80%, 90%, or 100%. In some embodiments, the mRNA expression of B2M, CD74 and/or NLRC5 is eliminated (e.g., 0% expression of B2M, CD74 and/or NLRC5 mRNA). In some embodiments, the modification that reduces B2M, CD74 and/or NLRC5 mRNA expression eliminates B2M, CD74 and/or NLRC5 gene activity. In some embodiments, the mRNA expression of TAP1, CD74 and/or NLRC5 is reduced by any of about 5%, 10%, 20%, 30%, 40%, 50%, 60%, 70%, 80%, 90%, or 100%. In some embodiments, the mRNA expression of TAP1, CD74 and/or NLRC5 is eliminated (e.g., 0% expression of TAP1, CD74 and/or NLRC5 mRNA). In some embodiments, the modification that reduces TAP1, CD74 and/or NLRC5 mRNA expression eliminates TAP1, CD74 and/or NLRC5 gene activity.

In some embodiments, the modification that reduces B2M, TAP1, CD74 and/or NLRC5 expression reduces B2M, TAP1, CD74 and/or NLRC5 protein expression. In some embodiments, the reduced protein expression of B2M, TAP1, CD74 and/or NLRC5 is relative to an unmodified or wild-type cell of the same cell type that does not comprise the modification. In some embodiments, the protein expression of B2M, TAP1, CD74 and/or NLRC5 is reduced by more than about 5%, such as reduced by more than about any of 10%, 20%, 30%, 40%, 50%, 60%, 70%, 80%, 90%, or more. In some embodiments, the protein expression of B2M, TAP1, CD74 and/or NLRC5 is reduced by up to about 100%, such as reduced by up to about any of 90%, 80%, 70%, 60%, 50%, 40%, 30%, 20%, 10%, 5%, or less. In some embodiments, the protein expression of B2M, TAP1, CD74 and/or NLRC5 is reduced by any of about 5%, 10%, 20%, 30%, 40%, 50%, 60%, 70%, 80%, 90%, or 100%. In some embodiments, the protein expression of B2M, TAP1, CD74 and/or NLRC5 is eliminated (e.g., 0% expression of B2M, CD74 and/or NLRC5 protein). In some embodiments, the modification that reduces B2M, TAP1, CD74 and/or NLRC5 protein expression eliminates B2M, CD74 and/or NLRC5 gene activity.

In some embodiments, the modification that reduces B2M, TAP1, CD74 and/or NLRC5 expression comprises inactivation or disruption of the B2M, TAP1, CD74 and/or NLRC5 gene. In some embodiments, the modification that reduces B2M, TAP1, CD74 and/or NLRC5 expression comprises inactivation or disruption of one allele of the B2M, TAP1, CD74 and/or NLRC5 gene. In some embodiments, the modification that reduces B2M, TAP1, CD74 and/or NLRC5 expression comprises inactivation or disruption comprises inactivation or disruption of both alleles of the B2M, TAP1, CD74 and/or NLRC5 gene.

In some embodiments, the modification comprises inactivation or disruption of one or more B2M, TAP1, CD74 and/or NLRC5 coding sequences in the cell. In some embodiments, the modification comprises inactivation or disruption of all B2M, TAP1, CD74 and/or NLRC5 coding sequences in the cell. In some embodiments, the inactivation or disruption comprises an indel in the B2M, TAP1, CD74 and/or NLRC5 gene. In some embodiments, the modification is a frameshift mutation of genomic DNA of the B2M, TAP1, CD74 and/or NLRC5 gene. In some embodiments, the modification is a deletion of genomic DNA of the B2M, TAP1, CD74 and/or NLRC5 gene. In some embodiments, the modification is a deletion of a contiguous stretch of genomic DNA of the B2M, TAP1, CD74 and/or NLRC5 gene. In some embodiments, the B2M, TAP1, CD74 and/or NLRC5 gene is knocked out.

In some embodiments, the engineered hypoimmune cell comprises reduced expression of MHC class I, or a component thereof, wherein reduced is as described herein, such as relative to prior to engineering to reduce expression of MHC class I molecule or a component thereof, a reference cell or a reference cell population (such as a cell having a desired lack of an immunogenic response), or a measured value.

In some embodiments, the engineered hypoimmune cell is engineered to reduce cell surface expression of the MHC class I molecule polypeptide, or a component thereof (such as B2M). In some embodiments, cell surface expression of the MHC class I molecule polypeptide, or a component thereof (such as B2M), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as B2M), cell surface expression prior to being engineered to reduce cell surface presentation of the MHC class I molecule polypeptide, or a component thereof (such as B2M). In some embodiments, cell surface expression of the MHC class I molecule polypeptide, or a component thereof (such as B2M), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as B2M), cell surface expression on a reference cell or a reference cell population (such as an average amount of MHC class I molecule polypeptide, or a component thereof (such as B2M), cell surface expression). In some embodiments, there is no cell surface presentation of the MHC class I molecule polypeptide, or a component thereof (such as B2M), on the engineered cell (including no detectable cell surface expression, including as measured using known techniques, e.g., flow cytometry). In some embodiments, the engineered cell exhibits reduced protein expression of the MHC class I molecule polypeptide, or a component thereof (such as B2M). In some embodiments, protein expression of the MHC class I molecule polypeptide, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as B2M), protein expression prior to being engineered to reduce protein expression of the MHC class I molecule polypeptide, or a component thereof (such as B2M). In some embodiments, protein expression of the MHC class I molecule polypeptide, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as B2M), prior to being engineered to reduce protein expression of the MHC class I molecule polypeptide, or a component thereof (such as B2M). In some embodiments, the engineered cell exhibits no protein expression of the MHC class I molecule polypeptide, or a component thereof (such as B2M), (including no detectable protein expression, including as measured using known techniques, e.g., western blot or mass spectrometry). In some embodiments, the engineered cell does not comprise the MHC class I molecule polypeptide, or a component thereof (such as B2M) (including no detectable protein, including as measured using known techniques, e.g., western blot or mass spectrometry). In some embodiments, the engineered cell exhibits reduced mRNA expression encoding the MHC class I molecule polypeptide, or a component thereof (such as B2M). In some embodiments, mRNA expression encoding the MHC class I molecule polypeptide, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the MHC class I molecule polypeptide, or a component thereof (such as B2M), prior to being engineered to reduce mRNA expression of the MHC class I molecule polypeptide, or a component thereof (such as B2M). In some embodiments, mRNA expression encoding the MHC class I molecule polypeptide, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding a MHC class I molecule polypeptide, or a component thereof (including no detectable mRNA expression, including as measured using known techniques, e.g., sequencing techniques or PCR). In some embodiments, the engineered cell does not comprise mRNA encoding a MHC class I molecule polypeptide, or a component thereof (including no detectable mRNA, including as measured using known techniques, e.g., sequencing techniques or PCR). In some embodiments, the engineered cell comprises a gene inactivation or disruption of the MHC class I molecule gene. In some embodiments, the engineered cell comprises a gene inactivation or disruption of the MHC class I molecule gene in both alleles. In some embodiments, the engineered cell comprises a gene inactivation or disruption of the MHC class I molecule gene in all alleles. In some embodiments, the engineered cell is a MHC class I molecule knockout or a MHC class I molecule component (such as B2M) knockout.

In some embodiments, the engineered hypoimmune cell is engineered to reduce cell surface expression of the MHC class I molecule polypeptide, or a component thereof (such as TAP1). In some embodiments, cell surface expression of the MHC class I molecule polypeptide, or a component thereof (such as TAP1), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as TAP1), cell surface expression prior to being engineered to reduce cell surface presentation of the MHC class I molecule polypeptide, or a component thereof (such as TAP1). In some embodiments, cell surface expression of the MHC class I molecule polypeptide, or a component thereof (such as TAP1), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as TAP1), cell surface expression on a reference cell or a reference cell population (such as an average amount of MHC class I molecule polypeptide, or a component thereof (such as TAP1), cell surface expression). In some embodiments, there is no cell surface presentation of the MHC class I molecule polypeptide, or a component thereof (such as TAP1), on the engineered cell (including no detectable cell surface expression, including as measured using known techniques, e.g., flow cytometry). In some embodiments, the engineered cell hypoimmune exhibits reduced protein expression of the MHC class I molecule polypeptide, or a component thereof (such as TAP1). In some embodiments, protein expression of the MHC class I molecule polypeptide, or a component thereof (such as TAP1), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as TAP1), protein expression prior to being engineered to reduce protein expression of the MHC class I molecule polypeptide, or a component thereof (such as TAP1). In some embodiments, protein expression of the MHC class I molecule polypeptide, or a component thereof (such as TAP1), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class I molecule polypeptide, or a component thereof (such as TAP1), prior to being engineered to reduce protein expression of the MHC class I molecule polypeptide, or a component thereof (such as TAP1). In some embodiments, the engineered cell exhibits no protein expression of the MHC class I molecule polypeptide, or a component thereof (such as TAP1), (including no detectable protein expression, including as measured using known techniques, e.g., western blot or mass spectrometry). In some embodiments, the engineered cell does not comprise the MHC class I molecule polypeptide, or a component thereof (such as TAP1) (including no detectable protein, including as measured using known techniques, e.g., western blot or mass spectrometry). In some embodiments, the engineered cell exhibits reduced mRNA expression encoding the MHC class I molecule polypeptide, or a component thereof (such as TAP1). In some embodiments, mRNA expression encoding the MHC class I molecule polypeptide, or a component thereof (such as TAP1), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the MHC class I molecule polypeptide, or a component thereof (such as TAP1), prior to being engineered to reduce mRNA expression of the MHC class I molecule polypeptide, or a component thereof (such as TAP1). In some embodiments, mRNA expression encoding the MHC class I molecule polypeptide, or a component thereof (such as TAP1), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding a MHC class I molecule polypeptide, or a component thereof (including no detectable mRNA expression, including as measured using known techniques, e.g., sequencing techniques or PCR). In some embodiments, the engineered cell does not comprise mRNA encoding a MHC class I molecule polypeptide, or a component thereof (including no detectable mRNA, including as measured using known techniques, e.g., sequencing techniques or PCR). In some embodiments, the engineered cell comprises a gene inactivation or disruption of the MHC class I molecule gene. In some embodiments, the engineered cell comprises a gene inactivation or disruption of the MHC class I molecule gene in both alleles. In some embodiments, the engineered cell comprises a gene inactivation or disruption of the MHC class I molecule gene in all alleles. In some embodiments, the engineered cell is a MHC class I molecule knockout or a MHC class I molecule component (such as TAP1) knockout.

In some embodiments, the engineered hypoimmune cell comprises reduced expression of MHC class II molecule, wherein reduced is as described herein, such as relative to prior to engineering to reduce MHC class II molecule expression, a reference cell or a reference cell population (such as a cell having a desired lack of an immunogenic response), or a measured value. In some embodiments, the engineered cell is engineered to reduced cell surface expression of the MHC class II molecule polypeptide. In some embodiments, cell surface expression of the MHC class II molecule polypeptide on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class II molecule polypeptide cell surface expression prior to being engineered to reduce cell surface presentation of the MHC class II molecule polypeptide. In some embodiments, cell surface expression of the MHC class II molecule polypeptide on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class II molecule polypeptide cell surface expression on a reference cell or a reference cell population (such as an average amount of MHC class II molecule polypeptide cell surface expression). In some embodiments, there is no cell surface presentation of the MHC class II molecule polypeptide on the engineered cell (including no detectable cell surface expression, including as measured using known techniques, e.g., flow cytometry). In some embodiments, the engineered cell exhibits reduced protein expression of the MHC class II molecule polypeptide. In some embodiments, protein expression of the MHC class II molecule polypeptide of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class II molecule polypeptide protein expression prior to being engineered to reduce protein expression of the MHC class II molecule polypeptide. In some embodiments, protein expression of the MHC class II molecule polypeptide of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the MHC class II molecule polypeptide prior to being engineered to reduce protein expression of the MHC class II molecule polypeptide. In some embodiments, the engineered cell exhibits no protein expression of the MHC class II molecule polypeptide (including no detectable protein expression, including as measured using known techniques, e.g., western blot or mass spectrometry). In some embodiments, the engineered cell does not comprise the MHC class II molecule polypeptide (including no detectable protein, including as measured using known techniques, e.g., western blot or mass spectrometry). In some embodiments, the engineered cell exhibits reduced mRNA expression encoding the MHC class II molecule polypeptide. In some embodiments, mRNA expression encoding the MHC class II molecule polypeptide of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the MHC class II molecule polypeptide prior to being engineered to reduce mRNA expression of the MHC class II molecule polypeptide. In some embodiments, mRNA expression encoding the MHC class II molecule polypeptide of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding a MHC class II molecule polypeptide (including no detectable mRNA expression, including as measured using known techniques, e.g., sequencing techniques or PCR). In some embodiments, the engineered cell does not comprise mRNA encoding a MHC class II molecule polypeptide (including no detectable mRNA, including as measured using known techniques, e.g., sequencing techniques or PCR). In some embodiments, the engineered cell comprises a gene inactivation or disruption of the MHC class II molecule gene. In some embodiments, the engineered cell comprises a gene inactivation or disruption of the MHC class II molecule gene in both alleles. In some embodiments, the engineered cell comprises a gene inactivation or disruption of the MHC class II molecule in all alleles. In some embodiments, the engineered cell is a MHC class II molecule knockout.

### B. Targets Having Increased Expression

In some embodiments, the provided engineered cells are genetically modified or engineered, such as by introduction of one or more modifications into a cell to overexpress a desired polynucleotide in the cell. In some embodiments, the engineered cell to be modified or engineered is an unmodified cell or non-engineered cell that has not previously been introduced with the one or more modifications, including any of the engineered cells described in Section II. In some embodiments, the engineered cells provided herein are genetically modified to include one or more exogenous polynucleotides encoding an exogenous protein (also interchangeably used with the term “transgene”). As described, in some embodiments, the cells are modified to increase expression of certain genes that are tolerogenic (e.g., immune) factors that affect immune recognition and tolerance in a recipient. In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

The one or more polynucleotides, e.g. exogenous polynucleotides, may be expressed (e.g. overexpressed) in the engineered cell together with one or more genetic modifications to reduce expression of a target polynucleotide described in Section III.A above, such as an MHC class I molecules and/or II molecule. In some embodiments, the provided engineered cells do not trigger or activate an immune response upon administration to a recipient subject.

In some embodiments, the engineered cell includes 1, 2, 3, 4, 5, 6, 7, 8, 9, 10 or more different overexpressed polynucleotides. In some embodiments, the engineered cell includes 1, 2, 3, 4, 5, 6, 7, 8, 9, 10 or more different overexpressed polynucleotides. In some embodiments, the overexpressed polynucleotide is an exogenous polynucleotide. In some embodiments, the engineered cell includes 1, 2, 3, 4, 5, 6, 7, 8, 9, 10 or more different exogenous polynucleotides. In some embodiments, the engineered cell includes 1, 2, 3, 4, 5, 6, 7, 8, 9, 10 or more different exogenous polynucleotides. In some embodiments, the overexpressed polynucleotide is an exogenous polynucleotide that is inserted or integrated into one or more genomic loci of the engineered cell.

In some embodiments, expression of a polynucleotide is increased, i.e. the polynucleotide is overexpressed, using a fusion protein containing a DNA-targeting domain and a transcriptional activator. Targeted methods of increasing expression using transactivator domains are known to a skilled artisan.

In some embodiments, engineered cell contains one or more exogenous polynucleotides in which the one or more exogenous polynucleotides are inserted or integrated into a genomic locus of the cell by non-targeted insertion methods, such as by transduction with a lentiviral vector. In some embodiments, the lentiviral vector comprises a piggyBac transposon. During transposition, the piggyback transposon recognizes transposon-specific inverted terminal repeats (ITRs) in a lentiviral vector, to allow for the efficient movement and integration of the vector contents into TTAA chromosomal sites. In some embodiments, the one or more exogenous polynucleotides are inserted or integrated into the genome of the cell by targeted insertion methods, such as by using homology directed repair (HDR). Any suitable method can be used to insert the exogenous polynucleotide into the genomic locus of the engineered cell by HDR including the gene editing methods described herein (e.g., a CRISPR/Cas system). In some embodiments, the one or more exogenous polynucleotides are inserted into one or more genomic locus, such as any genomic locus described herein (e.g. Table 4). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g. Table 4). In some embodiments, two or more exogenous polynucleotides are inserted into a different genomic loci, such as two or more genomic loci as described herein (e.g., Table 4).

Exemplary polynucleotides or overexpression, and methods for overexpressing the same, are described in the following subsections.

### C. Exemplary Embodiments of Engineered Hypoimmunogenic Cells

In some embodiments, the engineered cells and populations thereof are engineered PSCs, cardiomyocytes differentiated therefrom, or primary cardiac cells. In some embodiments, the engineered cell is a human cell or an animal cell. In some embodiments, the engineered cell is a cell isolated from a donor subject (e.g., a healthy donor subject not suspected of having a disease or condition at the time the donor sample is obtained from the individual donor subject). In some embodiments, the engineered is selected from a pluripotent stem cell (e.g., an embryonic stem cell or an induced pluripotent stem cell), a cardiomyocyte differentiated therefrom, or a primary cardiac cell (e.g., a primary cardiomyocyte). In some embodiments, the engineered cell is a PSC. In some embodiments, the PSC is an ESC. In some embodiments, the PSC is an iPSC. In some embodiments, the engineered cell is a cardiomyocyte differentiated from a PSC, including by any of the methods described in Section VI. In some embodiments, the engineered cell is any of the cells described in Section II. In some embodiments, the engineered cell is a primary cardiac cell. In some embodiments, the primary cardiac cell is a primary cardiomyocyte.

In some embodiments, the engineered cells and populations thereof exhibit increased expression of CD47, and reduced expression of one or more molecules of the MHC class I complex and/or MHC class II complex. In some embodiments, the engineered cells and populations thereof exhibit increased expression of CD47 and reduced expression of one or more molecules of the MHC class II complex. In some embodiments, the engineered cells and populations thereof exhibit increased expression of CD47 and reduced expression of one or more molecules of the MHC class II and MHC class II complexes.

In some embodiments, the engineered cells and populations thereof exhibit increased expression of CD47 and reduced expression of B2M. In some embodiments, the cells and populations thereof exhibit increased expression of CD47 and reduced expression of CIITA. In some embodiments, the cells and populations thereof exhibit increased expression of CD47 and reduced expression of CD74. In some embodiments, the cells and populations thereof exhibit increased expression of CD47 and reduced expression of NLRC5. In some embodiments, the cells and populations thereof exhibit increased expression of CD47 and reduced expression of one or more molecules of B2M and CIITA. In some embodiments, the cells and populations thereof exhibit increased expression of CD47 and reduced expression of one or more molecules of B2M and CD74. In some embodiments, the cells and populations thereof exhibit increased expression of CD47 and reduced expression of one or more molecules of B2M and NLRC5. In some embodiments, the cells and populations thereof exhibit increased expression of CD47 and reduced expression of one or more molecules of CIITA and NLRC5. In some embodiments, the cells and populations thereof exhibit increased expression of CD47 and reduced expression of one or more molecules of B2M, CIITA and NLRC5. In some embodiments, the cells and populations thereof exhibit increased expression of CD47 and reduced expression of one or more molecules of B2M, CD74 and NLRC5. Any of the engineered cells described herein can also exhibit increased expression of one or more factors selected from the group including, but not limited to, CD47, CD35, CD16 Fc receptor, CD16, CD52, IL15-RF, H2-M3, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

In some embodiments, the engineered cells and populations thereof exhibit increased expression of CD47, optionally at least one other tolerogenic factor, and reduced expression of one or more molecules of the MHC class I complex. In some embodiments, the engineered cells and populations thereof exhibit increased expression of CD47, optionally at least one other tolerogenic factor, and reduced expression of one or more molecules of the MHC class II complex. In some embodiments, the engineered cells and populations thereof exhibit increased expression of CD47, optionally at least one other tolerogenic factor, and reduced expression of one or more molecules of the MHC class II and MHC class II complexes. In some embodiments, the engineered cells and populations thereof exhibit increased expression of CD47, optionally at least one other tolerogenic factor, and reduced expression of B2M. In some embodiments, the engineered cells and populations thereof exhibit increased expression of CD47 optionally, at least one other tolerogenic factor, and reduced expression of CIITA. In some embodiments, the engineered cells and populations thereof exhibit increased expression of CD47, optionally at least one other tolerogenic factor, and reduced expression of one or more molecules of B2M and CIITA. In some embodiments, the cells and populations thereof exhibit increased expression of CD47 and at least one other tolerogenic factor, and reduced expression of one or more molecules of B2M and NLRC5. In some embodiments, the cells and populations thereof exhibit increased expression of CD47 and at least one other tolerogenic factor, and reduced expression of one or more molecules of CIITA and NLRC5. In some embodiments, the cells and populations thereof exhibit increased expression of CD47 and at least one other tolerogenic factor, and reduced expression of one or more molecules of B2M, CIITA and NLRC5. In some embodiments, a tolerogenic factor includes any from the group including, but not limited to CD47, CD35, CD16 Fc receptor, CD16, CD52, IL15-RF, H2-M3, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9).

In some embodiments, the engineered cells and populations thereof exhibit overexpression of CD47, reduced expression B2M, and reduced expression of CIITA. In some embodiments, the reduced expression of B2M comprises reduced protein expression of B2M. In some embodiments, the reduced expression of B2M comprises reduced protein expression of B2M. In some embodiments, the reduced expression of B2M comprises eliminated protein expression of B2M. In some embodiments, the reduced expression of B2M comprises inactivation or disruption of both alleles of the B2M gene. In some embodiments, the reduced expression of B2M comprises inactivation or disruption of all B2M coding sequences in the cell. In some embodiments, the inactivation or disruption of B2M comprises an indel in the B2M gene or a deletion of a contiguous stretch of genomic DNA of the B2M gene. In some embodiments, the B2M gene is knocked out. In some embodiments, the reduced expression of CIITA comprises reduced protein expression of CIITA. In some embodiments, the reduced expression of CIITA comprises eliminated protein expression of CIITA. In some embodiments, the reduced expression of CIITA comprises inactivation or disruption of both alleles of the CIITA gene. In some embodiments, the reduced expression of CIITA comprises inactivation or disruption of all CIITA coding sequences in the cell. In some embodiments, the inactivation or disruption of CIITA comprises an indel in the CIITA gene or a deletion of a contiguous stretch of genomic DNA of the CIITA gene. In some embodiments, the CIITA gene is knocked out. In some embodiments, the modification(s) that increase expression comprise increased surface expression, and/or the modifications that reduce expression comprise reduced surface expression.

One skilled in the art will appreciate that levels of expression such as increased (e.g., overexpression) or reduced expression of a gene, protein or molecule can be referenced or compared to a comparable cell. In some embodiments, an engineered cell having increased expression of CD47 refers to a modified cell having a higher level of CD47 protein compared to an unmodified cell. In some embodiments, an engineered cell having reduced expression of B2M refers to a modified cell having a lower level of B2M protein compared to an unmodified cell. In some embodiments, an engineered cell having reduced expression of CIITA refers to a modified cell having a lower level of CIITA protein compared to an unmodified cell.

In one embodiment, provided herein are engineered cells expressing exogenous CD47 polypeptides and having reduced expression of either one or more MHC class I complex proteins, one or more MHC class II complex proteins, or any combination of MHC class I and class II complex proteins. In another embodiment, the engineered cells express exogenous CD47 polypeptides and express reduced levels of B2M and CIITA polypeptides. In some embodiments, the engineered cells express exogenous CD47 polypeptides and possess modifications (e.g., genetic modifications) of the B2M and CIITA genes. In some instances, the modifications (e.g., genetic modifications) inactivate the B2M and CIITA genes.

In another embodiment, the engineered cells express exogenous CD47 polypeptides and express reduced levels of B2M and CD74 polypeptides. In some embodiments, the engineered cells express exogenous CD47 polypeptides and possess modifications (e.g., genetic modifications) of the B2M and CD74 genes. In some instances, the modifications (e.g., genetic modifications) inactivate the B2M and CD74 genes.

In some embodiments, the cell is further engineered to reduce or prevent engraftment arrhythmia as described in Section II, and can be used to treat a variety of indications with cell therapy, including any as described herein. In some embodiments, the engineered cell can be used to treat a heart disease or condition. In some embodiments, the heart disease or condition is myocardial infarction (MI).

Thus, in some embodiments, the engineered therapeutic cells (e.g., engineered primary cardiac cells or engineered cardiomyocytes differentiated from iPSCs) are also engineered to reduce or prevent engraftment arrhythmia by another of the methods described in Section II.

In some embodiments, the engineered cell comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the engineered cell comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the engineered cell comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the engineered cell comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

In some embodiments, the engineered primary cardiac cell is an engineered therapeutic cell, such as for use in a cardiac cell therapy.

In some embodiments, the cardiomyocyte differentiated from a PSC comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the cardiomyocyte differentiated from a PSC comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the cardiomyocyte differentiated from a PSC comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the cardiomyocyte differentiated from a PSC comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

In some embodiments, the primary cardiac cell comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cell of the same cell type that does not comprise the one or more modifications. In some embodiments, the primary cardiac cell comprises one or more modifications that: (a) reduce expression of one or more of CACNA1G, HCN4, and SLC8A1; (b) reduce expression of one or more of MHC HLA class I molecules and one or more MHC HLA class II molecules; and (c) increase expression of one or more of KCNJ2, TRDN, SRL, HRC, CASQ2, and CD47, relative to a cell of the same cell type that does not comprise the one or more modifications. In some embodiments, the primary cardiac cell comprises one or more modifications that: (a) reduce expression of CACNA1G, HCN4, and SLC8A1; (b) reduce expression of one or more of MHC HLA class I molecules and one or more MHC HLA class II molecules; and (c) increase expression of KCNJ2 and CD47, relative to a cell of the same cell type that does not comprise the one or more modifications. In some embodiments, the primary cardiac cell comprises one or more modifications that: (a) reduce expression of CACNA1G, HCN4, and SLC8A1; (b) reduce expression of one or more of B2M and CIITA; and (c) increase expression of KCNJ2 and CD47, relative to a cell of the same cell type that does not comprise the one or more modifications.

### IV. METHODS OF ENGINEERING

Provided herein are methods of producing engineered cells comprising introducing one or more modifications into a cell that reduces expression of one or more target polynucleotides, increases expression of one or more target polynucleotides, or both.

### A. Methods of Reducing Expression

In some embodiments, the cells provided herein are modified, such as genetically modified, to reduce expression of the one or more target polynucleotides as described. In some embodiments, the cell that is engineered with the one or more modifications to reduce (e.g. eliminate) expression of a polynucleotide or protein is any source cell as described herein. In some embodiments, the source cell is any cell described herein. In certain embodiments, the cells (e.g., PSCs or cardiomyocytes differentiated therefrom) disclosed herein comprise one or more modifications, such as genetic modifications, to reduce expression of one or more target polynucleotides. Non-limiting examples of the one or more target polynucleotides include any as described above, such as one or more of CACNA1G, CACNA1H, HCN4, and SLC8A1. Additional non-limiting examples of the one or more target polynucleotides include any as described above, such as one or more of MHC class I, or a component thereof, MHC class II molecule, CIITA, TAP1, B2M, NLRC5, HLA-A, HLA-B, HLA-C, LRC5, RFX-ANK, RFX5, RFX-AP, NFY-A, NFY-B, NFY-C, IRF1, and TAP1. In some embodiments, the one or more modifications, such as genetic modifications, to reduce expression of the one or more target polynucleotides is combined with one or more modifications to increase expression of a desired transgene, such as any described herein. In some embodiments, the one or more modifications, such as genetic modifications, create engineered cells that are immune-privileged or hypoimmunogenic cells. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

Any method for reducing expression of a target polynucleotide may be used. In some embodiments, the modifications (e.g., genetic modifications) result in permanent elimination or reduction in expression of the target polynucleotide. For instance, in some embodiments, the target polynucleotide or gene is disrupted by introducing a DNA break in the target polynucleotide, such as by using a targeting endonuclease. In some embodiments, the reducing expression comprises inactivating or disrupting one or more alleles of one or more target genes, such as any of the exemplary target genes described herein. In other embodiments, the modifications (e.g., genetic modifications) result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, the one or more modifications, e.g., that reduce expression of one or more MHC HLA class I molecules and/or one or more MHC class II molecules, is generated by nuclease-mediated gene editing. In some embodiments, the nuclease-mediated gene editing is mediated by a zinc finger nuclease (ZFN), a TAL-effector nuclease (TALEN), or a CRISPR-Cas system that targets the gene. In some embodiments, the nuclease-mediated gene editing uses a CRISPR-Cas system comprising a CRISPR-Cas nuclease and a guide RNA (gRNA) having a targeting domain that is complementary to at least one target site within the gene.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the genetic modification may induce a deletion, insertion or mutation of the nucleotide sequence of the target gene. In some cases, the genetic modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, genetic modification with a targeted nuclease, such as using a CRISPR/Cas system, leads to complete knockout of the gene.

In some embodiments, the nuclease, such as a rare-cutting endonuclease, is introduced into a cell containing the target polynucleotide sequence. The nuclease may be introduced into the cell in the form of a nucleic acid encoding the nuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid that is introduced into the cell is DNA. In some embodiments, the nuclease is introduced into the cell in the form of a protein. For instance, in the case of a CRISPR/Cas system a ribonucleoprotein (RNP) may be introduced into the cell.

In some embodiments, the modification (e.g., genetic modification) occurs using a CRISPR/Cas system. Any CRISPR/Cas system that is capable of altering a target polynucleotide sequence in a cell can be used. Such CRISPR-Cas systems can employ a variety of Cas proteins (Haft et al. PLOS Comput Biol. 2005; 1 (6) e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems includes targeted systems that can be used to alter any target polynucleotide sequence in a cell. In some embodiments, a CRISPR/Cas system provided herein includes a Cas protein and one or more, such as at least one to two, ribonucleic acids (e.g., guide RNA (gRNA)) that are capable of directing the Cas protein to and hybridizing to a target motif of a target polynucleotide sequence.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to, Cas1, Cas1b, Cas2, Cas3, Cas4, Cas5, Cas6, Cas7, Cas8, Cas9, Cas12a, Cas12b, Cas1212, Cas13, and Mad7. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA: tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a below.

TABLE la
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas12a (Cpf1) | Acidaminococcus sp. | TTTV |
| AacCas12b | Alicyclobacillus acidiphilus | TTN |
| BhCas12b v4 | Bacillus hisashii | ATTN, TTTN, or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, suitable Cas proteins include, but are not limited to, Cas0, Cas12a (i.e. Cpf1), Cas12b, Cas12i, CasX, and Mad7.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5 (8): 747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In provided embodiments, a CRISPR/Cas system generally includes two components: one or more guide RNA (gRNA) and a Cas protein. In some embodiments, the Cas protein is complexed with the one or more, such as one to two, ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In some embodiments, gRNAs are short synthetic RNAs composed of a scaffold sequence for Cas binding and a user-designed spacer or complementary portion designated crRNA. The cRNA is composed of a crRNA targeting sequence (herein after also called a gRNA targeting sequence; usually about 20 nucleotides in length) that defines the genomic target to be modified and a region of crRNA repeat (e.g. GUUUUAGAGCUA; SEQ ID NO: 23). One can change the genomic target of the Cas protein by simply changing the complementary portion sequence (e.g. gRNA targeting sequence) present in the gRNA. In some embodiments the scaffold sequence for Cas binding is made up of a tracrRNA sequence (e.g.

UAGCAAGUUAAAAUAAGGCUAGUCCGUUAUCAACUUGAAAAAGUGGCACCGAGUCGG UGCUUU; SEQ ID NO: 24) that hybridizes to the crRNA through its anti-repeat sequence. The complex between crRNA: tracrRNA recruits the Cas nuclease (e.g. Cas9) and cleaves upstream of a protospacer-adjacent motif (PAM). In order for the Cas protein to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease, derived from S. pyogenes, recognizes a PAM sequence of NGG. Other Cas9 variants and other nucleases with alternative PAMs have also been characterized and successfully used for genome editing. Thus, the CRISPR/Cas system can be used to create targeted DSBs at specified genomic loci that are complementary to the gRNA designed for the target loci. The crRNA and tracrRNA can be linked together with a loop sequence (e.g. a tetraloop; GAAA, SEQ ID NO: 25) for generation of a gRNA that is a chimeric single guide RNA (sgRNA; Hsu et al. 2013). sgRNA can be generated for DNA-based expression or by chemical synthesis.

In some embodiments, the complementary portion sequences (e.g. gRNA targeting sequence) of the gRNA will vary depending on the target site of interest. In some embodiments, the gRNAs comprise complementary portions specific to a sequence of a gene set forth in Table 1b. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs. In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA targeting sequences useful for CRISPR/Cas-based targeting of genes described herein are provided in Table 1b. The sequences can be found in WO2016183041 filed May 9, 2016, the disclosure including the Tables, Appendices, and Sequence Listing is incorporated herein by reference in its entirety.

TABLE 1b
Exemplary gRNA targeting sequences useful for targeting genes

| Gene Name | SEQ ID NO | WO2016183041 |
|---|---|---|
| HLA-A | SEQ ID NOS: 2-1418 | Table 8, Appendix 1 |
| HLA-B | SEQ ID NOS: 1419-3277 | Table 9, Appendix 2 |
| HLA-C | SEQ ID NOS: 3278-5183 | Table 10, Appendix 3 |
| RFX-ANK | SEQ ID NOS: 95636-102318 | Table 11, Appendix 4 |
| NFY-A | SEQ ID NOS: 102319-121796 | Table 13, Appendix 6 |
| RFX5 | SEQ ID NOS: 85645-90115 | Table 16, Appendix 9 |
| RFX-AP | SEQ ID NOS: 90116-95635 | Table 17, Appendix 10 |
| NFY-B | SEQ ID NOS: 121797-135112 | Table 20, Appendix 13 |
| NFY-C | SEQ ID NOS: 135113-176601 | Table 22, Appendix 15 |
| IRF1 | SEQ ID NOS: 176602-182813 | Table 23, Appendix 16 |
| TAP1 | SEQ ID NOS: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |

In some embodiments, it is within the level of a skilled artisan to identify new loci and/or gRNA targeting sequences for use in methods of genetic disruption to reduce or eliminate expression of a gene as described. For example, for CRISPR/Cas systems, when an existing gRNA targeting sequence for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any gene-editing approaches as described can be used in this method of identifying new loci, including those using ZFNs, TALENS, meganucleases and transposases.

Additional exemplary Cas9 guide RNA sequences useful for CRISPR/Cas-based targeting of genes described herein are provided in Table 2. In some embodiments, the guide RNA targets a target gene selected from the group consisting of the ABO, FUT1, RHD, F3 (CD142), B2M, CIITA, and TRAC genes. In some embodiments, the guide RNA comprises the nucleic acid sequence of any one of SEQ ID Nos: 29-35. In some embodiments, the guide RNA targets the ABO gene and comprises the nucleic acid sequence of SEQ ID NO: 29. In some embodiments, the guide RNA targets the FUT1 gene and comprises the nucleic acid sequence of SEQ ID NO: 30. In some embodiments, the guide RNA targets the RHD gene and comprises the nucleic acid sequence of SEQ ID NO: 31. In some embodiments, the guide RNA targets the F3 (CD142) gene and comprises the nucleic acid sequence of SEQ ID NO: 32. In some embodiments, the guide RNA targets the B2M gene and comprises the nucleic acid sequence of SEQ ID NO: 33. In some embodiments, the guide RNA targets the CIITA gene and comprises the nucleic acid sequence of SEQ ID NO: 34. In some embodiments, the guide RNA targets the TRAC gene and comprises the nucleic acid sequence of SEQ ID NO: 35.

TABLE 2
Additional exemplary Cas9 guide RNA sequences useful for targeting genes

| Gene | guide sequence | PAM | Target site | gRNA cut location | SEQ ID NO |
|---|---|---|---|---|---|
| ABO | UCUCUCCAUGUGCAGUAGGA | AGG | Exon 7 | chr9: 133,257,541 | 29 |
| FUT1 | CUGGAUGUCGGAGGAGUACG | CGG | Exon 4 | chr19: 48,750,822 | 30 |
| RHD | GUCUCCGGAAACUCGAGGUG | AGG | Exon 2 | chr1: 25,284,622 | 31 |
| F3 | ACAGUGUAGACUUGAUUGAC | GGG | Exon 2 | chr1: 94,540,281 | 32 |
| (CD142) | | | | | |
| B2M | CGUGAGUAAACCUGAAUCUU | TGG | Exon 2 | chr15: 44,715,434 | 33 |
| CIITA | GAUAUUGGCAUAAGCCUCCC | TGG | Exon 3 | chr16: 10,895,747 | 34 |
| TRAC | AGAGUCUCUCAGCUGGUACA | | | | 35 |

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells provided herein are made using RNA silencing or RNA interference (RNAi) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, a target polynucleotide, such as any described above, e.g. CIITA, B2M, or NLRC5, can be knocked down in a cell by RNA interference by introducing an inhibitory nucleic acid complementary to a target motif of the target polynucleotide, such as an siRNA, into the cells. In some embodiments, a target polynucleotide, such as any described above, e.g. CIITA, B2M, or NLRC5, can be knocked down in a cell by transducing a shRNA-expressing virus into the cell. In some embodiments, RNA interference is employed to reduce or inhibit the expression of at least one selected from the group consisting of CIITA, B2M, and NLRC5.

### 1. Exemplary Target Polynucleotides and Methods for Reducing Expression

a. Genes Associated with Reducing Engraftment Arrhythmia

In some embodiments, expression of one or more of CACNA1G, CACNA1H, HCN4, and SLC8A1 is decreased or eliminated in the cell. In some embodiments, the engineered cell includes decreased expression of at least one of CACNA1G, CACNA1H, HCN4, and SLC8A1. In some embodiments, expression of one or more of CACNA1G, HCN4, and SLC8A1 is decreased or eliminated in the cell. In some embodiments, the engineered cell includes decreased expression of at least one of CACNA1G, HCN4, and SLC8A1. Provided herein are cells that do not trigger or activate engraftment arrhythmia upon administration to a recipient subject.

In some embodiments, the expression of one or more of Cav3.1, Cav3.2, HCN4, and SLC8A1 is decreased by about 10% or higher compared to a cell of the same cell type that does not comprise the modification, such as decreased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of one or more of Cav3.1, Cav3.2, HCN4, and SLC8A1 is decreased by about 99% or lower compared to a cell of the same cell type that does not comprise the modification, such as decreased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of one or more of Cav3.1, Cav3.2, HCN4, and SLC8A1 is decreased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression of one or more of Cav3.1, HCN4, and SLC8A1 is decreased by about 10% or higher compared to a cell of the same cell type that does not comprise the modification, such as decreased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of one or more of Cav3.1, HCN4, and SLC8A1 is decreased by about 99% or lower compared to a cell of the same cell type that does not comprise the modification, such as decreased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of one or more of Cav3.1, HCN4, and SLC8A1 is decreased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In certain embodiments, the modification, such as the genetic modification, reduces or eliminates, such as knocks out, the expression of the Cav3.1 T-type calcium channel by targeting the CACNA1G gene. In some embodiments, the genetic modification occurs using a CRISPR/Cas system. By reducing or eliminating, such as knocking out, expression of CACNA1G, expression of Cav3.1 is reduced or eliminated.

In some embodiments, the target polynucleotide sequence provided herein is a variant of CACNA1G. In some embodiments, the target polynucleotide sequence is a homolog of CACNA1G. In some embodiments, the target polynucleotide sequence is an ortholog of CACNA1G.

In some embodiments, decreased or eliminated expression of CACNA1G is a modification that reduces expression of Cav3.1. In some embodiments, decreased or eliminated expression of CACNA1G reduces expression of Cav3.1. In some embodiments, decreased or eliminated expression of CACNA1G eliminates expression of Cav3.1. In some embodiments, decreased or eliminated expression of CACNA1G reduces or eliminates expression of Cav3.1, by knocking out CACNA1G.

In some embodiments, the engineered cell comprises a modification (e.g., genetic modification) targeting the CACNA1G gene. In some embodiments, the modification (e.g., genetic modification) targeting the CACNA1G gene is by using a targeted nuclease system that comprises a Cas protein or a polynucleotide encoding a Cas protein, and at least one guide ribonucleic acid sequence for specifically targeting the CACNA1G gene.

In some embodiments, an exogenous nucleic acid or transgene encoding a polypeptide as disclosed herein is inserted at the CACNA1G gene. Exemplary transgenes for targeted insertion at the CACNA1G locus include any as described herein.

Assays to test whether the CACNA1G gene has been inactivated are known and described herein. In one embodiment, the resulting genetic modification of the CACNA1G gene is assessed by PCR. In some embodiments, the reduction of Cav3.1 can be assayed by flow cytometry, such as by FACS analysis. In another embodiment, Cav3.1 protein expression is detected using a Western blot of cells lysates probed with antibodies to the Cav3.1 calcium channel. In another embodiment, reverse transcriptase polymerase chain reactions (RT-PCR) are used to confirm the presence of the inactivating modification, such as genetic modification. In some embodiments, the reduction in Cav3.1 is assessed using an immunoaffinity technique, such as immunohistochemistry or immunocytochemistry.

In some embodiments, the reduction of the Cav3.1 expression or function in the engineered cells can be measured using techniques known in the art; for example, FACS techniques using labeled antibodies that bind the HLA complex; for example, using commercially available Cav3.1 antibodies. In addition, the cells can be tested to confirm that Cav3.1 is not expressed on the cell surface. This may be assayed by FACS analysis using antibodies to Cav3.1.

In some embodiments, the modification (e.g., genetic modification) that reduces CACNA1G expression reduces CACNA1G mRNA expression. In some embodiments, the reduced mRNA expression of CACNA1G is relative to an unmodified or wild-type cell of the same cell type that does not comprise the modification. In some embodiments, the mRNA expression of CACNA1G is reduced by more than about 5%, such as reduced by more than about any of 10%, 20%, 30%, 40%, 50%, 60%, 70%, 80%, 90%, or more. In some embodiments, the mRNA expression of CACNA1G is reduced by up to about 100%, such as reduced by up to about any of 90%, 80%, 70%, 60%, 50%, 40%, 30%, 20%, 10%, 5%, or less. In some embodiments, the mRNA expression of CACNA1G is reduced by any of about 5%, 10%, 20%, 30%, 40%, 50%, 60%, 70%, 80%, 90%, or 100%. In some embodiments, the mRNA expression of CACNA1G is eliminated (e.g., 0% expression of CACNA1G mRNA). In some embodiments, the modification that reduces CACNA1G mRNA expression eliminates CACNA1G gene activity.

In some embodiments, the modification (e.g., genetic modification) that reduces CACNA1G expression reduces Cav3.1 protein expression. In some embodiments, the reduced protein expression of Cav3.1 is relative to an unmodified or wild-type cell of the same cell type that does not comprise the modification. In some embodiments, the protein expression of Cav3.1 is reduced by more than about 5%, such as reduced by more than about any of 10%, 20%, 30%, 40%, 50%, 60%, 70%, 80%, 90%, or more. In some embodiments, the protein expression of Cav3.1 is reduced by up to about 100%, such as reduced by up to about any of 90%, 80%, 70%, 60%, 50%, 40%, 30%, 20%, 10%, 5%, or less. In some embodiments, the protein expression of Cav3.1 is reduced by any of about 5%, 10%, 20%, 30%, 40%, 50%, 60%, 70%, 80%, 90%, or 100%. In some embodiments, the protein expression of Cav3.1 is eliminated (e.g., 0% expression of Cav3.1 protein). In some embodiments, the modification that reduces Cav3.1 protein expression eliminates CACNA1G gene activity.

In some embodiments, the modification (e.g., genetic modification) that reduces CACNA1G expression comprises inactivation or disruption of the CACNA1G gene. In some embodiments, the modification that reduces CACNA1G expression comprises inactivation or disruption of one allele of the CACNA1Ggene. In some embodiments, the modification that reduces CACNA1G expression comprises inactivation or disruption comprises inactivation or disruption of both alleles of the CACNA1G gene.

In some embodiments, the modification (e.g., genetic modification) comprises inactivation or disruption of one or more CACNA1G coding sequences in the cell. In some embodiments, the modification comprises inactivation or disruption of all CACNA1G coding sequences in the cell. In some embodiments, the modification comprises inactivation or disruption comprises an indel in one allele of the CACNA1G gene. In some embodiments, the modification comprises inactivation or disruption comprises an indel in both alleles of the CACNA1G gene. In some embodiments, the modification is a frameshift mutation of genomic DNA of the CACNA1G gene. In some embodiments, the modification is a deletion of genomic DNA of the CACNA1G gene. In some embodiments, the modification is a deletion of a contiguous stretch of genomic DNA of the CACNA1G gene. In some embodiments, the CACNA1G gene is knocked out.

In certain embodiments, the modification, such as the genetic modification, reduces or eliminates, such as knocks out, the expression of the Cav3.2 T-type calcium channel by targeting the CACNA1H gene. In some embodiments, the genetic modification occurs using a CRISPR/Cas system. By reducing or eliminating, such as knocking out, expression of CACNA1H, expression of Cav3.2 is reduced or eliminated.

In some embodiments, the target polynucleotide sequence provided herein is a variant of CACNA1H. In some embodiments, the target polynucleotide sequence is a homolog of CACNA1H. In some embodiments, the target polynucleotide sequence is an ortholog of CACNA1H.

In some embodiments, decreased or eliminated expression of CACNA1H is a modification that reduces expression of Cav3.2. In some embodiments, decreased or eliminated expression of CACNA1H reduces expression of Cav3.2. In some embodiments, decreased or eliminated expression of CACNA1H eliminates expression of Cav3.2. In some embodiments, decreased or eliminated expression of CACNA1H reduces or eliminates expression of Cav3.2, by knocking out CACNA1H.

In some embodiments, the engineered cell comprises a modification (e.g., genetic modification) targeting the CACNA1H gene. In some embodiments, the modification (e.g., genetic modification) targeting the CACNA1H gene is by using a targeted nuclease system that comprises a Cas protein or a polynucleotide encoding a Cas protein, and at least one guide ribonucleic acid sequence for specifically targeting the CACNA1H gene.

In some embodiments, an exogenous nucleic acid or transgene encoding a polypeptide as disclosed herein is inserted at the CACNA1H gene. Exemplary transgenes for targeted insertion at the CACNA1H locus include any as described herein.

Assays to test whether the CACNA1H gene has been inactivated are known and described herein. In one embodiment, the resulting genetic modification of the CACNA1H gene is assessed by PCR. In some embodiments, the reduction of Cav3.2 can be assayed by flow cytometry, such as by FACS analysis. In another embodiment, Cav3.2 protein expression is detected using a Western blot of cells lysates probed with antibodies to the Cav3.2 calcium channel. In another embodiment, reverse transcriptase polymerase chain reactions (RT-PCR) are used to confirm the presence of the inactivating modification, such as genetic modification. In some embodiments, the reduction in Cav3.2 is assessed using an immunoaffinity technique, such as immunohistochemistry or immunocytochemistry.

In some embodiments, the reduction of the Cav3.2 expression or function in the engineered cells can be measured using techniques known in the art; for example, FACS techniques using labeled antibodies that bind the HLA complex; for example, using commercially available Cav3.2 antibodies. In addition, the cells can be tested to confirm that Cav3.2 is not expressed on the cell surface. This may be assayed by FACS analysis using antibodies to Cav3.2.

In some embodiments, the modification (e.g., genetic modification) that reduces CACNA1H expression reduces CACNA1H mRNA expression. In some embodiments, the reduced mRNA expression of CACNA1H is relative to an unmodified or wild-type cell of the same cell type that does not comprise the modification. In some embodiments, the mRNA expression of CACNA1H is reduced by more than about 5%, such as reduced by more than about any of 10%, 20%, 30%, 40%, 50%, 60%, 70%, 80%, 90%, or more. In some embodiments, the mRNA expression of CACNA1H is reduced by up to about 100%, such as reduced by up to about any of 90%, 80%, 70%, 60%, 50%, 40%, 30%, 20%, 10%, 5%, or less. In some embodiments, the mRNA expression of CACNA1H is reduced by any of about 5%, 10%, 20%, 30%, 40%, 50%, 60%, 70%, 80%, 90%, or 100%. In some embodiments, the mRNA expression of CACNA1H is eliminated (e.g., 0% expression of CACNA1H mRNA). In some embodiments, the modification that reduces CACNA1H mRNA expression eliminates CACNA1H gene activity.

In some embodiments, the modification (e.g., genetic modification) that reduces CACNA1H expression reduces Cav3.2 protein expression. In some embodiments, Cav3.2 is human Cav3.2. In some embodiments, Cav3.2 is human Cav3.2 and is or comprises the amino acid sequence of SEQ ID NO: 5. In some embodiments, the reduced protein expression of Cav3.2 is relative to an unmodified or wild-type cell of the same cell type that does not comprise the modification. In some embodiments, the protein expression of Cav3.2 is reduced by more than about 5%, such as reduced by more than about any of 10%, 20%, 30%, 40%, 50%, 60%, 70%, 80%, 90%, or more. In some embodiments, the protein expression of Cav3.2 is reduced by up to about 100%, such as reduced by up to about any of 90%, 80%, 70%, 60%, 50%, 40%, 30%, 20%, 10%, 5%, or less. In some embodiments, the protein expression of Cav3.2 is reduced by any of about 5%, 10%, 20%, 30%, 40%, 50%, 60%, 70%, 80%, 90%, or 100%. In some embodiments, the protein expression of Cav3.2 is eliminated (e.g., 0% expression of Cav3.2 protein). In some embodiments, the modification that reduces Cav3.2 protein expression eliminates CACNA1H gene activity.

In some embodiments, the modification (e.g., genetic modification) that reduces CACNA1H expression comprises inactivation or disruption of the CACNA1H gene. In some embodiments, the modification that reduces CACNA1H expression comprises inactivation or disruption of one allele of the CACNA1H gene. In some embodiments, the modification that reduces CACNA1H expression comprises inactivation or disruption comprises inactivation or disruption of both alleles of the CACNA1H gene.

In some embodiments, the modification (e.g., genetic modification) comprises inactivation or disruption of one or more CACNA1H coding sequences in the cell. In some embodiments, the modification comprises inactivation or disruption of all CACNA1H coding sequences in the cell. In some embodiments, the modification comprises inactivation or disruption comprises an indel in one allele of the CACNA1H gene. In some embodiments, the modification comprises inactivation or disruption comprises an indel in both alleles of the CACNA1H gene. In some embodiments, the modification is a frameshift mutation of genomic DNA of the CACNA1H gene. In some embodiments, the modification is a deletion of genomic DNA of the CACNA1H gene. In some embodiments, the modification is a deletion of a contiguous stretch of genomic DNA of the CACNA1H gene. In some embodiments, the CACNA1H gene is knocked out.

In certain embodiments, the modification, such as the genetic modification, reduces or eliminates, such as knocks out, the expression of the HCN4 protein by targeting the HCN4 gene. In some embodiments, the genetic modification occurs using a CRISPR/Cas system. By reducing or eliminating, such as knocking out, expression of HCN4, expression of HCN4 protein is reduced or eliminated.

In some embodiments, the target polynucleotide sequence provided herein is a variant of HCN4. In some embodiments, the target polynucleotide sequence is a homolog of HCN4. In some embodiments, the target polynucleotide sequence is an ortholog of HCN4.

In some embodiments, the HCN4 is human HCN4. In some embodiments, the HCN4 is human HCN4 and is or comprises the amino acid sequence of SEQ ID NO: 6.

In some embodiments, decreased or eliminated expression of HCN4 is a modification that reduces expression of HCN4 protein. In some embodiments, decreased or eliminated expression of HCN4 reduces expression of HCN4 protein1. In some embodiments, decreased or eliminated expression of HCN4 eliminates expression of HCN4 protein. In some embodiments, decreased or eliminated expression of HCN4 reduces or eliminates expression of HCN4 protein, by knocking out HCN4.

In some embodiments, the engineered cell comprises a modification (e.g., genetic modification) targeting the HCN4 gene. In some embodiments, the modification (e.g., genetic modification) targeting the HCN4 gene is by using a targeted nuclease system that comprises a Cas protein or a polynucleotide encoding a Cas protein, and at least one guide ribonucleic acid sequence for specifically targeting the HCN4 gene.

In some embodiments, an exogenous nucleic acid or transgene encoding a polypeptide as disclosed herein is inserted at the HCN4 gene. Exemplary transgenes for targeted insertion at the HCN4 locus include any as described herein.

Assays to test whether the HCN4 gene has been inactivated are known and described herein. In one embodiment, the resulting genetic modification of the HCN4 gene is assessed by PCR. In some embodiments, the reduction of HCN4 protein can be assayed by flow cytometry, such as by FACS analysis. In another embodiment, HCN4 protein expression is detected using a Western blot of cells lysates probed with antibodies to HCN4 protein. In another embodiment, reverse transcriptase polymerase chain reactions (RT-PCR) are used to confirm the presence of the inactivating modification, such as genetic modification. In some embodiments, the reduction in HCN4 protein is assessed using an immunoaffinity technique, such as immunohistochemistry or immunocytochemistry.

In some embodiments, the reduction of the HCN4 protein expression or function in the engineered cells can be measured using techniques known in the art; for example, FACS techniques using labeled antibodies that bind HCN4 protein; for example, using commercially available HCN4 protein antibodies. In addition, the cells can be tested to confirm that HCN4 protein is not expressed on the cell surface. This may be assayed by FACS analysis using antibodies to HCN4 protein.

In some embodiments, the modification (e.g., genetic modification) that reduces HCN4 expression reduces HCN4 mRNA expression. In some embodiments, the reduced mRNA expression of HCN4 is relative to an unmodified or wild-type cell of the same cell type that does not comprise the modification. In some embodiments, the mRNA expression of HCN4 is reduced by more than about 5%, such as reduced by more than about any of 10%, 20%, 30%, 40%, 50%, 60%, 70%, 80%, 90%, or more. In some embodiments, the mRNA expression of HCN4 is reduced by up to about 100%, such as reduced by up to about any of 90%, 80%, 70%, 60%, 50%, 40%, 30%, 20%, 10%, 5%, or less. In some embodiments, the mRNA expression of HCN4 is reduced by any of about 5%, 10%, 20%, 30%, 40%, 50%, 60%, 70%, 80%, 90%, or 100%. In some embodiments, the mRNA expression of HCN4 is eliminated (e.g., 0% expression of HCN4 mRNA). In some embodiments, the modification that reduces HCN4 mRNA expression eliminates HCN4 gene activity.

In some embodiments, the modification (e.g., genetic modification) that reduces HCN4 expression reduces HCN4 protein expression. In some embodiments, the reduced protein expression of HCN4 is relative to an unmodified or wild-type cell of the same cell type that does not comprise the modification. In some embodiments, the protein expression of HCN4 is reduced by more than about 5%, such as reduced by more than about any of 10%, 20%, 30%, 40%, 50%, 60%, 70%, 80%, 90%, or more. In some embodiments, the protein expression of HCN4 is reduced by up to about 100%, such as reduced by up to about any of 90%, 80%, 70%, 60%, 50%, 40%, 30%, 20%, 10%, 5%, or less. In some embodiments, the protein expression of HCN4 is reduced by any of about 5%, 10%, 20%, 30%, 40%, 50%, 60%, 70%, 80%, 90%, or 100%. In some embodiments, the protein expression HCN4 is eliminated (e.g., 0% expression of HCN4 protein). In some embodiments, the modification that reduces HCN4 protein expression eliminates HCN4 gene activity.

In some embodiments, the modification (e.g., genetic modification) that reduces HCN4 expression comprises inactivation or disruption of the HCN4 gene. In some embodiments, the modification that reduces HCN4 expression comprises inactivation or disruption of one allele of the HCN4gene. In some embodiments, the modification that reduces HCN4 expression comprises inactivation or disruption comprises inactivation or disruption of both alleles of the HCN4 gene.

In some embodiments, the modification (e.g., genetic modification) comprises inactivation or disruption of one or more HCN4 coding sequences in the cell. In some embodiments, the modification comprises inactivation or disruption of all HCN4 coding sequences in the cell. In some embodiments, the modification comprises inactivation or disruption comprises an indel in one allele of the HCN4 gene. In some embodiments, the modification comprises inactivation or disruption comprises an indel in both alleles of the HCN4 gene. In some embodiments, the modification is a frameshift mutation of genomic DNA of the HCN4 gene. In some embodiments, the modification is a deletion of genomic DNA of the HCN4 gene. In some embodiments, the modification is a deletion of a contiguous stretch of genomic DNA of the HCN4 gene. In some embodiments, the HCN4 gene is knocked out.

In certain embodiments, the modification, such as the genetic modification, reduces or eliminates, such as knocks out, the expression of the HCN4 protein by targeting the HCN4 gene. In some embodiments, the genetic modification occurs using a CRISPR/Cas system. By reducing or eliminating, such as knocking out, expression of HCN4, expression of HCN4 protein is reduced or eliminated.

In some embodiments, the target polynucleotide sequence provided herein is a variant of HCN4. In some embodiments, the target polynucleotide sequence is a homolog of HCN4. In some embodiments, the target polynucleotide sequence is an ortholog of HCN4.

In some embodiments, decreased or eliminated expression of HCN4 is a modification that reduces expression of HCN4 protein. In some embodiments, decreased or eliminated expression of HCN4 reduces expression of HCN4 protein1. In some embodiments, decreased or eliminated expression of HCN4 eliminates expression of HCN4 protein. In some embodiments, decreased or eliminated expression of HCN4 reduces or eliminates expression of HCN4 protein, by knocking out HCN4.

In some embodiments, the engineered cell comprises a modification (e.g., genetic modification) targeting the HCN4 gene. In some embodiments, the modification (e.g., genetic modification) targeting the HCN4 gene is by using a targeted nuclease system that comprises a Cas protein or a polynucleotide encoding a Cas protein, and at least one guide ribonucleic acid sequence for specifically targeting the HCN4 gene.

In some embodiments, an exogenous nucleic acid or transgene encoding a polypeptide as disclosed herein is inserted at the HCN4 gene. Exemplary transgenes for targeted insertion at the HCN4 locus include any as described herein.

Assays to test whether the HCN4 gene has been inactivated are known and described herein. In one embodiment, the resulting genetic modification of the HCN4 gene is assessed by PCR. In some embodiments, the reduction of HCN4 protein can be assayed by flow cytometry, such as by FACS analysis. In another embodiment, HCN4 protein expression is detected using a Western blot of cells lysates probed with antibodies to HCN4 protein. In another embodiment, reverse transcriptase polymerase chain reactions (RT-PCR) are used to confirm the presence of the inactivating modification, such as genetic modification. In some embodiments, the reduction in HCN4 protein is assessed using an immunoaffinity technique, such as immunohistochemistry or immunocytochemistry.

In some embodiments, the reduction of the HCN4 protein expression or function in the engineered cells can be measured using techniques known in the art; for example, FACS techniques using labeled antibodies that bind HCN4 protein; for example, using commercially available HCN4 protein antibodies. In addition, the cells can be tested to confirm that HCN4 protein is not expressed on the cell surface. This may be assayed by FACS analysis using antibodies to HCN4 protein.

In some embodiments, the modification (e.g., genetic modification) that reduces HCN4 expression reduces HCN4 mRNA expression. In some embodiments, the reduced mRNA expression of HCN4 is relative to an unmodified or wild-type cell of the same cell type that does not comprise the modification. In some embodiments, the mRNA expression of HCN4 is reduced by more than about 5%, such as reduced by more than about any of 10%, 20%, 30%, 40%, 50%, 60%, 70%, 80%, 90%, or more. In some embodiments, the mRNA expression of HCN4 is reduced by up to about 100%, such as reduced by up to about any of 90%, 80%, 70%, 60%, 50%, 40%, 30%, 20%, 10%, 5%, or less. In some embodiments, the mRNA expression of HCN4 is reduced by any of about 5%, 10%, 20%, 30%, 40%, 50%, 60%, 70%, 80%, 90%, or 100%. In some embodiments, the mRNA expression of HCN4 is eliminated (e.g., 0% expression of HCN4 mRNA). In some embodiments, the modification that reduces HCN4 mRNA expression eliminates HCN4 gene activity.

In some embodiments, the modification (e.g., genetic modification) that reduces HCN4 expression reduces HCN4 protein expression. In some embodiments, the reduced protein expression of HCN4 is relative to an unmodified or wild-type cell of the same cell type that does not comprise the modification. In some embodiments, the protein expression of HCN4 is reduced by more than about 5%, such as reduced by more than about any of 10%, 20%, 30%, 40%, 50%, 60%, 70%, 80%, 90%, or more. In some embodiments, the protein expression of HCN4 is reduced by up to about 100%, such as reduced by up to about any of 90%, 80%, 70%, 60%, 50%, 40%, 30%, 20%, 10%, 5%, or less. In some embodiments, the protein expression of HCN4 is reduced by any of about 5%, 10%, 20%, 30%, 40%, 50%, 60%, 70%, 80%, 90%, or 100%. In some embodiments, the protein expression HCN4 is eliminated (e.g., 0% expression of HCN4 protein). In some embodiments, the modification that reduces HCN4 protein expression eliminates HCN4 gene activity.

In some embodiments, the modification (e.g., genetic modification) that reduces HCN4 expression comprises inactivation or disruption of the HCN4 gene. In some embodiments, the modification that reduces HCN4 expression comprises inactivation or disruption of one allele of the HCN4gene. In some embodiments, the modification that reduces HCN4 expression comprises inactivation or disruption comprises inactivation or disruption of both alleles of the HCN4 gene.

In some embodiments, the modification (e.g., genetic modification) comprises inactivation or disruption of one or more HCN4 coding sequences in the cell. In some embodiments, the modification comprises inactivation or disruption of all HCN4 coding sequences in the cell. In some embodiments, the modification comprises inactivation or disruption comprises an indel in one allele of the HCN4 gene. In some embodiments, the modification comprises inactivation or disruption comprises an indel in both alleles of the HCN4 gene. In some embodiments, the modification is a frameshift mutation of genomic DNA of the HCN4 gene. In some embodiments, the modification is a deletion of genomic DNA of the HCN4 gene. In some embodiments, the modification is a deletion of a contiguous stretch of genomic DNA of the HCN4 gene. In some embodiments, the HCN4 gene is knocked out.

In certain embodiments, the modification, such as the genetic modification, reduces or eliminates, such as knocks out, the expression of the SLC8A1 protein by targeting the SLC8A1 gene. In some embodiments, the genetic modification occurs using a CRISPR/Cas system. By reducing or eliminating, such as knocking out, expression of SLC8A1, expression of SLC8A1 protein is reduced or eliminated.

In some embodiments, the target polynucleotide sequence provided herein is a variant of SLC8A1. In some embodiments, the target polynucleotide sequence is a homolog of SLC8A1. In some embodiments, the target polynucleotide sequence is an ortholog of SLC8A1.

In some embodiments, SLC8A1 is human SLC8A1. In some embodiments, SLC8A1 is human SLC8A1 and is or comprises the amino acid sequence of SEQ ID NO: 7.

In some embodiments, decreased or eliminated expression of SLC8A1 is a modification that reduces expression of SLC8A1 protein. In some embodiments, decreased or eliminated expression of SLC8A1 reduces expression of SLC8A1 protein1. In some embodiments, decreased or eliminated expression of SLC8A1 eliminates expression of SLC8A1 protein. In some embodiments, decreased or eliminated expression of SLC8A1 reduces or eliminates expression of SLC8A1 protein, by knocking out SLC8A1.

In some embodiments, the engineered cell comprises a modification (e.g., genetic modification) targeting the SLC8A1 gene. In some embodiments, the modification (e.g., genetic modification) targeting the SLC8A1 gene is by using a targeted nuclease system that comprises a Cas protein or a polynucleotide encoding a Cas protein, and at least one guide ribonucleic acid sequence for specifically targeting the SLC8A1 gene.

In some embodiments, an exogenous nucleic acid or transgene encoding a polypeptide as disclosed herein is inserted at the SLC8A1 gene. Exemplary transgenes for targeted insertion at the SLC8A1 locus include any as described herein.

Assays to test whether the SLC8A1 gene has been inactivated are known and described herein. In one embodiment, the resulting genetic modification of the SLC8A1 gene is assessed by PCR. In some embodiments, the reduction of SLC8A1 protein can be assayed by flow cytometry, such as by FACS analysis. In another embodiment, SLC8A1 protein expression is detected using a Western blot of cells lysates probed with antibodies to SLC8A1 protein. In another embodiment, reverse transcriptase polymerase chain reactions (RT-PCR) are used to confirm the presence of the inactivating modification, such as genetic modification. In some embodiments, the reduction in SLC8A1 protein is assessed using an immunoaffinity technique, such as immunohistochemistry or immunocytochemistry.

In some embodiments, the reduction of the SLC8A1 protein expression or function in the engineered cells can be measured using techniques known in the art; for example, FACS techniques using labeled antibodies that bind SLC8A1 protein; for example, using commercially available SLC8A1 protein antibodies. In addition, the cells can be tested to confirm that SLC8A1 protein is not expressed on the cell surface. This may be assayed by FACS analysis using antibodies to SLC8A1 protein.

In some embodiments, the modification (e.g., genetic modification) that reduces SLC8A1 expression reduces SLC8A1 mRNA expression. In some embodiments, the reduced mRNA expression of SLC8A1 is relative to an unmodified or wild-type cell of the same cell type that does not comprise the modification. In some embodiments, the mRNA expression of SLC8A1 is reduced by more than about 5%, such as reduced by more than about any of 10%, 20%, 30%, 40%, 50%, 60%, 70%, 80%, 90%, or more. In some embodiments, the mRNA expression of SLC8A1 is reduced by up to about 100%, such as reduced by up to about any of 90%, 80%, 70%, 60%, 50%, 40%, 30%, 20%, 10%, 5%, or less. In some embodiments, the mRNA expression of SLC8A1 is reduced by any of about 5%, 10%, 20%, 30%, 40%, 50%, 60%, 70%, 80%, 90%, or 100%. In some embodiments, the mRNA expression of SLC8A1 is eliminated (e.g., 0% expression of SLC8A1 mRNA). In some embodiments, the modification that reduces SLC8A1 mRNA expression eliminates SLC8A1 gene activity.

In some embodiments, the modification (e.g., genetic modification) that reduces SLC8A1 expression reduces SLC8A1 protein expression. In some embodiments, the reduced protein expression of SLC8A1 is relative to an unmodified or wild-type cell of the same cell type that does not comprise the modification. In some embodiments, the protein expression of SLC8A1 is reduced by more than about 5%, such as reduced by more than about any of 10%, 20%, 30%, 40%, 50%, 60%, 70%, 80%, 90%, or more. In some embodiments, the protein expression of SLC8A1 is reduced by up to about 100%, such as reduced by up to about any of 90%, 80%, 70%, 60%, 50%, 40%, 30%, 20%, 10%, 5%, or less. In some embodiments, the protein expression of SLC8A1 is reduced by any of about 5%, 10%, 20%, 30%, 40%, 50%, 60%, 70%, 80%, 90%, or 100%. In some embodiments, the protein expression SLC8A1 is eliminated (e.g., 0% expression of SLC8A1 protein). In some embodiments, the modification that reduces SLC8A1 protein expression eliminates SLC8A1 gene activity.

In some embodiments, the modification (e.g., genetic modification) that reduces SLC8A1 expression comprises inactivation or disruption of the SLC8A1 gene. In some embodiments, the modification that reduces SLC8A1 expression comprises inactivation or disruption of one allele of the SLC8A1gene. In some embodiments, the modification that reduces SLC8A1 expression comprises inactivation or disruption comprises inactivation or disruption of both alleles of the SLC8A1 gene.

In some embodiments, the modification (e.g., genetic modification) comprises inactivation or disruption of one or more SLC8A1 coding sequences in the cell. In some embodiments, the modification comprises inactivation or disruption of all SLC8A1 coding sequences in the cell. In some embodiments, the modification comprises inactivation or disruption comprises an indel in one allele of the SLC8A1 gene. In some embodiments, the modification comprises inactivation or disruption comprises an indel in both alleles of the SLC8A1 gene. In some embodiments, the modification is a frameshift mutation of genomic DNA of the SLC8A1 gene. In some embodiments, the modification is a deletion of genomic DNA of the SLC8A1 gene. In some embodiments, the modification is a deletion of a contiguous stretch of genomic DNA of the SLC8A1 gene. In some embodiments, the SLC8A1 gene is knocked out.

b. MHC HLA Class I Molecules

In certain embodiments, the modification, such as the genetic modification, reduces or eliminates, such as knocks out, the expression of MHC class I molecule genes by targeting the accessory chain B2M. In some embodiments, the genetic modification occurs using a CRISPR/Cas system. By reducing or eliminating, such as knocking out, expression of B2M, surface trafficking of MHC class I molecules is blocked and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of MHC class I molecules is a modification that reduces expression of one or more of the following MHC class I molecules: HLA-A, HLA-B, and HLA-C. In some embodiments, decreased or eliminated expression of B2M reduces or eliminates expression of one or more of the following MHC class I molecules: HLA-A, HLA-B, and HLA-C. In some embodiments, decreased or eliminated expression of B2M reduces or eliminates expression of an HLA-A protein. In some embodiments, decreased or eliminated expression of B2M reduces or eliminates expression of an HLA-B protein. In some embodiments, decreased or eliminated expression of B2M reduces or eliminates expression of an HLA-C protein. In some embodiments, decreased or eliminated expression of B2M reduces or eliminates expression of one or more of the following MHC class I molecules: HLA-A, HLA-B, and HLA-C, by knocking out a gene encoding said molecule. In some embodiments, the gene encoding an HLA-A protein is knocked out to reduce or eliminate expression of said HLA-A protein. In some embodiments, the gene encoding an HLA-B protein is knocked out to reduce or eliminate expression of said HLA-B protein. In some embodiments, the gene encoding an HLA-C protein is knocked out to reduce or eliminate expression of said HLA-C protein.

In some embodiments, the engineered cell comprises a modification (e.g., genetic modification) targeting the B2M gene. In some embodiments, the modification (e.g., genetic modification) targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid sequence (e.g. gRNA targeting sequence) for specifically targeting the B2M gene is selected from the group consisting of SEQ ID NOS: 81240-85644 of Appendix 2 or Table 15 of WO2016/183041, the disclosure is incorporated by reference in its entirety. In some embodiments, the gRNA targeting sequence for specifically targeting the B2M gene is CGUGAGUAAACCUGAAUCUU (SEQ ID NO: 33).

In some embodiments, an exogenous nucleic acid or transgene encoding a polypeptide as disclosed herein (e.g., CD47, or another tolerogenic factor disclosed herein) is inserted at the B2M gene. Exemplary transgenes for targeted insertion at the B2M locus include any as described herein.

Assays to test whether the B2M gene has been inactivated are known and described herein. In one embodiment, the resulting genetic modification of the B2M gene is assessed by PCR. In some embodiments, the reduction of MHC class I, such as HLA-I, expression can be assayed by flow cytometry, such as by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating modification, such as genetic modification. In some embodiments, the reduction in MHC class I molecule expression is assessed using an immunoaffinity technique, such as immunohistochemistry or immunocytochemistry.

In some embodiments, the reduction of the MHC class I molecule expression or function (HLA I when the cells are derived from human cells) in the engineered cells can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the engineered cells provided herein have a reduced susceptibility to macrophage phagocytosis and NK cell killing. Methods to assay for hypoimmunogenic phenotypes of the engineered cells are described further below.

In some embodiments, the modification (e.g., genetic modification) that reduces B2M expression reduces B2M mRNA expression. In some embodiments, the reduced mRNA expression of B2M is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M is reduced by up to about 100%, such as reduced by up to about any of 90%, 80%, 70%, 60%, 50%, 40%, 30%, 20%, 10%, 5%, or less. In some embodiments, the mRNA expression of B2M is reduced by any of about 5%, 10%, 20%, 30%, 40%, 50%, 60%, 70%, 80%, 90%, or 100%. In some embodiments, the mRNA expression of B2M is eliminated (e.g., 0% expression of B2M mRNA). In some embodiments, the modification that reduces B2M mRNA expression eliminates B2M gene activity.

In some embodiments, B2M is human B2M. In some embodiments, B2M is human B2M and is or comprises the amino acid sequence of SEQ ID NO: 9.

In some embodiments, the modification (e.g., genetic modification) that reduces B2M expression reduces B2M protein expression. In some embodiments, the reduced protein expression of B2M is relative to an unmodified or wild-type cell of the same cell type that does not comprise the modification. In some embodiments, the protein expression of B2M is reduced by more than about 5%, such as reduced by more than about any of 10%, 20%, 30%, 40%, 50%, 60%, 70%, 80%, 90%, or more. In some embodiments, the protein expression of B2M is reduced by up to about 100%, such as reduced by up to about any of 90%, 80%, 70%, 60%, 50%, 40%, 30%, 20%, 10%, 5%, or less. In some embodiments, the protein expression of B2M is reduced by any of about 5%, 10%, 20%, 30%, 40%, 50%, 60%, 70%, 80%, 90%, or 100%. In some embodiments, the protein expression of B2M is eliminated (e.g., 0% expression of B2M protein). In some embodiments, the modification that reduces B2M protein expression eliminates B2M gene activity.

In some embodiments, the modification (e.g., genetic modification) that reduces B2M expression comprises inactivation or disruption of the B2M gene. In some embodiments, the modification that reduces B2M expression comprises inactivation or disruption of one allele of the B2M gene. In some embodiments, the modification that reduces B2M expression comprises inactivation or disruption comprises inactivation or disruption of both alleles of the B2M gene.

In some embodiments, the modification (e.g., genetic modification) comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the B2M gene is knocked out. In certain embodiments, the modification, such as the genetic modification, reduces or eliminates, such as knocks out, the expression of MHC class I molecule genes by targeting TAP1. In some embodiments, the genetic modification occurs using a CRISPR/Cas system. By reducing or eliminating, such as knocking out, expression of TAP1, expression of MHC class I molecules is reduced or eliminated, thereby also reducing or eliminating surface tracking of MHC class I molecules. Cells with such modifications exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of TAP1. In some embodiments, the target polynucleotide sequence is a homolog of TAP1. In some embodiments, the target polynucleotide sequence is an ortholog of TAP1.

In some embodiments, decreased or eliminated expression of MHC class I molecules is a modification that reduces expression of one or more of the following MHC class I molecules: HLA-A, HLA-B, and HLA-C. In some embodiments, decreased or eliminated expression of TAP1 reduces or eliminates expression of one or more of the following MHC class I molecules: HLA-A, HLA-B, and HLA-C. In some embodiments, decreased or eliminated expression of TAP1 reduces or eliminates expression of an HLA-A protein. In some embodiments, decreased or eliminated expression of TAP1 reduces or eliminates expression of an HLA-B protein. In some embodiments, decreased or eliminated expression of TAP1 reduces or eliminates expression of an HLA-C protein. In some embodiments, decreased or eliminated expression of TAP1 reduces or eliminates expression of HLA-A, HLA-B, and HLA-C. In some embodiments, the expression of one or more of the following MHC class I molecules: HLA-A, HLA-B, and HLA-C, is reduced or eliminated by knocking out a gene encoding said molecule. In some embodiments, the gene encoding an HLA-A protein is knocked out to reduce or eliminate expression of said HLA-A protein. In some embodiments, the gene encoding an HLA-B protein is knocked out to reduce or eliminate expression of said HLA-B protein. In some embodiments, the gene encoding an HLA-C protein is knocked out to reduce or eliminate expression of said HLA-C protein.

In some embodiments, the engineered cell comprises a modification (e.g., genetic modification) targeting the TAP1 gene. In some embodiments, the modification (e.g., genetic modification) targeting the TAP1 gene is by using a targeted nuclease system that comprises a Cas protein or a polynucleotide encoding a Cas protein, and at least one guide ribonucleic acid sequence for specifically targeting the TAP1 gene.

In some embodiments, an exogenous nucleic acid or transgene encoding a polypeptide as disclosed herein (e.g., CD47, or another tolerogenic factor disclosed herein) is inserted at the TAP1 gene. Exemplary transgenes for targeted insertion at the TAP1 locus include any as described herein.

Assays to test whether the TAP1 gene has been inactivated are known and described herein. In one embodiment, the resulting genetic modification of the TAP1 gene is assessed by PCR. In some embodiments, the reduction of MHC class I, such as HLA-I, expression can be assayed by flow cytometry, such as by FACS analysis. In another embodiment, TAP1 protein expression is detected using a Western blot of cells lysates probed with antibodies to the TAP1 protein. In another embodiment, reverse transcriptase polymerase chain reactions (RT-PCR) are used to confirm the presence of the inactivating modification, such as genetic modification. In some embodiments, the reduction in MHC class I molecule expression is assessed using an immunoaffinity technique, such as immunohistochemistry or immunocytochemistry.

In some embodiments, the reduction of the MHC class I molecule expression or function (HLA I when the cells are derived from human cells) in the engineered cells can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the engineered cells provided herein have a reduced susceptibility to macrophage phagocytosis and NK cell killing. Methods to assay for hypoimmunogenic phenotypes of the engineered cells are described further below.

In some embodiments, the modification (e.g., genetic modification) that reduces TAP1 expression reduces TAP1 mRNA expression. In some embodiments, the reduced mRNA expression of TAP1 is relative to an unmodified or wild-type cell of the same cell type that does not comprise the modification. In some embodiments, the mRNA expression of TAP1 is reduced by more than about 5%, such as reduced by more than about any of 10%, 20%, 30%, 40%, 50%, 60%, 70%, 80%, 90%, or more. In some embodiments, the mRNA expression of TAP1 is reduced by up to about 100%, such as reduced by up to about any of 90%, 80%, 70%, 60%, 50%, 40%, 30%, 20%, 10%, 5%, or less. In some embodiments, the mRNA expression of TAP1 is reduced by any of about 5%, 10%, 20%, 30%, 40%, 50%, 60%, 70%, 80%, 90%, or 100%. In some embodiments, the mRNA expression of TAP1 is eliminated (e.g., 0% expression of TAP1 mRNA). In some embodiments, the modification that reduces TAP1 mRNA expression eliminates TAP1 gene activity.

In some embodiments, the modification (e.g., genetic modification) that reduces TAP1 expression reduces TAP1 protein expression. In some embodiments, the reduced protein expression of TAP1 is relative to an unmodified or wild-type cell of the same cell type that does not comprise the modification. In some embodiments, the protein expression of TAP1 is reduced by more than about 5%, such as reduced by more than about any of 10%, 20%, 30%, 40%, 50%, 60%, 70%, 80%, 90%, or more. In some embodiments, the protein expression of TAP1 is reduced by up to about 100%, such as reduced by up to about any of 90%, 80%, 70%, 60%, 50%, 40%, 30%, 20%, 10%, 5%, or less. In some embodiments, the protein expression of TAP1 is reduced by any of about 5%, 10%, 20%, 30%, 40%, 50%, 60%, 70%, 80%, 90%, or 100%. In some embodiments, the protein expression of TAP1 is eliminated (e.g., 0% expression of TAP1 protein). In some embodiments, the modification that reduces TAP1 protein expression eliminates TAP1 gene activity.

In some embodiments, the modification (e.g., genetic modification) that reduces TAP1 expression comprises inactivation or disruption of the TAP1 gene. In some embodiments, the modification that reduces TAP1 expression comprises inactivation or disruption of one allele of the TAP1 gene. In some embodiments, the modification that reduces TAP1 expression comprises inactivation or disruption comprises inactivation or disruption of both alleles of the TAP1 gene.

In some embodiments, the modification (e.g., genetic modification) comprises inactivation or disruption of one or more TAP1 coding sequences in the cell. In some embodiments, the modification comprises inactivation or disruption of all TAP1 coding sequences in the cell. In some embodiments, the modification comprises inactivation or disruption comprises an indel in one allele of the TAP1 gene. In some embodiments, the modification comprises inactivation or disruption comprises an indel in both alleles of the TAP1 gene. In some embodiments, the modification is a frameshift mutation of genomic DNA of the TAP1 gene. In some embodiments, the modification is a deletion of genomic DNA of the TAP1 gene. In some embodiments, the modification is a deletion of a contiguous stretch of genomic DNA of the TAP1 gene. In some embodiments, the TAP1 gene is knocked out.

c. MHC HLA Class II Molecules

In certain aspects, the modification, such as genetic modification, reduces or eliminates, such as knocks out, the expression of MHC class II molecule genes by targeting Class II molecule transactivator (CIITA) expression. In some embodiments, the genetic modification occurs using a CRISPR/Cas system. CIITA is a member of the LR or nucleotide binding domain (NBD) leucine-rich repeat (LRR) family of proteins and regulates the transcription of MHC class II molecule by associating with the MHC enhanceosome. By reducing or eliminating, such as knocking out, expression of CIITA, expression of MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, decreased or eliminated expression of MHC class II molecule is a modification that reduces expression of one or more of the following MHC class II molecules-HLA-DP, HLA-DM, HLA-DOA, HLA-DOB, HLA-DQ, and HLA-DR. In some embodiments, reduced or eliminated expression of CIITA reduces or eliminates expression of one or more of the following MHC class II molecules-HLA-DP, HLA-DM, HLA-DOA, HLA-DOB, HLA-DQ, and HLA-DR. In some embodiments, decreased or eliminated expression of CIITA reduces or eliminates expression of an HLA-DP protein. In some embodiments, decreased or eliminated expression of CIITA reduces or eliminates expression of an HLA-DM protein. In some embodiments, decreased or eliminated expression of CIITA reduces or eliminates expression of an HLA-DOA protein. In some embodiments, decreased or eliminated expression of CIITA reduces or eliminates expression of an HLA-DOB protein. In some embodiments, decreased or eliminated expression of CIITA reduces or eliminates expression of an HLA-DQ protein. In some embodiments, decreased or eliminated expression of CIITA reduces or eliminates expression of an HLA-DR protein. In some embodiments, decreased or eliminated expression of CIITA reduces or eliminates expression of one or more of the following MHC class II molecules-HLA-DP, HLA-DM, HLA-DOA, HLA-DOB, HLA-DQ, and HLA-DR, by knocking out a gene encoding said molecule. In some embodiments, the gene encoding an HLA-DP protein is knocked out to reduce or eliminate expression of said HLA-DP protein. In some embodiments, the gene encoding an HLA-DM protein is knocked out to reduce or eliminate expression of said HLA-DM protein. In some embodiments, the gene encoding an HLA-DOA protein is knocked out to reduce or eliminate expression of said HLA-DOA protein. In some embodiments, the gene encoding an HLA-DOB protein is knocked out to reduce or eliminate expression of said HLA-DOB protein. In some embodiments, the gene encoding an HLA-DQ protein is knocked out to reduce or eliminate expression of said HLA-DQ protein. In some embodiments, the gene encoding an HLA-DR protein is knocked out to reduce or eliminate expression of said HLA-DR protein.

In some embodiments, the engineered cell comprises a modification (e.g., genetic modification) targeting the CIITA gene. In some embodiments, the modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence (e.g. gRNA targeting sequence) for specifically targeting the CIITA gene is selected from the group consisting of SEQ ID NOS: 5184-36352 of Appendix 1 or Table 12 of WO2016183041, the disclosure is incorporated by reference in its entirety. In some embodiments, the gRNA targeting sequence for specifically targeting the CIITA gene is GAUAUUGGCAUAAGCCUCCC (SEQ ID NO: 34).

In some embodiments, an exogenous nucleic acid or transgene encoding a polypeptide as disclosed herein (e.g., CD47, or another tolerogenic factor disclosed herein) is inserted at the CIITA gene. Exemplary transgenes for targeted insertion at the CIITA locus include any as described in herein.

Assays to test whether the CIITA gene has been inactivated are known and described herein. In one embodiment, the resulting genetic modification of the CIITA gene is assessed by PCR. In some embodiments, the reduction of MHC class II molecule, such as HLA-II, expression can be assays by flow cytometry, such as by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating modification, such as genetic modification. In some embodiments, the reduction in MHC class II molecule expression is assessed using an immunoaffinity technique, such as immunohistochemistry or immunocytochemistry.

In some embodiments, the reduction of the MHC class II molecule expression or function (HLA II when the cells are derived from human cells) in the engineered cells can be measured using techniques known in the art, such as Western blotting using antibodies to the protein, FACS techniques, and RT-PCR techniques. In some embodiments, the engineered cells can be tested to confirm that the HLA II complex is not expressed on the cell surface. Methods to assess surface expression include methods known in the art (See FIG. 21 of WO2018132783, for example) and generally is done using either Western Blots or FACS analysis based on commercial antibodies that bind to human HLA Class II molecule HLA-DR, DP and most DQ antigens. In addition to the reduction of HLA II (or MHC class II molecule), the engineered cells provided herein have a reduced susceptibility to macrophage phagocytosis and NK cell killing. Methods to assay for hypoimmunogenic phenotypes of the engineered cells are described further below.

In some embodiments, the modification (e.g., genetic modification) that reduces CIITA expression reduces CIITA mRNA expression. In some embodiments, the reduced mRNA expression of CIITA is relative to an unmodified or wild-type cell of the same cell type that does not comprise the modification. In some embodiments, the mRNA expression of CIITA is reduced by more than about 5%, such as reduced by more than about any of 10%, 20%, 30%, 40%, 50%, 60%, 70%, 80%, 90%, or more. In some embodiments, the mRNA expression of CIITA is reduced by up to about 100%, such as reduced by up to about any of 90%, 80%, 70%, 60%, 50%, 40%, 30%, 20%, 10%, 5%, or less. In some embodiments, the mRNA expression of CIITA is reduced by any of about 5%, 10%, 20%, 30%, 40%, 50%, 60%, 70%, 80%, 90%, or 100%. In some embodiments, the mRNA expression of CIITA is eliminated (e.g., 0% expression of CIITA mRNA). In some embodiments, the modification that reduces CIITA mRNA expression eliminates CIITA gene activity.

In some embodiments, CIITA is human CIITA. In some embodiments, CIITA is human CIITA and is or comprises the amino acid sequence of SEQ ID NO: 10.

In some embodiments, the modification (e.g., genetic modification) that reduces CIITA expression reduces CIITA protein expression. In some embodiments, the reduced protein expression of CIITA is relative to an unmodified or wild-type cell of the same cell type that does not comprise the modification. In some embodiments, the protein expression of CIITA is reduced by more than about 5%, such as reduced by more than about any of 10%, 20%, 30%, 40%, 50%, 60%, 70%, 80%, 90%, or more. In some embodiments, the protein expression of CIITA is reduced by up to about 100%, such as reduced by up to about any of 90%, 80%, 70%, 60%, 50%, 40%, 30%, 20%, 10%, 5%, or less. In some embodiments, the protein expression of CIITA is reduced by any of about 5%, 10%, 20%, 30%, 40%, 50%, 60%, 70%, 80%, 90%, or 100%. In some embodiments, the protein expression of CIITA is eliminated (e.g., 0% expression of CIITA protein). In some embodiments, the modification that reduces CIITA protein expression eliminates CIITA gene activity.

In some embodiments, the modification (e.g., genetic modification) that reduces CIITA expression comprises inactivation or disruption of the CIITA gene. In some embodiments, the modification that reduces CIITA expression comprises inactivation or disruption of one allele of the CIITA gene. In some embodiments, the modification that reduces CIITA expression comprises inactivation or disruption comprises inactivation or disruption of both alleles of the CIITA gene.

In some embodiments, the modification (e.g., genetic modification) comprises inactivation or disruption of one or more CIITA coding sequences in the cell. In some embodiments, the modification comprises inactivation or disruption of all CIITA coding sequences in the cell. In some embodiments, the modification comprises inactivation or disruption comprises an indel in the CIITA gene. In some embodiments, the modification is a frameshift mutation of genomic DNA of the CIITA gene. In some embodiments, the modification is a deletion of genomic DNA of the CIITA gene. In some embodiments, the modification is a deletion of a contiguous stretch of genomic DNA of the CIITA gene. In some embodiments, the CIITA gene is knocked out.

### B. Methods of Increasing Expression

In some embodiments, increased expression of a polynucleotide may be carried out by any of a variety of techniques. For instance, methods for modulating expression of genes and factors (proteins) include genome editing technologies, and, RNA or protein expression technologies and the like. For all of these technologies, well known recombinant techniques are used, to generate recombinant nucleic acids as outlined herein. In some embodiments, the cell that is engineered with the one or more modification for overexpression or increased expression of a polynucleotide is any source cell as described herein. In some embodiments, the source cell is any cell described in Section II.C.

### 1. DNA-Binding Fusion Proteins

In some embodiments, expression of a target gene is increased by expression of fusion protein or a protein complex containing (1) a site-specific binding domain specific for the endogenous target gene, or other gene and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue.

In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31 (7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain (e.g. targeting sequence) comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the gRNA may be any as described herein.

In particular embodiments, the gRNA has a targeting sequence that is complementary to a target site of KCNJ2. In particular embodiments, the gRNA has a targeting sequence that is complementary to a target site of TRDN. In particular embodiments, the gRNA has a targeting sequence that is complementary to a target site of SRL. In particular embodiments, the gRNA has a targeting sequence that is complementary to a target site of HRC. In particular embodiments, the gRNA has a targeting sequence that is complementary to a target site of CASQ2.

In particular embodiments, the gRNA has a targeting sequence that is complementary to a target site of CD47, such as set forth in any one of SEQ ID NOS: 200784-231885 (Table 29, Appendix 22 of WO2016183041); HLA-E, such as set forth in any one of SEQ ID NOS: 189859-193183 (Table 19, Appendix 12 of WO2016183041); HLA-F, such as set forth in any one of SEQ ID NOS: 688808-699754 (Table 45, Appendix 38 of WO2016183041); HLA-G, such as set forth in any one of SEQ ID NOS: 188372-189858 (Table 18, Appendix 11 of WO2016183041); or PD-L1, such as set forth in any one of SEQ ID NOS: 193184-200783 (Table 21, Appendix 14 of WO2016183041).

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence (i.e. gRNA targeting sequence) that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a targeting sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, October 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, CI, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18 (6): 682-689). In other instances, the domain is a histone deacetylase (HD AC) such as the class I (HDAC-1, 2, 3, and 8), class II molecule (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20 (3): 3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

### 2. Exogenous Polypeptides

In some embodiments, increased expression (i.e. overexpression) of the polynucleotide is mediated by introducing into the cell an exogenous polynucleotide encoding the polynucleotide to be overexpressed. In some embodiments, the exogenous polynucleotide is a recombinant nucleic acid. Well-known recombinant techniques can be used to generate recombinant nucleic acids as outlined herein.

In certain embodiments, the recombinant nucleic acids encoding an exogenous polynucleotide may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

In some embodiments, the exogenous polynucleotide is operably linked to a promoter for expression of the exogenous polynucleotide in the engineered cell. Examples of suitable mammalian promoters include, for example, promoters from the following genes: elongation factor 1 alpha (EF1α) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273:113-120 (1978)). The immediate early promoter of the human cytomegalovirus is conveniently obtained as a HindIII restriction enzyme fragment (Greenaway et al, Gene 18:355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

In some embodiments, an expression vector or construct herein is a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more genes (e.g., two or more transgenes). The multi-cistronic construct is referred to as bicistronic construct if it encodes two genes, and tricistronic construct if it encodes three genes, and quadrocistronic construct if it encodes four genes, and so on.

In some embodiments, two or more exogenous polynucleotides comprised by a vector or construct (e.g., a transgene) are each separated by a multicistronic separation element. In some embodiments, the multicistronic separation element is an IRES or a sequence encoding a cleavable peptide or ribosomal skip element. In some embodiments, the multicistronic separation element is an IRES, such as an encephalomyocarditis (EMCV) virus IRES. In some embodiments, the multicistronic separation element is a cleavable peptide such as a 2A peptide. Exemplary 2A peptides include a P2A peptide, a T2A peptide, an E2A peptide, and an F2Apeptide. In some embodiments, the cleavable peptide is a T2A. In some embodiments, the two or more exogenous polynucleotides (e.g. the first exogenous polynucleotide and second exogenous polynucleotide) are operably linked to a promoter. In some embodiments, the first exogenous polynucleotide and the second exogenous polynucleotide are each operably linked to a promoter. In some embodiments, the promoter is the same promoter. In some embodiments, the promoter is an EF1 promoter.

In some cases, an exogenous polynucleotide encoding an exogenous polypeptide (e.g., an exogenous polynucleotide encoding a tolerogenic factor or complement inhibitor described herein) encodes a cleavable peptide or ribosomal skip element, such as T2A at the N-terminus or C-terminus of an exogenous polypeptide encoded by a multicistronic vector. In some embodiments, inclusion of the cleavable peptide or ribosomal skip element allows for expression of two or more polypeptides from a single translation initiation site. In some embodiments, the cleavable peptide is a T2A. In some embodiments, the T2A is or comprises the amino acid sequence set forth by SEQ ID NO: 15. In some embodiments, the T2A is or comprises the amino acid sequence set forth by SEQ ID NO: 16. In some embodiments, the T2A is or comprises the amino acid sequence set forth by SEQ ID NO: 21. In some embodiments, the T2A is or comprises the amino acid sequence set forth by SEQ ID NO: 22.

In some embodiments, the vector or construct includes a single promoter that drives the expression of one or more transcription units of an exogenous polynucleotide. In some embodiments, such vectors or constructs can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products from an RNA transcribed from a single promoter. In some embodiments, the vectors or constructs provided herein are bicistronic, allowing the vector or construct to express two separate polypeptides.

In some cases, the two separate polypeptides encoded by the vector or construct are encoded by any two of KCNJ2, TRDN, SRL, HRC, and CASQ2.

In some cases, the two separate polypeptides encoded by the vector or construct are tolerogenic factors (e.g., two factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof)). In some cases, the two separate polypeptides encoded by the vector or construct are tolerogenic factors (e.g., two factors selected from DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, CI-Inhibitor, IL-10, IL-35, FASL, CCL21, CCL22, MFGE8, SERPINB9, IL-39, CD16 Fc Receptor, IL15-RF, and H2-M3 (including any combination thereof)). In some embodiments, the tolerogenic factor is two or more of CD47, PD-L1, HLA-E or HLA-G, CCL21, FasL, Serpinb9, CD200, and Mfge8 (including any combination thereof). In some embodiments, the two separate polypeptides encoded by the vector or construct are a tolerogenic factor (e.g., CD47). In some embodiments, the vectors or constructs provided herein are tricistronic, allowing the vector or construct to express three separate polypeptides. In some cases, the three nucleic acid sequences of the tricistronic vector or construct are a tolerogenic factor such as CD47. In some cases, the three nucleic acid sequences of the tricistronic vector or construct are three tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the three tolerogenic factor are selected from CD47, PD-L1, HLA-E or HLA-G, CCL21, FasL, Serpinb9, CD200, and Mfge8 (including any combination thereof). In some embodiments, the vectors or constructs provided herein are quadrocistronic, allowing the vector or construct to express four separate polypeptides. In some cases, the four separate polypeptides of the quadrocistronic vector or construct are four tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the four tolerogenic factor are selected from CD47, PD-L1, HLA-E or HLA-G, CCL21, FasL, Serpinb9, CD200, and Mfge8 (including any combination thereof). In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, a single promoter directs expression of an RNA that contains, in a single open reading frame (ORF), two, three, or four genes separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A, e.g., SEQ ID NO: 20), equine rhinitis A virus (E2A, e.g., SEQ ID NO: 19), thoseaasigna virus (T2A, e.g., SEQ ID NO: 15, 16, 21, or 22), and porcine teschovirus-1 (P2A, e.g., SEQ ID NO: 17 or 18) as described in U.S. Patent Publication No. 20070116690.

In cases where the vector or construct (e.g., transgene) contains more than one nucleic acid sequence encoding a protein, and second exogenous polynucleotide encoding a second transgene, the vector or construct (e.g., transgene) may further include a nucleic acid sequence encoding a peptide between the first and second exogenous polynucleotide sequences. In some cases, the nucleic acid sequence positioned between the first and second exogenous polynucleotides encodes a peptide that separates the translation products of the first and second exogenous polynucleotides during or after translation. In some embodiments, the peptide contains a self-cleaving peptide or a peptide that causes ribosome skipping (a ribosomal skip element), such as a T2A peptide. In some embodiments, inclusion of the cleavable peptide or ribosomal skip element allows for expression of two or more polypeptides from a single translation initiation site. In some embodiments, the peptide is a self-cleaving peptide that is a T2A peptide. In some embodiments, the T2A is or comprises the amino acid sequence set forth by SEQ ID NO: 15. In some embodiments, the T2A is or comprises the amino acid sequence set forth by SEQ ID NO: 16. In some embodiments, the T2A is or comprises the amino acid sequence set forth by SEQ ID NO: 21. In some embodiments, the T2A is or comprises the amino acid sequence set forth by SEQ ID NO: 22.

The process of introducing the polynucleotides described herein into cells can be achieved by any suitable technique. Suitable techniques include calcium phosphate or lipid-mediated transfection, electroporation, transposase-mediated delivery, and transduction or infection using a viral vector. In some embodiments, the polynucleotides are introduced into a cell via viral transduction (e.g., lentiviral transduction) or otherwise delivered on a viral vector (e.g., fusogen-mediated delivery). In some embodiments, vectors that package a polynucleotide encoding an exogenous polynucleotide may be used to deliver the packaged polynucleotides to a cell or population of cells. These vectors may be of any kind, including DNA vectors, RNA vectors, plasmids, viral vectors and particles. In some embodiments, lipid nanoparticles can be used to deliver an exogenous polynucleotide to a cell. In some embodiments, viral vectors can be used to deliver an exogenous polynucleotide to a cell. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like. In some embodiments, the introduction of the exogenous polynucleotide into the cell can be specific (targeted) or non-specific (e.g. non-targeted). In some embodiments, the introduction of the exogenous polynucleotide into the cell can result in integration or insertion into the genome in the cell. In other embodiments, the introduced exogenous polynucleotide may be non-integrating or episomal in the cell. A skilled artisan is familiar with methods of introducing nucleic acid transgenes into a cell, including any of the exemplary methods described herein, and can choose a suitable method.

a. Non-Targeted Delivery

In some embodiments, an exogenous polynucleotide is introduced into a cell (e.g., a PSC or a cardiomyocyte differentiated therefrom) by any of a variety of non-targeted methods. In some embodiments, the exogenous polynucleotide is inserted into a random genomic locus of a host cell. As known to a person skilled in the art, viral vectors, including, for example, retroviral vectors and lentiviral vectors are commonly used to deliver genetic material into host cells and randomly insert the foreign or exogenous gene into the host cell genome to facilitate stable expression and replication of the gene. In some embodiments, the non-targeted introduction of the exogenous polynucleotide into the cell is under conditions for stable expression of the exogenous polynucleotide in the cell. In some embodiments, methods for introducing a nucleic acid for stable expression in a cell involves any method that results in stable integration of the nucleic acid into the genome of the cell, such that it may be propagated if the cell it has integrated into divides.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9:457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomyocin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the exogenous polynucleotide. The recombinant viral particles are recovered from the culture media and titrated by standard methods used by those of skill in the art. The recombinant lentiviral vehicles can be used to infect target cells, such source cells including any described in Section II.C.

Cells that can be used to produce high-titer lentiviral particles may include, but are not limited to, HEK293T cells, 293G cells, STAR cells (Relander et al., Mol Ther. 2005, 11:452-459), FreeStyle™ 293 Expression System (ThermoFisher, Waltham, MA), and other HEK293T-based producer cell lines (e.g., Stewart et al., Hum Gene Ther. 2011, 2,2. (3): 357˜369; Lee et al, Biotechnol Bioeng, 2012, 10996): 1551-1560; Throm et al., Blood. 2009, 113 (21): 5104-5110).

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516; and 5,994,136; International Patent Publication NO.: WO2012079000).

In some embodiments, the exogenous polynucleotide is introduced into the cell under conditions for transient expression of the cell, such as by methods that result in episomal delivery of an exogenous polynucleotide.

In some embodiments, polynucleotides encoding the exogenous polynucleotide may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19 (6): 1070-1078; U.S. Pat. Nos. 6,156,303; 7,198,951; U.S. Patent Publication Nos.: US2015/0159173 and US2014/0359799; and International Patent Publication NOs.: WO1998/011244, WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hyrdoporation; chemical carriers such as inorganic particles (e.g. calcium phosphate, silica, gold) and/or chemical methods. In other aspects, synthetic or natural biodegradable agents may be used for delivery such as cationic lipids, lipid nanoemulsions, nanoparticles, peptide-based vectors, or polymer-based vectors.

b. Non-Targeted Delivery

The exogenous polynucleotide can be inserted into any suitable target genomic loci of the cell. In some embodiments, the exogenous polynucleotide is introduced into the cell by targeted integration into a target loci. In some embodiments, targeted integration can be achieved by gene editing using one or more nucleases and/or nickases and a donor template in a process involving homology-dependent or homology-independent recombination.

A number of gene editing methods can be used to insert an exogenous polynucleotide into the specific genomic locus of choice, including for example homology-directed repair (HOR), homology-mediated end-joining (HMEJ), homology-independent targeted integration (HITI), obligate ligation-gated recombination (ObliGaRe), or precise integration into target chromosome (PITCh).

In some embodiments, the nucleases create specific double-strand breaks (DSBs) at desired locations (e.g. target sites) in the genome, and harness the cell's endogenous mechanisms to repair the induced break. The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, when a nuclease or a nickase is introduced with a donor template containing an exogenous polynucleotide sequence (also called a transgene) flanked by homology sequences (e.g. homology arms) that are homologous to sequences at or near the endogenous genomic target locus, e.g. a safe harbor locus, DNA damage repair pathways can result in integration of the transgene sequence at the target site in the cell. This can occur by a homology-dependent process. In some embodiments, the donor template is a circular double-stranded plasmid DNA, single-stranded donor oligonucleotide (ssODN), linear double-stranded polymerase chain reaction (PCR) fragments, or the homologous sequences of the intact sister chromatid. Depending on the form of the donor template, the homology-mediated gene insertion and replacement can be carried out via specific DNA repair pathways such as homology-directed repair (HDR), synthesis-dependent strand annealing (SDSA), microhomology-mediated end joining (MMEJ), and homology-mediated end joining (HMEJ) pathways.

For instance, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break. Upon cleavage by one of these agents, the target locus with the SSBs or the DSB undergoes one of two major pathways for DNA damage repair: (1) the error-prone non-homologous end joining (NHEJ), or (2) the high-fidelity homology-directed repair (HDR) pathway. In some embodiments, a donor template (e.g. circular plasmid DNA or a linear DNA fragment, such as a ssODN) introduced into cells in which there are SSBs or a DSB can result in HDR and integration of the donor template into the target locus. In general, in the absence of a donor template, the NHEJ process re-ligates the ends of the cleaved DNA strands, which frequently results in nucleotide deletions and insertions at the cleavage site.

In some embodiments, site-directed insertion of the exogenous polynucleotide into a cell may be achieved through HDR-based approaches. HDR is a mechanism for cells to repair double-strand breaks (DSBs) in DNA and can be utilized to modify genomes in many organisms using various gene editing systems, including clustered regularly interspaced short palindromic repeat (CRISPR)/Cas systems, zinc finger nucleases (ZFNs), transcription activator-like effector nucleases (TALENs), meganucleases, and transposases.

In some embodiments, the targeted integration is carried by introducing one or more sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to at least one target site(s) sequence of a target gene. Exemplary ZFNs, TALEs, and TALENs are described in, e.g., Lloyd et al., Frontiers in Immunology, 4 (221): 1-7 (2013). In particular embodiments, targeted genetic disruption at or near the target site is carried out using clustered regularly interspaced short palindromic repeats (CRISPR) and CRISPR-associated (Cas) proteins. See Sander and Joung, (2014) Nature Biotechnology, 32 (4): 347-355.

Any of the systems for gene disruption described in Section IV. A can be used and, when also introduced with an appropriate donor template having with an exogenous polynucleotide, e.g. transgene sequences, can result in targeted integration of the exogenous polynucleotide at or near the target site of the genetic disruption. In particular embodiments, the genetic disruption is mediated using a CRISPR/Cas system containing one or more guide RNAs (gRNA) and a Cas protein. Exemplary Cas proteins and gRNA are described in Section IV.A above, any of which can be used in HDR mediated integration of an exogenous polynucleotide into a target locus to which the Crispr/Cas system is specific for. It is within the level of a skilled artisan to choose an appropriate Cas nuclease and gRNA, such as depending on the particular target locus and target site for cleavage and integration of the exogenous polynucleotide by HDR. Further, depending on the target locus a skilled artisan can readily prepare an appropriate donor template, such as described further below.

In some embodiments, the DNA editing system is an RNA-guided CRISPR/Cas system (such as RNA-based CRISPR/Cas system), wherein the CRISPR/Cas system is capable of creating a double-strand break in the target locus (e.g. safe harbor locus) to induce insertion of the transgene into the target locus. In some embodiments, the nuclease system is a CRISPR/Cas9 system. In some embodiments, the CRISPR/Cas9 system comprises a plasmid-based Cas9. In some embodiments, the CRISPR/Cas9 system comprises a RNA-based Cas9. In some embodiments, the CRISPR/Cas9 system comprises a Cas9 mRNA and gRNA. In some embodiments, the CRISPR/Cas9 system comprises a protein/RNA complex, or a plasmid/RNA complex, or a protein/plasmid complex. In some embodiments, there are provided methods for generating engineered cells, which comprises introducing into a source cell (e.g. a PSC or a cardiomyocyte differentiated therefrom) a donor template containing a transgene or exogenous polynucleotide sequence and a DNA nuclease system including a DNA nuclease system (e.g. Cas9) and a locus-specific gRNA. In some embodiments, the Cas9 is introduced as an mRNA. In some embodiments, the Cas9 is introduced as a ribonucleoprotein complex with the gRNA.

Generally, the donor template to be inserted would comprise at least the transgene cassette containing the exogenous polynucleotide of interest and would optionally also include the promoter. In certain of these embodiments, the transgene cassette containing the exogenous polynucleotide and/or promoter to be inserted would be flanked in the donor template by homology arms with sequences homologous to sequences immediately upstream and downstream of the target cleavage site, i.e., left homology arm (LHA) and right homology arm (RHA). Typically, the homology arms of the donor template are specifically designed for the target genomic locus to serve as template for HDR. The length of each homology arm is generally dependent on the size of the insert being introduced, with larger insertions requiring longer homology arms.

In some embodiments, a donor template (e.g., a recombinant donor repair template) comprises: (i) a transgene cassette comprising an exogenous polynucleotide sequence (for example, a transgene operably linked to a promoter, for example, a heterologous promoter); and (ii) two homology arms that flank the transgene cassette and are homologous to portions of a target locus (e.g. safe harbor locus) at either side of a DNA nuclease (e.g., Cas nuclease, such as Cas9 or Cas12) cleavage site. The donor template can further comprise a selectable marker, a detectable marker, and/or a purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about I kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about I kb, about 500 bp to about 2 kb, about 500 bp to about 4 kb, about 1 kb to about 2 kb, about 1 kb to about 2 kb, about 1 kb to about 4 kb, or about 2 kb to about 4 kb.

In some embodiments, the donor template can be cloned into an expression vector. Conventional viral and non-viral based expression vectors known to those of ordinary skill in the art can be used.

In some embodiments, the target locus targeted for integration may be any locus in which it would be acceptable or desired to target integration of an exogenous polynucleotide or transgene. Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a CCR5 gene, a F3 (i.e., CD142) gene, a LRP1 gene, a HMGB1 gene, an ABO gene, a RHD gene, a FUT1 gene, a KDM5D (i.e., HY) gene, a PDGFRa gene, a OLIG2 gene, a TRAC gene, and/or a GFAP gene. In some embodiments, the exogenous polynucleotide can be inserted in a suitable region of the target locus (e.g. safe harbor locus), including, for example, an intron, an exon, and/or gene coding region (also known as a Coding Sequence, or “CDS”). In some embodiments, the insertion occurs in one allele of the target genomic locus. In some embodiments, the insertion occurs in both alleles of the target genomic locus. In either of these embodiments, the orientation of the transgene inserted into the target genomic locus can be either the same or the reverse of the direction of the gene in that locus.

In some embodiments, the exogenous polynucleotide is integrated into an intron, exon, or coding sequence region of the safe harbor gene locus. In some embodiments, the exogenous polynucleotide is inserted into an endogenous gene wherein the insertion causes silencing or reduced expression of the endogenous gene. Exemplary genomic loci for insertion of an exogenous polynucleotide are depicted in Table 4.

TABLE 4
Exemplary genomic loci for insertion of exogenous polynucleotides

| | | | | Target region | |
|---|---|---|---|---|---|
| Number | species | name | Ensembl ID | for cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, and | |
| | | | | CDS | |
| 7 | human | THUMPD3-AS1 | ENSG00000206573 | Intron 1 | ROSA26 |
| 8 | human | Ch-4: 58,976,613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the target locus is a safe harbor locus. In some embodiments, a safe harbor locus is a genomic location that allows for stable expression of integrated DNA with minimal impact on nearby or adjacent endogenous genes, regulatory element and the like. In some cases, a safe harbor gene enables sustainable gene expression and can be targeted by engineered nuclease for gene modification in various cell types including primary cells, PSCs, and differentiated cells thereof. Non-limiting examples of a safe harbor locus include, but are not limited to, a CCR5 gene locus, a PPP1R12C (also known as AAVS1) gene locus, a CLYBL gene locus, and/or a Rosa gene locus (e.g., ROSA26 gene locus). In some embodiments, the safe harbor locus is selected from the group consisting of the AAVS1 locus, the CCR5 locus, and the CLYBL locus. In some cases SHS231 can be targeted as a safe harbor locus in many cell types. In some cases, certain loci can function as a safe harbor locus in certain cell types. For instance, PDGFRa is a safe harbor for glial progenitor cells (GPCs), OLIG2 is a safe harbor locus for oligodendrocytes, and GFAP is a safe harbor locus for astrocytes. It is within the level of a skilled artisan to choose an appropriate safe harbor locus depending on the particular engineered cell type. In some cases, more than one safe harbor gene can be targeted, thereby introducing more than one transgene into the genetically modified cell.

In some embodiments, there are provided methods for generating engineered cells, which comprises introducing into a source cell (e.g. a PSC or a cardiomyocyte differentiated therefrom) a donor template containing a transgene or exogenous polynucleotide sequence and a DNA nuclease system including a DNA nuclease system (e.g. Cas9) and a locus-specific gRNA that comprise complementary portions (e.g. gRNA targeting sequence) specific to a CCR5 gene locus, a PPP1R12C (also known as AAVS1) gene locus, a CLYBL gene locus, and/or a Rosa gene locus (e.g., ROSA26 gene locus). In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In some embodiments, the gRNAs used herein for HDR-mediated insertion of a transgene comprise a complementary portion (e.g. gRNA targeting sequence) that recognizes a target sequence in AAVS1. In certain of these embodiments, the target sequence is located in intron 1 of AAVS 1. AAVS1 is located at Chromosome 19:55,090,918-55,117,637 reverse strand, and AAVS1 intron 1 (based on transcript ENSG00000125503) is located at Chromosome 19:55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19:55, 117,222-55, 112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19:55,115,674. In certain embodiments, the gRNA is configured to produce a cut site at Chromosome 19:55, 115,674, or at a position within 5, 10, 15, 20, 30, 40 or 50 nucleotides of Chromosome 19:55, 115,674. In certain embodiments, the gRNA s GET000046, also known as “sgAAVS1-1,” described in Li et al., Nat. Methods 16:866-869 (2019). This gRNA comprises a complementary portion (e.g. gRNA targeting sequence) having the nucleic acid sequence set forth in SEQ ID NO: 26 (e.g. Table 5) and targets intron 1 of AAVS1 (also known as PPP1R12C).

In some embodiments, the gRNAs used herein for HDR-mediated insertion of a transgene comprise a complementary portion (e.g. gRNA targeting sequence) that recognizes a target sequence in CLYBL. In certain of these embodiments, the target sequence is located in intron 2 of CL YBL. CLYBL is located at Chromosome 13:99,606,669-99,897, 134 forward strand, and CLYBL intron 2 (based on transcript ENST00000376355.7) is located at Chromosome 13:99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13:99,773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13:99,822,980. In certain embodiments, the gRNA is configured to produce a cut site at Chromosome 13:99,822,980, or at a position within 5, 0, 15, 20, 30, 40 or 50 nucleotides of Chromosome 13:99,822,980. In certain embodiments, the gRNA is GET000047, which comprises a complementary portion (e.g. gRNA targeting sequence) having the nucleic acid sequence set forth in SEQ ID NO: 27 (e.g. Table 5) and targets intron 2 of CLYBL. The target site is similar to the target site of the TALENs as described in Cerbini et al., PLOS One, 10 (1): e0116032 (2015).

In some embodiments, the gRNAs used herein for HDR-mediated insertion of a transgene comprise a complementary portion (e.g. gRNA targeting sequence) that recognizes a target sequence in CCR5. In certain of these embodiments, the target sequence is located in exon 3 of CCR5. CCR5 is located at Chromosome 3:46,370,854-46,376,206 forward strand, and CCR5 exon 3 (based on transcript ENST00000292303.4) is located at Chromosome 3:46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3:46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3:46,373,180. In certain embodiments, the gRNA is configured to produce a cut site at Chromosome 3:46,373,180, or at a position within 5, 10, 15, 20, 30, 40, or 50 nucleotides of Chromosome 3:46,373,180. In certain embodiments the gRNA is GET000048, also known as “crCCR5_D,” described in Mandal et al., Cell Stem Cell 15:643-652 (2014). This gRNA comprises a complementary portion having the nucleic acid sequence set forth in SEQ ID NO: 28 (e.g. Table 5) and targets exon 3 of CCR5 (alternatively annotated as exon 2 in the Ensembl genome database). See Gomez-Ospina et al., Nat. Comm. 10(1):4045 (2019).

Table 5 sets forth exemplary gRNA targeting sequences. In some embodiments, the gRNA targeting sequence may contain one or more thymines in the complementary portion sequences set forth in Table 5 are substituted with uracil.

| | | SEQ |
|---|---|---|
| Description | Nucleic Acid Sequence | ID NO |
| GET000046 guide | (5′→3′) accccacagtggggccacta | 26 |
| GET000047 guide | (5′→3′) tgttggaaggatgaggaaat | 27 |
| G ET000048 guide | (5′→3′) tcactatgctgccgcccagt | 28 |

In some embodiments, the target locus is a locus that is desired to be knocked out in the cells. In such embodiments, such a target locus is any target locus whose disruption or elimination is desired in the cell, such as to modulate a phenotype or function of the cell. For instance, any of the gene modifications described in Section IV.A to reduce expression of a target gene may be a desired target locus for targeted integration of an exogenous polynucleotide, in which the genetic disruption or knockout of a target gene and overexpression by targeted insertion of an exogenous polynucleotide may be achieved at the same target site or locus in the cell. For instance, the HDR process may be used to result in a genetic disruption to eliminate or reduce expression of (e.g. knock out) any target gene set forth in Table 1b while also integrating (e.g. knocking in) an exogenous polynucleotide into the target gene by using a donor template with flanking homology arms that are homologous to nucleic acid sequences at or near the target site of the genetic disruption.

In some embodiments, there are provided methods for generating engineered cells, which comprises introducing into a source cell (e.g. a PSC or a cardiomyocyte differentiated therefrom) a donor template containing a transgene or exogenous polynucleotide sequence and a DNA nuclease system including a DNA nuclease system (e.g. Cas9) and a locus-specific gRNA that comprise complementary portions specific to the B2M locus, the CIITA locus, the CACNA1G locus, the CACNA1H locus, the HCN4 locus, or the SLC8A1 locus. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In particular embodiments, the target locus is B2M. In some embodiments, the engineered cell comprises a genetic modification targeting the B2M gene. In some embodiments, the genetic modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid (gRNA) sequence for specifically targeting the B2M gene is selected from the group consisting of SEQ ID NOS: 81240-85644 of Appendix 2 or Table 15 of WO2016/183041, the disclosure is incorporated by reference in its entirety. In some embodiments, an exogenous polynucleotide is integrated into the disrupted B2M locus by HDR by introducing a donor template containing the exogenous polynucleotide sequence with flanking homology arms homologous to sequences adjacent to the target site targeted by the gRNA.

In particular embodiments, the target locus is CIITA. In some embodiments, the engineered cell comprises a genetic modification targeting the CIITA gene. In some embodiments, the genetic modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Appendix 1 or Table 12 of WO2016183041, the disclosure is incorporated by reference in its entirety. In some embodiments, an exogenous polynucleotide is integrated into the disrupted CIITA locus by HDR by introducing a donor template containing the exogenous polynucleotide sequence with flanking homology arms homologous to sequences adjacent to the target site targeted by the gRNA.

In particular embodiments, the target locus is CACNA1G. In some embodiments, the engineered cell comprises a genetic modification targeting the CACNA1G gene. In some embodiments, the genetic modification targeting the CACNA1G gene is by a targeted nuclease system that comprises a Cas protein or a polynucleotide encoding a Cas protein, and at least one guide ribonucleic acid sequence for specifically targeting the CACNA1G gene. In some embodiments, an exogenous polynucleotide is integrated into the disrupted CACNA1G locus by HDR by introducing a donor template containing the exogenous polynucleotide sequence with flanking homology arms homologous to sequences adjacent to the target site targeted by the gRNA.

In particular embodiments, the target locus is CACNA1H. In some embodiments, the engineered cell comprises a genetic modification targeting the CACNA1H gene. In some embodiments, the genetic modification targeting the CACNA1H gene is by a targeted nuclease system that comprises a Cas protein or a polynucleotide encoding a Cas protein, and at least one guide ribonucleic acid sequence for specifically targeting the CACNA1H gene. In some embodiments, an exogenous polynucleotide is integrated into the disrupted CACNA1H locus by HDR by introducing a donor template containing the exogenous polynucleotide sequence with flanking homology arms homologous to sequences adjacent to the target site targeted by the gRNA.

In particular embodiments, the target locus is HCN4. In some embodiments, the engineered cell comprises a genetic modification targeting the HCN4 gene. In some embodiments, the genetic modification targeting the HCN4 gene is by a targeted nuclease system that comprises a Cas protein or a polynucleotide encoding a Cas protein, and at least one guide ribonucleic acid sequence for specifically targeting the HCN4 gene. In some embodiments, an exogenous polynucleotide is integrated into the disrupted HCN4 locus by HDR by introducing a donor template containing the exogenous polynucleotide sequence with flanking homology arms homologous to sequences adjacent to the target site targeted by the gRNA.

In particular embodiments, the target locus is SLC8A1. In some embodiments, the engineered cell comprises a genetic modification targeting the SLC8A1 gene. In some embodiments, the genetic modification targeting the SLC8A1 gene is by a targeted nuclease system that comprises a Cas protein or a polynucleotide encoding a Cas protein, and at least one guide ribonucleic acid sequence for specifically targeting the SLC8A1 gene. In some embodiments, an exogenous polynucleotide is integrated into the disrupted SLC8A1 locus by HDR by introducing a donor template containing the exogenous polynucleotide sequence with flanking homology arms homologous to sequences adjacent to the target site targeted by the gRNA.

In some embodiments, it is within the level of a skilled artisan to identify new loci and/or gRNA sequences for use in HDR-mediated integration approaches as described. For example, for CRISPR/Cas systems, when an existing gRNA for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any HDR-mediated approaches as described can be used in this method of identifying new loci, including those using ZFNs, TALENS, meganucleases and transposases.

In some embodiments, the exogenous polynucleotide encodes an exogenous KCNJ2 polypeptide (e.g., a human KCNJ2 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding KCNJ2 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

In some embodiments, the exogenous polynucleotide encodes an exogenous triadin polypeptide (e.g., a human triadin polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding triadin is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

In some embodiments, the exogenous polynucleotide encodes an exogenous sarcalumenin polypeptide (e.g., a human sarcalumenin polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding sarcalumenin is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

In some embodiments, the exogenous polynucleotide encodes an exogenous HRC polypeptide (e.g., a human HRC polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding HRC is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

In some embodiments, the exogenous polynucleotide encodes an exogenous polypeptide (e.g., a human calsequestrin-2 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding calsequestrin-2 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

In some embodiments, the exogenous polynucleotide encodes an exogenous CD47 polypeptide (e.g., a human CD47 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding CD47 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, CACNA1G, CACNA1H, HCN4, SLC8A1, PD1 or CTLA4 gene locus.

### 3. Exemplary Target Polynucleotides and Methods for Increasing Expression

a. Genes Associated with Reducing Engraftment Arrhythmia

In some embodiments, expression of one or more of KCNJ2, TRDN, SRL, HRC, and CASQ2 is overexpressed or increased in the cell. In some embodiments, the engineered cell includes increased expression, i.e. overexpression, of at least one of KCNJ2, TRDN, SRL, HRC, and CASQ2. In some embodiments, the cell includes at least one exogenous polynucleotide that includes a polynucleotide that encodes for one or more of KCNJ2 (KCNJ2), triadin (TRDN), sarcalumenin (SRL), HRC (HRC), and calsequestrin-2 (CASQ2). For instance, in some embodiments, at least one of the exogenous polynucleotides is a polynucleotide that encodes KCNJ2. For instance, in some embodiments, at least one of the exogenous polynucleotides is a polynucleotide that encodes triadin. For instance, in some embodiments, at least one of the exogenous polynucleotides is a polynucleotide that encodes sarcalumenin. For instance, in some embodiments, at least one of the exogenous polynucleotides is a polynucleotide that encodes HRC. For instance, in some embodiments, at least one of the exogenous polynucleotides is a polynucleotide that encodes calsequestrin-2. Provided herein are cells that do not trigger or activate engraftment arrhythmia upon administration to a recipient subject.

In some embodiments, the expression of one or more of KCNJ2, triadin, sarcalumenin, HRC, and calsequestrin-2 is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of one or more of KCNJ2, triadin, sarcalumenin, HRC, and calsequestrin-2 is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of one or more of KCNJ2, triadin, sarcalumenin, HRC, and calsequestrin-2 is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression of one or more of KCNJ2, triadin, sarcalumenin, HRC, and calsequestrin-2 is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of one or more of KCNJ2, triadin, sarcalumenin, HRC, and calsequestrin-2 is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of one or more of KCNJ2, triadin, sarcalumenin, HRC, and calsequestrin-2 is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the present disclosure provides a cell or population thereof that has been modified to express one or more of KCNJ2, TRDN, SRL, HRC, and CASQ2, such as KCNJ2. In some embodiments, the present disclosure provides a method for altering a cell genome to express the one or more of KCNJ2, TRDN, SRL, HRC, and CASQ2, such as KCNJ2. In some embodiments, the engineered cell expresses one or more of exogenous KCNJ2, TRDN, SRL, HRC, and CASQ2, such as an exogenous KCNJ27. In some instances, overexpression or increasing expression of the exogenous polynucleotide is achieved by introducing into the cell (e.g. transducing the cell with) an expression vector comprising a nucleotide sequence encoding a human KCNJ2 polypeptide. In some embodiments, the expression vector may be a viral vector, such as a lentiviral vector) or may be a non-viral vector. In some embodiments, the cell is engineered to contain one or more exogenous polynucleotides in which at least one of the exogenous polynucleotides includes a polynucleotide that encodes for one or more of KCNJ2, triadin, sarcalumenin, HRC, and calsequestrin-2. For instance, in some embodiments, at least one of the exogenous polynucleotides is a polynucleotide that encodes KCNJ2. In some embodiments, at least one of the exogenous polynucleotides is a polynucleotide that encodes triadin. In some embodiments, at least one of the exogenous polynucleotides is a polynucleotide that encodes sarcalumenin. In some embodiments, at least one of the exogenous polynucleotides is a polynucleotide that encodes HRC. In some embodiments, at least one of the exogenous polynucleotides is a polynucleotide that encodes calsequestrin-2.

In some embodiments, the engineered cell contains an exogenous polynucleotide that encodes KCNJ2, such as human KCNJ2. In some embodiments, KCNJ2 is overexpressed in the cell. In some embodiments, the expression of KCNJ2 is increased in the engineered cell compared to a similar reference or unmodified cell (including with any other modifications, such as genetic modifications) except that the reference or unmodified cell does not include the exogenous polynucleotide encoding KCNJ2. Useful genomic, polynucleotide and polypeptide information about human KCNJ2 are provided in, for example, the HGNC No. 6263 and Uniprot No. P63252. In certain embodiments, the polynucleotide encoding KCNJ2 is operably linked to a promoter. In some embodiments, KCNJ2 is human KCNJ2. In some embodiments, KCNJ2 is human KCNJ2 and is or comprises the amino acid sequence of SEQ ID NO: 8.

In some embodiments, the polynucleotide encoding KCNJ2 is inserted into any one of the gene loci depicted in Table 1B, 2 or 4. In some cases, the polynucleotide encoding KCNJ2 is inserted into a safe harbor locus, such as but not limited to, a gene locus selected from AAVS1, CCR5, CLYBL, ROSA26, and SHS231. In particular embodiments, the polynucleotide encoding KCNJ2 is inserted into the CCR5 gene locus, the PPP1R12C (also known as AAVS1) gene locus or the CLYBL gene locus. In some embodiments, the polynucleotide encoding KCNJ2 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding KCNJ2 into a genomic locus of the cell.

In some embodiments, KCNJ2 protein expression is detected using a Western blot of cell lysates probed with antibodies against the KCNJ2 protein. In another embodiment, reverse transcriptase polymerase chain reactions (RT-PCR) are used to confirm the presence of the exogenous KCNJ2 mRNA.

In some embodiments, the engineered cell contains an exogenous polynucleotide that encodes triadin, such as human triadin. In some embodiments, triadin is overexpressed in the cell. In some embodiments, the expression of triadin is increased in the engineered cell compared to a similar reference or unmodified cell (including with any other modifications, such as genetic modifications) except that the reference or unmodified cell does not include the exogenous polynucleotide encoding triadin. Useful genomic, polynucleotide and polypeptide information about human triadin are provided in, for example, the HGNC No. 12261 and Uniprot No. Q13061. In certain embodiments, the polynucleotide encoding triadin is operably linked to a promoter.

In some embodiments, the triadin is human triadin. In some embodiments, the triadin is human triadin and is or comprises the amino acid sequence of SEQ ID NO: 11.

In some embodiments, the polynucleotide encoding triadin is inserted into any one of the gene loci depicted in Table 1B, 2 or 4. In some cases, the polynucleotide encoding triadin is inserted into a safe harbor locus, such as but not limited to, a gene locus selected from AAVS1, CCR5, CLYBL, ROSA26, and SHS231. In particular embodiments, the polynucleotide encoding triadin is inserted into the CCR5 gene locus, the PPP1R12C (also known as AAVS1) gene locus or the CLYBL gene locus. In some embodiments, the polynucleotide encoding triadin is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding triadin into a genomic locus of the cell.

In some embodiments, triadin protein expression is detected using a Western blot of cell lysates probed with antibodies against the triadin protein. In another embodiment, reverse transcriptase polymerase chain reactions (RT-PCR) are used to confirm the presence of the exogenous TRDN mRNA.

In some embodiments, the engineered cell contains an exogenous polynucleotide that encodes sarcalumenin, such as human sarcalumenin. In some embodiments, sarcalumenin is overexpressed in the cell. In some embodiments, the expression of sarcalumenin is increased in the engineered cell compared to a similar reference or unmodified cell (including with any other modifications, such as genetic modifications) except that the reference or unmodified cell does not include the exogenous polynucleotide encoding sarcalumenin. Useful genomic, polynucleotide and polypeptide information about human sarcalumenin are provided in, for example, the HGNC No. 11295 and Uniprot No. Q86TD4. In certain embodiments, the polynucleotide encoding sarcalumenin is operably linked to a promoter.

In some embodiments, the sarcalumenin is human sarcalumenin. In some embodiments, the sarcalumenin is human sarcalumenin and is or comprises the amino acid sequence of SEQ ID NO: 12.

In some embodiments, the polynucleotide encoding sarcalumenin is inserted into any one of the gene loci depicted in Table 1B, 2 or 4. In some cases, the polynucleotide encoding sarcalumenin is inserted into a safe harbor locus, such as but not limited to, a gene locus selected from AAVS1, CCR5, CLYBL, ROSA26, and SHS231. In particular embodiments, the polynucleotide encoding sarcalumenin is inserted into the CCR5 gene locus, the PPP1R12C (also known as AAVS1) gene locus or the CLYBL gene locus. In some embodiments, the polynucleotide encoding sarcalumenin is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding sarcalumenin into a genomic locus of the cell.

In some embodiments, sarcalumenin protein expression is detected using a Western blot of cell lysates probed with antibodies against the sarcalumenin protein. In another embodiment, reverse transcriptase polymerase chain reactions (RT-PCR) are used to confirm the presence of the exogenous SRL mRNA.

In some embodiments, the engineered cell contains an exogenous polynucleotide that encodes HRC, such as human HRC. In some embodiments, HRC is overexpressed in the cell. In some embodiments, the expression of HRC is increased in the engineered cell compared to a similar reference or unmodified cell (including with any other modifications, such as genetic modifications) except that the reference or unmodified cell does not include the exogenous polynucleotide encoding HRC. Useful genomic, polynucleotide and polypeptide information about human HRC are provided in, for example, the HGNC No. 5178 and Uniprot No. P23327. In certain embodiments, the polynucleotide encoding HRC is operably linked to a promoter.

In some embodiments, the HRC is human HRC. In some embodiments, the HRC is human HRC and is or comprises the amino acid sequence of SEQ ID NO: 13.

In some embodiments, the polynucleotide encoding HRC is inserted into any one of the gene loci depicted in Table 1B, 2 or 4. In some cases, the polynucleotide encoding HRC is inserted into a safe harbor locus, such as but not limited to, a gene locus selected from AAVS1, CCR5, CLYBL, ROSA26, and SHS231. In particular embodiments, the polynucleotide encoding HRC is inserted into the CCR5 gene locus, the PPP1R12C (also known as AAVS1) gene locus or the CLYBL gene locus. In some embodiments, the polynucleotide encoding HRC is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding HRC into a genomic locus of the cell.

In some embodiments, HRC protein expression is detected using a Western blot of cell lysates probed with antibodies against the HRC protein. In another embodiment, reverse transcriptase polymerase chain reactions (RT-PCR) are used to confirm the presence of the exogenous HRC mRNA.

In some embodiments, the engineered cell contains an exogenous polynucleotide that encodes calsequestrin-2, such as human calsequestrin-2. In some embodiments, calsequestrin-2 is overexpressed in the cell. In some embodiments, the expression of calsequestrin-2 is increased in the engineered cell compared to a similar reference or unmodified cell (including with any other modifications, such as genetic modifications) except that the reference or unmodified cell does not include the exogenous polynucleotide encoding calsequestrin-2. Useful genomic, polynucleotide and polypeptide information about human calsequestrin-2 are provided in, for example, the HGNC No. 1513 and Uniprot No. 014958. In certain embodiments, the polynucleotide encoding calsequestrin-2 is operably linked to a promoter.

In some embodiments, the calsequestrin-2 is human calsequestrin-2. In some embodiments, the calsequestrin-2 is human calsequestrin-2 and is or comprises the amino acid sequence of SEQ ID NO: 14.

In some embodiments, the polynucleotide encoding calsequestrin-2 is inserted into any one of the gene loci depicted in Table 1B, 2 or 4. In some cases, the polynucleotide encoding calsequestrin-2 is inserted into a safe harbor locus, such as but not limited to, a gene locus selected from AAVS1, CCR5, CLYBL, ROSA26, and SHS231. In particular embodiments, the polynucleotide encoding calsequestrin-2 is inserted into the CCR5 gene locus, the PPP1R12C (also known as AAVS1) gene locus or the CLYBL gene locus. In some embodiments, the polynucleotide encoding calsequestrin-2 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding calsequestrin-2 into a genomic locus of the cell.

In some embodiments, calsequestrin-2 protein expression is detected using a Western blot of cell lysates probed with antibodies against the calsequestrin-2 protein. In another embodiment, reverse transcriptase polymerase chain reactions (RT-PCR) are used to confirm the presence of the exogenous CASQ2 mRNA.

b. Tolerogenic Factors

In some embodiments, expression of a tolerogenic factor is overexpressed or increased in the cell. In some embodiments, the engineered cell includes increased expression, i.e. overexpression, of at least one tolerogenic factor. In some embodiments, the tolerogenic factor is any factor that promotes or contributes to promoting or inducing tolerance to the engineered cell by the immune system (e.g. innate or adaptive immune system). In some embodiments, the tolerogenic factor is DUX4, B2M-HLA-E, CD24, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, CCL22, MFGE8, SERPINB9, IL-39, CD16 Fc Receptor, IL15-RF, H2-M3, or any combination thereof. In some embodiments, the tolerogenic factor is DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, or H2-M3. In some embodiments, the tolerogenic factor is CD47, PD-L1, HLA-E or HLA-G, CCL21, FasL, Serpinb9, CD200 or Mfge8, or any combination thereof. In some embodiments, the cell includes at least one exogenous polynucleotide that includes a polynucleotide that encodes for a tolerogenic factor. For instance, in some embodiments, at least one of the exogenous polynucleotides is a polynucleotide that encodes CD47. Provided herein are cells that do not trigger or activate an immune response upon administration to a recipient subject. As described above, in some embodiments, the cells are modified to increase expression of genes and tolerogenic (e.g., immune) factors that affect immune recognition and tolerance in a recipient.

In some embodiments, the expression of a tolerogenic factor is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of a tolerogenic factor is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of a tolerogenic factor is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression of a tolerogenic factor is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of a tolerogenic factor is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of a tolerogenic factor is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the present disclosure provides a cell or population thereof that has been modified to express the tolerogenic factor (e.g., immunomodulatory polypeptide), such as CD47. In some embodiments, the present disclosure provides a method for altering a cell genome to express the tolerogenic factor (e.g. immunomodulatory polypeptide), such as CD47. In some embodiments, the engineered cell expresses an exogenous tolerogenic factor (e.g. immunomodulatory polypeptide), such as an exogenous CD47. In some instances, overexpression or increasing expression of the exogenous polynucleotide is achieved by introducing into the cell (e.g. transducing the cell) with an expression vector comprising a nucleotide sequence encoding a human CD47 polypeptide. In some embodiments, the expression vector may be a viral vector, such as a lentiviral vector) or may be a non-viral vector. In some embodiments, the cell is engineered to contain one or more exogenous polynucleotides in which at least one of the exogenous polynucleotides includes a polynucleotide that encodes for a tolerogenic factor. In some embodiments, the DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). For instance, in some embodiments, at least one of the exogenous polynucleotides is a polynucleotide that encodes CD47.

In some embodiments, the tolerogenic factor is CD47. In some embodiments, the engineered cell contains an exogenous polynucleotide that encodes CD47, such as human CD47. In some embodiments, CD47 is overexpressed in the cell. In some embodiments, the expression of CD47 is overexpressed or increased in the engineered cell compared to a similar cell of the same cell type that has not been engineered with the modification, such as a reference or unmodified cell, e.g. a cell not engineered with an exogenous polynucleotide encoding CD47. CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP_942088.1, NM_001777.3 and NM_198793.2.

In some embodiments, the expression of CD47 is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of CD47 is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of CD47 is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression of CD47 is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of CD47 is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of CD47 is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

In some embodiments, the cell comprises a CD47 polypeptide having at least 95% sequence identity (e.g., 95%, 96%, 97%, 98%, 99%, or more) to an amino acid sequence as set forth in SEQ ID NO: 1. In some embodiments, the cell comprises a CD47 polypeptide having the amino acid sequence as set forth in SEQ ID NO: 1. In some embodiments, the cell comprises a CD47 polypeptide having at least 95% sequence identity (e.g., 95%, 96%, 97%, 98%, 99%, or more) to an amino acid sequence as set forth in SEQ ID NO: 2. In some embodiments, the cell comprises a CD47 polypeptide having the amino acid sequence as set forth in SEQ ID NO: 2.

In certain embodiments, the polynucleotide encoding CD47 is operably linked to a promoter.

In some embodiments, an exogenous polynucleotide encoding CD47 is integrated into the genome of the cell by targeted or non-targeted methods of insertion, such as described further below. In some embodiments, targeted insertion is by homology-dependent insertion into a target loci, such as by insertion into any one of the gene loci depicted in Table 1b, 2 or 4, e.g. a B2M gene, a CIITA gene, a CACNA1G gene, a CACNA1H gene, a HCN4 gene, and aSLC8A1 gene. In some embodiments, targeted insertion is by homology-independent insertion, such as by insertion into a safe harbor locus. In some cases, the polynucleotide encoding CD47 is inserted into a safe harbor locus, such as but not limited to, a gene locus selected from AAVS1, CCR5, CLYBL, ROSA26, and SHS231. In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into any one of the gene loci depicted in Table 4. In some cases, the polynucleotide encoding CD47 is inserted into a safe harbor locus.

In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding CD47, into a genomic locus of the cell. In some embodiments, a suitable gene editing system (e.g., CRISPR/Cas system or any of the gene editing systems described herein) is used to facilitate the insertion of a polynucleotide encoding CD47, into a genomic locus of the cell.

In some embodiments, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

In some embodiments, the engineered cell contains an exogenous polynucleotide that encodes CD200, such as human CD200. In some embodiments, CD200 is overexpressed in the cell. In some embodiments, the expression of CD200 is increased in the engineered cell compared to a similar reference or unmodified cell (including with any other modifications, such as genetic modifications) except that the reference or unmodified cell does not include the exogenous polynucleotide encoding CD200. Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2. In certain embodiments, the polynucleotide encoding CD200 is operably linked to a promoter.

In some embodiments, the polynucleotide encoding CD200 is inserted into any one of the gene loci depicted in Table 1B, 2 or 4. In some cases, the polynucleotide encoding CD200 is inserted into a safe harbor locus, such as but not limited to, a gene locus selected from AAVS1, CCR5, CLYBL, ROSA26, and SHS231. In particular embodiments, the polynucleotide encoding CD200 is inserted into the CCR5 gene locus, the PPP1R12C (also known as AAVS1) gene locus or the CLYBL gene locus. In some embodiments, the polynucleotide encoding CD200 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding CD200, into a genomic locus of the cell.

In some embodiments, CD200 protein expression is detected using a Western blot of cell lysates probed with antibodies against the CD200 protein. In another embodiment, reverse transcriptase polymerase chain reactions (RT-PCR) are used to confirm the presence of the exogenous CD200 mRNA.

In some embodiments, the engineered cell contains an exogenous polynucleotide that encodes HLA-E, such as human HLA-E. In some embodiments, HLA-E is overexpressed in the cell. In some embodiments, the expression of HLA-E is increased in the engineered cell compared to a similar reference or unmodified cell (including with any other modifications) except that the reference or unmodified cell does not include the exogenous polynucleotide encoding HLA-E. Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the polynucleotide encoding HLA-E is operably linked to a promoter.

In some embodiments, the polynucleotide encoding HLA-E is inserted into any one of the gene loci depicted in Table 1B, 2 or 4. In some cases, the polynucleotide encoding HLA-E is inserted into a safe harbor locus, such as but not limited to, a gene locus selected from AAVS1, CCR5, CLYBL, ROSA26, SHS231. In particular embodiments, the polynucleotide encoding HLA-E is inserted into the CCR5 gene locus, the PPP1R12C (also known as AAVS1) gene locus or the CLYBL gene locus. In some embodiments, the polynucleotide encoding HLA-E is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding HLA-E, into a genomic locus of the cell.

In some embodiments, HLA-E protein expression is detected using a Western blot of cell lysates probed with antibodies against the HLA-E protein. In another embodiment, reverse transcriptase polymerase chain reactions (RT-PCR) are used to confirm the presence of the exogenous HLA-E mRNA.

In some embodiments, the engineered cell contains an exogenous polynucleotide that encodes HLA-G, such as human HLA-G. In some embodiments, HLA-G is overexpressed in the cell. In some embodiments, the expression of HLA-G is increased in the engineered cell compared to a similar reference or unmodified cell (including with any other modifications, such as genetic modifications) except that the reference or unmodified cell does not include the exogenous polynucleotide encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the polynucleotide encoding HLA-G is operably linked to a promoter.

In some embodiments, the polynucleotide encoding HLA-G is inserted into any one of the gene loci depicted in Table 1b, 2 or 4. In some cases, the polynucleotide encoding HLA-G is inserted into a safe harbor locus, such as but not limited to, a gene locus selected from AAVS1, CCR5, CLYBL, ROSA26, and SHS231. In particular embodiments, the polynucleotide encoding HLA-G is inserted into the CCR5 gene locus, the PPP1R12C (also known as AAVS1) gene locus or the CLYBL gene locus. In some embodiments, the polynucleotide encoding HLA-G is inserted into a B2M gene locus or; a CIITA gene locus. In some embodiments, a suitable gene editing system (e.g., CRISPR/Cas system or any of the gene editing systems described herein) is used to facilitate the insertion of a polynucleotide encoding HLA-G, into a genomic locus of the cell.

In some embodiments, HLA-G protein expression is detected using a Western blot of cell lysates probed with antibodies against the HLA-G protein. In another embodiment, reverse transcriptase polymerase chain reactions (RT-PCR) are used to confirm the presence of the exogenous HLA-G mRNA.

In some embodiments, the engineered cell contains an exogenous polynucleotide that encodes PD-L1, such as human PD-L1. In some embodiments, PD-L1 is overexpressed in the cell. In some embodiments, the expression of PD-L1 is increased in the engineered cell compared to a similar reference or unmodified cell (including with any other modifications) except that the reference or unmodified cell does not include the exogenous polynucleotide encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3. In certain embodiments, the polynucleotide encoding PD-L1 is operably linked to a promoter.

In some embodiments, the polynucleotide encoding PD-L1 is inserted into any one of the gene loci depicted in Table 1B, 2 or 4. In some cases, the polynucleotide encoding PD-L1 is inserted into a safe harbor locus, such as but not limited to, a gene locus selected from AAVS1, CCR5, CLYBL, ROSA26, and SHS231. In particular embodiments, the polynucleotide encoding PD-L1 is inserted into the CCR5 gene locus, the PPP1R12C (also known as AAVS1) gene locus or the CLYBL gene locus. In some embodiments, the polynucleotide encoding PD-L1 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding PD-L1, into a genomic locus of the cell.

In some embodiments, PD-L1 protein expression is detected using a Western blot of cell lysates probed with antibodies against the PD-L1 protein. In another embodiment, reverse transcriptase polymerase chain reactions (RT-PCR) are used to confirm the presence of the exogenous PD-L1 mRNA.

In some embodiments, the engineered cell contains an exogenous polynucleotide that encodes FasL, such as human FasL. In some embodiments, FasL is overexpressed in the cell. In some embodiments, the expression of FasL is increased in the engineered cell compared to a similar reference or unmodified cell (including with any other modifications, such as genetic modifications) except that the reference or unmodified cell does not include the exogenous polynucleotide encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1. In certain embodiments, the polynucleotide encoding Fas-L is operably linked to a promoter.

In some embodiments, the polynucleotide encoding Fas-L is inserted into any one of the gene loci depicted in Table 1B, 2 or 4. In some cases, the polynucleotide encoding Fas-L is inserted into a safe harbor locus, such as but not limited to, a gene locus selected from AAVS1, CCR5, CLYBL, ROSA26, and SHS231. In particular embodiments, the polynucleotide encoding Fas-L is inserted into the CCR5 gene locus, the PPP1R12C (also known as AAVS1) gene locus or the CLYBL gene locus. In some embodiments, the polynucleotide encoding Fas-L is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding Fas-L, into a genomic locus of the cell.

In some embodiments, Fas-L protein expression is detected using a Western blot of cell lysates probed with antibodies against the Fas-L protein. In another embodiment, reverse transcriptase polymerase chain reactions (RT-PCR) are used to confirm the presence of the exogenous Fas-L mRNA.

In some embodiments, the engineered cell contains an exogenous polynucleotide that encodes CCL21, such as human CCL21. In some embodiments, CCL21 is overexpressed in the cell. In some embodiments, the expression of CCL21 is increased in the engineered cell compared to a similar reference or unmodified cell (including with any other modifications, such as genetic modifications) except that the reference or unmodified cell does not include the exogenous polynucleotide encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the polynucleotide encoding CCL21 is operably linked to a promoter.

In some embodiments, the polynucleotide encoding CCL21 is inserted into any one of the gene loci depicted in Table 1B, 2 or 4. In some cases, the polynucleotide encoding CCL21 is inserted into a safe harbor locus, such as but not limited to, a gene locus selected from AAVS1, CCR5, CLYBL, ROSA26, and SHS231. In particular embodiments, the polynucleotide encoding CCL21 is inserted into the CCR5 gene locus, the PPP1R12C (also known as AAVS1) gene locus or the CLYBL gene locus. In some embodiments, the polynucleotide encoding CCL21 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding CCL21, into a genomic locus of the cell.

In some embodiments, CCL21 protein expression is detected using a Western blot of cell lysates probed with antibodies against the CCL21 protein. In another embodiment, reverse transcriptase polymerase chain reactions (RT-PCR) are used to confirm the presence of the exogenous CCL21 mRNA.

In some embodiments, the engineered cell contains an exogenous polynucleotide that encodes CCL22, such as human CCL22. In some embodiments, CCL22 is overexpressed in the cell. In some embodiments, the expression of CCL22 is increased in the engineered cell compared to a similar reference or unmodified cell (including with any other modifications, such as genetic modifications) except that the reference or unmodified cell does not include the exogenous polynucleotide encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1. In certain embodiments, the polynucleotide encoding CCL22 is operably linked to a promoter.

In some embodiments, the polynucleotide encoding CCL22 is inserted into any one of the gene loci depicted in Table 1B, 2 or 4. In some cases, the polynucleotide encoding CCL22 is inserted into a safe harbor locus, such as but not limited to, a gene locus selected from AAVS1, CCR5, CLYBL, ROSA26, and SHS231. In particular embodiments, the polynucleotide encoding CCL22 is inserted into the CCR5 gene locus, the PPP1R12C (also known as AAVS1) gene locus or the CLYBL gene locus. In some embodiments, the polynucleotide encoding CCL22 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding CCL22, into a genomic locus of the cell.

In some embodiments, CCL22 protein expression is detected using a Western blot of cell lysates probed with antibodies against the CCL22 protein. In another embodiment, reverse transcriptase polymerase chain reactions (RT-PCR) are used to confirm the presence of the exogenous CCL22 mRNA.

In some embodiments, the engineered cell contains an exogenous polynucleotide that encodes Mfge8, such as human Mfge8. In some embodiments, Mfge8 is overexpressed in the cell. In some embodiments, the expression of Mfge8 is increased in the engineered cell compared to a similar reference or unmodified cell (including with any other modifications, such as genetic modifications) except that the reference or unmodified cell does not include the exogenous polynucleotide encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3. In certain embodiments, the polynucleotide encoding Mfge8 is operably linked to a promoter.

In some embodiments, the polynucleotide encoding Mfge8 is inserted into any one of the gene loci depicted in Table 1B, 2 or 4. In some cases, the polynucleotide encoding Mfge8 is inserted into a safe harbor locus, such as but not limited to, a gene locus selected from AAVS1, CCR5, CLYBL, ROSA26, and SHS231. In particular embodiments, the polynucleotide encoding Mfge8 is inserted into the CCR5 gene locus, the PPP1R12C (also known as AAVS1) gene locus or the CLYBL gene locus. In some embodiments, the polynucleotide encoding Mfge8 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding Mfge8, into a genomic locus of the cell.

In some embodiments, Mfge8 protein expression is detected using a Western blot of cell lysates probed with antibodies against the Mfge8 protein. In another embodiment, reverse transcriptase polymerase chain reactions (RT-PCR) are used to confirm the presence of the exogenous Mfge8 mRNA.

In some embodiments, the engineered cell contains an exogenous polynucleotide that encodes SerpinB9, such as human SerpinB9. In some embodiments, SerpinB9 is overexpressed in the cell. In some embodiments, the expression of SerpinB9 is increased in the engineered cell compared to a similar reference or unmodified cell (including with any other modifications, such as genetic modifications) except that the reference or unmodified cell does not include the exogenous polynucleotide encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4. In certain embodiments, the polynucleotide encoding SerpinB9 is operably linked to a promoter.

In some embodiments, the polynucleotide encoding SerpinB9 is inserted into any one of the gene loci depicted in Table 1B, 2 or 4. In some cases, the polynucleotide encoding SerpinB9 is inserted into a safe harbor locus, such as but not limited to, a gene locus selected from AAVS1, CCR5, CLYBL, ROSA26, and SHS231. In particular embodiments, the polynucleotide encoding SerpinB9 is inserted into the CCR5 gene locus, the PPP1R12C (also known as AAVS1) gene locus or the CLYBL gene locus. In some embodiments, the polynucleotide encoding SerpinB9 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding SerpinB9, into a genomic locus of the cell.

In some embodiments, SerpinB9 protein expression is detected using a Western blot of cell lysates probed with antibodies against the SerpinB9 protein. In another embodiment, reverse transcriptase polymerase chain reactions (RT-PCR) are used to confirm the presence of the exogenous SerpinB9 mRNA.

### V. POPULATIONS OF ENGINEERED CELLS AND PHARMACEUTICAL COMPOSITIONS

Provided herein are populations of engineered cells containing a plurality of the provided engineered cells.

In some embodiments, at least about any of 50%, 60%, 70%, 80%, 90%, 95%, 96%, 97%, 98%, 99%, 99.9%, or 99.99% of cells in the population comprise reduced expression of one or more of CACNA1G, CACNA1H, HCN4, and SLC8A1 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of one or more of CACNA1G, HCN4, and SLC8A1 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of CACNA1G relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of CACNA1H relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of HCN4 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of SLC8A1 relative to an unmodified or unaltered cell of the same cell type that does not comprise the one or more modifications.

In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a CACNA1G gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a CACNA1H gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a HCN4 gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a SLC8A1 gene relative to an unmodified or unaltered cell of the same cell type that does not comprise the one or more modifications.

In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CACNA1G gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CACNA1G gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous HCN4 gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous SLC8A1 gene.

In some embodiments, at least about any of 50%, 60%, 70%, 80%, 90%, 95%, 96%, 97%, 98%, 99%, 99.9%, or 99.99% of cells in the population comprise reduced expression of CACNA1G, CACNA1H, HCN4, and SLC8A1 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of CACNA1G, HCN4, and SLC8A1 relative to an unmodified or unaltered cell of the same cell type that does not comprise the one or more modifications.

In some embodiments, at least about any of 50%, 60%, 70%, 80%, 90%, 95%, 96%, 97%, 98%, 99%, 99.9%, or 99.99% of cells in the population comprise reduced expression of MHC class I molecule and/or MHC class II molecule relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of B2M, TIP1, and/or CIITA relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of B2M and CIITA relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of TIP1 and CIITA relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a B2M gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a TIP1 gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a CIITA gene relative to an unmodified or unaltered cell of the same cell type that does not comprise the one or more modifications.

In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous B2M gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous TIP1 gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CIITA gene.

In some embodiments, at least about any of 50%, 60%, 70%, 80%, 90%, 95%, 96%, 97%, 98%, 99%, 99.9%, or 99.99% of cells in the population comprise increased expression of CD47 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding CD47.

In some embodiments, at least about any of 50%, 60%, 70%, 80%, 90%, 95%, 96%, 97%, 98%, 99%, 99.9%, or 99.99% of cells in the population comprise (a) reduced expression of B2M and CIITA and (b) increased expression of CD47, relative to an unmodified or unaltered cell of the same cell type that does not comprise the one or more modifications.

In some embodiments, at least about any of 50%, 60%, 70%, 80%, 90%, 95%, 96%, 97%, 98%, 99%, 99.9%, or 99.99% of cells in the population comprise increased expression of KCNJ2 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding KCNJ2.

In some embodiments, at least about any of 50%, 60%, 70%, 80%, 90%, 95%, 96%, 97%, 98%, 99%, 99.9%, or 99.99% of cells in the population comprise increased expression of triadin relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding triadin.

In some embodiments, at least about any of 50%, 60%, 70%, 80%, 90%, 95%, 96%, 97%, 98%, 99%, 99.9%, or 99.99% of cells in the population comprise increased expression of sarcalumenin relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding sarcalumenin.

In some embodiments, at least about any of 50%, 60%, 70%, 80%, 90%, 95%, 96%, 97%, 98%, 99%, 99.9%, or 99.99% of cells in the population comprise increased expression of HRC relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding HRC.

In some embodiments, at least about any of 50%, 60%, 70%, 80%, 90%, 95%, 96%, 97%, 98%, 99%, 99.9%, or 99.99% of cells in the population comprise increased expression of calsequestrin-2 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise an exogenous polynucleotide encoding calsequestrin-2.

In some embodiments, at least about any of 50%, 60%, 70%, 80%, 90%, 95%, 96%, 97%, 98%, 99%, 99.9%, or 99.99% of cells in the population comprise (a) reduced expression of B2M, CIITA CACNA1G, CACNA1H, HCN4, and SLC8A1 and (b) increased expression of KCNJ2 and CD47 relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise (a) reduced expression of B2M, CIITA CACNA1G, HCN4, and SLC8A1 and (b) increased expression of KCNJ2 and CD47 relative to an unmodified or unaltered cell of the same cell type that does not comprise the one or more modifications.

Also provided herein are compositions comprising the engineered cells or populations of engineered cells.

In some embodiments the compositions comprising the engineered cells or populations of the engineered cells are therapeutic compositions. In some embodiments, the therapeutic composition comprises engineered primary cardiac cells that are engineered to prevent or reduce EA, and optionally, to be hypoimmunmogenic. In some embodiments, the therapeutic composition comprises engineered cardiomyocytes differentiated from PSCs that are engineered to prevent or reduce EA, and optionally, to be hypoimmunmogenic.

In some embodiments, the compositions are pharmaceutical compositions. In some embodiments, the pharmaceutical composition provided herein further include a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The pharmaceutical composition in some embodiments contains engineered cells as described herein in amounts effective to treat or prevent the disease or condition, such as a therapeutically effective or prophylactically effective amount. In some embodiments, the pharmaceutical composition contains engineered cells as described herein in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, engineered cells as described herein are administered using standard administration techniques, formulations, and/or devices. In some embodiments, engineered cells as described herein are administered using standard administration techniques, formulations, and/or devices. Provided are formulations and devices, such as syringes and vials, for storage and administration of the compositions. Engineered cells can be administered via localized injection, including catheter administration, systemic injection, localized injection, intravenous injection, or parenteral administration. When administering a therapeutic composition (e.g., a pharmaceutical composition containing an engineered cell), it will generally be formulated in a unit dosage injectable form (solution, suspension, emulsion).

Formulations include those for intravenous, intraperitoneal, or subcutaneous, administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the engineered cells, such as a saline solution, a dextrose solution or a solution comprising human serum albumin. In some embodiments, the pharmaceutically acceptable carrier or vehicle for such compositions is any non-toxic aqueous solution in which the engineered cells can be maintained, or remain viable, for a time sufficient to allow administration of live cells. For example, the pharmaceutically acceptable carrier or vehicle can be a saline solution or buffered saline solution.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided engineered cells. In some embodiments, the provided engineered cells are cryopreserved in a cryopreservation medium. In some embodiments, the cryopreservation medium is a serum free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the pharmaceutical composition comprises engineered cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

In some embodiments, the cryopreserved engineered cells are prepared for administration by thawing. In some cases, the engineered cells can be administered to a subject immediately after thawing. In such an embodiment, the composition is ready-to-use without any further processing. In other cases, the engineered cells are further processed after thawing, such as by resuspension with a pharmaceutically acceptable carrier, incubation with an activating or stimulating agent, or are activated washed and resuspended in a pharmaceutically acceptable buffer prior to administration to a subject.

### VI. THERAPEUTIC COMPOSITIONS AND GENERATION THEREOF

Provided herein are cardiac cell therapy compositions comprising any of the engineered cells provided herein, including for use in reducing or preventing engraftment arrhythmia in a subject administered the composition. In some embodiments, the cardiac cell therapy compositions includes an engineered cell as described in Section II or Section III. In some embodiments, the cardiac cell therapy compositions includes a population of engineered cells as described in Section VI.

In some embodiments, the cardiac cell therapy is a pharmaceutical composition comprising engineered cardiomyocytes and a pharmaceutically acceptable carrier. In some embodiments, the cardiac cell therapy is a suspension of engineered cardiomyocytes. In some embodiments, the cardiac cell therapy is a tissue graft comprising engineered cardiomyocytes.

In some embodiments, the cardiac cell therapy is a pharmaceutical composition comprising engineered primary cardiac cells and a pharmaceutically acceptable carrier. In some embodiments, the cardiac cell therapy is a suspension of engineered primary cardiac cells. In some embodiments, the cardiac cell therapy is a tissue graft comprising engineered primary cardiac cells.

In some embodiments, the cardiomyocytes of the cardiac cell therapy are primary cardiomyocytes derived from a donor, such as a human donor. In some embodiments, the primary cardiomyocytes are allogeneic to the recipient.

In some embodiments, the engineered cardiomyocytes of the cardiac cell therapy are derived from pluripotent stem cells (PSCs). In some embodiments, the engineered cardiomyocytes are differentiated from PSCs, such as embryonic stem cells (ESCs) or induced PSCs (iPSCs). In some cases, the engineered cardiomyocytes are differentiated from iPSCs derived from a donor, such as a human donor. In some embodiments, the engineered cardiomyocytes of the cardiac cell therapy are primary cardiomyocytes from a donor.

A variety of different methods of generating pluripotent stem cells (generally referred to as iPSCs; miPSCs for murine cells or hiPSCs for human cells) are known. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7 (1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

The number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4, (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, iPSCs are made from non-pluripotent cells such as CD34+ cord blood cells, fibroblasts, etc., by transiently expressing the reprogramming factors as described herein. For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015 Jan. 28; 5:8081 (doi: 10.1038/srep08081), hereby incorporated by reference in its entirety and specifically for the methods and reagents for the generation of the miPSCs. See also, e.g., Burridge et al., PLOS One, 2011 6 (4): 18293, hereby incorporated by reference in its entirety and specifically for the methods outlined therein.

In some embodiments, PSCs (e.g. iPSCs) generated by any of the methods described herein and/or known in the art are differentiated into cardiomyocytes, such as to produce a composition highly enriched in cardiomyocytes.

The PSCs (e.g. iPSCs) can be differentiated into cardiomyocytes by any known methods, including but not limited to those described in Murry and Keller, Cell (2008) 132 (4): 661-80; Burridge et al., Cell Stem Cell (2012) 10:16-28; Lian et al., Nature Protocols (2013) 8:162-65; Batalov and Feiberg, Biomark. Insight (2015) 10 (Suppl. 1): 71-6; Denning et al., Biochim. Biophys. Acta Mol. Cell Res. (2016) 1863:1728-48; Breckwoldt et al., Nature Protocols (2017) 12:1177-97; Guo et al., Stem Cell Res. And Ther. (2018) 9:44; and Leitolis et al., Front. Cell Dev. Biol. (2019) 8:164. In some embodiments, the PSCs are differentiated into cardiomyocytes by a method comprising adherent (i.e., monolayer) culture. In some embodiments, the PSCs are differentiated into cardiomyocytes by a method comprising non-adherent (e.g., suspension) culture.

In some embodiments, the engineered cell comprises one or more modifications that reduce expression of CACNA1G and/or CACNA1H. In some embodiments, the engineered cell comprises one or more modifications that reduce expression of CACNA1G. In some embodiments, the engineered cell comprises one or more modifications that reduce expression of CACNA1G and does not comprise one or more modifications that reduce expression of CACNA1H. In some embodiments, the engineered cell comprises one or more modifications that reduce expression of CACNA1H. In some embodiments, the engineered cell comprises one or more modifications that reduce expression of CACNA1H and does not comprise one or more modifications that reduce expression of CACNA1G. In some embodiments, the engineered cell comprises one or more modifications that reduce expression of CACNA1G and CACNA1H.

In some embodiments, the engineered cell is a PSC that is differentiated into a cardiomyocyte by a method comprising adherent (i.e., monolayer) culture and/or non-adherent (e.g., suspension) culture. In some embodiments, the engineered cell is a PSC that is differentiated into a cardiomyocyte by a method comprising adherent (i.e., monolayer) culture. In some embodiments, the engineered cell is a PSC that is differentiated into a cardiomyocyte by a method that does not comprise non-adherent (e.g., suspension) culture. In some embodiments, the engineered cell is a PSC that is differentiated into a cardiomyocyte by a method comprising non-adherent (e.g., suspension) culture. In some embodiments, the engineered cell is a PSC that is differentiated into a cardiomyocyte by a method that does not comprise adherent (i.e., monolayer) culture. In some embodiments, the engineered cell is a PSC that is differentiated into a cardiomyocyte by a method comprising adherent (i.e., monolayer) culture and non-adherent (e.g., suspension) culture.

In some embodiments, the engineered cell is a PSC comprising one or more modifications that reduce expression of CACNA1G, and the PSC is differentiated into a cardiomyocyte by a method comprising non-adherent (e.g., suspension) culture. In some embodiments, the engineered cell does not comprise one or more modifications that reduce expression of CACNA1H. In some embodiments, the method does not comprise adherent (i.e., monolayer) culture.

In some embodiments, the engineered cell is a PSC comprising one or more modifications that reduce expression of CACNA1H, and the PSC is differentiated into a cardiomyocyte by a method comprising adherent (i.e., monolayer) culture. In some embodiments, the engineered cell does not comprise one or more modifications that reduce expression of CACNA1G. In some embodiments, the method does not comprise non-adherent (i.e., suspension) culture.

In some embodiments, the engineered cardiomyocytes are allogeneic to a subject receiving a transplant of the cardiomyocytes. In some embodiments, the PSCs (e.g. iPSCs) from which cardiomyocytes are derived are engineered to be hypoimmunogenic by any known methods, including any of those described in Sections III or IV. In some embodiments, the cardiomyocytes have been previously engineered to be hypoimmunogenic by any known methods, including any of those described in Sections III or IV.

For example, nucleic acid sequences may be modified within PSCs (e.g. iPSCs) to generated hypoimmunogenic PSCs. Technologies to modify nucleic acid sequences within cells include homologous recombination, knock-in, knock-out, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

A number of different techniques can be used to engineer the PSCs (e.g. iPSCs) to be hypo-immunogenic, including those described in WO 2020/018615, incorporated herein by reference in its entirety. In some embodiments, engineering of the PSCs (iPSCs) to be hypoimmunogenic reduces an immune response of the recipient to the cells, including cardiomyocytes differentiated from the hypoimmunogenic PSCs (e.g. iPSCs).

### VII. ADMINISTRATION OF A CARDIAC CELL THERAPY

Provided herein are methods of administering and uses of a cardiac cell therapy to a subject in need thereof. In some embodiments, the subject has a condition or disease, such as a heart condition or disease.

Methods for administration of cardiomyocyte compositions are known and may be used in connection with the provided methods and compositions.

In some embodiments, the engineered cardiomyocytes or composition comprising the same is administered in an effective amount or dose to treat a heart condition or disease. Provided herein are uses of any of the provided engineered cardiomyocytes or composition comprising the same in such methods and treatments, and in the preparation of a medicament in order to carry out such therapeutic methods. In some embodiments, the methods are carried out by administering the engineered cardiomyocytes or compositions comprising the same, to the subject having, having had, or suspected of having the disease or condition or disorder. In some embodiments, the methods thereby treat the heart condition or disease in the subject. Also provided herein are uses of any of the compositions, such as pharmaceutical compositions provided herein, for treating a heart disease or condition.

In some embodiments, the engineered cardiomyocytes are any as described herein, such as in Section II or Section III, and compositions containing the same. In some embodiments, the composition includes a population of engineered cells as described in Section VI.

In some embodiments, administration of the cardiac cell therapy comprises delivery into a subject's heart tissue. In some embodiments, delivery into a subject's heart tissue comprises intravenous injection, intraarterial injection, intracoronary injection, intramuscular injection, intraperitoneal injection, intramyocardial injection, trans-endocardial injection, trans-epicardial injection, and/or infusion. In some embodiments, delivery into a subject's heart tissue comprises intramyocardial injection. In some embodiments, delivery into a subject's heart tissue comprises trans-epicardial injection. In some embodiments, delivery into a subject's heart tissue comprises trans-endocardial injection. In some embodiments, delivery into a subject's heart tissue comprises delivery at the site of a myocardial infarct (MI). In some embodiments, delivery into a subject's heart tissue comprises delivery near the site of a myocardial infarct (MI). In some embodiments, delivery into a subject's heart tissue comprises delivery within about 1 mm, 5 mm, 10 mm, 20 mm, 30 mm, 40 mm, 50 mm, 60 mm, 70 mm, 80 mm, or 90 mm of the site of an injury, such as a MI. In some embodiments, delivery into a subject's heart tissue comprises delivery within about 0.1 cm, 0.2 cm, 0.3 cm, 0.4 cm, 0.5 cm, 0.6 cm, 0.7 cm, 0.8 cm, 0.9 cm, 1.0 cm, 1.2 cm, 1.4 cm, 1.6 cm, 1.8 cm, 2.0 cm, 2.5 cm, 3.0 cm, 3.5 cm, 4.0 cm, 4.5 cm, or 5.0 of the site of an injury such as a MI.

In some embodiments, the cardiac cell therapy is a pharmaceutical composition comprising cardiomyocytes and a pharmaceutically acceptable carrier. In some embodiments the pharmaceutically acceptable carrier comprises cell culture medium. In some embodiments, the pharmaceutical acceptable carrier is cell culture medium. In some embodiments, the pharmaceutically acceptable carrier comprises between about 1% and 20% of the total volume of the cardiac cell therapy composition. In some embodiments, the pharmaceutically acceptable carrier comprises between about 5% and 10% of the total volume of the cardiac cell therapy composition. In some embodiments, the pharmaceutically acceptable carrier comprises about 5% of the total volume of the cardiac cell therapy composition. In some embodiments, the pharmaceutically acceptable carrier comprises about 10% of the total volume of the cardiac cell therapy composition. In some embodiments, the pharmaceutically acceptable carrier comprises about 15% of the total volume of the cardiac cell therapy composition.

In some embodiments, the cardiac cell therapy is a suspension of cardiomyocytes, including those differentiated from PSCs, or primary cardiac cells. In some embodiments, the cardiac cell therapy is an engineered tissue graft comprising cardiomyocytes, including those differentiated from PSCs, or primary cardiac cells.

In any of the provided embodiments, the subject administered the cardiac cell therapy has a condition or disease, such as a heart condition or disease. In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction (MI), myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart condition or disease is myocardial infarction (MI). Thus, in some embodiments, the cardiac cell therapy is administered to a subject to treat a MI (e.g. as a composition comprising cardiomyocytes).

In some embodiments, the subject is a candidate for a left ventricular assist device (LVAD). In some embodiments, the subject is a candidate for a LVAD at the time of administration of the cardiac cell therapy. In some embodiments, the subject has a LVAD at the time of administration of the cardiac cell therapy. In some embodiments, the subject receives a LVAD at a time subsequent to administration of the cardiac cell therapy.

In some embodiments, the subject is a human. In some embodiments, the subject is a non-human primate (NHP).

In some embodiments, one or more immunosuppressive agents is further administered to the subject. In some embodiments, the subject has been administered one or more immunosuppressive agents.

In some embodiments, an immunosuppressive and/or immunomodulatory agent is not administered to the patient before the first administration of the population of modified cells, or in a composition containing the same.

In some embodiments, an immunosuppressive and/or immunomodulatory agent may be administered to a patient received administration of modified cells. In some embodiments, the immunosuppressive and/or immunomodulatory agent is administered prior to administration of the modified cells. In some embodiments, the immunosuppressive and/or immunomodulatory agent is administered prior to administration of a first and/or second administration of modified cells.

Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-a and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the first administration of the cells, the administration is at a lower dosage than would be required for cells with MHC class I molecules and/or MHC class II molecules expression and without exogenous expression of CD47.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent can be administered to the patient before the first administration of the population of modified cells. In some embodiments, an immunosuppressive and/or immunomodulatory agent is administered at least 1, 2, 3, 4, 5, 6, 7, 8, 9, 10, 11, 12, 13, 14 days or more before the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the cells.

In particular embodiments, an immunosuppressive and/or immunomodulatory agent is not administered to the patient after the first administration of the cells, or is administered at least 1, 2, 3, 4, 5, 6, 7, 8, 9, 10, 11, 12, 13, 14 days or more after the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the cells.

In some embodiments, an immunosuppressive and/or immunomodulatory agent is not administered to the patient before the administration of the population of engineered cells. In many embodiments, an immunosuppressive and/or immunomodulatory agent is administered to the patient before the first and/or second administration of the population of modified cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the cells. In particular embodiments, an immunosuppressive and/or immunomodulatory agent is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the cells.

In some embodiments where an immunosuppressive and/or immunomodulatory agent is administered to the patient before or after the administration of the cells, the administration is at a lower dosage than would be required for immunogenic cells (e.g. a population of cells of the same or similar cell type or phenotype but that do not contain the modifications, e.g. genetic modifications, of the modified cells, e.g. with endogenous levels of CD142, MHC class I molecules, and/or MHC class II molecules expression and without increased (e.g., exogenous) expression of CD47).

### VIII. ARTICLES OF MANUFACTURE AND KITS

Also provided are articles of manufacture containing a cardiac cell therapy and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing a disease or condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include a container with a composition contained therein, wherein the composition is a cardiac cell therapy (e.g. a pharmaceutical composition comprising cardiomyocytes and a pharmaceutically acceptable carrier)

The article of manufacture may further include a package insert indicating that the composition can be used to treat a particular condition (e.g. heart disease or condition, such as myocardial infarction). Alternatively, or additionally, the article of manufacture may further include another or the same container comprising a pharmaceutically-acceptable buffer or excipient. It may further include other materials such as other buffers, diluents, filters, needles, and/or syringes.

### IX. EXEMPLARY EMBODIMENTS

Among the provided embodiments are:

1. An engineered cell comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications.
2. The engineered cell of embodiment 1, wherein the engineered cell comprises one or more modifications that reduce expression of CACNA1G.
3. The engineered cell of embodiment 1 or embodiment 2, wherein the engineered cell comprises one or more modifications that reduce expression of HCN4 and/or SLC8A1.
4. The engineered cell of any of embodiments 1-3, wherein the engineered cell comprises one or more modifications that increase expression of KCNJ2.
5. The engineered cell of any of embodiments 1-4, wherein the engineered cell comprises one or more modifications that (a) reduce expression of CACNA1G, HCN4, and SLC8A1; and (b) increase expression of KCNJ2.
6. The engineered cell of any of embodiments 1-5, wherein the engineered cell is a pluripotent stem cell (PSC).
7. The engineered cell of embodiment 6, wherein the PSC is an induced pluripotent stem cell (iPSC).
8. The engineered cell of embodiment 6, wherein the PSC is an embryonic stem cell (ESC).
9. The engineered cell of any of embodiments 1-5, wherein the engineered cell is a primary cardiac cell.
10. The engineered cell of any of embodiments 1-5 and 9, wherein the engineered cell is a cardiomyocyte or a precursor thereof.
11. The engineered cell of any of embodiments 1-5, 9 and 10, wherein the engineered cell is a cardiomyocyte.
12. The engineered cell of any of embodiments 1-5 and 9-11, wherein the engineered cell is a primary cardiomyocyte.
13. The engineered cell of embodiment 10 or embodiment 11, wherein the cardiomyocyte or a precursor thereof has been differentiated from a pluripotent stem cell (PSC) in vitro.
14. The engineered cell of embodiment 13, wherein the in vitro differentiation of the cardiomyocyte or a precursor thereof from a PSC comprises differentiation in suspension culture.
15. The engineered cell of any of embodiments 1-14, wherein the engineered cell comprises one or more modifications that (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell of the same cell type that does not comprise the one or more modifications.
16. The engineered cell of embodiment 15, wherein MHC HLA class I molecules are selected from the group consisting of HLA-A, HLA-B, HLA-C, and a combination thereof.
17. The engineered cell of embodiment 15 or embodiment 16, wherein the one or more modifications in (ii) reduce expression of one or more MHC HLA class I molecules.
18. The engineered cell of any of embodiments 15-17, wherein the one or more modifications in (ii) reduce expression of MHC HLA class I molecules HLA-A, HLA-B, and HLA-C.
19. The engineered cell of any of embodiments 15-18, wherein the one or more modifications in (ii) reduce protein expression of one or more MHC HLA class I molecules.
20. The engineered cell of embodiment 19, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
21. The engineered cell of any of embodiments 1-20, wherein the engineered cell comprises one or more modifications that reduce cell surface expression of one or more MHC HLA class I molecules.
22. The engineered cell of any of embodiments 1-21, wherein the engineered cell comprises one or more modifications that reduce a function of one or more MHC HLA class I molecules, optionally wherein the function is antigen presentation.
23. The engineered cell of any of embodiments 15-22 wherein the one or more modifications in (ii) reduce expression of the B-2 microglobulin (B2M) gene and/or the transporter 1, ATP binding cassette subfamily B member (TAP1) gene.
24. The engineered cell of embodiment 23, wherein the one or more modifications that reduce expression in (ii) reduce expression of the B2M gene.
25. The engineered cell of embodiment 23 or embodiment 24, wherein the one or more modifications that reduce expression reduces mRNA expression of the gene.
26. The engineered cell of any of embodiments 23-25, wherein the one or more modifications that reduce expression reduces protein expression of a protein encoded by the gene.
27. The engineered cell of any of embodiments 23-26, wherein the one or more modifications that reduce expression comprises inactivation or disruption of one allele of the gene.
28. The engineered cell of any of embodiments 23-27, wherein the one or more modifications that reduce expression comprises inactivation or disruption of both alleles of the gene.
29. The engineered cell of any of embodiments 23-28, wherein the one or more modifications that reduce expression comprises inactivation or disruption of all coding sequences of the gene in the cell.
30. The engineered cell of any of embodiments 27-29, wherein the inactivation or disruption comprises an indel in one allele of the gene.
31. The engineered cell of any of embodiments 27-30, wherein the inactivation or disruption comprises an indel in both alleles of the gene.
32. The engineered cell of any of embodiments 23-31, wherein the one or more modifications that reduce expression comprises a frameshift mutation or a deletion of a contiguous stretch of genomic DNA of the gene.
33. The engineered cell of any of embodiments 23-32, wherein the gene is knocked out.
34. The engineered cell of any of embodiments 17-33, wherein the one or more modifications that reduce expression of one or more MHC HLA class I molecules is generated by nuclease-mediated gene editing.
35. The engineered cell of embodiment 34, wherein the nuclease-mediated gene editing is mediated by a zinc finger nuclease (ZFN), a TAL-effector nuclease (TALEN), or a CRISPR-Cas system that targets the gene.
36. The engineered cell of embodiment 34 or 35, wherein the nuclease-mediated gene editing uses a CRISPR-Cas system comprising a CRISPR-Cas nuclease and a guide RNA (gRNA) having a targeting domain that is complementary to at least one target site within the gene.
37. The engineered cell of embodiment 36, wherein the CRISPR-Cas system is a ribonucleoprotein (RNP) complex comprising the gRNA and a Cas protein.
38. The engineered cell of any of embodiments 15-37, wherein the one or more modifications in (ii) reduce expression of MHC HLA class I and class II molecules.
39. The engineered cell of any of embodiments 15-38, wherein the one or more modifications in (ii) reduce expression of MHC HLA class II molecules HLA-DP, HLA-DQ, and/or HLA-DR.
40. The engineered cell of any of embodiments 15-39, wherein the one or more modifications in (ii) reduce protein expression of one or more MHC class II molecules.
41. The engineered cell of embodiment 40, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
42. The engineered cell of any of embodiments 1-41, wherein the engineered cell comprises one or more modifications that reduce cell surface expression of one or more MHC HLA class II molecules.
43. The engineered cell of any of embodiments 1-42, wherein the engineered cell comprises one or more modifications that reduce a function of one or more MHC HLA class II molecules, optionally wherein the function is antigen presentation.
44. The engineered cell of any of embodiments 15-43, wherein the one or more modifications in (ii) reduce expression of the CIITA gene.
45. The engineered cell of embodiment 44, wherein the one or more modifications that reduce expression reduce mRNA expression of the CIITA gene.
46. The engineered cell of embodiment 44 or embodiment 45, wherein the one or more modifications that reduce expression reduces expression of a CIITA protein.
47. The engineered cell of any of embodiments 44-46, wherein the one or more modifications that reduce expression comprises inactivation or disruption of one allele of the CIITA gene.
48. The engineered cell of any of embodiments 44-47, wherein the one or more modifications that reduce expression comprises inactivation or disruption of both alleles of the CIITA gene.
49. The engineered cell of any of embodiments 44-48, wherein the one or more modifications that reduce expression comprises inactivation or disruption of all CIITA coding sequences in the cell.
50. The engineered cell of any of embodiments 47-49, wherein the inactivation or disruption comprises an indel in one allele of the CIITA gene.
51. The engineered cell of any of embodiments 47-50, wherein the inactivation or disruption comprises an indel in both alleles of the CIITA gene.
52. The engineered cell of any of embodiments 44-51, wherein the one or more modifications that reduce expression comprises a frameshift mutation or a deletion of a contiguous stretch of genomic DNA of the CIITA gene.
53. The engineered cell of any of embodiments 15-52, wherein the CIITA gene is knocked out.
54. The engineered cell of any of embodiments 51-53, wherein the one or more modifications that reduce expression of one or more MHC HLA class II molecules is generated by nuclease-mediated gene editing.
55. The engineered cell of embodiment 54, wherein the nuclease-mediated gene editing is mediated by a zinc finger nuclease (ZFN), a TAL-effector nuclease (TALEN), or a CRISPR-Cas system that targets the CIITA gene.
56. The engineered cell of embodiment 54 or embodiment 55, wherein the nuclease-mediated gene editing uses a CRISPR-Cas system comprising a CRISPR-Cas nuclease and a guide RNA (gRNA) having a targeting domain that is complementary to at least one target site within the CIITA gene.
57. The engineered cell of embodiment 56, wherein the CRISPR-Cas system is a ribonucleoprotein (RNP) complex comprising the gRNA and a Cas protein.
58. The engineered cell of any of embodiments 15-57, wherein the one or more tolerogenic factors in (i) are selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof.
59. The engineered cell of any of embodiments 15-58, wherein the one or more tolerogenic factors in (i) are selected from the group consisting of CD47, PD-L1, HLA-E, HLA-G, CCL21, FASL, SERPINB9, CD200, MFGE8, and any combination thereof.
60. The engineered cell of any of embodiments 15-59, wherein the one or more tolerogenic factors in (i) comprise CD47.
61. The engineered cell of any of embodiments 58-60, wherein the one or more tolerogenic factors in (i) comprise CD47, and wherein the one or more modifications that increases expression of CD47 comprise an exogenous polynucleotide encoding the CD47 protein.
62. The engineered cell of embodiment 61, wherein the exogenous polynucleotide encoding the CD47 protein is integrated into the genome of the engineered cell.
63. The engineered cell of embodiment 61 or embodiment 62, wherein the exogenous polynucleotide encoding the CD47 protein encodes an amino acid sequence having at least 85% sequence identity to the amino acid sequence set forth in SEQ ID NO: 2, optionally wherein the exogenous polynucleotide encoding the CD47 protein encodes the amino acid sequence set forth in SEQ ID NO:2.
64. The engineered cell of any of embodiments 61-63, wherein the exogenous polynucleotide is integrated by non-targeted insertion into the genome of the engineered cell, optionally by introduction of the exogenous polynucleotide into the cell using a lentiviral vector.
65. The engineered cell of any of embodiments 61-63, wherein the exogenous polynucleotide is integrated by targeted insertion into a target genomic locus of the engineered cell.
66. The engineered cell of embodiment 65, wherein the target genomic locus is a safe harbor locus, a B2M gene locus, or a CIITA gene locus.
67. The engineered cell of embodiment 65 or embodiment 66, wherein the target genomic locus is selected from the group consisting of: a CCR5 gene locus, a CXCR4 gene locus, a PPP1R12C (also known as AAVS1) gene locus, an albumin gene locus, a SHS231 locus, a CLYBL gene locus, and a ROSA26 gene locus.
68. The engineered cell of any of embodiments 15-67, wherein the one or more modifications that reduce expression in (a) comprise reduced surface protein expression; and/or the one or more modifications that increase expression in (b) comprise increased surface protein expression.
69. An engineered cell comprising one or more modifications that: (a) reduce expression of CACNA1G, HCN4, and SLC8A1, one or more MHC HLA class I molecules, and/or one or more MHC HLA class II molecules; and (b) increase expression of KCNJ2 and CD47, relative to a cell of the same cell type that does not comprise the one or more modifications.
70. An engineered cell comprising one or more modifications that: (a) reduce expression of CACNA1G, HCN4, and SLC8A1, one or more MHC HLA class I molecules, and one or more MHC HLA class II molecules; and (b) increase expression of KCNJ2 and CD47, relative to a cell of the same cell type that does not comprise the one or more modifications.
71. The engineered cell of embodiment 69 or embodiment 70, wherein the one or more modifications that reduce expression of one or more MHC HLA class I molecules and/or one or more MHC class II molecules reduce expression of B2M and CIITA.
72. An engineered cell comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.
73. An engineered primary human cell comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.
74. An engineered induced pluripotent stem cell (iPSC) or embryonic stem cell (ESC) comprising one or more modifications that: (a) reduce expression of CACNA1G, HCN4, SLC8A1, B2M, and CIITA; and (b) increase expression of KCNJ2 and CD47, relative to a cell of the same cell type that does not comprise the one or more modifications.
75. An engineered cardiomyocyte that has been differentiated in vitro from an engineered cell of any of embodiments 1-74.
76. An engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cardiomyocyte that does not comprise the one or more modifications.
77. An engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) reduce expression of one or more of MHC HLA class I molecules and one or more MHC HLA class II molecules; (c) increase expression of one or more of KCNJ2, TRDN, SRL, HRC, CASQ2, and CD47; or (d) a combination thereof, relative to a cardiomyocyte that does not comprise the one or more modifications.
78. The engineered cardiomyocyte of embodiment 77, wherein the one or more modifications that reduce expression of one or more MHC HLA class I molecules and one or more MHC class II molecules reduce expression of B2M and CIITA.
79. An engineered cardiomyocyte that has been differentiated in vitro from a pluripotent stem cell (PSC), wherein the engineered cardiomyocyte comprises one or more modifications that: (a) reduce expression of one or more of CACNA1G, HCN4, and SLC8A1; (b) reduce expression of one or more of B2M, TAP1, and CIITA; (c) increase expression of one or more of KCNJ2, TRDN, SRL, HRC, CASQ2, and CD47; or (d) a combination thereof, relative to a cardiomyocyte that does not comprise the one or more modifications.
80. The engineered cell or cardiomyocyte of any of embodiments 69 and 75-79, wherein the engineered cell or cardiomyocyte comprises one or more modifications that reduce expression of CACNA1G.
81. The engineered cell or cardiomyocyte of any of embodiments 69 and 75-80, wherein the engineered cell or cardiomyocyte comprises one or more modifications that reduce expression of HCN4 and/or SLC8A1.
82. The engineered cell or cardiomyocyte of any of embodiments 69 and 75-81, wherein the engineered cell or cardiomyocyte comprises one or more modifications that increase expression of KCNJ2.
83. The engineered cell or cardiomyocyte of any of embodiments 69 and 75-82, wherein the engineered cell or cardiomyocyte comprises one or more modifications that (a) reduce expression of CACNA1G, HCN4, and SLC8A1; and (b) increase expression of KCNJ2.
84. The engineered cell or cardiomyocyte of any of embodiments 69, 75, 76, and 80-83, wherein the engineered cell or cardiomyocyte comprises one or more modifications that: (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.
85. The engineered cell or cardiomyocyte of any of embodiments 69-71 75, 77, 78, and 80-84, wherein MHC HLA class I molecules are selected from the group consisting of HLA-A, HLA-B, HLA-C, and a combination thereof.
86. The engineered cell or cardiomyocyte of embodiment 69-75 and 77-85, wherein the one or more modifications reduce expression of one or more MHC HLA class I molecules.
87. The engineered cell or cardiomyocyte of any of embodiments 69-75 and 77-86, wherein the one or more modifications reduce expression of MHC HLA class I molecules HLA-A, HLA-B, and HLA-C.
88 The engineered cell or cardiomyocyte of any of embodiments 69-75 and 77-87, wherein the one or more modifications reduce protein expression of one or more MHC HLA class I molecules.
89. The engineered cell or cardiomyocyte of embodiment 88, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
90. The engineered cell or cardiomyocyte of any of embodiments 69-89, wherein the engineered cell comprises one or more modifications that reduce cell surface expression of one or more MHC HLA class I molecules.
91. The engineered cell or cardiomyocyte of any of embodiments 69-90, wherein the engineered cell comprises one or more modifications that reduce a function of one or more MHC HLA class I molecules, optionally wherein the function is antigen presentation.
92. The engineered cell or cardiomyocyte of any of embodiments 69-91, wherein the one or more modifications that reduce expression reduce expression of the B2M gene.
92. The engineered cell or cardiomyocyte of any of embodiments 69-75 and 77-92, wherein the one or more modifications reduce expression of MHC HLA class I and class II molecules.
94. The engineered cell or cardiomyocyte of any of embodiments 69-75 and 77-93, wherein the one or more modifications reduce expression of MHC HLA class II molecules HLA-DP, HLA-DQ, or HLA-DR.
95. The engineered cell or cardiomyocyte of any of embodiments 69-75 and 77-94, wherein the one or more modifications reduce protein expression of one or more MHC class II molecules.
96. The engineered cell or cardiomyocyte of embodiment 95, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
97. The engineered cell or cardiomyocyte of any of 69-96, wherein the engineered cell or cardiomyocyte comprises one or more modifications that reduce cell surface expression of one or more MHC HLA class II molecules.
98. The engineered cell or cardiomyocyte of any of embodiments 69-97, wherein the engineered cell or cardiomyocyte comprises one or more modifications that reduce a function of one or more MHC HLA class II molecules, optionally wherein the function is antigen presentation.
99. The engineered cell or cardiomyocyte of any of embodiments 69-75 and 77-98, wherein the one or more modifications reduce expression of the CIITA gene.
100. The engineered cell or cardiomyocyte of embodiment 75, wherein at least one of the one or more tolerogenic factors is CD47.
101. The engineered cell or cardiomyocyte of any of embodiments 69-75 and 77-100, wherein the one or more modifications that increases expression of CD47 comprise an exogenous polynucleotide encoding the CD47 protein.
102. The engineered cell or cardiomyocyte of any of embodiments 69-101, wherein the phenotype of the engineered cell or cardiomyocyte comprises B2M^{indel/indel}; CIITA^{indel/indel}; and CD47^{tg}.
103. The engineered cell or cardiomyocyte of any of embodiments 1-102, which is human.
104. A composition comprising a plurality of the engineered cardiomyocytes of any of embodiments 10-68 and 75-103 105. The composition of embodiment 104, wherein the composition comprises between about 5×10⁸ and 1×10¹⁰ engineered cardiomyocytes, inclusive of each.
106. The composition of embodiment 104 or embodiment 105, wherein the composition comprises between about 1×10⁹ and about 5×10⁹ engineered cardiomyocytes, inclusive of each.
107. The composition of any of embodiments 104-106, wherein the composition comprises a pharmaceutically acceptable carrier.
108. A method of producing an engineered cell, the method comprising: (a) reducing expression of one or more of CACNA1G, HCN4, and SLC8A1; (b) increasing expression of one or more of KCNJ2, TRDN, SRL, HRC, and CASQ2; or (c) a combination thereof, in the cell.
109. The method of embodiment 108, wherein the method comprises reducing expression of CACNA1G in the cell.
110. The method of embodiment 108 or embodiment 109, wherein the method comprises reducing expression of HCN4 and/or SLC8A1 in the cell.
111. The method of any of embodiments 108-110, wherein the method comprises increasing expression of KCNJ2 in the cell.
112. The method of any of embodiments 108-111, wherein the method comprises: (a) reducing expression of CACNA1G, HCN4, and SLC8A1; and (b) increasing expression of KCNJ2, in the cell.
113. The method of any of embodiments 108-112, wherein the engineered cell is a pluripotent stem cell (PSC).
114. The method of embodiment 113, wherein the PSC is an induced pluripotent stem cell (iPSC).
115. The method of embodiment 113, wherein the PSC is an embryonic stem cell (ESC).
116. The method of any of embodiments 108-112, wherein the engineered cell is a primary cardiac cell.
117. The method of any of embodiments 108-112 and 116, wherein the engineered cell is a cardiomyocyte or a precursor thereof.
118. The method of any of embodiments 108-112, 116, and 117, wherein the engineered cell is a cardiomyocyte.
119. The method of any of embodiments 108-112 and 116-118, wherein the engineered cell is a primary cardiomyocyte.
120. The method of embodiment 117 or embodiment 118, wherein the cardiomyocyte or a precursor thereof has been differentiated from a pluripotent stem cell (PSC) in vitro.
121. The method of embodiment 120, wherein the in vitro differentiation of the cardiomyocyte or a precursor thereof from a PSC comprises differentiation in suspension culture.
122. The method of any of embodiments 108-115, wherein the method further comprises differentiating the PSC into a cardiomyocyte.
123. The method of embodiment 122, wherein differentiation of the cardiomyocyte from the PSC comprises differentiation in suspension culture.
124. The method of any of embodiments 108-123, wherein the engineered cell comprises one or more modifications that: (i) increase expression of one or more tolerogenic factors; and/or (ii) reduce expression of one or more major histocompatibility complex (MHC) class I molecules and/or MHC class II, relative to a cell of the same cell type that does not comprise the one or more modifications.
125. The method of embodiment 124, wherein MHC HLA class I molecules are selected from the group consisting of HLA-A, HLA-B, HLA-C, and a combination thereof.
126. The method of embodiment 124 or embodiment 125, wherein the one or more modifications in (ii) reduce expression of one or more MHC HLA class I molecules.
127. The method of any of embodiments 124-126, wherein the one or more modifications in (ii) reduce expression of MHC HLA class I molecules HLA-A, HLA-B, and HLA-C.
128. The method of any of embodiments 124-127, wherein the one or more modifications in (ii) reduce protein expression of one or more MHC HLA class I molecules.
129. The method of embodiment 128, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
130. The method of any of embodiments 108-129, wherein the engineered cell comprises one or more modifications that reduce cell surface expression of one or more MHC HLA class I molecules.
131. The method of any of embodiments 108-130, wherein the engineered cell comprises one or more modifications that reduce a function of one or more MHC HLA class I molecules, optionally wherein the function is antigen presentation.
132. The method of any of embodiments 124-131, wherein the one or more modifications that reduce expression in (ii) reduce expression of the B2M gene.
133. The method of any of embodiments 124-132, wherein the one or more modifications in (ii) reduce expression of MHC HLA class I and class II molecules.
134. The method of any of embodiments 124-133, wherein the one or more modifications in (ii) reduce expression of MHC HLA class II molecule HLA-DP, HLA-DQ, or HLA-DR.
135. The method of any of embodiments 124-134, wherein the one or more modifications in (ii) reduce protein expression of one or more MHC class II molecules.
136. The method of embodiment 135, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
137. The method of any of embodiments 108-136, wherein the engineered cell comprises one or more modifications that reduce cell surface expression of one or more MHC HLA class II molecules.
138. The method of any of embodiments 108-137, wherein the engineered cell comprises one or more modifications that reduce a function of one or more MHC HLA class II molecules, optionally wherein the function is antigen presentation.
139. The method of any of embodiments 124-138, wherein the one or more modifications in (ii) reduce expression of the CIITA gene.
140. The method of any of embodiments 124-139, wherein at least one of the one or more tolerogenic factors is CD47.
141. The method of any of embodiments 124-140, wherein at least one of the one or more tolerogenic factors is CD47, and wherein the one or more modifications that increases expression of CD47 comprise an exogenous polynucleotide encoding the CD47 protein.
142. The method of any of embodiments 108-141, wherein the phenotype of the engineered cell comprises B2M^{indel/indel}; CIITA^{indel/indel}; and CD47^{tg}.
143. A cardiac cell therapy comprising a plurality of cardiomyocytes produced by the method of any of embodiments 117-142.
144. A method of treatment comprising administering the cardiac cell therapy of embodiment 143 to a subject.
145. A method of treatment comprising administering a cardiac cell therapy comprising a plurality of cardiomyocytes of any of embodiments 10-68 and 75-103 to a subject.
146. A method of treatment comprising administering a cardiac cell therapy to a subject, wherein the cardiac cell therapy comprises engineered cardiomyocytes comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to cardiomyocytes that do not comprise the one or more modifications.
147. The method of embodiment 146, wherein the engineered cardiomyocytes comprise one or more modifications that reduce expression of CACNA1G.
148. The method of embodiment 146 or embodiment 147, wherein the engineered cardiomyocytes comprise one or more modifications that reduce expression of HCN4 and/or SLC8A1.
149. The method of any of embodiments 146-148, wherein the engineered cardiomyocytes comprise one or more modifications that increase expression of KCNJ2.
150. The method of any of embodiments 146-149, wherein the engineered cardiomyocytes comprise one or more modifications that (a) reduce expression of CACNA1G, HCN4, and SLC8A1; and (b) increase expression of KCNJ2.
151. The method of any of embodiments 144-150, wherein the cardiac cell therapy is administered as a suspension of cardiomyocytes or as an engineered tissue graft comprising cardiomyocytes and a matrix.
152. The method of any of embodiments 144-151, wherein administration of the cardiac cell therapy comprises delivery into a subject's heart tissue, optionally by intravenous injection, intraarterial injection, intracoronary injection, intramuscular injection, intraperitoneal injection, intramyocardial injection, trans-endocardial injection, trans-epicardial injection, and/or infusion.
153. The method of any of embodiments 144-152, wherein administration of the cardiac cell therapy to the subject results in less engraftment arrhythmia (EA) in the subject, relative to a cardiac cell therapy comprising cardiomyocytes not having the one or more modifications.
154. The method of any of embodiments 144-153, wherein administration of the cardiac cell therapy to the subject does not cause engraftment arrhythmia (EA) in the subject.
155. The method of any of embodiments 143-154, wherein the cardiac cell therapy comprises between about 5×10⁸ and 1×10¹⁰ engineered cardiomyocytes, inclusive of each.
156. The method of any of embodiments 143-155, wherein the cardiac cell therapy comprises between about 1×10⁹ and about 5×10⁹ engineered cardiomyocytes, inclusive of each.
157. The method of any of embodiments 143-156, wherein the cardiac cell therapy comprises a pharmaceutically acceptable carrier.
158. The method of any of embodiments 143-157, wherein the subject has a heart disease or condition.
159. The method of embodiment 158, wherein the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.
160. The method of embodiment 158 or embodiment 159, wherein the heart disease or condition is myocardial infarction (MI).
161. The method of any of embodiments 146-161, wherein the engineered cardiomyocytes comprise one or more modifications that: (i) increase expression of one or more tolerogenic factors; and/or (ii) reduce expression of one or more major histocompatibility complex (MHC) class I molecules and/or MHC class II, relative to cardiomyocytes that do not comprise the one or more modifications that make the engineered cardiomyocytes hypoimmunogenic.
162. The method of embodiment 161, wherein MHC HLA class I molecules are selected from the group consisting of HLA-A, HLA-B, HLA-C, and a combination thereof.
163. The method of embodiment 161 or embodiment 162, wherein the one or more modifications in (ii) reduce expression of one or more MHC HLA class I molecules.
164. The method of any of embodiments 161-163, wherein the one or more modifications in (ii) reduce expression of MHC HLA class I molecules HLA-A, HLA-B, and HLA-C.
165. The method of any of embodiments 161-164, wherein the one or more modifications in (ii) reduce protein expression of one or more MHC HLA class I molecules.
166. The method of embodiment 165, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
167. The method of any of embodiments 146-166, wherein the engineered cardiomyocytes comprise one or more modifications that reduce cell surface expression of one or more MHC HLA class I molecules.
168. The method of any of embodiments 146-167, wherein the engineered cardiomyocytes comprise one or more modifications that reduce a function of one or more MHC HLA class I molecules, optionally wherein the function is antigen presentation.
169. The method of any of embodiments 161-168, wherein the one or more modifications in (ii) that reduce expression reduce expression of the B2M gene.
170. The method of any of embodiments 161-169, wherein the one or more modifications in (ii) reduce expression of MHC HLA class I and class II molecules.
171. The method of any of embodiments 161-170, wherein the one or more modifications in (ii) reduce expression of MHC HLA class II molecules HLA-DP, HLA-DQ, or HLA-DR.
172. The method of any of embodiments 161-171, wherein the one or more modifications in (ii) reduce protein expression of one or more MHC class II molecules.
173. The method of embodiment 172, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
174. The method of any of embodiments 146-173, wherein the engineered cardiomyocytes comprise one or more modifications that reduce cell surface expression of one or more MHC HLA class II molecules.
175. The method of any of embodiments 146-174, wherein the engineered cardiomyocytes comprise one or more modifications that reduce a function of one or more MHC HLA class II molecules, optionally wherein the function is antigen presentation.
176. The method of any of embodiments 161-175, wherein the one or more modifications in (ii) reduce expression of the CIITA gene.
177. The method of any of embodiments 161-176, wherein the one or more tolerogenic factors comprise CD47.
178. The method of any of embodiments 161-177, wherein the one or more tolerogenic factors comprise CD47, and wherein the one or more modifications that increases expression of CD47 comprise an exogenous polynucleotide encoding the CD47 protein.
179. The method of any of embodiments 146-178, wherein the phenotype of the engineered cardiomyocytes comprises B2M^{indel/indel}; CIITA^{indel/indel}; and CD47^{tg}.
180. The method of any of embodiments 144-160, wherein the cardiomyocytes are autologous to the subject.
181. The method of any of embodiments 144-180, wherein the subject is a human.

Also among the provided embodiments are:

1. An engineered cell comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications.
2. The engineered cell of embodiment 1, wherein the engineered cell comprises one or more modifications that reduce expression of CACNA1G.
3. The engineered cell of embodiment 1 or embodiment 2, wherein the engineered cell comprises one or more modifications that reduce expression of HCN4 and/or SLC8A1.
4. The engineered cell of any of embodiments 1-3, wherein the engineered cell comprises one or more modifications that increase expression of KCNJ2.
5. The engineered cell of any of embodiments 1-4, wherein the engineered cell comprises one or more modifications that increase expression of TRDN.
6. The engineered cell of any of embodiments 1-5, wherein the engineered cell comprises one or more modifications that increase expression of SRL.
7. The engineered cell of any of embodiments 1-6, wherein the engineered cell comprises one or more modifications that increase expression of HRC.
8. The engineered cell of any of embodiments 1-7, wherein the engineered cell comprises one or more modifications that increase expression of CASQ2.
9. The engineered cell of any of embodiments 1-8, wherein the engineered cell comprises one or more modifications that (a) reduce expression of CACNA1G, HCN4, and SLC8A1; and (b) increase expression of KCNJ2.
10. The engineered cell of any of embodiments 1-9, wherein the engineered cell is a pluripotent stem cell (PSC).
11. The engineered cell of embodiment 10, wherein the PSC is an induced pluripotent stem cell (iPSC).
12. The engineered cell of embodiment 10, wherein the PSC is an embryonic stem cell (ESC).
13. The engineered cell of any of embodiments 1-9, wherein the engineered cell is a primary cardiac cell.
14. The engineered cell of any of embodiments 1-9 and 13, wherein the engineered cell is a cardiomyocyte or a precursor thereof.
15. The engineered cell of any of embodiments 1-9, 13, and 14, wherein the engineered cell is a cardiomyocyte.
16. The engineered cell of any of embodiments 1-9 and 13-15, wherein the engineered cell is a primary cardiomyocyte.
17. The engineered cell of embodiment 14 or embodiment 15, wherein the cardiomyocyte or a precursor thereof has been differentiated from a pluripotent stem cell (PSC) in vitro.
18. The engineered cell of embodiment 17, wherein the in vitro differentiation of the cardiomyocyte or a precursor thereof from a PSC comprises differentiation in suspension culture.
19. The engineered cell of any of embodiments 1-18, wherein the engineered cell comprises one or more modifications that:
(a) inactivate or disrupt one or more alleles of:
   (i) one or more major histocompatibility complex (MHC) class I molecules or one or more molecules that regulate expression of the one or more MHC class I molecules; and/or
   (ii) one or more MHC class II molecules or one or more molecules that regulate expression of the one or more MHC class II molecules; and/or
(b) increase expression of one or more tolerogenic factors in the engineered cell, relative to a cell of the same cell type that does not comprise the one or more modifications.
   20. The engineered cell of embodiment 19, wherein the one or more modifications in (a) reduce expression of one or more major histocompatibility complex (MHC) human leukocyte antigen (HLA) class I molecules and/or MHC HLA class II molecules, relative to a cell of the same cell type that does not comprise the one or more modifications.
   21. The engineered cell of embodiment 19, wherein the one or more MHC class I molecules is one or more human leukocyte antigen (HLA) class I molecules.
   22. The engineered cell of any of embodiments 19-21, wherein the one or more MHC HLA class I molecules is selected from the group consisting of HLA-A, HLA-B, and HLA-C.
   23. The engineered cell of any of embodiments 19-22, the one or more molecules that regulate expression of the one or more MHC class I molecules is/are selected from the group consisting of B-2 microglobulin (B2M) gene and/or the transporter 1, ATP binding cassette subfamily B member (TAP1).
   24. The engineered cell of any of embodiments 19-23, wherein the one or more molecules that regulate expression of the one or more MHC class I molecules regulate cell surface protein expression of the one or more MHC class I molecules.
   25. The engineered cell of any of embodiments 19-24, wherein the one or more modifications in (a)(i) reduce expression of the one or more MHC HLA class I molecules.
   26. The engineered cell of any of embodiments 19-25, wherein the one or more modifications in (a)(i) reduce cell surface trafficking of the one or more MHC HLA class I molecules.
   27. The engineered cell of any of embodiments 19-26, wherein the one or more modifications in (a)(i) reduce expression of MHC HLA class I molecules HLA-A, HLA-B, and HLA-C.
   28. The engineered cell of any of embodiments 19-27, wherein the one or more modifications in (a)(i) reduce protein expression of the one or more MHC HLA class I molecules.
   29. The engineered cell of any of embodiments 19-28, wherein the one or more molecules that regulate cell surface protein expression of the one or more MHC class I molecules is B2M.
   30. The engineered cell of any of embodiments 19-29, wherein the one or more modifications
   31. The engineered cell of any of embodiments 19-30, wherein cell surface trafficking of the one or more MHC class I molecules is reduced in the engineered cell relative to the cell of the same cell type that does not comprise the one or more modifications.
   32. The engineered cell of any of embodiments 28-31, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
   33. The engineered cell of any of embodiments 1-32, wherein the engineered cell comprises one or more modifications that reduce cell surface expression of one or more MHC HLA class I molecules.
   34. The engineered cell of any of embodiments 1-33, wherein the engineered cell comprises one or more modifications that reduce a function of one or more MHC HLA class I molecules, optionally wherein the function is antigen presentation.
   35. The engineered cell of any of embodiments 19-34, wherein the one or more modifications in (a) inactivates or disrupts one or more alleles of B2M, NLRC5, or TAP1.
   36. The engineered cell of embodiment 35, wherein the one or more modifications in (a) inactivates or disrupts one or more alleles of B2M.
   37. The engineered cell of embodiment 36, wherein the modification that inactivates or disrupts one or more alleles of B2M reduces mRNA expression of the B2M gene.
   38. The engineered cell of any of embodiments 35-37, wherein the modification that inactivates or disrupts one or more alleles of B2M reduces protein expression of B2M.
   39. The engineered cell of any of embodiments 35-38, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.
   40. The engineered cell of any of embodiments 36-39, wherein the inactivation or disruption comprises an indel in the B2M gene.
   41. The engineered cell of any of embodiments 36-40, wherein the inactivation or disruption comprises a frameshift mutation or a deletion of a contiguous stretch of genomic DNA of the B2M gene.
   42. The engineered cell of any of embodiments 19-41 wherein the one or more modifications in (a) reduce expression of the B-2 microglobulin (B2M) gene and/or the transporter 1, ATP binding cassette subfamily B member (TAP1) gene.
   43. The engineered cell of embodiment 42, wherein the one or more modifications that reduce expression in (a) reduce expression of the B2M gene.
   44. The engineered cell of embodiment 42 or embodiment 43, wherein the one or more modifications that reduce expression reduces mRNA expression of the gene.
   45. The engineered cell of any of embodiments 42-44, wherein the one or more modifications that reduce expression reduces protein expression of a protein encoded by the gene.
   46. The engineered cell of any of embodiments 42-45, wherein the one or more modifications that reduce expression comprises inactivation or disruption of one allele of the gene.
   47. The engineered cell of any of embodiments 42-46, wherein the one or more modifications that reduce expression comprises inactivation or disruption of both alleles of the gene.
   48. The engineered cell of any of embodiments 42-47, wherein the one or more modifications that reduce expression comprises inactivation or disruption of all coding sequences of the gene in the cell.
   49. The engineered cell of any of embodiments 46-48, wherein the inactivation or disruption comprises an indel in one allele of the gene.
   50. The engineered cell of any of embodiments 46-49, wherein the inactivation or disruption comprises an indel in both alleles of the gene.
   51. The engineered cell of any of embodiments 42-50, wherein the one or more modifications that reduce expression comprises a frameshift mutation or a deletion of a contiguous stretch of genomic DNA of the gene.
   52. The engineered cell of any of embodiments 42-51, wherein the gene is knocked out.
   53. The engineered cell of any of embodiments 25-52, wherein the one or more modifications that reduce expression of one or more MHC HLA class I molecules is generated by nuclease-mediated gene editing.
   54. The engineered cell of embodiment 53, wherein the nuclease-mediated gene editing is mediated by a zinc finger nuclease (ZFN), a TAL-effector nuclease (TALEN), or a CRISPR-Cas combination that targets the gene.
   55. The engineered cell of embodiment 53 or 54, wherein the nuclease-mediated gene editing uses a CRISPR-Cas system comprising a CRISPR-Cas nuclease and a guide RNA (gRNA) having a targeting domain that is complementary to at least one target site within the gene.
   56. The engineered cell of embodiment 55, wherein the CRISPR-Cas system is a ribonucleoprotein (RNP) complex comprising the gRNA and a Cas protein.
   57. The engineered cell of any of embodiments 19-56, wherein the one or more MHC class II molecules is one or more human leukocyte antigen (HLA) class II molecules.
   58. The engineered cell of any of embodiments 19-56, wherein the one or more modifications in (a) reduce expression of MHC HLA class I and class II molecules.
   59. The engineered cell of embodiment 57 or embodiment 58, wherein the one or more MHC HLA class II molecules is selected from the group consisting of HLA-DP, HLA-DQ, and/or HLA-DR.
   60. The engineered cell of any of embodiments 19-59, wherein the one or more modifications in (a) reduce protein expression of one or more MHC class II molecules.
   61. The engineered cell of any of embodiments 19-60, wherein the one or more modifications in (a) reduce cell surface trafficking of the one or more MHC class II molecules.
   62. The engineered cell of any of embodiments 19-61, wherein the one or more modifications in (a) reduce a function of the one or more MHC class II molecules, optionally wherein the function is antigen presentation.
   63. The engineered cell of embodiment 60, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
   64. The engineered cell of any of embodiments 1-63, wherein the engineered cell comprises one or more modifications that reduce cell surface expression of one or more MHC HLA class II molecules.
   65. The engineered cell of any of embodiments 1-64, wherein the engineered cell comprises one or more modifications that reduce a function of one or more MHC HLA class II molecules, optionally wherein the function is antigen presentation.
   66. The engineered cell of any of embodiments 19-65, wherein the one or more molecules that regulate expression of the one or more MHC class II molecules is/are selected from the group consisting of CIITA and CD74.
   67. The engineered cell of any of embodiments 19-66, wherein the modification is a modification that regulates expression of the one or more MHC class II molecules, and the modification inactivates or disrupts one or more alleles of CIITA.
   68. The engineered cell of embodiment 67, wherein the modification that inactivates or disrupts one or more alleles of CIITA reduces mRNA expression of the CIITA gene.
   69. The engineered cell of embodiment 67 or embodiment 68, wherein the modification that inactivates or disrupts one or more alleles of CIITA reduces protein expression of CIITA.
   70. The engineered cell of any of embodiments 67-69, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the cell.
   71. The engineered cell of any of embodiments 67-70, wherein the inactivation or disruption comprises an indel in the CIITA gene.
   72. The engineered cell of any of embodiments 67-71, wherein the inactivation or disruption is a frameshift mutation or a deletion of a contiguous stretch of genomic DNA of the CIITA gene.
   73. The engineered cell of any of embodiments 1-72, wherein expression of HLA-A, HLA-B, HLA-C, HLA-DP, HLA-DQ, and HLA-DR are reduced in the engineered cell.
   74. The engineered cell of any of embodiments 19-73, wherein the one or more modifications in (a) reduce expression of the CIITA gene.
   75. The engineered cell of embodiment 74, wherein the one or more modifications that reduce expression reduce mRNA expression of the CIITA gene.
   76. The engineered cell of embodiment 74 or embodiment 75, wherein the one or more modifications that reduce expression reduces expression of a CIITA protein.
   77. The engineered cell of any of embodiments 74-76, wherein the one or more modifications that reduce expression comprises inactivation or disruption of one allele of the CIITA gene.
   78. The engineered cell of any of embodiments 74-77, wherein the one or more modifications that reduce expression comprises inactivation or disruption of both alleles of the CIITA gene.
   79. The engineered cell of any of embodiments 74-78, wherein the one or more modifications that reduce expression comprises inactivation or disruption of all CIITA coding sequences in the cell.
   80. The engineered cell of any of embodiments 77-79, wherein the inactivation or disruption comprises an indel in one allele of the CIITA gene.
   81. The engineered cell of any of embodiments 77-80, wherein the inactivation or disruption comprises an indel in both alleles of the CIITA gene.
   82. The engineered cell of any of embodiments 74-81, wherein the one or more modifications that reduce expression comprises a frameshift mutation or a deletion of a contiguous stretch of genomic DNA of the CIITA gene.
   83. The engineered cell of any of embodiments 19-82, wherein the CIITA gene is knocked out.
   84. The engineered cell of any of embodiments 81-83, wherein the one or more modifications that reduce expression of one or more MHC HLA class II molecules is generated by nuclease-mediated gene editing.
   85. The engineered cell of embodiment 84, wherein the nuclease-mediated gene editing is mediated by a zinc finger nuclease (ZFN), a TAL-effector nuclease (TALEN), or a CRISPR-Cas combination that targets the CIITA gene.
   86. The engineered cell of embodiment 84 or embodiment 85, wherein the nuclease-mediated gene editing uses a CRISPR-Cas system comprising a CRISPR-Cas nuclease and a guide RNA (gRNA) having a targeting domain that is complementary to at least one target site within the CIITA gene.
   87. The engineered cell of embodiment 86, wherein the CRISPR-Cas system is a ribonucleoprotein (RNP) complex comprising the gRNA and a Cas protein.
   88. The engineered cell of any of embodiments 1-87, wherein expression of HLA-A, HLA-B, HLA-C, HLA-DP, HLA-DQ, and HLA-DR are reduced in the engineered cell.
   89. The engineered cell of any of embodiments 19-88, wherein the one or more tolerogenic factors in (i) are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF, and any combination thereof.
   90. The engineered cell of any of embodiments 19-89, wherein the one or more tolerogenic factors in (i) are selected from the group consisting of CD47, PD-L1, HLA-E, HLA-G, CCL21, FASL, SERPINB9, CD200, MFGE8, and any combination thereof.
   91. The engineered cell of any of embodiments 19-90, wherein the one or more tolerogenic factors in (i) comprise CD47.
   92. The engineered cell of any of embodiments 89-91, wherein the one or more tolerogenic factors in (i) comprise CD47, and wherein the one or more modifications that increases expression of CD47 comprise an exogenous polynucleotide encoding the CD47 protein.
   93. The engineered cell of embodiment 92, wherein the exogenous polynucleotide encoding the CD47 protein is integrated into the genome of the engineered cell.
   94. The engineered cell of embodiment 92 or embodiment 93, wherein the exogenous polynucleotide encoding the CD47 protein encodes an amino acid sequence having at least 85% sequence identity to the amino acid sequence set forth in SEQ ID NO: 2, optionally wherein the exogenous polynucleotide encoding the CD47 protein encodes the amino acid sequence set forth in SEQ ID NO:2.
   95. The engineered cell of any of embodiments 92-94, wherein the exogenous polynucleotide is integrated by non-targeted insertion into the genome of the engineered cell, optionally by introduction of the exogenous polynucleotide into the cell using a lentiviral vector.
   96. The engineered cell of any of embodiments 92-94, wherein the exogenous polynucleotide is integrated by targeted insertion into a target genomic locus of the engineered cell.
   97. The engineered cell of embodiment 96, wherein the target genomic locus is a safe harbor locus, a B2M gene locus, a CIITA gene locus, a CACNA1G locus, a HCN4 locus, or a SLC8A1 locus.
   98. The engineered cell of embodiment 96 or embodiment 97, wherein the target genomic locus is selected from the group consisting of: a CCR5 gene locus, a CXCR4 gene locus, a PPP1R12C (also known as AAVS1) gene locus, an albumin gene locus, a SHS231 locus, a CLYBL gene locus, and a ROSA26 gene locus.
   99. The engineered cell of any of embodiments 19-98, wherein the one or more modifications that reduce expression in (a) comprise reduced surface protein expression; and/or the one or more modifications that increase expression in (b) comprise increased surface protein expression.
   100. An engineered cell comprising one or more modifications that:
(a) inactivate or disrupt one or more alleles of CACNA1G, HCN4, and SLC8A1, one or more MHC HLA class I molecules, and/or one or more MHC HLA class II molecules; and
(b) increase expression of KCNJ2 and CD47, relative to a cell of the same cell type that does not comprise the one or more modifications.
   101. The engineered cell of embodiment 100, wherein the one or more modifications of (a) reduce expression of CACNA1G, HCN4, and SLC8A1, one or more MHC HLA class I molecules, and/or one or more MHC HLA class II molecules, relative to a cell of the same cell type that does not comprise the one or more modifications.
   102. An engineered cell comprising one or more modifications that:
(a) inactivate or disrupt one or more alleles of CACNA1G, HCN4, and SLC8A1, one or more MHC HLA class I molecules, and one or more MHC HLA class II molecules, and
(b) increase expression of KCNJ2 and CD47, relative to a cell of the same cell type that does not comprise the one or more modifications.
   103. The engineered cell of embodiment 102, wherein the one or more modifications of (a) reduce expression of CACNA1G, HCN4, and SLC8A1, one or more MHC HLA class I molecules, and one or more MHC HLA class II molecules, relative to a cell of the same cell type that does not comprise the one or more modifications.
   104. The engineered cell of any of embodiments 100-103, wherein the one or more modifications that reduce expression of one or more MHC HLA class I molecules and/or one or more MHC class II molecules reduce expression of B2M and CIITA.
   105. An engineered cell comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.
   106. The engineered cell of embodiment 105, wherein the one or more modifications of (a) reduce expression of CACNA1G, HCN4, SLC8A1, B2M, and CIITA, relative to a cell of the same cell type that does not comprise the one or more modifications.
   107. An engineered primary human cell comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.
   108. The engineered cell of embodiment 107, wherein the one or more modifications of (a) reduce expression of CACNA1G, HCN4, SLC8A1, B2M, and CIITA, relative to a cell of the same cell type that does not comprise the one or more modifications.
   109. An engineered induced pluripotent stem cell (iPSC) or embryonic stem cell (ESC) comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.
   110. The engineered iPSC or ESC of embodiment 109, wherein the one or more modifications of (a) reduce expression of CACNA1G, HCN4, SLC8A1, B2M, and CIITA, relative to a cell of the same cell type that does not comprise the one or more modifications.
   111. An engineered cardiomyocyte that has been differentiated in vitro from an engineered cell of any of embodiments 1-110.
   112. An engineered cardiomyocyte that has been differentiated in vitro from a pluripotent stem cell (PSC), wherein the engineered cardiomyocyte comprises one or more modifications that:
(a) inactivate or disrupt one or more alleles of CACNA1G, HCN4, and SLC8A1;
(b) increase expression of one or more of KCNJ2, TRDN, SRL, HRC, and CASQ2, relative to a cardiomyocyte differentiated in vitro from a PSC that does not comprise the one or more modifications.
   113. The engineered cardiomyocyte of embodiment 112, wherein the one or more modifications of (a) reduce expression of one or more of CACNA1G, HCN4, and SLC8A1, relative to a cardiomyocyte differentiated in vitro from a PSC that does not comprise the one or more modifications.
   114. An engineered cardiomyocyte that has been differentiated in vitro from a pluripotent stem cell (PSC), wherein the engineered cardiomyocyte comprises one or more modifications that:
(a) inactivate or disrupt one or more alleles of:
   (i) CACNA1G, HCN4, and SLC8A1;
   (ii) MHC HLA class I molecules and one or more MHC HLA class II molecules;
(b) increase expression of one or more of KCNJ2, TRDN, SRL, HRC, CASQ2, and CD47; or (d) a combination thereof; or
(c) a combination thereof, relative to a cardiomyocyte differentiated in vitro from a PSC that does not comprise the one or more modifications.
   115. The engineered cardiomyocyte of embodiment 114, wherein the one or more modifications of (a) reduce expression of one or more of CACNA1G, HCN4, and SLC8A1; and reduce expression of one or more of MHC HLA class I molecules and one or more MHC HLA class II molecules, relative to a cardiomyocyte that does not comprise the one or more modifications.
   116. The engineered cardiomyocyte of embodiment 114 or embodiment 115, wherein the one or more modifications that reduce expression of one or more MHC HLA class I molecules and one or more MHC class II molecules reduce expression of B2M and CIITA.
   117. An engineered cardiomyocyte that has been differentiated in vitro from a pluripotent stem cell (PSC), wherein the engineered cardiomyocyte comprises one or more modifications that:
(a) inactivate or disrupt one or more alleles of:
   (i) CACNA1G, HCN4, and SLC8A1;
   (ii) B2M, TAP1, and CIITA;
(b) increase expression of one or more of KCNJ2, TRDN, SRL, HRC, CASQ2, and CD47; or
(c) a combination thereof, relative to a cardiomyocyte differentiated in vitro from a PSC that does not comprise the one or more modifications.
   118. The engineered cardiomyocyte of embodiment 117, wherein the one or more modifications of (a) reduce expression of one or more of CACNA1G, HCN4, and SLC8A1; and reduce expression of one or more of B2M, TAP1, and CIITA, relative to a cardiomyocyte differentiated in vitro from a PSC that does not comprise the one or more modifications.
   119. An engineered cardiomyocyte that has been differentiated in vitro from a pluripotent stem cell (PSC), wherein the engineered cardiomyocyte comprises one or more modifications that:
(a) inactivate or disrupt one or more alleles of CACNA1G, HCN4, and SLC8A1;
(b) increase expression of one or more of KCNJ2, TRDN, SRL, HRC, and CASQ2, relative to a primary cardiomyocyte that does not comprise the one or more modifications.
   120. The engineered cardiomyocyte of embodiment 119, wherein the one or more modifications of (a) reduce expression of one or more of CACNA1G, HCN4, and SLC8A1, relative to a primary cardiomyocyte that does not comprise the one or more modifications.
   121. An engineered cardiomyocyte that has been differentiated in vitro from a pluripotent stem cell (PSC), wherein the engineered cardiomyocyte comprises one or more modifications that:
(a) inactivate or disrupt one or more alleles of:
   (i) CACNA1G, HCN4, and SLC8A1;
   (ii) MHC HLA class I molecules and one or more MHC HLA class II molecules;
(b) increase expression of one or more of KCNJ2, TRDN, SRL, HRC, CASQ2, and CD47; or (d) a combination thereof; or
(c) a combination thereof, relative to a primary cardiomyocyte that does not comprise the one or more modifications.
   122. The engineered cardiomyocyte of embodiment 121, wherein the one or more modifications of (a) reduce expression of one or more of CACNA1G, HCN4, and SLC8A1; and reduce expression of one or more of MHC HLA class I molecules and one or more MHC HLA class II molecules, relative to a primary cardiomyocyte that does not comprise the one or more modifications.
   123. The engineered cardiomyocyte of embodiment 122, wherein the one or more modifications that reduce expression of one or more MHC HLA class I molecules and one or more MHC class II molecules reduce expression of B2M and CIITA.
   124. An engineered cardiomyocyte that has been differentiated in vitro from a pluripotent stem cell (PSC), wherein the engineered cardiomyocyte comprises one or more modifications that:
(a) inactivate or disrupt one or more alleles of:
   (i) CACNA1G, HCN4, and SLC8A1;
   (ii) B2M, TAP1, and CIITA;
(b) increase expression of one or more of KCNJ2, TRDN, SRL, HRC, CASQ2, and CD47; or
(c) a combination thereof, relative to a primary cardiomyocyte that does not comprise the one or more modifications.
   125. The engineered cardiomyocyte of embodiment 124, wherein the one or more modifications of (a) reduce expression of one or more of CACNA1G, HCN4, and SLC8A1; and reduce expression of one or more of B2M, TAP1, and CIITA, relative to a primary cardiomyocyte that does not comprise the one or more modifications.
   126. The engineered cell or cardiomyocyte of any of embodiments 100, 101, and 111-125, wherein the engineered cell or cardiomyocyte comprises one or more modifications that reduce expression of CACNA1G.
   127. The engineered cell or cardiomyocyte of any of embodiments 100, 101, and 111-126, wherein the engineered cell or cardiomyocyte comprises one or more modifications that reduce expression of HCN4 and/or SLC8A1.
   128. The engineered cell or cardiomyocyte of any of embodiments 100, 101, and 111-127, wherein the engineered cell or cardiomyocyte comprises one or more modifications that increase expression of KCNJ2.
   129. The engineered cell or cardiomyocyte of any of embodiments 100, 101, and 111-128, wherein the engineered cell or cardiomyocyte comprises one or more modifications that increase expression of TRDN.
   130. The engineered cell or cardiomyocyte of any of embodiments 100, 101, and 111-129, wherein the engineered cell or cardiomyocyte comprises one or more modifications that increase expression of SRL.
   131. The engineered cell or cardiomyocyte of any of embodiments 100, 101, and 111-130, wherein the engineered cell or cardiomyocyte comprises one or more modifications that increase expression of HRC.
   132. The engineered cell or cardiomyocyte of any of embodiments 100, 101, and 111-131, wherein the engineered cell or cardiomyocyte comprises one or more modifications that increase expression of CASQ2.
   133. The engineered cell or cardiomyocyte of any of embodiments 100, 101, and 111-132, wherein the engineered cell or cardiomyocyte comprises one or more modifications that (a) reduce expression of CACNA1G, HCN4, and SLC8A1; and (b) increase expression of KCNJ2.
   134. The engineered cell or cardiomyocyte of any of embodiments 100, 111, 112, and 126-133, wherein the engineered cell or cardiomyocyte comprises one or more modifications that:
(a) inactivate or disrupt one or more alleles of:
   (i) one or more major histocompatibility complex (MHC) class I molecules or one or more molecules that regulate expression of the one or more MHC class I molecules; and/or
   (ii) one or more MHC class II molecules or one or more molecules that regulate expression of the one or more MHC class II molecules; and/or
(b) increase expression of one or more tolerogenic factors in the engineered cell or cardiomyocyte, relative to a cell of the same cell type that does not comprise the one or more modifications, optionally wherein the one or more modifications (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.
   135. The engineered cell or cardiomyocyte of any of embodiments 100-104, 111, 114-116, and 126-134, wherein the one or more MHC HLA class I molecules is selected from the group consisting of HLA-A, HLA-B, and HLA-C.
   136. The engineered cell or cardiomyocyte of any of embodiments 100-135, wherein the one or more molecules that regulate expression of the one or more MHC class I molecules is/are selected from the group consisting of B-2 microglobulin (B2M) gene and/or the transporter 1, ATP binding cassette subfamily B member (TAP1).
   137. The engineered cell or cardiomyocyte of any of embodiments 100-136, wherein the one or more molecules that regulate expression of the one or more MHC class I molecules regulate cell surface protein expression of the one or more MHC class I molecules.
   138. The engineered cell or cardiomyocyte of any of embodiments 100-111 and 114-137, wherein the one or more modifications reduce expression of the one or more MHC HLA class I molecules.
   139. The engineered cell or cardiomyocyte of any of embodiments 100-111 and 114-138, wherein the one or more modifications reduce cell surface trafficking of the one or more MHC HLA class I molecules.
   140. The engineered cell or cardiomyocyte of any of embodiments 100-111 and 114-139, wherein the one or more modifications reduce expression of MHC HLA class I molecules HLA-A, HLA-B, and HLA-C.
   141. The engineered cell or cardiomyocyte of any of embodiments 100-111 and 114-140, wherein the one or more modifications reduce protein expression of one or more MHC HLA class I molecules.
   142. The engineered cell or cardiomyocyte of embodiment 141, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
   143. The engineered cell or cardiomyocyte of any of embodiments 100-142, wherein the engineered cell comprises one or more modifications that reduce cell surface expression of one or more MHC HLA class I molecules.
   144. The engineered cell or cardiomyocyte of any of embodiments 100-143, wherein the engineered cell comprises one or more modifications that reduce a function of one or more MHC HLA class I molecules, optionally wherein the function is antigen presentation.
   145. The engineered cell of any of embodiments 100-144, wherein the one or more modifications in (a) inactivates or disrupts one or more alleles of B2M, NLRC5, or TAP1.
   146. The engineered cell of embodiment 145, wherein the one or more modifications in (a) inactivates or disrupts one or more alleles of B2M.
   147. The engineered cell of embodiment 146, wherein the modification that inactivates or disrupts one or more alleles of B2M reduces mRNA expression of the B2M gene.
   148. The engineered cell of any of embodiments 145-147, wherein the modification that inactivates or disrupts one or more alleles of B2M reduces protein expression of B2M.
   149. The engineered cell of any of embodiments 145-148, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.
   150. The engineered cell of any of embodiments 145-149, wherein the inactivation or disruption comprises an indel in the B2M gene.
   151. The engineered cell of any of embodiments 145-150, wherein the inactivation or disruption comprises a frameshift mutation or a deletion of a contiguous stretch of genomic DNA of the B2M gene.
   152. The engineered cell or cardiomyocyte of any of embodiments 100-151, wherein the one or more modifications that reduce expression reduce expression of the B2M gene.
   153. The engineered cell or cardiomyocyte of any of embodiments 100-111 and 114-152, wherein the one or more modifications reduce expression of MHC HLA class I and class II molecules.
   154. The engineered cell or cardiomyocyte of any of embodiments 100-111 and 114-153, wherein the one or more modifications reduce expression of MHC HLA class II molecules HLA-DP, HLA-DQ, or HLA-DR.
   155. The engineered cell or cardiomyocyte of any of embodiments 100-111 and 114-154, wherein the one or more modifications reduce protein expression of one or more MHC class II molecules.
   156. The engineered cell or cardiomyocyte of embodiment 155, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
   157. The engineered cell or cardiomyocyte of any of embodiments 100-156, wherein the engineered cell or cardiomyocyte comprises one or more modifications that reduce cell surface expression of one or more MHC HLA class II molecules.
   158. The engineered cell or cardiomyocyte of any of embodiments 100-157, wherein the engineered cell or cardiomyocyte comprises one or more modifications that reduce a function of one or more MHC HLA class II molecules, optionally wherein the function is antigen presentation.
   159. The engineered cell or cardiomyocyte of any of embodiments 100-111 and 114-158, wherein: the one or more modifications reduce expression of the CIITA gene; and/or the modification that inactivates or disrupts one or more alleles of CIITA comprises:
(i) inactivation or disruption of one allele of the CIITA gene;
(ii) inactivation or disruption of both alleles of the CIITA gene; or
(iii) inactivation or disruption of all CIITA coding alleles in the cell.
   160. The engineered cell or cardiomyocyte of embodiment 111, wherein the one or more tolerogenic factors comprises CD47.
   161. The engineered cell or cardiomyocyte of any of embodiments 100-111 and 114-160, wherein the one or more modifications that increases expression of CD47 comprise an exogenous polynucleotide encoding the CD47 protein.
   162. The engineered cell or cardiomyocyte of any of embodiments 100-161, wherein the phenotype of the engineered cell or cardiomyocyte comprises B2M^{indel/indel}; CIITA^{indel/indel}; and CD47^{tg}.
   163. The engineered cell or cardiomyocyte of any of embodiments 1-162, wherein the engineered cell or cardiomyocyte further comprises a modification for expression of an exogenous safety switch.
   164. The engineered cell or cardiomyocyte of embodiment 163, wherein the safety switch is a system wherein upon activation, cells downregulate expression of the one or more tolerogenic factors and/or upregulate expression of one or more immune signaling molecules thereby marking the engineered cell or cardiomyocyte for elimination by the host immune system.
   165. The engineered cell or cardiomyocyte of embodiment 163 or embodiment 164, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.
   166. The engineered cell or cardiomyocyte of embodiment 164 or embodiment 165, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.
   167. The engineered cell or cardiomyocyte of embodiment 163, wherein the safety switch is a suicide gene.
   168. The engineered cell or cardiomyocyte of embodiment 167, wherein the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).
   169. The engineered cell or cardiomyocyte of any of embodiments 163-168, wherein the safety switch and the one or more tolerogenic factors are expressed from a bicistronic cassette integrated into the genome of the engineered cell or cardiomyocyte.
   170. The engineered cell or cardiomyocyte of embodiment 169, wherein the bicistronic cassette is integrated at a non-target locus in the genome of the engineered cell or cardiomyocyte.
   171. The engineered cell or cardiomyocyte of embodiment 169, wherein the bicistronic cassette is integrated into a target genomic locus of the engineered cell or cardiomyocyte.
   172. The engineered cell or cardiomyocyte of any of embodiments 1-171, wherein the engineered cell or cardiomyocyte comprises an exogenous polynucleotide encoding a safety switch.
   173. The engineered cell or cardiomyocyte of embodiment 172, wherein the safety switch is a system wherein upon activation, cells downregulate expression of the one or more tolerogenic factors and/or upregulate expression of one or more immune signaling molecules thereby marking the cell for elimination by the host immune system.
   174. The engineered cell or cardiomyocyte of embodiment 172 or embodiment 173, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.
   175. The engineered cell or cardiomyocyte of embodiment 173 or embodiment 174, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.
   176. The engineered cell or cardiomyocyte of embodiment 163 or embodiment 172, wherein the safety switch is a suicide gene.
   177. The engineered cell or cardiomyocyte of embodiment 176, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).
   178. The engineered cell or cardiomyocyte of any of embodiments 172-177, wherein the safety switch and genes associated with the safety switch are expressed from a bicistronic cassette integrated into the genome of the engineered cell or cardiomyocyte.
   179. The engineered cell or cardiomyocyte of any of embodiments 172-177, wherein the safety switch and the one or more tolerogenic factors are expressed from a bicistronic cassette integrated into the genome of the engineered cell or cardiomyocyte.
   180. The engineered cell or cardiomyocyte of embodiment 178 or embodiment 179, wherein the bicistronic cassette is integrated by non-targeted insertion into the genome of the engineered cell or cardiomyocyte.
   181. The engineered cell or cardiomyocyte of embodiment 178 or embodiment 179, wherein the bicistronic cassette is integrated by targeted insertion into a target genomic locus of the engineered cell or cardiomyocyte.
   182. The engineered cell or cardiomyocyte of any of embodiments 172-181, wherein the one or more tolerogenic factors is CD47.
   183. The engineered cell or cardiomyocyte of any of embodiments 1-182, wherein the inactivation or disruption is by one or more gene edits.
   184. The engineered cell or cardiomyocyte of any of embodiments 1-183, wherein the cell comprises a genome editing complex.
   185. The engineered cell or cardiomyocyte of embodiment 183 or embodiment 184, wherein the one or more gene edits are made by a genome editing complex.
   186. The engineered cell or cardiomyocyte of embodiment 184 or embodiment 185, wherein the genome editing complex comprises a genome targeting entity and a genome modifying entity.
   187. The engineered cell or cardiomyocyte of embodiment 186, wherein the genome targeting entity localizes the genome editing complex to the one or more alleles that are inactivated or disrupted, optionally wherein the genome targeting entity is a nucleic acid-guided targeting entity.
   188. The engineered cell or cardiomyocyte of embodiment 186 or embodiment 187, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
   189. The engineered cell or cardiomyocyte of any of embodiments 186-188, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
   190. The engineered cell or cardiomyocyte of any of embodiments 186-189, wherein the genome modifying entity cleaves, deaminates, nicks, polymerizes, interrogates, integrates, cuts, unwinds, breaks, alters, methylates, demethylates, or otherwise destabilizes the target locus.
   191. The engineered cell or cardiomyocyte of any of embodiments 186-190, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
   192. The engineered cell or cardiomyocyte of any of embodiments 186-191, wherein the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
   193. The engineered cell or cardiomyocyte of any of embodiments 186-192, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
   194. The engineered cell or cardiomyocyte of any of embodiments 186-193, wherein the genome targeting entity and the genome modifying entity are different domains of a single polypeptide.
   195. The engineered cell or cardiomyocyte of any of embodiments 186-194, wherein the genome editing entity and genome modifying entity are two different polypeptides that are operably linked together.
   196. The engineered cell or cardiomyocyte of any of embodiments 186-194, wherein the genome editing entity and genome modifying entity are two different polypeptides that are not linked together.
   197. The engineered cell or cardiomyocyte of any of embodiments 186-196, wherein the genome editing complex comprises a guide nucleic acid having a targeting domain that is complementary to at least one target locus, optionally wherein the guide nucleic acid is a guide RNA (gRNA).
   198. The engineered cell or cardiomyocyte of any of embodiments 186-197, wherein the one or more modifications are made by the genome editing complex.
   199. The engineered cell or cardiomyocyte of embodiment 198, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).
   200. The engineered cell or cardiomyocyte of embodiment 198 or embodiment 199, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).
   201. The engineered cell or cardiomyocyte of any of embodiments 198-200, wherein the modifications made by the genome editing complex are made using a guide RNA (gRNA) having a targeting domain that is complementary to at least one target site.
   202. The engineered cell or cardiomyocyte of any of embodiments 183-185, wherein the genome editing complex is an RNA-guided nuclease.
   203. The engineered cell or cardiomyocyte of embodiment 202, wherein the RNA-guided nuclease comprises a Cas nuclease and a guide RNA (CRISPR-Cas combination).
   204. The engineered cell or cardiomyocyte of embodiment 203, wherein the CRISPR-Cas combination is a ribonucleoprotein (RNP) complex comprising the gRNA and the Cas nuclease.
   205. The engineered cell or cardiomyocyte of embodiment 203 or embodiment 204, wherein the Cas nuclease is a Type II or Type V Cas protein.
   206. The engineered cell or cardiomyocyte of any of embodiments 203-205, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.
   207. The engineered cell or cardiomyocyte of any of embodiments 1-206, wherein the engineered cell or cardiomyocyte has been differentiated from a pluripotent stem cell (PSC) in vitro.
   208. The engineered cell or cardiomyocyte of embodiment 207, wherein the in vitro differentiation of the engineered cell or cardiomyocyte from a PSC comprises differentiation in suspension culture.
   209. The engineered cell or cardiomyocyte of embodiment 208, wherein differentiation of the cardiomyocyte from the PSC comprises differentiation in suspension culture.
   210. The engineered cell or cardiomyocyte of any of embodiments 207-209, wherein one or more modifications that reduce expression and/or the one or more modifications that increase expression is carried out prior to the differentiation.
   211. The engineered cell or cardiomyocyte of any of embodiments 207-209, wherein one or more modifications that reduce expression and/or the one or more modifications that increase expression is carried out subsequent to the differentiation.
   212. The engineered cell or cardiomyocyte of any of embodiments 207-209, wherein one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.
   213. The engineered cell or cardiomyocyte of any of embodiments 1-212, which is human.
   214. A composition comprising a plurality of the engineered cardiomyocytes of any of embodiments 14-99 and 111-213.
   215. The composition of embodiment 214, wherein the composition comprises between about 5×10⁸ and 1×10¹⁰ engineered cardiomyocytes, inclusive of each.
   216. The composition of embodiment 214 or embodiment 215, wherein the composition comprises between about 1×10⁹ and about 5×10⁹ engineered cardiomyocytes, inclusive of each.
   217. The composition of any of embodiments 214-216, wherein the composition comprises a pharmaceutically acceptable carrier.
   218. The composition of any of embodiments 214-217, wherein at least at or about 50%, at least at or about 60%, at least at or about 70%, at least at or about 80%, or at least at or about 90% of the cells in the plurality of the engineered cardiomyocytes are reduced for expression of one or more MHC class I molecules and/or for expression of B2M.
   219. The composition of any of embodiments 214-218, wherein at least at or about 50%, at least at or about 60%, at least at or about 70%, at least at or about 80%, or at least at or about 90% of the cells in the plurality of the engineered cardiomyocytes are reduced for expression of one or more MHC class II molecules and/or for expression of CIITA.
   220. The composition of any of embodiments 214-219, wherein at least at or about 50%, at least at or about 60%, at least at or about 70%, at least at or about 80%, or at least at or about 90% of the cells in the plurality of the engineered cardiomyocytes comprise inactivation or disruption of one or more alleles of: one or more MHC class I molecules and/or B2M.
   221. The composition of any of embodiments 214-220, wherein at least at or about 50%, at least at or about 60%, at least at or about 70%, at least at or about 80%, or at least at or about 90% of the cells in the plurality of the engineered cardiomyocytes comprise inactivation or disruption of one or more alleles of: one or more MHC class II molecules and/or CIITA.
   222. The composition of any of embodiments 214-221, wherein at least at or about 50%, at least at or about 60%, at least at or about 70%, at least at or about 80%, or at least at or about 90% of the cells in the plurality of the engineered cardiomyocytes express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications.
   223. The composition of any of embodiments 214-222, wherein at least at or about 50%, at least at or about 60%, at least at or about 70%, at least at or about 80%, or at least at or about 90% of the cells in the plurality of the engineered cardiomyocytes express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications.
   224. The composition of any of embodiments 214-223, wherein at least at or about 50%, at least at or about 60%, at least at or about 70%, at least at or about 80%, or at least at or about 90% of the cells in the plurality of the engineered cardiomyocytes expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.
   225. The composition of any of embodiments 214-224, wherein the inactivation or disruption is by one or more gene edits.
   226. The composition of any of embodiments 214-225, wherein the cells of the plurality of the engineered cardiomyocytes comprise a genome editing complex.
   227. The composition of embodiment 225 or embodiment 226, wherein the one or more gene edits are made by a genome editing complex.
   228. The composition of embodiment 226 or embodiment 227, wherein the genome editing complex comprises a genome targeting entity and a genome modifying entity.
   229. The composition of embodiment 228, wherein the genome targeting entity localizes the genome editing complex to the one or more alleles that are inactivated or disrupted, optionally wherein the genome targeting entity is a nucleic acid-guided targeting entity.
   230. The composition of embodiment 228 or embodiment 229, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
   231. The composition of any of embodiments 228-230, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
   232. The composition of any of embodiments 228-231, wherein the genome modifying entity cleaves, deaminates, nicks, polymerizes, interrogates, integrates, cuts, unwinds, breaks, alters, methylates, demethylates, or otherwise destabilizes the target locus.
   233. The composition of any of embodiments 228-232, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
   234. The composition of any of embodiments 228-233, wherein the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
   235. The composition of any of embodiments 228-234, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
   236. The composition of any of embodiments 228-235, wherein the genome targeting entity and the genome modifying entity are different domains of a single polypeptide.
   237. The composition of any of embodiments 228-235, wherein the genome editing entity and genome modifying entity are two different polypeptides that are operably linked together.
   238. The composition of any of embodiments 228-235, wherein the genome editing entity and genome modifying entity are two different polypeptides that are not linked together.
   239. The composition of any of embodiments 228-238, wherein the genome editing complex comprises a guide nucleic acid having a targeting domain that is complementary to at least one target locus, optionally wherein the guide nucleic acid is a guide RNA (gRNA).
   240. The composition of any of embodiments 228-239, wherein the one or more modifications are made by the genome editing complex.
   241. The composition of embodiment 240, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).
   242. The composition of embodiment 240 or embodiment 241, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).
   243. The composition of any of embodiments 240-242, wherein the modifications made by the genome editing complex are made using a guide RNA (gRNA) having a targeting domain that is complementary to at least one target site.
   244. The composition of any of embodiments 226-228, wherein the genome editing complex is an RNA-guided nuclease.
   245. The composition of embodiment 244, wherein the RNA-guided nuclease comprises a Cas nuclease and a guide RNA (CRISPR-Cas combination).
   246. The composition of embodiment 245, wherein the CRISPR-Cas combination is a ribonucleoprotein (RNP) complex comprising the gRNA and the Cas nuclease.
   247. The composition of embodiment 245 or embodiment 246, wherein the Cas nuclease is a Type II or Type V Cas protein.
   248. The composition of any of embodiments 245-247, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.
   249. The composition of any of embodiments 214-248, comprising a pharmaceutically acceptable excipient.
   250. The composition of any of embodiments 214-249, comprising a cryoprotectant.
   251. A method of producing an engineered cell, the method comprising: (a) reducing expression of one or more of CACNA1G, HCN4, and SLC8A1; (b) increasing expression of one or more of KCNJ2, TRDN, SRL, HRC, and CASQ2; or (c) a combination thereof, in the cell. 252. The method of embodiment 251, wherein the method comprises reducing expression of CACNA1G in the cell.
   253. The method of embodiment 251 or embodiment 252, wherein the method comprises reducing expression of HCN4 and/or SLC8A1 in the cell.
   254. The method of any of embodiments 251-253, wherein the method comprises increasing expression of KCNJ2 in the cell.
   255. The method of any of embodiments 251-254, wherein the method comprises increasing expression of TRDN in the cell.
   256. The method of any of embodiments 251-255, wherein the method comprises increasing expression of SRL in the cell.
   257. The method of any of embodiments 251-256, wherein the method comprises increasing expression of HRC in the cell.
   258. The method of any of embodiments 251-257, wherein the method comprises increasing expression of CASQ2 in the cell.
   259. The method of any of embodiments 251-258, wherein the method comprises: (a) reducing expression of CACNA1G, HCN4, and SLC8A1; and (b) increasing expression of KCNJ2, in the cell.
   260. The method of any of embodiments 251-259, wherein the engineered cell is a pluripotent stem cell (PSC).
   261. The method of embodiment 260, wherein the PSC is an induced pluripotent stem cell (iPSC).
   262. The method of embodiment 260, wherein the PSC is an embryonic stem cell (ESC).
   263. The method of any of embodiments 251-259, wherein the engineered cell is a primary cardiac cell.
   264. The method of any of embodiments 251-259 and 263, wherein the engineered cell is a cardiomyocyte or a precursor thereof.
   265. The method of any of embodiments 251-259, 263, and 264, wherein the engineered cell is a cardiomyocyte.
   266. The method of any of embodiments 251-259 and 263-265, wherein the engineered cell is a primary cardiomyocyte.
   267. The method of embodiment 264 or embodiment 265, wherein the cardiomyocyte or a precursor thereof has been differentiated from a pluripotent stem cell (PSC) in vitro.
   268. The method of embodiment 267, wherein the in vitro differentiation of the cardiomyocyte or a precursor thereof from a PSC comprises differentiation in suspension culture.
   269. The method of any of embodiments 251-262, wherein the method further comprises differentiating the PSC into a cardiomyocyte.
   270. The method of embodiment 269, wherein differentiation of the cardiomyocyte from the PSC comprises differentiation in suspension culture.
   271. The method of any of embodiments 267-270, wherein the reducing expression and/or the increasing expression is carried out prior to the differentiation.
   272. The method of any of embodiments 267-270, wherein the reducing expression and/or the increasing expression is carried out subsequent to the differentiation.
   273. The method of any of embodiments 267-270, wherein part of the reducing expression and/or the increasing expression is carried out prior to the differentiation; and part of the reducing expression and/or the increasing expression is carried out subsequent to the differentiation.
   274. The method of any of embodiments 267-270, wherein one or more modifications that reduce expression and/or the one or more modifications that increase expression is carried out prior to the differentiation.
   275. The method of any of embodiments 267-270, wherein one or more modifications that reduce expression and/or the one or more modifications that increase expression is carried out subsequent to the differentiation.
   276. The method of any of embodiments 267-270, wherein one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.
   277. The method of any of embodiments 251-276, wherein the engineered cell comprises one or more modifications that:
(a) inactivate or disrupt one or more alleles of:
   (i) one or more major histocompatibility complex (MHC) class I molecules or one or more molecules that regulate expression of the one or more MHC class I molecules; and/or
   (ii) one or more MHC class II molecules or one or more molecules that regulate expression of the one or more MHC class II molecules; and/or
(b) increase expression of one or more tolerogenic factors in the engineered cell or cardiomyocyte, relative to a cell of the same cell type that does not comprise the one or more modifications, optionally wherein the one or more modifications (i) increase expression of one or more tolerogenic factors; and/or (ii) reduce expression of one or more major histocompatibility complex (MHC) class I molecules and/or MHC class II, relative to a cell of the same cell type that does not comprise the one or more modifications.
   278. The method of embodiment 277, wherein the one or more MHC HLA class I molecules is selected from the group consisting of HLA-A, HLA-B, and HLA-C.
   279. The method of embodiment 277 or embodiment 278, wherein the one or more molecules that regulate expression of the one or more MHC class I molecules is/are selected from the group consisting of B-2 microglobulin (B2M) gene and/or the transporter 1, ATP binding cassette subfamily B member (TAP1).
   280. The method of any of embodiments 277-279, wherein the one or more molecules that regulate expression of the one or more MHC class I molecules regulate cell surface protein expression of the one or more MHC class I molecules.
   281. The method of any of embodiments 277-280, wherein the one or more modifications in (a) reduce expression of the one or more MHC HLA class I molecules.
   282. The method of any of embodiments 277-281, wherein the one or more modifications in (a)(i) reduce cell surface trafficking of the one or more MHC HLA class I molecules.
   283. The method of any of embodiments 277-282, wherein the one or more modifications in (a) reduce expression of MHC HLA class I molecules HLA-A, HLA-B, and HLA-C.
   284. The method of any of embodiments 277-283, wherein the one or more modifications in (a) reduce protein expression of one or more MHC HLA class I molecules.
   285. The method of any of embodiments 277-284, wherein the one or more molecules that regulate cell surface protein expression of the one or more MHC class I molecules is B2M.
   286. The method of any of embodiments 277-285, wherein the one or more modifications comprise a modification that regulates cell surface protein expression of the one or more MHC class I molecules and the modification inactivates or disrupts one or more alleles of B2M.
   287. The method of any of embodiments 277-286, wherein cell surface trafficking of the one or more MHC class I molecules is reduced in the engineered cell relative to the cell of the same cell type that does not comprise the one or more modifications.
   288. The method of any of embodiments 277-287, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
   289. The method of any of embodiments 251-288, wherein the engineered cell comprises one or more modifications that reduce cell surface expression of one or more MHC HLA class I molecules.
   290. The method of any of embodiments 251-289, wherein the engineered cell comprises one or more modifications that reduce a function of one or more MHC HLA class I molecules, optionally wherein the function is antigen presentation.
   291. The method of any of embodiments 277-290, wherein the one or more modifications in (a) inactivates or disrupts one or more alleles of B2M, NLRC5, or TAP1.
   292. The method of embodiment 291, wherein the one or more modifications in (a) inactivates or disrupts one or more alleles of B2M.
   293. The method of embodiment 291 or embodiment 292, wherein the modification that inactivates or disrupts one or more alleles of B2M reduces mRNA expression of the B2M gene.
   294. The method of any of embodiments 291-293, wherein the modification that inactivates or disrupts one or more alleles of B2M reduces protein expression of B2M.
   295. The method of any of embodiments 291-294, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.
   296. The method of any of embodiments 291-295, wherein the inactivation or disruption comprises an indel in the CIITA gene.
   297. The method of any of embodiments 291-296, wherein the inactivation or disruption is a frameshift mutation or a deletion of a contiguous stretch of genomic DNA of the CIITA gene.
   298. The method of any of embodiments 277-297, wherein expression of HLA-A, HLA-B, HLA-C, HLA-DP, HLA-DQ, and HLA-DR are reduced in the engineered cell.
   299. The method of any of embodiments 277-298, wherein the one or more modifications in (a) reduce expression of the CIITA gene.
   300. The method of any of embodiments 277-299, wherein the one or more tolerogenic factors comprises CD47.
   301. The method of any of embodiments 277-300, wherein the one or more tolerogenic factors comprise CD47, and wherein the one or more modifications that increases expression of CD47 comprise an exogenous polynucleotide encoding the CD47 protein.
   302. The method of any of embodiments 251-301, wherein the phenotype of the engineered cell comprises B2M^{indel/indel}; CIITA^{indel/indel}; and CD47^{tg}.
   303. The method of any of embodiments 108-142, wherein the reducing in (a) is by one or more gene edits.
   304. The engineered cell or cardiomyocyte of any of embodiments 19-213, wherein the inactivating or disrupting of the one or more alleles is by one or more gene edits.
   305. The engineered cell or cardiomyocyte of any of embodiments 1-213 or the method of any of embodiments 251-303, wherein the cell comprises a genome editing complex.
   306. The engineered cell or cardiomyocyte or the method of embodiment 304 or embodiment 305, wherein the one or more gene edits are made by a genome editing complex.
   307. The engineered cell or cardiomyocyte or the method of embodiment 306, wherein the genome editing complex comprises a genome targeting entity and a genome modifying entity.
   308. The engineered cell or cardiomyocyte or the method of embodiment 307, wherein the genome targeting entity localizes the genome editing complex to the one or more alleles that are inactivated or disrupted, optionally wherein the genome targeting entity is a nucleic acid-guided targeting entity.
   309. The engineered cell or cardiomyocyte or the method of embodiment 307 or embodiment 308, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
   310. The engineered cell or cardiomyocyte or the method of any of embodiments 307-309, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
   311. The engineered cell or cardiomyocyte or the method of any of embodiments 307-309, wherein the genome modifying entity cleaves, deaminates, nicks, polymerizes, interrogates, integrates, cuts, unwinds, breaks, alters, methylates, demethylates, or otherwise destabilizes the target locus.
   312. The engineered cell or cardiomyocyte or the method of any of embodiments 307-311, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
   313. The engineered cell or cardiomyocyte or the method of any of embodiments 307-312, wherein the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
   314. The engineered cell or cardiomyocyte or the method of any of embodiments 307-313, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.
   315. The engineered cell or cardiomyocyte or the method of any of embodiments 307-314, wherein the genome targeting entity and the genome modifying entity are different domains of a single polypeptide.
   316. The engineered cell or cardiomyocyte or the method of any of embodiments 307-315, wherein the genome editing entity and genome modifying entity are two different polypeptides that are operably linked together.
   317. The engineered cell or cardiomyocyte or the method of any of embodiments 307-316, wherein the genome editing entity and genome modifying entity are two different polypeptides that are not linked together.
   318. The engineered cell or cardiomyocyte or the method of any of embodiments 307-317, wherein the genome editing complex comprises a guide nucleic acid having a targeting domain that is complementary to at least one target locus, optionally wherein the guide nucleic acid is a guide RNA (gRNA).
   319. The engineered cell or cardiomyocyte or the method of any of embodiments 307-318, wherein the one or more modifications are made by the genome editing complex.
   320. The engineered cell or cardiomyocyte or the method of embodiment 319, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).
   321. The engineered cell or cardiomyocyte or the method of embodiment 319 or embodiment 320, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).
   322. The engineered cell or cardiomyocyte or the method of any of embodiments 318-321, wherein the modifications made by the genome editing complex are made using a guide RNA (gRNA) having a targeting domain that is complementary to at least one target site.
   323. The engineered cell or cardiomyocyte or the method of embodiment 304 or embodiment 305, wherein the genome editing complex is an RNA-guided nuclease.
   324. The engineered cell or cardiomyocyte or the method of embodiment 323, wherein the RNA-guided nuclease comprises a Cas nuclease and a guide RNA (CRISPR-Cas combination).
   325. The engineered cell or cardiomyocyte or the method of embodiment 324, wherein the CRISPR-Cas combination is a ribonucleoprotein (RNP) complex comprising the gRNA and the Cas nuclease.
   326. The engineered cell or cardiomyocyte or the method of embodiment 324 or embodiment 325, wherein the Cas nuclease is a Type II or Type V Cas protein.
   327. The engineered cell or cardiomyocyte or the method of any of embodiments 324-326, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.
   328. A cardiac cell therapy comprising a plurality of cardiomyocytes produced by the method of any of embodiments 251-327.
   329. A method of treatment comprising administering the cardiac cell therapy of embodiment 328 to a subject.
   330. A method of treatment comprising administering a cardiac cell therapy comprising a plurality of cardiomyocytes of any of embodiments 14-99 and 111-250 to a subject.
   331. A method of treatment comprising administering a cardiac cell therapy to a subject, wherein the cardiac cell therapy comprises engineered cardiomyocytes comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to cardiomyocytes that do not comprise the one or more modifications.
   332. The method of embodiment 331, wherein the engineered cardiomyocytes comprise one or more modifications that reduce expression of CACNA1G.
   333. The method of embodiment 331 or embodiment 332, wherein the engineered cardiomyocytes comprise one or more modifications that reduce expression of HCN4 and/or SLC8A1.
   334. The method of any of embodiments 331-333, wherein the engineered cardiomyocytes comprise one or more modifications that increase expression of KCNJ2.
   335. The method of any of embodiments 331-334, wherein the engineered cardiomyocytes comprise one or more modifications that increase expression of TRDN.
   336. The method of any of embodiments 331-335, wherein the engineered cardiomyocytes comprise one or more modifications that increase expression of SRL.
   337. The method of any of embodiments 331-336, wherein the engineered cardiomyocytes comprise one or more modifications that increase expression of HRC.
   338. The method of any of embodiments 331-337, wherein the engineered cardiomyocytes comprise one or more modifications that increase expression of CASQ2.
   339. The method of any of embodiments 331-338, wherein the engineered cardiomyocytes comprise one or more modifications that (a) reduce expression of CACNA1G, HCN4, and SLC8A1; and (b) increase expression of KCNJ2.
   340. The method of any of embodiments 329-339, wherein the cardiac cell therapy is administered as a suspension of cardiomyocytes or as an engineered tissue graft comprising cardiomyocytes and a matrix.
   341. The method of any of embodiments 329-340, wherein administration of the cardiac cell therapy comprises delivery into a subject's heart tissue, optionally by intravenous injection, intraarterial injection, intracoronary injection, intramuscular injection, intraperitoneal injection, intramyocardial injection, trans-endocardial injection, trans-epicardial injection, and/or infusion.
   342. The method of any of embodiments 329-341, wherein administration of the cardiac cell therapy to the subject results in less engraftment arrhythmia (EA) in the subject, relative to a cardiac cell therapy comprising cardiomyocytes not having the one or more modifications.
   343. The method of any of embodiments 329-342, wherein administration of the cardiac cell therapy to the subject does not cause engraftment arrhythmia (EA) in the subject.
   344. The method of any of embodiments 329-343 or the cardiac cell therapy of embodiment 328, wherein the cardiac cell therapy comprises between about 5×10⁸ and 1×10¹⁰ engineered cardiomyocytes, inclusive of each.
   345. The method of any of embodiments 329-344 or the cardiac cell therapy of embodiment 328, wherein the cardiac cell therapy comprises between about 1×10⁹ and about 5×10⁹ engineered cardiomyocytes, inclusive of each.
   346. The method of any of embodiments 329-345 or the cardiac cell therapy of embodiment 328, wherein the cardiac cell therapy comprises a pharmaceutically acceptable carrier.
   347. The method of any of embodiments 329-346 or the cardiac cell therapy of embodiment 328, wherein the subject has a heart disease or condition.
   348. The method or cardiac cell therapy of embodiment 347, wherein the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.
   349. The method or cardiac cell therapy of embodiment 347 or embodiment 348, wherein the heart disease or condition is myocardial infarction (MI).
   350. The method of any of embodiments 329-349, further comprising administering one or more immunosuppressive agents to the subject.
   351. The method of any of embodiments 329-350, wherein the subject has been administered one or more immunosuppressive agents.
   352. The method of embodiment 350 or embodiment 351, wherein the one or more immunosuppressive agents are a small molecule or an antibody.
   353. The method of any of embodiments 350-352, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody.
   354. The method of any of embodiments 350-353, wherein the one or more immunosuppressive agents comprise cyclosporine.
   355. The method of any of embodiments 350-354, wherein the one or more immunosuppressive agents comprise mycophenolate mofetil.
   356. The method of any of embodiments 350-355, wherein the one or more immunosuppressive agents comprise a corticosteroid 357. The method of any of embodiments 350-356, wherein the one or more immunosuppressive agents comprise cyclophosphamide.
   358. The method of any of embodiments 350-357, wherein the one or more immunosuppressive agents comprise rapamycin.
   359. The method of any of embodiments 350-358, wherein the one or more immunosuppressive agents comprise tacrolimus (FK-506).
   360. The method of any of embodiments 350-359, wherein the one or more immunosuppressive agents comprise anti-thymocyte globulin.
   361. The method of any of embodiments 350-360, wherein the one or more immunosuppressive agents are one or more immunomodulatory agents.
   362. The method of embodiment 361, wherein the one or more immunomodulatory agents are a small molecule or an antibody.
   363. The method of embodiment 352 or embodiment 362, wherein the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.
   364. The method of any of embodiments 350-363, wherein the one or more immunosuppressive agents are or have been administered to the subject prior to administration of the cardiac cell therapy.
   365. The method of any of embodiments 350-364, wherein the one or more immunosuppressive agents are or have been administered to the subject at least 1, 2, 3, 4, 5, 6, 7, 8, 9, 10, 11, 12, 13, or 14 days prior to administration of the cardiac cell therapy.
   366. The method of any of embodiments 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the cardiac cell therapy.
   367. The method of any of embodiments 350-365, wherein the one or more immunosuppressive agents are or have been administered to the subject at least 1, 2, 3, 4, 5, 6, 7, 8, 9, 10, 11, 12, 13, or 14 days after administration of the cardiac cell therapy.
   368. The method of any of embodiments 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the cardiac cell therapy.
   369. The method of any of embodiments 350-365, wherein the one or more immunosuppressive agents are or have been administered to the subject on the same day as the first administration of the cardiac cell therapy.
   370. The method of any of embodiments 350-365, wherein the one or more immunosuppressive agents are or have been administered to the subject after administration of the cardiac cell therapy.
   371. The method of any of embodiments 350-365, wherein the one or more immunosuppressive agents are or have been administered to the subject after administration of a first and/or second administration of the cardiac cell therapy.
   372. The method of any of embodiments 350-365, wherein the one or more immunosuppressive agents are or have been administered to the subject prior to administration of a first and/or second administration of the cardiac cell therapy.
   373. The method of any of embodiments 350-365, wherein the one or more immunosuppressive agents are or have been administered to the subject at least 1, 2, 3, 4, 5, 6, 7, 8, 9, 10, 11, 12, 13, or 14 days prior to administration of a first and/or second administration of the cardiac cell therapy.
   374. The method of any of embodiments 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the cardiac cell therapy.
   375. The method of any of embodiments 350-365, wherein the one or more immunosuppressive agents are or have been administered to the subject at least 1, 2, 3, 4, 5, 6, 7, 8, 9, 10, 11, 12, 13, or 14 days after administration of a first and/or second administration of the cardiac cell therapy.
   376. The method of any of embodiments 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the cardiac cell therapy.
   377. The method of any of embodiments 350-365, wherein the one or more immunosuppressive agents are administered at a lower dosage compared to the dosage of one or more immunosuppressive agents administered to reduce immune rejection of immunogenic cells that do not comprise the modifications of the cardiac cell therapy.
   378. The method of any of embodiments 329-377, wherein the engineered cardiomyocyte of the plurality of engineered cardiomyocytes is capable of controlled killing of the engineered cardiomyocyte.
   379. The method of any of embodiments 329-378, wherein the engineered cardiomyocyte of the plurality of engineered cardiomyocytes comprises a safety switch.
   380. The method of embodiment 379, wherein the safety switch induces controlled cell death in the presence of a drug or prodrug, or upon activation by a selective exogenous compound.
   381. The method of embodiment 379 or embodiment 380, wherein the safety switch is a system wherein upon activation, cells downregulate expression of the one or more tolerogenic factors and/or upregulate expression of one or more immune signaling molecules thereby marking the cell for elimination by the host immune system.
   382. The method of embodiment 381, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.
   383. The method of embodiment 381 or embodiment 382, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.
   384. The method of embodiment 382 or embodiment 383, wherein the safety switch is an inducible protein capable of inducing apoptosis of the engineered cardiomyocyte.
   385. The method of embodiment 384, wherein the inducible protein capable of inducing apoptosis of the engineered cardiomyocyte is a caspase protein.
   386. The method of embodiment 385, wherein the caspase protein is caspase 9.
   387. The method of embodiment 379 or embodiment 380, wherein the safety switch is a suicide gene.
   388. The method of embodiment 387, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).
   389. The method of embodiment 387, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).
   390. The method of any of embodiments 379-389, wherein the safety switch is activated to induce controlled cell death after the administration of the one or more immunosuppressive agents to the subject.
   391. The method of any of embodiments 379-389, wherein the safety switch is activated to induce controlled cell death prior to the administration of the one or more immunosuppressive agents to the subject.
   392. The method of any of embodiments 379-391, wherein the safety switch is activated to induce controlled cell death after the administration of the cardiac cell therapy to the subject.
   393. The method of any of embodiments 379-392, wherein the safety switch is activated to induce controlled cell death in the event of cytotoxicity or other negative consequences to the subject.
   394. The method of any of embodiments 379-393, comprising administering an agent that allows for depletion of an engineered cardiomyocyte of the plurality of cardiomyocytes.
   395. The method of embodiment 394, wherein the agent that allows for depletion of the engineered cardiomyocyte is an antibody that recognizes a protein expressed on the surface of the engineered cardiomyocyte.
   396. The method of embodiment 395, wherein the antibody is selected from the group consisting of an antibody that recognizes CCR4, CD16, CD19, CD20, CD30, EGFR, GD2, HER1, HER2, MUC1, PSMA, and RQR8.
   397. The method of embodiment 395, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.
   398. The method of any of embodiments 329-397, comprising administering an agent that recognizes the one or more tolerogenic factors on the surface of the engineered cardiomyocyte.
   399. The method of embodiment 398, wherein the engineered cardiomyocyte is engineered to express the one or more tolerogenic factors.
   400. The method of embodiment 398 or embodiment 399, wherein the one or more tolerogenic factors is CD47.
   401. The method of any of embodiments 329-400, further comprising administering one or more additional therapeutic agents to the subject.
   402. The method of any of embodiments 329-401, wherein the subject has been administered one or more additional therapeutic agents.
   403. The method of any of embodiments 329-402, further comprising monitoring the therapeutic efficacy of the method.
   404. The method of any of embodiments 329-403, further comprising monitoring the prophylactic efficacy of the method.
   405. The method of any of embodiments 329-404, wherein the method is repeated until a desired suppression of one or more disease symptoms occurs.
   406. The method of any of embodiments 329-405, wherein the engineered cardiomyocytes comprise one or more modifications that:
(a) inactivate or disrupt one or more alleles of:
   (i) one or more major histocompatibility complex (MHC) class I molecules or one or more molecules that regulate expression of the one or more MHC class I molecules; and/or
   (ii) one or more MHC class II molecules or one or more molecules that regulate expression of the one or more MHC class II molecules; and/or
(b) increase expression of one or more tolerogenic factors in the engineered cell, relative to a cell of the same cell type that does not comprise the one or more modifications
   407. The method of embodiment 406, wherein the one or more modifications in (a) increase expression of one or more tolerogenic factors, relative to cardiomyocytes that do not comprise the one or more modifications that make the engineered cardiomyocytes hypoimmunogenic.
   408. The method of embodiment 406 or embodiment 407, wherein the one or more MHC class I molecules is one or more human leukocyte antigen (HLA) class I molecules.
   409. The method of embodiment 408, wherein the one or more MHC HLA class I molecules is selected from the group consisting of HLA-A, HLA-B, and HLA-C.
   410. The method of any of embodiments 406-409, wherein the one or more molecules that regulate expression of the one or more MHC class I molecules is/are selected from the group consisting of B-2 microglobulin (B2M) gene and/or the transporter 1, ATP binding cassette subfamily B member (TAP1).
   411. The method of any of embodiments 406-410, wherein the one or more molecules that regulate expression of the one or more MHC class I molecules regulate cell surface protein expression of the one or more MHC class I molecules.
   412. The method of any of embodiments 406-411, wherein the one or more modifications in (a) reduce expression of the one or more MHC HLA class I molecules.
   413. The method of any of embodiments 406-412, wherein the one or more modifications in (a) reduce cell surface trafficking of the one or more MHC HLA class I molecules.
   414. The method of any of embodiments 406-413, wherein the one or more modifications in (a) reduce expression of MHC HLA class I molecules HLA-A, HLA-B, and HLA-C.
   415. The method of any of embodiments 406-414, wherein the one or more modifications in (a) reduce protein expression of the one or more MHC HLA class I molecules.
   416. The method of any of embodiments 406-415, wherein the one or more molecules that regulate cell surface protein expression of the one or more MHC class I molecules is B2M.
   417. The method of any of embodiments 406-416, wherein the one or more modifications comprise a modification that regulates cell surface protein expression of the one or more MHC class I molecules and the modification inactivates or disrupts one or more alleles of B2M.
   418. The method of any of embodiments 406-417, wherein cell surface trafficking of the one or more MHC class I molecules is reduced in the engineered cell relative to the cell of the same cell type that does not comprise the one or more modifications.
   419. The method of embodiment 415, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.
   420. The method of any of embodiments 406-419, wherein the engineered cardiomyocytes comprise one or more modifications that reduce cell surface expression of one or more MHC HLA class I molecules.
   421. The method of any of embodiments 406-420, wherein the engineered cardiomyocytes comprise one or more modifications that reduce a function of one or more MHC HLA class I molecules, optionally wherein the function is antigen presentation.
   422. The method of any of embodiments 406-421, wherein the one or more modifications in (a) inactivates or disrupts one or more alleles of B2M, NLRC5, or TAP1.
   423. The method of embodiment 422, wherein the one or more modifications in (a) inactivates or disrupts one or more alleles of B2M.
   424. The method of embodiment 423, wherein the modification that inactivates or disrupts one or more alleles of B2M reduces mRNA expression of the B2M gene.
   425. The method of any of embodiments 422-424, wherein the modification that inactivates or disrupts one or more alleles of B2M reduces protein expression of B2M.
   426. The method of any of embodiments 422-425, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.
   427. The method of any of embodiments 422-426, wherein the inactivation or disruption comprises an indel in the B2M gene.
   428. The method of any of embodiments 422-427, wherein the inactivation or disruption comprises a frameshift mutation or a deletion of a contiguous stretch of genomic DNA of the B2M gene.
   429. The method of any of embodiments 406-428, wherein the one or more modifications in (a) that reduce expression reduce expression of the B2M gene.
   430. The method of any of embodiments 406-429, wherein the one or more modifications in (a) reduce expression of MHC HLA class I and class II molecules.
   431. The method of any of embodiments 406-430, wherein the one or more modifications in (a) reduce expression of MHC HLA class II molecule HLA-DP, HLA-DQ, or HLA-DR.
   432. The method of any of embodiments 406-431, wherein the one or more modifications in (a) reduce protein expression of one or more MHC class II molecules.
   433. The method of embodiment 432, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.
   434. The method of any of embodiments 329-433, wherein the engineered cardiomyocytes comprise one or more modifications that reduce cell surface expression of one or more MHC HLA class II molecules.
   435. The method of any of embodiments 329-434, wherein the engineered cardiomyocytes comprise one or more modifications that reduce a function of one or more MHC HLA class II molecules, optionally wherein the function is antigen presentation.
   436. The method of any of embodiments 406-435, wherein the one or more modifications in (a) reduce expression of the CIITA gene.
   437. The method of any of embodiments 406-436, wherein the one or more tolerogenic factors comprise CD47.
   438. The method of any of embodiments 406-437, wherein the one or more tolerogenic factors comprise CD47, and wherein the one or more modifications that increases expression of CD47 comprise an exogenous polynucleotide encoding the CD47 protein.
   439. The method of any of embodiments 329-438, wherein the phenotype of the engineered cardiomyocytes comprises B2M^{indel/indel}; CIITA^{indel/indel}; and CD47^{tg}.
   440. The method of any of embodiments 329-439, wherein the cardiomyocytes are autologous to the subject.
   441. The method of any of embodiments 329-439, wherein the cardiomyocytes are allogeneic to the subject.
   442. The method of any of embodiments 329-441, wherein the subject is a human.

### X. EXAMPLES

The following example is included for illustrative purposes only and is not intended to limit the scope of the invention.

### Example 1: Gene Expression Analysis of Cardiac Cells and Tissue by Spatial Transcriptomics

Expression of genes, including the CACNA1G, CACNA1H, and CACNA1I genes encoding T-type calcium channels Cav3.1, Cav3.2, and Cav3.3, respectively, was assessed in cardiac tissue and cardiomyocytes differentiated from stem cells.

A. Expression of T-Type Calcium Channels in Cardiomyocytes by scRNASeq Analysis

Human induced pluripotent stem cells (iPSCs) or embryonic stem cells (ESCs) were differentiated into cardiomyocytes by a suspension differentiation method and spatiotemporal gene expression, including T-type calcium channel gene expression, was determined by single cell RNAseq (scRNAseq) analysis.

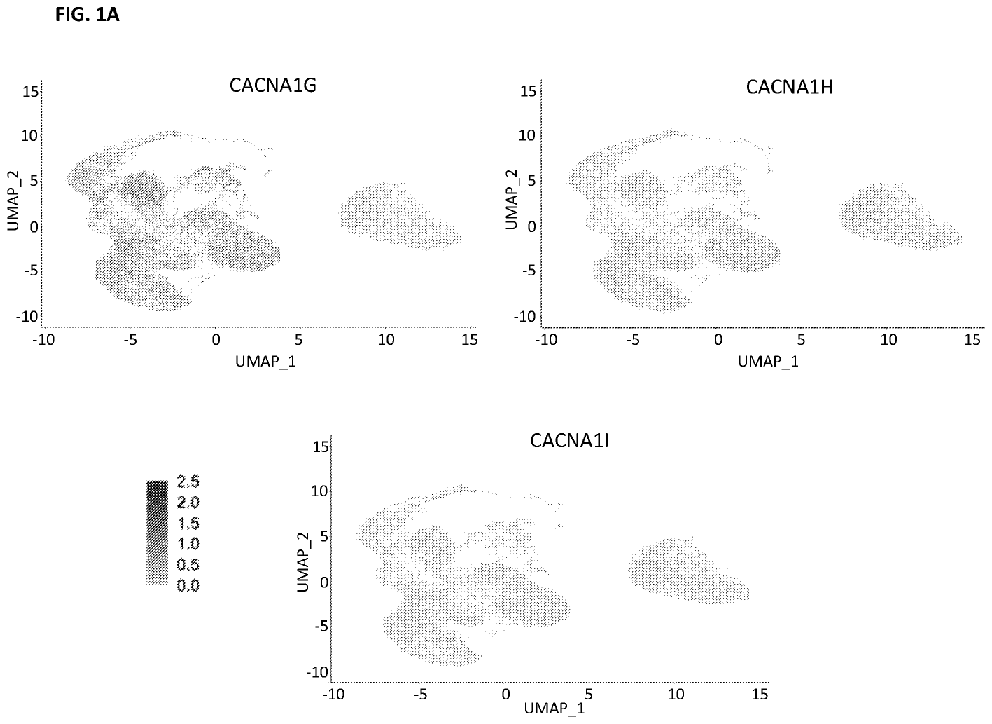

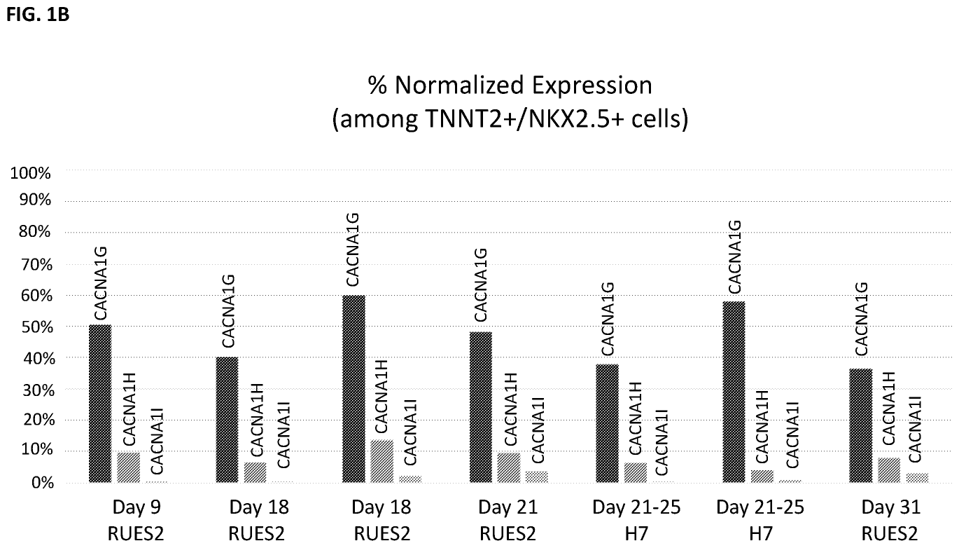

Gene expression, including expression of CACNA1G, CACNA1H, and CACNA1I, was assessed by scRNAseq in cardiomyocytes differentiated from human ESC lines RUES2 and H7, during days 9-31 of in vitro differentiation. Cells were collected and processed by scRNAseq generally as described in Friedman et al., Cell Stem Cell (2018) 23 (4): 586-598.e8. Gene expression across all time points was visualized by Uniform Manifold Approximation Projection (UMAP). With respect to expression of CACNA1G, CACNA1H, and CACNA1I, CACNA1G was observed to be most highly expressed in cells during days 9-31 of the differentiation period (FIG. 1A), though no substantial differences were observed among different time points when expression was analyzed at each of days 9, 18, 21, 21-25, and 31 (FIG. 1B).

B. Expression of T-Type Calcium Channels in Cardiomyocyte Grafts by scRNASeq Analysis

Human embryonic stem cells (ESCs) were differentiated into cardiomyocytes by a suspension differentiation method, and the resulting cardiomyocytes were harvested and cryopreserved. Cryopreserved cardiomyocytes were thawed and transplanted into the hearts of immunosuppressed pigs as a graft.

Gene expression, including expression of CACNA1G, CACNA1H, and CACNA1I T-type calcium channels was assessed by spatial transcriptomics at 6 days, 19 days, and 4 months post-transplantation. Briefly, hearts were cryosectioned, permeabilized, and processed by scRNAseq generally as described in Friedman et al., Cell Stem Cell (2018) 23(4):586-598.e8. Non-biased clustering was performed, and cluster identity was determined based on the presence of human- or pig-specific reads mapping. Differential expression among human graft spots was performed to compare gene expression levels at different time points.

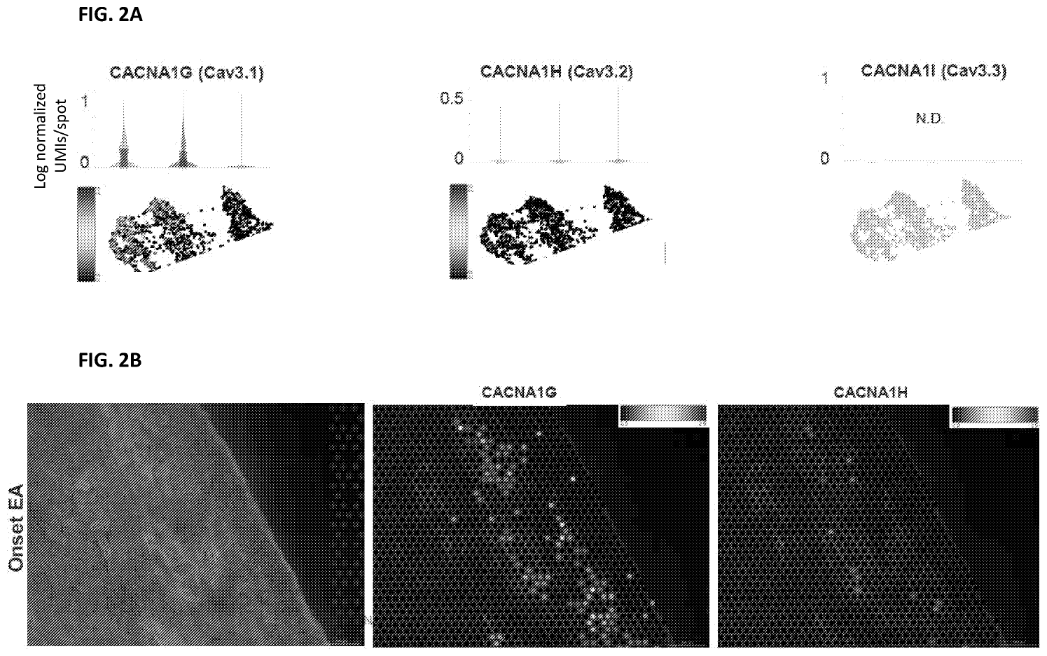

Spatial quantification of CACNA1G, CACNA1H, and CACNA1I expression at onset of engraftment arrhythmia (EA; left of each graph), mid-EA (middle of each graph), and post-EA (right of each graph) revealed that CACNA1G was most highly expressed among the three T-type calcium channels at all three time points assessed (FIG. 2A). Mapping the sequenced mRNAs to the region of the tissue from which they were expressed confirmed that the human CACNA transcripts originated from regions of human grafts showing higher levels of CACNA1G expression compared to CACNA1H expression (FIG. 2B).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO | SEQUENCE | Annotation |
|---|---|---|
| 1 | QLLFNKTKSVEFTFCNDTVVIPCFVTNMEAQNTTEVYVKWKFK | Human CD47 |
| | GRDIYTFDGALNKSTVPTDFSSAKIEVSQLLKGDASLKMDKSD | (without signal |
| | AVSHTGNYTCEVTELTREGETIIELKYRVVSWFSPNENILIVIFPI | sequence) |
| | FAILLFWGQFGIKTLKYRSGGMDEKTIALLVAGLVITVIVIVGAI | |
| | LFVPGEYSLKNATGLGLIVTSTGILILLHYYVFSTAIGLTSFVIAI | |
| | LVIQVIAYILAVVGLSLCIAACIPMHGPLLISGLSILALAQLLGLV | |
| | YMKFVASNQKTIQPPRKAVEEPLNAFKESKGMMNDE | |
| 2 | MWPLVAALLLGSACCGSAQLLENKTKSVEFTFCNDTVVIPCFV | Human CD47 |
| | TNMEAQNTTEVYVKWKFKGRDIYTFDGALNKSTVPTDFSSAKI | (with signal |
| | EVSQLLKGDASLKMDKSDAVSHTGNYTCEVTELTREGETIIEL | sequence) |
| | KYRVVSWFSPNENILIVIFPIFAILLFWGQFGIKTLKYRSGGMDE | |
| | KTIALLVAGLVITVIVIVGAILFVPGEYSLKNATGLGLIVTSTGIL | |
| | ILLHYYVFSTAIGLTSFVIAILVIQVIAYILAVVGLSLCIAACIPMH | |
| | GPLLISGLSILALAQLLGLVYMKFVASNQKTIQPPRKAVEEPLN | |
| | AFKESKGMMNDE | |
| 3 | MDEEEDGAGAEESGQPRSFMRLNDLSGAGGRPGPGSAEKDPG | Human Cav3.1 |
| | SADSEAEGLPYPALAPVVFFYLSQDSRPRSWCLRTVCNPWFERI | |
| | SMLVILLNCVTLGMFRPCEDIACDSQRCRILQAFDDFIFAFFAVE | |
| | MVVKMVALGIFGKKCYLGDTWNRLDFFIVIAGMLEYSLDLQN | |
| | VSFSAVRTVRVLRPLRAINRVPSMRILVTLLLDTLPMLGNVLLL | |
| | CFFVFFIFGIVGVQLWAGLLRNRCFLPENFSLPLSVDLERYYQT | |
| | ENEDESPFICSQPRENGMRSCRSVPTLRGDGGGGPPCGLDYEA | |
| | YNSSSNTTCVNWNQYYTNCSAGEHNPFKGAINEDNIGYAWIAI | |
| | FQVITLEGWVDIMYFVMDAHSFYNFIYFILLIIVGSFFMINLCLV | |
| | VIATQFSETKQRESQLMREQRVRFLSNASTLASFSEPGSCYEEL | |
| | LKYLVYILRKAARRLAQVSRAAGVRVGLLSSPAPLGGQETQPS | |
| | SSCSRSHRRLSVHHLVHHHHHHHHHYHLGNGTLRAPRASPEIQ | |
| | DRDANGSRRLMLPPPSTPALSGAPPGGAESVHSFYHADCHLEP | |
| | VRCQAPPPRSPSEASGRTVGSGKVYPTVHTSPPPETLKEKALVE | |
| | VAASSGPPTLTSLNIPPGPYSSMHKLLETQSTGACQSSCKISSPC | |
| | LKADSGACGPDSCPYCARAGAGEVELADREMPDSDSEAVYEF | |
| | TQDAQHSDLRDPHSRRQRSLGPDAEPSSVLAFWRLICDTFRKIV | |
| | DSKYFGRGIMIAILVNTLSMGIEYHEQPEELTNALEISNIVFTSLF | |
| | ALEMLLKLLVYGPFGYIKNPYNIFDGVIVVISVWEIVGQQGGGL | |
| | SVLRTFRLMRVLKLVRFLPALQRQLVVLMKTMDNVATFCMLL | |
| | MLFIFIFSILGMHLFGCKFASERDGDTLPDRKNFDSLLWAIVTVF | |
| | QILTQEDWNKVLYNGMASTSSWAALYFIALMTFGNYVLFNLL | |
| | VAILVEGFQAEEISKREDASGQLSCIQLPVDSQGGDANKSESEP | |
| | DFFSPSLDGDGDRKKCLALVSLGEHPELRKSLLPPLIIHTAATPM | |
| | SLPKSTSTGLGEALGPASRRTSSSGSAEPGAAHEMKSPPSARSSP | |
| | HSPWSAASSWTSRRSSRNSLGRAPSLKRRSPSGERRSLLSGEGQ | |
| | ESQDEEESSEEERASPAGSDHRHRGSLEREAKSSFDLPDTLQVP | |
| | GLHRTASGRGSASEHQDCNGKSASGRLARALRPDDPPLDGDD | |
| | ADDEGNLSKGERVRAWIRARLPACCLERDSWSAYIFPPQSRFR | |
| | LLCHRIITHKMFDHVVLVIIFLNCITIAMERPKIDPHSAERIFLTLS | |
| | NYIFTAVFLAEMTVKVVALGWCFGEQAYLRSSWNVLDGLLVL | |
| | ISVIDILVSMVSDSGTKILGMLRVLRLLRTLRPLRVISRAQGLKL | |
| | VVETLMSSLKPIGNIVVICCAFFIIFGILGVQLFKGKFFVCQGEDT | |
| | RNITNKSDCAEASYRWVRHKYNFDNLGQALMSLFVLASKDG | |
| | WVDIMYDGLDAVGVDQQPIMNHNPWMLLYFISFLLIVAFFVL | |
| | NMFVGVVVENFHKCRQHQEEEEARRREEKRL | |
| | RRLEKKRRNLMLDDVIASGSSASAASEAQCKPYYSDYSRFRLL | |
| | VHHLCTSHYLDLFITGVIGLNVVTMAMEHYQQPQILDEALKIC | |
| | NYIFTVIFVLESVFKLVAFGFRRFFQDRWNQLDLAIVLLSIMGIT | |
| | LEEIEVNASLPINPTIIRIMRVLRIARVLKLLKMAVGMRALLDTV | |
| | MQALPQVGNLGLLFMLLFFIFAALGVELFGDLECDETHPCEGL | |
| | GRHATFRNFGMAFLTLFRVSTGDNWNGIMKDTLRDCDQESTC | |
| | YNTVISPIYFVSFVLTAQFVLVNVVIAVLMKHLEESNKEAKEEA | |
| | ELEAELELEMKTLSPQPHSPLGSPFLWPGVEGPDSPDSPKPGAL | |
| | HPAAHARSASHESLEHPTDRQLFDTISLLIQGSLEWELKLMDEL | |
| | AGPGGQPSAFPSAPSLGGSDPQIPLAEMEALSLTSEIVSEPSCSL | |
| | ALTDDSLPDDMHTLLLSALESNMQPHPTELPGPDLLTVRKSGV | |
| | SRTHSLPNDSYMCRHGSTAEGPLGHRGWGLPKAQSGSVLSVH | |
| | SQPADTSYILQLPKDAPHLLQPHSAPTWGTIPKLPPPGRSPLAQR | |
| | PLRRQAAIRTDSLDVQGLGSREDLLAEVSGPSPPLARAYSFWG | |
| | QSSTQAQQHSRSHSKISKHMTPPAPCPGPEPNWGKGPPETRSSL | |
| | ELDTELSWISGDLLPPGGQEEPPSPRDLKKCYSVEAQSCQRRPT | |
| | SWLDEQRRHSIAVSCLDSGSQPHLGTDPSNLGGQPLGGPGSRP | |
| | KKKLSPPSITIDPPESQGPRTPPSPGICLRRRAPSSDSKDPLASGPP | |
| | DSMAASPSPKKDVLSLSGLSSDPADLDP | |
| 5 | MTEGARAADEVRVPLGAPPPGPAALVGASPESPGAPGREAERG | Human Cav3.2 |
| | SELGVSPSESPAAERGAELGADEEQRVPYPALAATVFFCLGQTT | |
| | RPRSWCLRLVCNPWFEHVSMLVIMLNCVTLGMFRPCEDVECG | |
| | SERCNILEAFDAFIFAFFAVEMVIKMVALGLFGQKCYLGDTWN | |
| | RLDFFIVVAGMMEYSLDGHNVSLSAIRTVRVLRPLRAINRVPS | |
| | MRILVTLLLDTLPMLGNVLLLCFFVFFIFGIVGVQLWAGLLRNR | |
| | CFLDSAFVRNNNLTFLRPYYQTEEGEENPFICSSRRDNGMQKCS | |
| | HIPGRRELRMPCTLGWEAYTQPQAEGVGAARNACINWNQYYN | |
| | VCRSGDSNPHNGAINFDNIGYAWIAIFQVITLEGWVDIMYYVM | |
| | DAHSFYNFIYFILLIIVGSFFMINLCLVVIATQFSETKQRESQLMR | |
| | EQRARHLSNDSTLASFSEPGSCYEELLKYVGHIFRKVKRRSLRL | |
| | YARWQSRWRKKVDPSAVQGQGPGHRQRRAGRHTASVHHLV | |
| | YHHHHHHHHHYHFSHGSPRRPGPEPGACDTRLVRAGAPPSPPS | |
| | PGRGPPDAESVHSIYHADCHIEGPQERARVAHAAATAAASLRL | |
| | ATGLGTMNYPTILPSGVGSGKGSTSPGPKGKWAGGPPGTGGH | |
| | GPLSLNSPDPYEKIPHVVGEHGLGQAPGHLSGLSVPCPLPSPPA | |
| | GTLTCELKSCPYCTRALEDPEGELSGSESGDSDGRGVYEFTQD | |
| | VRHGDRWDPTRPPRATDTPGPGPGSPQRRAQQRAAPGEPGWM | |
| | GRLWVTFSGKLRRIVDSKYFSRGIMMAILVNTLSMGVEYHEQP | |
| | EELTNALEISNIVFTSMFALEMLLKLLACGPLGYIRNPYNIFDGII | |
| | VVISVWEIVGQADGGLSVLRTFRLLRVLKLVRFLPALRRQLVV | |
| | LVKTMDNVATFCTLLMLFIFIFSILGMHLFGCKFSLKTDTGDTV | |
| | PDRKNFDSLLWAIVTVFQILTQEDWNVVLYNGMASTSSWAAL | |
| | YFVALMTFGNYVLFNLLVAILVEGFQAEGDANRSDTDEDKTSV | |
| | HFEEDFHKLRELQTTELKMCSLAVTPNGHLEGRGSLSPPLIMCT | |
| | AATPMPTPKSSPFLDAAPSLPDSRRGSSSSGDPPLGDQKPPASLR | |
| | SSPCAPWGPSGAWSSRRSSWSSLGRAPSLKRRGQCGERESLLS | |
| | GEGKGSTDDEAEDGRAAPGPRATPLRRAESLDPRPLRPAALPPT | |
| | KCRDRDGQVVALPSDFFLRIDSHREDAAELDDDSEDSCCLRLH | |
| | KVLEPYKPQWCRSREAWALYLFSPQNRFRVSCQKVITHKMFD | |
| | HVVLVFIFLNCVTIALERPDIDPGSTERVFLSVSNYIFTAIFVAEM | |
| | MVKVVALGLLSGEHAYLQSSWNLLDGLLVLVSLVDIVVAMAS | |
| | AGGAKILGVLRVLRLLRTLRPLRVISRAPGLKLVVETLISSLRPI | |
| | GNIVLICCAFFIIFGILGVQLFKGKFYYCEGPDTRNISTKAQCRA | |
| | AHYRWVRRKYNFDNLGQALMSLFVLSSKDGWVNIMYDGLDA | |
| | VGVDQQPVQNHNPWMLLYFISFLLIVSFFVLNMFVGVVVENFH | |
| | KCRQHQEAEEARRREEKRLRRLERRRRSTFPSPEAQRRPYYAD | |
| | YSPTRRSIHSLCTSHYLDLFITFIICVNVITMSMEHYNQPKSLDE | |
| | ALKYCNYVFTIVFVFEAALKLVAFGFRRFFKDRWNQLDLAIVL | |
| | LSLMGITLEEIEMSAALPINPTIIRIMRVLRIARVLKLLKMATGM | |
| | RALLDTVVQALPQVGNLGLLFMLLFFIYAALGVELFGRLECSE | |
| | DNPCEGLSRHATFSNFGMAFLTLFRVSTGDNWNGIMKDTLREC | |
| | SREDKHCLSYLPALSPVYFVTFVLVAQFVLVNVVVAVLMKHL | |
| | EESNKEAREDAELDAEIELEMAQGPGSARRVDADRPPLPQESP | |
| | GARDAPNLVARKVSVSRMLSLPNDSYMFRPVVPASAPHPRPLQ | |
| | EVEMETYGAGTPLGSVASVHSPPAESCASLQIPLAVSSPARSGE | |
| | PLHALSPRGTARSPSLSRLLCRQEAVHTDSLEGKIDSPRDTLDP | |
| | AEPGEKTPVRPVTQGGSLQSPPRSPRPASVRTRKHTFGQRCVSS | |
| | RPAAPGGEEAEASDPADEEVSHITSSACPWQPTAEPHGPEASPV | |
| | AGGERDLRRLYSVDAQGFLDKPGRADEQWRPSAELGSGEPGE | |
| | AKAWGPEAEPALGARRKKKMSPPCISVEPPAEDEGSARPSAAE | |
| | GGSTTLRRRTPSCEATPHRDSLEPTEGSGAGGDPAAKGERWGQ | |
| | ASCRAEHLTVPSFAFEPLDLGVPSGDPFLDGSHSVTPESRASSSG | |
| | AIVPLEPPESEPPMPVGDPPEKRRGLYLTVPQCPLEKPGSPSATP | |
| | APGGGADDPV | |
| 6 | MDKLPPSMRKRLYSLPQQVGAKAWIMDEEEDAEEEGAGGRQ | Human HCN4 |
| | DPSRRSIRLRPLPSPSPSAAAGGTESRSSALGAADSEGPARGAG | |
| | KSSTNGDCRRFRGSLASLGSRGGGSGGTGSGSSHGHLHDSAEE | |
| | RRLIAEGDASPGEDRTPPGLAAEPERPGASAQPAASPPPPQQPP | |
| | QPASASCEQPSVDTAIKVEGGAAAGDQILPEAEVRLGQAGFMQ | |
| | RQFGAMLQPGVNKFSLRMFGSQKAVEREQERVKSAGFWIIHPY | |
| | SDFRFYWDLTMLLLMVGNLIIIPVGITFFKDENTTPWIVFNVVS | |
| | DTFFLIDLVLNFRTGIVVEDNTEIILDPQRIKMKYLKSWFMVDFI | |
| | SSIPVDYIFLIVETRIDSEVYKTARALRIVRFTKILSLLRLLRLSRL | |
| | IRYIHQWEEIFHMTYDLASAVVRIVNLIGMMLLLCHWDGCLQF | |
| | LVPMLQDFPDDCWVSINNMVNNSWGKQYSYALFKAMSHMLC | |
| | IGYGRQAPVGMSDVWLTMLSMIVGATCYAMFIGHATALIQSL | |
| | DSSRRQYQEKYKQVEQYMSFHKLPPDTRQRIHDYYEHRYQGK | |
| | MFDEESILGELSEPLREEIINFNCRKLVASMPLFANADPNFVTSM | |
| | LTKLRFEVFQPGDYIIREGTIGKKMYFIQHGVVSVLTKGNKETK | |
| | LADGSYFGEICLLTRGRRTASVRADTYCRLYSLSVDNFNEVLEE | |
| | YPMMRRAFETVALDRLDRIGKKNSILLHKVQHDLNSGVFNYQ | |
| | ENEIIQQIVQHDREMAHCAHRVQAAASATPTPTPVIWTPLIQAP | |
| | LQAAAATTSVAIALTHHPRLPAAIFRPPPGSGLGNLGAGQTPRH | |
| | LKRLQSLIPSALGSASPASSPSQVDTPSSSSFHIQQLAGFSAPAGL | |
| | SPLLPSSSSSPPPGACGSPSAPTPSAGVAATTIAGFGHFHKALGG | |
| | SLSSSDSPLLTPLQPGARSPQAAQPSPAPPGARGGLGLPEHFLPP | |
| | PPSSRSPSSSPGQLGQPPGELSLGLATGPLSTPETPPRQPEPPSLV | |
| | AGASGGASPVGFTPRGGLSPPGHSPGPPRTFPSAPPRASGSHGSL | |
| | LLPPASSPPPPQVPQRRGTPPLTPGRLTQDLKLISASQPALPQDG | |
| | AQTLRRASPHSSGESMAAFPLFPRAGGGSGGSGSSGGLGPPGRP | |
| | YGAIPGQHVTLPRKTSSGSLPPPLSLFGARATSSGGPPLTAGPQR | |
| | EPGARPEPVRSKLPSNL | |
| 7 | MYNMRRLSLSPTFSMGFHLLVTVSLLFSHVDHVIAETEMEGEG | Human |
| | NETGECTGSYYCKKGVILPIWEPQDPSFGDKIARATVYFVAMV | SLC8A1 |
| | YMFLGVSIIADRFMSSIEVITSQEKEITIKKPNGETTKTTVRIWNE | |
| | TVSNLTLMALGSSAPEILLSVIEVCGHNFTAGDLGPSTIVGSAAF | |
| | NMFIIIALCVYVVPDGETRKIKHLRVFFVTAAWSIFAYTWLYIIL | |
| | SVISPGVVEVWEGLLTFFFFPICVVFAWVADRRLLFYKYVYKR | |
| | YRAGKQRGMIIEHEGDRPSSKTEIEMDGKVVNSHVENFLDGAL | |
| | VLEVDERDQDDEEARREMARILKELKQKHPDKEIEQLIELANY | |
| | QVLSQQQKSRAFYRIQATRLMTGAGNILKRHAADQARKAVSM | |
| | HEVNTEVTENDPVSKIFFEQGTYQCLENCGTVALTIIRRGGDLT | |
| | NTVFVDFRTEDGTANAGSDYEFTEGTVVFKPGDTQKEIRVGIID | |
| | DDIFEEDENFLVHLSNVKVSSEASEDGILEANHVSTLACLGSPST | |
| | ATVTIFDDDHAGIFTFEEPVTHVSESIGIMEVKVLRTSGARGNVI | |
| | VPYKTIEGTARGGGEDFEDTCGELEFQNDEIVKTISVKVIDDEE | |
| | YEKNKTFFLEIGEPRLVEMSEKKALLLNELGGFTITGKYLFGQP | |
| | VFRKVHAREHPILSTVITIADEYDDKQPLTSKEEEERRIAEMGRP | |
| | ILGEHTKLEVIIEESYEFKSTVDKLIKKTNLALVVGTNSWREQFI | |
| | EAITVSAGEDDDDDECGEEKLPSCFDYVMHFLTVFWKVLFAFV | |
| | PPTEYWNGWACFIVSILMIGLLTAFIGDLASHFGCTIGLKDSVT | |
| | AVVFVALGTSVPDTFASKVAATQDQYADASIGNVTGSNAVNV | |
| | FLGIGVAWSIAAIYHAANGEQFKVSPGTLAFSVTLFTIFAFINVG | |
| | VLLYRRRPEIGGELGGPRTAKLLTSCLFVLLWLLYIFFSSLEAYC | |
| | HIKGF | |
| 8 | MGSVRTNRYSIVSSEEDGMKLATMAVANGFGNGKSKVHTRQ | Human KCNJ2 |
| | QCRSRFVKKDGHCNVQFINVGEKGQRYLADIFTTCVDIRWRW | |
| | MLVIFCLAFVLSWLFFGCVFWLIALLHGDLDASKEGKACVSEV | |
| | NSFTAAFLFSIETQTTIGYGFRCVTDECPIAVFMVVFQSIVGCIID | |
| | AFIIGAVMAKMAKPKKRNETLVFSHNAVIAMRDGKLCLMWR | |
| | VGNLRKSHLVEAHVRAQLLKSRITSEGEYIPLDQIDINVGFDSGI | |
| | DRIFLVSPITIVHEIDEDSPLYDLSKQDIDNADFEIVVILEGMVEA | |
| | TAMTTQCRSSYLANEILWGHRYEPVLFEEKHYYKVDYSRFHK | |
| | TYEVPNTPLCSARDLAEKKYILSNANSFCYENEVALTSKEEDDS | |
| | ENGVPESTSTDTPPDIDLHNQASVPLEPRPLRRESEI | |
| 9 | MSRSVALAVLALLSLSGLEAIQRTPKIQVYSRHPAENGKSNFLN | Human B2M |
| | CYVSGFHPSDIEVDLLKNGERIEKVEHSDLSFSKDWSFYLLYYT | |
| | EFTPTEKDEYACRVNHVTLSQPKIVKWDRDM | |
| 10 | MRCLAPRPAGSYLSEPQGSSQCATMELGPLEGGYLELLNSDAD | Human CIITA |
| | PLCLYHFYDQMDLAGEEEIELYSEPDTDTINCDQFSRLLCDMEG | |
| | DEETREAYANIAELDQYVFQDSQLEGLSKDIFKHIGPDEVIGES | |
| | MEMPAEVGQKSQKRPFPEELPADLKHWKPAEPPTVVTGSLLV | |
| | RPVSDCSTLPCLPLPALFNQEPASGQMRLEKTDQIPMPFSSSSLS | |
| | CLNLPEGPIQFVPTISTLPHGLWQISEAGTGVSSIFIYHGEVPQAS | |
| | QVPPPSGFTVHGLPTSPDRPGSTSPFAPSATDLPSMPEPALTSRA | |
| | NMTEHKTSPTQCPAAGEVSNKLPKWPEPVEQFYRSLQDTYGA | |
| | EPAGPDGILVEVDLVQARLERSSSKSLERELATPDWAERQLAQ | |
| | GGLAEVLLAAKEHRRPRETRVIAVLGKAGQGKSYWAGAVSRA | |
| | WACGRLPQYDFVFSVPCHCLNRPGDAYGLQDLLFSLGPQPLVA | |
| | ADEVFSHILKRPDRVLLILDGFEELEAQDGFLHSTCGPAPAEPCS | |
| | LRGLLAGLFQKKLLRGCTLLLTARPRGRLVQSLSKADALFELS | |
| | GFSMEQAQAYVMRYFESSGMTEHQDRALTLLRDRPLLLSHSH | |
| | SPTLCRAVCQLSEALLELGEDAKLPSTLTGLYVGLLGRAALDSP | |
| | PGALAELAKLAWELGRRHQSTLQEDQFPSADVRTWAMAKGL | |
| | VQHPPRAAESELAFPSFLLQCFLGALWLALSGEIKDKELPQYLA | |
| | LTPRKKRPYDNWLEGVPRFLAGLIFQPPARCLGALLGPSAAAS | |
| | VDRKQKVLARYLKRLQPGTLRARQLLELLHCAHEAEEAGIWQ | |
| | HVVQELPGRLSFLGTRLTPPDAHVLGKALEAAGQDFSLDLRST | |
| | GICPSGLGSLVGLSCVTRFRAALSDTVALWESLQQHGETKLLQ | |
| | AAEEKFTIEPFKAKSLKDVEDLGKLVQTQRTRSSSEDTAGELPA | |
| | VRDLKKLEFALGPVSGPQAFPKLVRILTAFSSLQHLDLDALSEN | |
| | KIGDEGVSQLSATFPQLKSLETLNLSQNNITDLGAYKLAEALPS | |
| | LAASLLRLSLYNNCICDVGAESLARVLPDMVSLRVMDVQYNK | |
| | FTAAGAQQLAASLRRCPHVETLAMWTPTIPFSVQEHLQQQDSR | |
| | ISLR | |
| 11 | MTEITAEGNASTTTTVIDSKNGSVPKSPGKVLKRTVTEDIVTTES | Human triadin |
| | SPAAWLLVIALIITWSAVAIVMFDLVDYKNFSASSIAKIGSDPLK | |
| | LVRDAMEETTDWIYGFFSLLSDIISSEDEEDDDGDEDTDKGEID | |
| | EPPLRKKEIHKDKTEKQEKPERKIQTKVTHKEKEKGKEKVREK | |
| | EKPEKKATHKEKIEKKEKPETKTLAKEQKKAKTAEKSEEKTKK | |
| | EVKGGKQEKVKQTAAKVKEVQKTPSKPKEKEDKEKAAVSKH | |
| | EQKDQYAFCRYMIDIFVHGDLKPGQSPAIPPPLPTEQASRPTPAS | |
| | PALEEKEGEKKKAEKKVTSETKKKEKEDIKKKSEKETAIDVEK | |
| | KEPGKASETKQGTVKIAAQAAAKKDEKKEDSKKTKKPAEVEQ | |
| | PKGKKQEKKEKHVEPAKSPKKEHSVPSDKQVKAKTERAKEEIG | |
| | AVSIKKAVPGKKEEKTTKTVEQEIRKEKSGKTSSILKDKEPIKG | |
| | KEEKVPASLKEKEPETKKDEKMSKAGKEVKPKPPQLQGKKEE | |
| | KPEPQIKKEAKPAISEKVQIHKQDIVKPEKTVSHGKPEEKVLKQ | |
| | VKAVTIEKTAKPKPTKKAEHREREPPSIKTDKPKPTPKGTSEVT | |
| | ESGKKKTEISEKESKEKADMKHLREEKVSTRKESLQLHNVTKA | |
| | EKPARVSKDVEDVPASKKAKEGTEDVSPTKQKSPISFFQCVYL | |
| | DGYNGYGFQFPFTPADRPGESSGQANSPGQKQQGQ | |
| 12 | MRALVLLGCLLASLLFSGQAEETEDANEEAPLRDRSHIEKTLM | Human |
| | LNEDKPSDDYSAVLQRLRKIYHSSIKPLEQSYKYNELRQHEITD | sarcalumenin |
| | GEITSKPMVLFLGPWSVGKSTMINYLLGLENTRYQLYTGAEPT | |
| | TSEFTVLMHGPKLKTIEGIVMAADSARSFSPLEKFGQNFLEKLI | |
| | GIEVPHKLLERVTFVDTPGIIENRKQQERGYPFNDVCQWFIDRA | |
| | DLIFVVFDPTKLDVGLELEMLFRQLKGRESQIRIILNKADNLAT | |
| | QMLMRVYGALFWSLAPLINVTEPPRVYVSSFWPQEYKPDTHQ | |
| | ELFLQEEISLLEDLNQVIENRLENKIAFIRQHAIRVRIHALLVDRY | |
| | LQTYKDKMTFFSDGELVFKDIVEDPDKFYIFKTILAKTNVSKFD | |
| | LPNREAYKDFFGINPISSFKLLSQQCSYMGGCFLEKIERAITQEL | |
| | PGLLGSLGLGKNPGALNCDKTGCSETPKNRYRKH | |
| 13 | MGHHRPWLHASVLWAGVASLLLPPAMTQQLRGDGLGFRNRN | Human HRC |
| | NSTGVAGLSEEASAELRHHLHSPRDHPDENKDVSTENGHHFWS | |
| | HPDREKEDEDVSKEYGHLLPGHRSQDHKVGDEGVSGEEVFAE | |
| | HGGQARGHRGHGSEDTEDSAEHRHHLPSHRSHSHQDEDEDEV | |
| | VSSEHHHHILRHGHRGHDGEDDEGEEEEEEEEEEEEASTEYGH | |
| | QAHRHRGHGSEEDEDVSDGHHHHGPSHRHQGHEEDDDDDDD | |
| | DDDDDDDDDVSIEYRHQAHRHQGHGIEEDEDVSDGHHHRDPS | |
| | HRHRSHEEDDNDDDDVSTEYGHQAHRHQDHRKEEVEAVSGE | |
| | HHHHVPDHRHQGHRDEEEDEDVSTERWHQGPQHVHHGLVDE | |
| | EEEEEEITVQFGHYVASHQPRGHKSDEEDFQDEYKTEVPHHHH | |
| | HRVPREEDEEVSAELGHQAPSHRQSHQDEETGHGQRGSIKEMS | |
| | HHPPGHTVVKDRSHLRKDDSEEEKEKEEDPGSHEEDDESSEQG | |
| | EKGTHHGSRDQEDEEDEEEGHGLSLNQEEEEEEDKEEEEEEED | |
| | EERREERAEVGAPLSPDHSEEEEEEEEGLEEDEPRFTIIPNPLDRR | |
| | EEAGGASSEEESGEDTGPQDAQEYGNYQPGSLCGYCSFCNRCT | |
| | ECESCHCDEENMGEHCDQCQHCQFCYLCPLVCETVCAPGSYV | |
| | DYFSSSLYQALADMLETPEP | |
| 14 | MKRTHLFIVGIYFLSSCRAEEGLNFPTYDGKDRVVSLSEKNFKQ | Human |
| | VLKKYDLLCLYYHEPVSSDKVTQKQFQLKEIVLELVAQVLEHK | calsequestrin-2 |
| | AIGFVMVDAKKEAKLAKKLGFDEEGSLYILKGDRTIEFDGEFA | |
| | ADVLVEFLLDLIEDPVEIISSKLEVQAFERIEDYIKLIGFFKSEDSE | |
| | YYKAFEEAAEHFQPYIKFFATFDKGVAKKLSLKMNEVDFYEPF | |
| | MDEPIAIPNKPYTEEELVEFVKEHQRPTLRRLRPEEMFETWEDD | |
| | LNGIHIVAFAEKSDPDGYEFLEILKQVARDNTDNPDLSILWIDPD | |
| | DFPLLVAYWEKTFKIDLFRPQIGVVNVTDADSVWMEIPDDDDL | |
| | PTAEELEDWIEDVLSGKINTEDDDEDDDDDDNSDEEDNDDSDD | |
| | DDDE | |
| 15 | EGRGSLLTCGDVEENPGP | T2A |
| 16 | LEGGGEGRGSLLTCGDVEENPGPR | T2A |
| 17 | GSGATNFSLLKQAGDVEENPGP | P2A |
| 18 | ATNFSLLKQAGDVEENPGP | P2A |
| 19 | QCTNYALLKLAGDVESNPGP | E2A |
| 20 | VKQTLNFDLLKLAGDVESNPGP | F2A |
| 21 | GSGEGRGSLLTCGDVEENPGP | T2A |
| 22 | AAGSGEGRGSLLTCGDVEENPGP | T2A |
| 23 | GUUUUAGAGCUA | crRNA repeat |
| 24 | UAGCAAGUUAAAAUAAGGCUAGUCCGUUAUCAACUUGAAA | tracrRNA |
| | AAGUGGCACCGAGUCGGUGCUUU | |
| 25 | GAAA | tetraloop |
| 26 | accccacagtggggccacta | GET000046 |
| | | guide |
| 27 | tgttggaaggatgaggaaat | GET000047 |
| | | guide |
| 28 | tcactatgctgccgcccagt | GET000048 |
| | | guide |
| 29 | UCUCUCCAUGUGCAGUAGGA | ABO gRNA |
| 30 | CUGGAUGUCGGAGGAGUACG | FUT1 gRNA |
| 31 | GUCUCCGGAAACUCGAGGUG | RHD gRNA |
| 32 | ACAGUGUAGACUUGAUUGAC | F3 (CD142) |
| | | gRNA |
| 33 | CGUGAGUAAACCUGAAUCUU | B2M gRNA |
| 34 | GAUAUUGGCAUAAGCCUCCC | CIITA gRNA |
| 35 | AGAGUCUCUCAGCUGGUACA | TRAC gRNA |

What is claimed:

1. An engineered cell comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to a cell of the same cell type that does not comprise the one or more modifications.

2. The engineered cell of claim 1, wherein the engineered cell comprises one or more modifications that reduce expression of CACNA1G.

3. The engineered cell of claim 1 or claim 2, wherein the engineered cell comprises one or more modifications that reduce expression of HCN4 and/or SLC8A1.

4. The engineered cell of any of claims 1-3, wherein the engineered cell comprises one or more modifications that increase expression of KCNJ2.

5. The engineered cell of any of claims 1-4, wherein the engineered cell comprises one or more modifications that increase expression of TRDN.

6. The engineered cell of any of claims 1-5, wherein the engineered cell comprises one or more modifications that increase expression of SRL.

7. The engineered cell of any of claims 1-6, wherein the engineered cell comprises one or more modifications that increase expression of HRC.

8. The engineered cell of any of claims 1-7, wherein the engineered cell comprises one or more modifications that increase expression of CASQ2.

9. The engineered cell of any of claims 1-8, wherein the engineered cell comprises one or more modifications that (a) reduce expression of CACNA1G, HCN4, and SLC8A1; and (b) increase expression of KCNJ2.

10. The engineered cell of any of claims 1-9, wherein the engineered cell is a pluripotent stem cell (PSC).

11. The engineered cell of claim 10, wherein the PSC is an induced pluripotent stem cell (iPSC).

12. The engineered cell of claim 10, wherein the PSC is an embryonic stem cell (ESC).

13. The engineered cell of any of claims 1-9, wherein the engineered cell is a primary cardiac cell.

14. The engineered cell of any of claims 1-9 and 13, wherein the engineered cell is a cardiomyocyte or a precursor thereof.

15. The engineered cell of any of claims 1-9, 13, and 14, wherein the engineered cell is a cardiomyocyte.

16. The engineered cell of any of claims 1-9 and 13-15, wherein the engineered cell is a primary cardiomyocyte.

17. The engineered cell of claim 14 or claim 15, wherein the cardiomyocyte or a precursor thereof has been differentiated from a pluripotent stem cell (PSC) in vitro.

18. The engineered cell of claim 17, wherein the in vitro differentiation of the cardiomyocyte or a precursor thereof from a PSC comprises differentiation in suspension culture.

19. The engineered cell of any of claims 1-18, wherein the engineered cell comprises one or more modifications that:
(a) inactivate or disrupt one or more alleles of:
(i) one or more major histocompatibility complex (MHC) class I molecules or one or more molecules that regulate expression of the one or more MHC class I molecules; and/or
(ii) one or more MHC class II molecules or one or more molecules that regulate expression of the one or more MHC class II molecules; and/or
(b) increase expression of one or more tolerogenic factors in the engineered cell, relative to a cell of the same cell type that does not comprise the one or more modifications.

20. The engineered cell of claim 19, wherein the one or more modifications in (a) reduce expression of one or more major histocompatibility complex (MHC) human leukocyte antigen (HLA) class I molecules and/or MHC HLA class II molecules, relative to a cell of the same cell type that does not comprise the one or more modifications.

21. The engineered cell of claim 19, wherein the one or more MHC class I molecules is one or more human leukocyte antigen (HLA) class I molecules.

22. The engineered cell of any of claims 19-21, wherein the one or more MHC HLA class I molecules is selected from the group consisting of HLA-A, HLA-B, and HLA-C.

23. The engineered cell of any of claims 19-22, the one or more molecules that regulate expression of the one or more MHC class I molecules is/are selected from the group consisting of B-2 microglobulin (B2M) gene and/or the transporter 1, ATP binding cassette subfamily B member (TAP1).

24. The engineered cell of any of claims 19-23, wherein the one or more molecules that regulate expression of the one or more MHC class I molecules regulate cell surface protein expression of the one or more MHC class I molecules.

25. The engineered cell of any of claims 19-24, wherein the one or more modifications in (a)(i) reduce expression of the one or more MHC HLA class I molecules.

26. The engineered cell of any of claims 19-25, wherein the one or more modifications in (a)(i) reduce cell surface trafficking of the one or more MHC HLA class I molecules.

27. The engineered cell of any of claims 19-26, wherein the one or more modifications in (a)(i) reduce expression of MHC HLA class I molecules HLA-A, HLA-B, and HLA-C.

28. The engineered cell of any of claims 19-27, wherein the one or more modifications in (a)(i) reduce protein expression of the one or more MHC HLA class I molecules.

29. The engineered cell of any of claims 19-28, wherein the one or more molecules that regulate cell surface protein expression of the one or more MHC class I molecules is B2M.

30. The engineered cell of any of claims 19-29, wherein the one or more modifications comprise a modification that regulates cell surface protein expression of the one or more MHC class I molecules and the modification inactivates or disrupts one or more alleles of B2M.

31. The engineered cell of any of claims 19-30, wherein cell surface trafficking of the one or more MHC class I molecules is reduced in the engineered cell relative to the cell of the same cell type that does not comprise the one or more modifications.

32. The engineered cell of any of claims 28-31, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.

33. The engineered cell of any of claims 1-32, wherein the engineered cell comprises one or more modifications that reduce cell surface expression of one or more MHC HLA class I molecules.

34. The engineered cell of any of claims 1-33, wherein the engineered cell comprises one or more modifications that reduce a function of one or more MHC HLA class I molecules, optionally wherein the function is antigen presentation.

35. The engineered cell of any of claims 19-34, wherein the one or more modifications in (a) inactivates or disrupts one or more alleles of B2M, NLRC5, or TAP1.

36. The engineered cell of claim 35, wherein the one or more modifications in (a) inactivates or disrupts one or more alleles of B2M.

37. The engineered cell of claim 36, wherein the modification that inactivates or disrupts one or more alleles of B2M reduces mRNA expression of the B2M gene.

38. The engineered cell of any of claims 35-37, wherein the modification that inactivates or disrupts one or more alleles of B2M reduces protein expression of B2M.

39. The engineered cell of any of claims 35-38, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

40. The engineered cell of any of claims 36-39, wherein the inactivation or disruption comprises an indel in the B2M gene.

41. The engineered cell of any of claims 36-40, wherein the inactivation or disruption comprises a frameshift mutation or a deletion of a contiguous stretch of genomic DNA of the B2M gene.

42. The engineered cell of any of claims 19-41 wherein the one or more modifications in (a) reduce expression of the B-2 microglobulin (B2M) gene and/or the transporter 1, ATP binding cassette subfamily B member (TAP1) gene.

43. The engineered cell of claim 42, wherein the one or more modifications that reduce expression in (a) reduce expression of the B2M gene.

44. The engineered cell of claim 42 or claim 43, wherein the one or more modifications that reduce expression reduces mRNA expression of the gene.

45. The engineered cell of any of claims 42-44, wherein the one or more modifications that reduce expression reduces protein expression of a protein encoded by the gene.

46. The engineered cell of any of claims 42-45, wherein the one or more modifications that reduce expression comprises inactivation or disruption of one allele of the gene.

47. The engineered cell of any of claims 42-46, wherein the one or more modifications that reduce expression comprises inactivation or disruption of both alleles of the gene.

48. The engineered cell of any of claims 42-47, wherein the one or more modifications that reduce expression comprises inactivation or disruption of all coding sequences of the gene in the cell.

49. The engineered cell of any of claims 46-48, wherein the inactivation or disruption comprises an indel in one allele of the gene.

50. The engineered cell of any of claims 46-49, wherein the inactivation or disruption comprises an indel in both alleles of the gene.

51. The engineered cell of any of claims 42-50, wherein the one or more modifications that reduce expression comprises a frameshift mutation or a deletion of a contiguous stretch of genomic DNA of the gene.

52. The engineered cell of any of claims 42-51, wherein the gene is knocked out.

53. The engineered cell of any of claims 25-52, wherein the one or more modifications that reduce expression of one or more MHC HLA class I molecules is generated by nuclease-mediated gene editing.

54. The engineered cell of claim 53, wherein the nuclease-mediated gene editing is mediated by a zinc finger nuclease (ZFN), a TAL-effector nuclease (TALEN), or a CRISPR-Cas combination that targets the gene.

55. The engineered cell of claim 53 or 54, wherein the nuclease-mediated gene editing uses a CRISPR-Cas system comprising a CRISPR-Cas nuclease and a guide RNA (gRNA) having a targeting domain that is complementary to at least one target site within the gene.

56. The engineered cell of claim 55, wherein the CRISPR-Cas system is a ribonucleoprotein (RNP) complex comprising the gRNA and a Cas protein.

57. The engineered cell of any of claims 19-56, wherein the one or more MHC class II molecules is one or more human leukocyte antigen (HLA) class II molecules.

58. The engineered cell of any of claims 19-56, wherein the one or more modifications in (a) reduce expression of MHC HLA class I and class II molecules.

59. The engineered cell of claim 57 or claim 58, wherein the one or more MHC HLA class II molecules is selected from the group consisting of HLA-DP, HLA-DQ, and/or HLA-DR.

60. The engineered cell of any of claims 19-59, wherein the one or more modifications in (a) reduce protein expression of one or more MHC class II molecules.

61. The engineered cell of any of claims 19-60, wherein the one or more modifications in (a) reduce cell surface trafficking of the one or more MHC class II molecules.

62. The engineered cell of any of claims 19-61, wherein the one or more modifications in (a) reduce a function of the one or more MHC class II molecules, optionally wherein the function is antigen presentation.

63. The engineered cell of claim 60, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.

64. The engineered cell of any of claims 1-63, wherein the engineered cell comprises one or more modifications that reduce cell surface expression of one or more MHC HLA class II molecules.

65. The engineered cell of any of claims 1-64, wherein the engineered cell comprises one or more modifications that reduce a function of one or more MHC HLA class II molecules, optionally wherein the function is antigen presentation.

66. The engineered cell of any of claims 19-65, wherein the one or more molecules that regulate expression of the one or more MHC class II molecules is/are selected from the group consisting of CIITA and CD74.

67. The engineered cell of any of claims 19-66, wherein the modification is a modification that regulates expression of the one or more MHC class II molecules, and the modification inactivates or disrupts one or more alleles of CIITA.

68. The engineered cell of claim 67, wherein the modification that inactivates or disrupts one or more alleles of CIITA reduces mRNA expression of the CIITA gene.

69. The engineered cell of claim 67 or claim 68, wherein the modification that inactivates or disrupts one or more alleles of CIITA reduces protein expression of CIITA.

70. The engineered cell of any of claims 67-69, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the cell.

71. The engineered cell of any of claims 67-70, wherein the inactivation or disruption comprises an indel in the CIITA gene.

72. The engineered cell of any of claims 67-71, wherein the inactivation or disruption is a frameshift mutation or a deletion of a contiguous stretch of genomic DNA of the CIITA gene.

73. The engineered cell of any of claims 1-72, wherein expression of HLA-A, HLA-B, HLA-C, HLA-DP, HLA-DQ, and HLA-DR are reduced in the engineered cell.

74. The engineered cell of any of claims 19-73, wherein the one or more modifications in (a) reduce expression of the CIITA gene.

75. The engineered cell of claim 74, wherein the one or more modifications that reduce expression reduce mRNA expression of the CIITA gene.

76. The engineered cell of claim 74 or claim 75, wherein the one or more modifications that reduce expression reduces expression of a CIITA protein.

77. The engineered cell of any of claims 74-76, wherein the one or more modifications that reduce expression comprises inactivation or disruption of one allele of the CIITA gene.

78. The engineered cell of any of claims 74-77, wherein the one or more modifications that reduce expression comprises inactivation or disruption of both alleles of the CIITA gene.

79. The engineered cell of any of claims 74-78, wherein the one or more modifications that reduce expression comprises inactivation or disruption of all CIITA coding sequences in the cell.

80. The engineered cell of any of claims 77-79, wherein the inactivation or disruption comprises an indel in one allele of the CIITA gene.

81. The engineered cell of any of claims 77-80, wherein the inactivation or disruption comprises an indel in both alleles of the CIITA gene.

82. The engineered cell of any of claims 74-81, wherein the one or more modifications that reduce expression comprises a frameshift mutation or a deletion of a contiguous stretch of genomic DNA of the CIITA gene.

83. The engineered cell of any of claims 19-82, wherein the CIITA gene is knocked out.

84. The engineered cell of any of claims 81-83, wherein the one or more modifications that reduce expression of one or more MHC HLA class II molecules is generated by nuclease-mediated gene editing.

85. The engineered cell of claim 84, wherein the nuclease-mediated gene editing is mediated by a zinc finger nuclease (ZFN), a TAL-effector nuclease (TALEN), or a CRISPR-Cas combination that targets the CIITA gene.

86. The engineered cell of claim 84 or claim 85, wherein the nuclease-mediated gene editing uses a CRISPR-Cas system comprising a CRISPR-Cas nuclease and a guide RNA (gRNA) having a targeting domain that is complementary to at least one target site within the CIITA gene.

87. The engineered cell of claim 86, wherein the CRISPR-Cas system is a ribonucleoprotein (RNP) complex comprising the gRNA and a Cas protein.

88. The engineered cell of any of claims 1-87, wherein expression of HLA-A, HLA-B, HLA-C, HLA-DP, HLA-DQ, and HLA-DR are reduced in the engineered cell.

89. The engineered cell of any of claims 19-88, wherein the one or more tolerogenic factors in (i) are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF, and any combination thereof.

90. The engineered cell of any of claims 19-89, wherein the one or more tolerogenic factors in (i) are selected from the group consisting of CD47, PD-L1, HLA-E, HLA-G, CCL21, FASL, SERPINB9, CD200, MFGE8, and any combination thereof.

91. The engineered cell of any of claims 19-90, wherein the one or more tolerogenic factors in (i) comprise CD47.

92. The engineered cell of any of claims 89-91, wherein the one or more tolerogenic factors in (i) comprise CD47, and wherein the one or more modifications that increases expression of CD47 comprise an exogenous polynucleotide encoding the CD47 protein.

93. The engineered cell of claim 92, wherein the exogenous polynucleotide encoding the CD47 protein is integrated into the genome of the engineered cell.

94. The engineered cell of claim 92 or claim 93, wherein the exogenous polynucleotide encoding the CD47 protein encodes an amino acid sequence having at least 85% sequence identity to the amino acid sequence set forth in SEQ ID NO: 2, optionally wherein the exogenous polynucleotide encoding the CD47 protein encodes the amino acid sequence set forth in SEQ ID NO:2.

95. The engineered cell of any of claims 92-94, wherein the exogenous polynucleotide is integrated by non-targeted insertion into the genome of the engineered cell, optionally by introduction of the exogenous polynucleotide into the cell using a lentiviral vector.

96. The engineered cell of any of claims 92-94, wherein the exogenous polynucleotide is integrated by targeted insertion into a target genomic locus of the engineered cell.

97. The engineered cell of claim 96, wherein the target genomic locus is a safe harbor locus, a B2M gene locus, a CIITA gene locus, a CACNA1G locus, a HCN4 locus, or a SLC8A1 locus.

98. The engineered cell of claim 96 or claim 97, wherein the target genomic locus is selected from the group consisting of: a CCR5 gene locus, a CXCR4 gene locus, a PPP1R12C (also known as AAVSI) gene locus, an albumin gene locus, a SHS231 locus, a CLYBL gene locus, and a ROSA26 gene locus.

99. The engineered cell of any of claims 19-98, wherein the one or more modifications that reduce expression in (a) comprise reduced surface protein expression; and/or the one or more modifications that increase expression in (b) comprise increased surface protein expression.

100. An engineered cell comprising one or more modifications that:
(a) inactivate or disrupt one or more alleles of CACNA1G, HCN4, and SLC8A1, one or more MHC HLA class I molecules, and/or one or more MHC HLA class II molecules; and
(b) increase expression of KCNJ2 and CD47, relative to a cell of the same cell type that does not comprise the one or more modifications.

101. The engineered cell of claim 100, wherein the one or more modifications of (a) reduce expression of CACNA1G, HCN4, and SLC8A1, one or more MHC HLA class I molecules, and/or one or more MHC HLA class II molecules, relative to a cell of the same cell type that does not comprise the one or more modifications.

102. An engineered cell comprising one or more modifications that:
(a) inactivate or disrupt one or more alleles of CACNA1G, HCN4, and SLC8A1, one or more MHC HLA class I molecules, and one or more MHC HLA class II molecules, and
(b) increase expression of KCNJ2 and CD47, relative to a cell of the same cell type that does not comprise the one or more modifications.

103. The engineered cell of claim 102, wherein the one or more modifications of (a) reduce expression of CACNA1G, HCN4, and SLC8A1, one or more MHC HLA class I molecules, and one or more MHC HLA class II molecules, relative to a cell of the same cell type that does not comprise the one or more modifications.

104. The engineered cell of any of claims 100-103, wherein the one or more modifications that reduce expression of one or more MHC HLA class I molecules and/or one or more MHC class II molecules reduce expression of B2M and CIITA.

105. An engineered cell comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.

106. The engineered cell of claim 105, wherein the one or more modifications of (a) reduce expression of CACNA1G, HCN4, SLC8A1, B2M, and CIITA, relative to a cell of the same cell type that does not comprise the one or more modifications.

107. An engineered primary human cell comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.

108. The engineered cell of claim 107, wherein the one or more modifications of (a) reduce expression of CACNA1G, HCN4, SLC8A1, B2M, and CIITA, relative to a cell of the same cell type that does not comprise the one or more modifications.

109. An engineered induced pluripotent stem cell (iPSC) or embryonic stem cell (ESC) comprising one or more modifications that:
(a) inactivate or disrupt one or more alleles of CACNA1G, HCN4, SLC8A1, B2M, and CIITA; and
(b) increase expression of KCNJ2 and CD47, relative to a cell of the same cell type that does not comprise the one or more modifications.

110. The engineered iPSC or ESC of claim 109, wherein the one or more modifications of (a) reduce expression of CACNA1G, HCN4, SLC8A1, B2M, and CIITA, relative to a cell of the same cell type that does not comprise the one or more modifications.

111. An engineered cardiomyocyte that has been differentiated in vitro from an engineered cell of any of claims 1-110.

112. An engineered cardiomyocyte that has been differentiated in vitro from a pluripotent stem cell (PSC), wherein the engineered cardiomyocyte comprises one or more modifications that:
(a) inactivate or disrupt one or more alleles of CACNA1G, HCN4, and SLC8A1;
(b) increase expression of one or more of KCNJ2, TRDN, SRL, HRC, and CASQ2, relative to a cardiomyocyte differentiated in vitro from a PSC that does not comprise the one or more modifications.

113. The engineered cardiomyocyte of claim 112, wherein the one or more modifications of (a) reduce expression of one or more of CACNA1G, HCN4, and SLC8A1, relative to a cardiomyocyte differentiated in vitro from a PSC that does not comprise the one or more modifications.

114. An engineered cardiomyocyte that has been differentiated in vitro from a pluripotent stem cell (PSC), wherein the engineered cardiomyocyte comprises one or more modifications that:
(a) inactivate or disrupt one or more alleles of:
(i) CACNA1G, HCN4, and SLC8A1;
(ii) MHC HLA class I molecules and one or more MHC HLA class II molecules;
(b) increase expression of one or more of KCNJ2, TRDN, SRL, HRC, CASQ2, and CD47; or (d) a combination thereof; or
(c) a combination thereof, relative to a cardiomyocyte differentiated in vitro from a PSC that does not comprise the one or more modifications.

115. The engineered cardiomyocyte of claim 114, wherein the one or more modifications of (a) reduce expression of one or more of CACNA1G, HCN4, and SLC8A1; and reduce expression of one or more of MHC HLA class I molecules and one or more MHC HLA class II molecules, relative to a cardiomyocyte that does not comprise the one or more modifications.

116. The engineered cardiomyocyte of claim 114 or claim 115, wherein the one or more modifications that reduce expression of one or more MHC HLA class I molecules and one or more MHC class II molecules reduce expression of B2M and CIITA.

117. An engineered cardiomyocyte that has been differentiated in vitro from a pluripotent stem cell (PSC), wherein the engineered cardiomyocyte comprises one or more modifications that:
(a) inactivate or disrupt one or more alleles of:
(i) CACNA1G, HCN4, and SLC8A1;
(ii) B2M, TAP1, and CIITA;
(b) increase expression of one or more of KCNJ2, TRDN, SRL, HRC, CASQ2, and CD47; or
(c) a combination thereof, relative to a cardiomyocyte differentiated in vitro from a PSC that does not comprise the one or more modifications.

118. The engineered cardiomyocyte of claim 117, wherein the one or more modifications of (a) reduce expression of one or more of CACNA1G, HCN4, and SLC8A1; and reduce expression of one or more of B2M, TAP1, and CIITA, relative to a cardiomyocyte differentiated in vitro from a PSC that does not comprise the one or more modifications.

119. An engineered cardiomyocyte that has been differentiated in vitro from a pluripotent stem cell (PSC), wherein the engineered cardiomyocyte comprises one or more modifications that:
(a) inactivate or disrupt one or more alleles of CACNA1G, HCN4, and SLC8A1;
(b) increase expression of one or more of KCNJ2, TRDN, SRL, HRC, and CASQ2, relative to a primary cardiomyocyte that does not comprise the one or more modifications.

120. The engineered cardiomyocyte of claim 119, wherein the one or more modifications of (a) reduce expression of one or more of CACNA1G, HCN4, and SLC8A1, relative to a primary cardiomyocyte that does not comprise the one or more modifications.

121. An engineered cardiomyocyte that has been differentiated in vitro from a pluripotent stem cell (PSC), wherein the engineered cardiomyocyte comprises one or more modifications that:
(a) inactivate or disrupt one or more alleles of:
(i) CACNA1G, HCN4, and SLC8A1;
(ii) MHC HLA class I molecules and one or more MHC HLA class II molecules;
(b) increase expression of one or more of KCNJ2, TRDN, SRL, HRC, CASQ2, and CD47; or (d) a combination thereof; or
(c) a combination thereof, relative to a primary cardiomyocyte that does not comprise the one or more modifications.

122. The engineered cardiomyocyte of claim 121, wherein the one or more modifications of (a) reduce expression of one or more of CACNA1G, HCN4, and SLC8A1; and reduce expression of one or more of MHC HLA class I molecules and one or more MHC HLA class II molecules, relative to a primary cardiomyocyte that does not comprise the one or more modifications.

123. The engineered cardiomyocyte of claim 122, wherein the one or more modifications that reduce expression of one or more MHC HLA class I molecules and one or more MHC class II molecules reduce expression of B2M and CIITA.

124. An engineered cardiomyocyte that has been differentiated in vitro from a pluripotent stem cell (PSC), wherein the engineered cardiomyocyte comprises one or more modifications that:
(a) inactivate or disrupt one or more alleles of:
(i) CACNA1G, HCN4, and SLC8A1;
(ii) B2M, TAP1, and CIITA;
(b) increase expression of one or more of KCNJ2, TRDN, SRL, HRC, CASQ2, and CD47; or
(c) a combination thereof, relative to a primary cardiomyocyte that does not comprise the one or more modifications.

125. The engineered cardiomyocyte of claim 124, wherein the one or more modifications of (a) reduce expression of one or more of CACNA1G, HCN4, and SLC8A1; and reduce expression of one or more of B2M, TAP1, and CIITA, relative to a primary cardiomyocyte that does not comprise the one or more modifications.

126. The engineered cell or cardiomyocyte of any of claims 100, 101, and 111-125, wherein the engineered cell or cardiomyocyte comprises one or more modifications that reduce expression of CACNA1G.

127. The engineered cell or cardiomyocyte of any of claims 100, 101, and 111-126, wherein the engineered cell or cardiomyocyte comprises one or more modifications that reduce expression of HCN4 and/or SLC8A1.

128. The engineered cell or cardiomyocyte of any of claims 100, 101, and 111-127, wherein the engineered cell or cardiomyocyte comprises one or more modifications that increase expression of KCNJ2.

129. The engineered cell or cardiomyocyte of any of claims 100, 101, and 111-128, wherein the engineered cell or cardiomyocyte comprises one or more modifications that increase expression of TRDN.

130. The engineered cell or cardiomyocyte of any of claims 100, 101, and 111-129, wherein the engineered cell or cardiomyocyte comprises one or more modifications that increase expression of SRL.

131. The engineered cell or cardiomyocyte of any of claims 100, 101, and 111-130, wherein the engineered cell or cardiomyocyte comprises one or more modifications that increase expression of HRC.

132. The engineered cell or cardiomyocyte of any of claims 100, 101, and 111-131, wherein the engineered cell or cardiomyocyte comprises one or more modifications that increase expression of CASQ2.

133. The engineered cell or cardiomyocyte of any of claims 100, 101, and 111-132, wherein the engineered cell or cardiomyocyte comprises one or more modifications that (a) reduce expression of CACNA1G, HCN4, and SLC8A1; and (b) increase expression of KCNJ2.

134. The engineered cell or cardiomyocyte of any of claims 100, 111, 112, and 126-133, wherein the engineered cell or cardiomyocyte comprises one or more modifications that:
(a) inactivate or disrupt one or more alleles of:
(i) one or more major histocompatibility complex (MHC) class I molecules or one or more molecules that regulate expression of the one or more MHC class I molecules; and/or
(ii) one or more MHC class II molecules or one or more molecules that regulate expression of the one or more MHC class II molecules; and/or
(b) increase expression of one or more tolerogenic factors in the engineered cell or cardiomyocyte, relative to a cell of the same cell type that does not comprise the one or more modifications, optionally wherein the one or more modifications (i) increase expression of one or more tolerogenic factors; and/or (ii) reduce expression of one or more major histocompatibility complex (MHC) human leukocyte antigen (HLA) class I molecules and/or MHC HLA class II molecules, relative to a cell or cardiomyocyte that does not comprise the one or more modifications.

135. The engineered cell or cardiomyocyte of any of claims 100-104, 111, 114-116, and 126-134, wherein the one or more MHC HLA class I molecules is selected from the group consisting of HLA-A, HLA-B, and HLA-C.

136. The engineered cell or cardiomyocyte of any of claims 100-135, wherein the one or more molecules that regulate expression of the one or more MHC class I molecules is/are selected from the group consisting of B-2 microglobulin (B2M) gene and/or the transporter 1, ATP binding cassette subfamily B member (TAP1).

137. The engineered cell or cardiomyocyte of any of claims 100-136, wherein the one or more molecules that regulate expression of the one or more MHC class I molecules regulate cell surface protein expression of the one or more MHC class I molecules.

138. The engineered cell or cardiomyocyte of any of claims 100-111 and 114-137, wherein the one or more modifications reduce expression of the one or more MHC HLA class I molecules.

139. The engineered cell or cardiomyocyte of any of claims 100-111 and 114-138, wherein the one or more modifications reduce cell surface trafficking of the one or more MHC HLA class I molecules.

140. The engineered cell or cardiomyocyte of any of claims 100-111 and 114-139, wherein the one or more modifications reduce expression of MHC HLA class I molecules HLA-A, HLA-B, and HLA-C.

141. The engineered cell or cardiomyocyte of any of claims 100-111 and 114-140, wherein the one or more modifications reduce protein expression of one or more MHC HLA class I molecules.

142. The engineered cell or cardiomyocyte of claim 141, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.

143. The engineered cell or cardiomyocyte of any of claims 100-142, wherein the engineered cell comprises one or more modifications that reduce cell surface expression of one or more MHC HLA class I molecules.

144. The engineered cell or cardiomyocyte of any of claims 100-143, wherein the engineered cell comprises one or more modifications that reduce a function of one or more MHC HLA class I molecules, optionally wherein the function is antigen presentation.

145. The engineered cell of any of claims 100-144, wherein the one or more modifications in (a) inactivates or disrupts one or more alleles of B2M, NLRC5, or TAP1.

146. The engineered cell of claim 145, wherein the one or more modifications in (a) inactivates or disrupts one or more alleles of B2M.

147. The engineered cell of claim 146, wherein the modification that inactivates or disrupts one or more alleles of B2M reduces mRNA expression of the B2M gene.

148. The engineered cell of any of claims 145-147, wherein the modification that inactivates or disrupts one or more alleles of B2M reduces protein expression of B2M.

149. The engineered cell of any of claims 145-148, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

150. The engineered cell of any of claims 145-149, wherein the inactivation or disruption comprises an indel in the B2M gene.

151. The engineered cell of any of claims 145-150, wherein the inactivation or disruption comprises a frameshift mutation or a deletion of a contiguous stretch of genomic DNA of the B2M gene.

152. The engineered cell or cardiomyocyte of any of claims 100-151, wherein the one or more modifications that reduce expression reduce expression of the B2M gene.

153. The engineered cell or cardiomyocyte of any of claims 100-111 and 114-152, wherein the one or more modifications reduce expression of MHC HLA class I and class II molecules.

154. The engineered cell or cardiomyocyte of any of claims 100-111 and 114-153, wherein the one or more modifications reduce expression of MHC HLA class II molecules HLA-DP, HLA-DQ, or HLA-DR.

155. The engineered cell or cardiomyocyte of any of claims 100-111 and 114-154, wherein the one or more modifications reduce protein expression of one or more MHC class II molecules.

156. The engineered cell or cardiomyocyte of claim 155, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.

157. The engineered cell or cardiomyocyte of any of claims 100-156, wherein the engineered cell or cardiomyocyte comprises one or more modifications that reduce cell surface expression of one or more MHC HLA class II molecules.

158. The engineered cell or cardiomyocyte of any of claims 100-157, wherein the engineered cell or cardiomyocyte comprises one or more modifications that reduce a function of one or more MHC HLA class II molecules, optionally wherein the function is antigen presentation.

159. The engineered cell or cardiomyocyte of any of claims 100-111 and 114-158, wherein: the one or more modifications reduce expression of the CIITA gene; and/or the modification that inactivates or disrupts one or more alleles of CIITA comprises:
(i) inactivation or disruption of one allele of the CIITA gene;
(ii) inactivation or disruption of both alleles of the CIITA gene; or
(iii) inactivation or disruption of all CIITA coding alleles in the cell.

160. The engineered cell or cardiomyocyte of claim 111, wherein the one or more tolerogenic factors comprises CD47.

161. The engineered cell or cardiomyocyte of any of claims 100-111 and 114-160, wherein the one or more modifications that increases expression of CD47 comprise an exogenous polynucleotide encoding the CD47 protein.

162. The engineered cell or cardiomyocyte of any of claims 100-161, wherein the phenotype of the engineered cell or cardiomyocyte comprises B2M^{indel/indel}; CIITA^{indel/indel}; and CD47^{tg}.

163. The engineered cell or cardiomyocyte of any of claims 1-162, wherein the engineered cell or cardiomyocyte further comprises a modification for expression of an exogenous safety switch.

164. The engineered cell or cardiomyocyte of claim 163, wherein the safety switch is a system wherein upon activation, cells downregulate expression of the one or more tolerogenic factors and/or upregulate expression of one or more immune signaling molecules thereby marking the engineered cell or cardiomyocyte for elimination by the host immune system.

165. The engineered cell or cardiomyocyte of claim 163 or claim 164, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

166. The engineered cell or cardiomyocyte of claim 164 or claim 165, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

167. The engineered cell or cardiomyocyte of claim 163, wherein the safety switch is a suicide gene.

168. The engineered cell or cardiomyocyte of claim 167, wherein the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

169. The engineered cell or cardiomyocyte of any of claims 163-168, wherein the safety switch and the one or more tolerogenic factors are expressed from a bicistronic cassette integrated into the genome of the engineered cell or cardiomyocyte.

170. The engineered cell or cardiomyocyte of claim 169, wherein the bicistronic cassette is integrated at a non-target locus in the genome of the engineered cell or cardiomyocyte.

171. The engineered cell or cardiomyocyte of claim 169, wherein the bicistronic cassette is integrated into a target genomic locus of the engineered cell or cardiomyocyte.

172. The engineered cell or cardiomyocyte of any of claims 1-171, wherein the engineered cell or cardiomyocyte comprises an exogenous polynucleotide encoding a safety switch.

173. The engineered cell or cardiomyocyte of claim 172, wherein the safety switch is a system wherein upon activation, cells downregulate expression of the one or more tolerogenic factors and/or upregulate expression of one or more immune signaling molecules thereby marking the cell for elimination by the host immune system.

174. The engineered cell or cardiomyocyte of claim 172 or claim 173, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

175. The engineered cell or cardiomyocyte of claim 173 or claim 174, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

176. The engineered cell or cardiomyocyte of claim 163 or claim 172, wherein the safety switch is a suicide gene.

177. The engineered cell or cardiomyocyte of claim 176, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

178. The engineered cell or cardiomyocyte of any of claims 172-177, wherein the safety switch and genes associated with the safety switch are expressed from a bicistronic cassette integrated into the genome of the engineered cell or cardiomyocyte.

179. The engineered cell or cardiomyocyte of any of claims 172-177, wherein the safety switch and the one or more tolerogenic factors are expressed from a bicistronic cassette integrated into the genome of the engineered cell or cardiomyocyte.

180. The engineered cell or cardiomyocyte of claim 178 or claim 179, wherein the bicistronic cassette is integrated by non-targeted insertion into the genome of the engineered cell or cardiomyocyte.

181. The engineered cell or cardiomyocyte of claim 178 or claim 179, wherein the bicistronic cassette is integrated by targeted insertion into a target genomic locus of the engineered cell or cardiomyocyte.

182. The engineered cell or cardiomyocyte of any of claims 172-181, wherein the one or more tolerogenic factors is CD47.

183. The engineered cell or cardiomyocyte of any of claims 1-182, wherein the inactivation or disruption is by one or more gene edits.

184. The engineered cell or cardiomyocyte of any of claims 1-183, wherein the cell comprises a genome editing complex.

185. The engineered cell or cardiomyocyte of claim 183 or claim 184, wherein the one or more gene edits are made by a genome editing complex.

186. The engineered cell or cardiomyocyte of claim 184 or claim 185, wherein the genome editing complex comprises a genome targeting entity and a genome modifying entity.

187. The engineered cell or cardiomyocyte of claim 186, wherein the genome targeting entity localizes the genome editing complex to the one or more alleles that are inactivated or disrupted, optionally wherein the genome targeting entity is a nucleic acid-guided targeting entity.

188. The engineered cell or cardiomyocyte of claim 186 or claim 187, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

189. The engineered cell or cardiomyocyte of any of claims 186-188, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

190. The engineered cell or cardiomyocyte of any of claims 186-189, wherein the genome modifying entity cleaves, deaminates, nicks, polymerizes, interrogates, integrates, cuts, unwinds, breaks, alters, methylates, demethylates, or otherwise destabilizes the target locus.

191. The engineered cell or cardiomyocyte of any of claims 186-190, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

192. The engineered cell or cardiomyocyte of any of claims 186-191, wherein the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

193. The engineered cell or cardiomyocyte of any of claims 186-192, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

194. The engineered cell or cardiomyocyte of any of claims 186-193, wherein the genome targeting entity and the genome modifying entity are different domains of a single polypeptide.

195. The engineered cell or cardiomyocyte of any of claims 186-194, wherein the genome editing entity and genome modifying entity are two different polypeptides that are operably linked together.

196. The engineered cell or cardiomyocyte of any of claims 186-194, wherein the genome editing entity and genome modifying entity are two different polypeptides that are not linked together.

197. The engineered cell or cardiomyocyte of any of claims 186-196, wherein the genome editing complex comprises a guide nucleic acid having a targeting domain that is complementary to at least one target locus, optionally wherein the guide nucleic acid is a guide RNA (gRNA).

198. The engineered cell or cardiomyocyte of any of claims 186-197, wherein the one or more modifications are made by the genome editing complex.

199. The engineered cell or cardiomyocyte of claim 198, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

200. The engineered cell or cardiomyocyte of claim 198 or claim 199, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

201. The engineered cell or cardiomyocyte of any of claims 198-200, wherein the modifications made by the genome editing complex are made using a guide RNA (gRNA) having a targeting domain that is complementary to at least one target site.

202. The engineered cell or cardiomyocyte of any of claims 183-185, wherein the genome editing complex is an RNA-guided nuclease.

203. The engineered cell or cardiomyocyte of claim 202, wherein the RNA-guided nuclease comprises a Cas nuclease and a guide RNA (CRISPR-Cas combination).

204. The engineered cell or cardiomyocyte of claim 203, wherein the CRISPR-Cas combination is a ribonucleoprotein (RNP) complex comprising the gRNA and the Cas nuclease.

205. The engineered cell or cardiomyocyte of claim 203 or claim 204, wherein the Cas nuclease is a Type II or Type V Cas protein.

206. The engineered cell or cardiomyocyte of any of claims 203-205, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

207. The engineered cell or cardiomyocyte of any of claims 1-206, wherein the engineered cell or cardiomyocyte has been differentiated from a pluripotent stem cell (PSC) in vitro.

208. The engineered cell or cardiomyocyte of claim 207, wherein the in vitro differentiation of the engineered cell or cardiomyocyte from a PSC comprises differentiation in suspension culture.

209. The engineered cell or cardiomyocyte of claim 208, wherein differentiation of the cardiomyocyte from the PSC comprises differentiation in suspension culture.

210. The engineered cell or cardiomyocyte of any of claims 207-209, wherein one or more modifications that reduce expression and/or the one or more modifications that increase expression is carried out prior to the differentiation.

211. The engineered cell or cardiomyocyte of any of claims 207-209, wherein one or more modifications that reduce expression and/or the one or more modifications that increase expression is carried out subsequent to the differentiation.

212. The engineered cell or cardiomyocyte of any of claims 207-209, wherein one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.

213. The engineered cell or cardiomyocyte of any of claims 1-212, which is human.

214. A composition comprising a plurality of the engineered cardiomyocytes of any of claims 14-99 and 111-213.

215. The composition of claim 214, wherein the composition comprises between about 5×10⁸ and 1×10¹⁰ engineered cardiomyocytes, inclusive of each.

216. The composition of claim 214 or claim 215, wherein the composition comprises between about 1×10⁹ and about 5×10⁹ engineered cardiomyocytes, inclusive of each.

217. The composition of any of claims 214-216, wherein the composition comprises a pharmaceutically acceptable carrier.

218. The composition of any of claims 214-217, wherein at least at or about 50%, at least at or about 60%, at least at or about 70%, at least at or about 80%, or at least at or about 90% of the cells in the plurality of the engineered cardiomyocytes are reduced for expression of one or more MHC class I molecules and/or for expression of B2M.

219. The composition of any of claims 214-218, wherein at least at or about 50%, at least at or about 60%, at least at or about 70%, at least at or about 80%, or at least at or about 90% of the cells in the plurality of the engineered cardiomyocytes are reduced for expression of one or more MHC class II molecules and/or for expression of CIITA.

220. The composition of any of claims 214-219, wherein at least at or about 50%, at least at or about 60%, at least at or about 70%, at least at or about 80%, or at least at or about 90% of the cells in the plurality of the engineered cardiomyocytes comprise inactivation or disruption of one or more alleles of: one or more MHC class I molecules and/or B2M.

221. The composition of any of claims 214-220, wherein at least at or about 50%, at least at or about 60%, at least at or about 70%, at least at or about 80%, or at least at or about 90% of the cells in the plurality of the engineered cardiomyocytes comprise inactivation or disruption of one or more alleles of: one or more MHC class II molecules and/or CIITA.

222. The composition of any of claims 214-221, wherein at least at or about 50%, at least at or about 60%, at least at or about 70%, at least at or about 80%, or at least at or about 90% of the cells in the plurality of the engineered cardiomyocytes express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications.

223. The composition of any of claims 214-222, wherein at least at or about 50%, at least at or about 60%, at least at or about 70%, at least at or about 80%, or at least at or about 90% of the cells in the plurality of the engineered cardiomyocytes express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a cell of the same cell type that does not comprise the one or more modifications.

224. The composition of any of claims 214-223, wherein at least at or about 50%, at least at or about 60%, at least at or about 70%, at least at or about 80%, or at least at or about 90% of the cells in the plurality of the engineered cardiomyocytes expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

225. The composition of any of claims 214-224, wherein the inactivation or disruption is by one or more gene edits.

226. The composition of any of claims 214-225, wherein the cells of the plurality of the engineered cardiomyocytes comprise a genome editing complex.

227. The composition of claim 225 or claim 226, wherein the one or more gene edits are made by a genome editing complex.

228. The composition of claim 226 or claim 227, wherein the genome editing complex comprises a genome targeting entity and a genome modifying entity.

229. The composition of claim 228, wherein the genome targeting entity localizes the genome editing complex to the one or more alleles that are inactivated or disrupted, optionally wherein the genome targeting entity is a nucleic acid-guided targeting entity.

230. The composition of claim 228 or claim 229, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

231. The composition of any of claims 228-230, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

232. The composition of any of claims 228-231, wherein the genome modifying entity cleaves, deaminates, nicks, polymerizes, interrogates, integrates, cuts, unwinds, breaks, alters, methylates, demethylates, or otherwise destabilizes the target locus.

233. The composition of any of claims 228-232, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

234. The composition of any of claims 228-233, wherein the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

235. The composition of any of claims 228-234, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

236. The composition of any of claims 228-235, wherein the genome targeting entity and the genome modifying entity are different domains of a single polypeptide.

237. The composition of any of claims 228-235, wherein the genome editing entity and genome modifying entity are two different polypeptides that are operably linked together.

238. The composition of any of claims 228-235, wherein the genome editing entity and genome modifying entity are two different polypeptides that are not linked together.

239. The composition of any of claims 228-238, wherein the genome editing complex comprises a guide nucleic acid having a targeting domain that is complementary to at least one target locus, optionally wherein the guide nucleic acid is a guide RNA (gRNA).

240. The composition of any of claims 228-239, wherein the one or more modifications are made by the genome editing complex.

241. The composition of claim 240, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

242. The composition of claim 240 or claim 241, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

243. The composition of any of claims 240-242, wherein the modifications made by the genome editing complex are made using a guide RNA (gRNA) having a targeting domain that is complementary to at least one target site.

244. The composition of any of claims 226-228, wherein the genome editing complex is an RNA-guided nuclease.

245. The composition of claim 244, wherein the RNA-guided nuclease comprises a Cas nuclease and a guide RNA (CRISPR-Cas combination).

246. The composition of claim 245, wherein the CRISPR-Cas combination is a ribonucleoprotein (RNP) complex comprising the gRNA and the Cas nuclease.

247. The composition of claim 245 or claim 246, wherein the Cas nuclease is a Type II or Type V Cas protein.

248. The composition of any of claims 245-247, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

249. The composition of any of claims 214-248, comprising a pharmaceutically acceptable excipient.

250. The composition of any of claims 214-249, comprising a cryoprotectant.

251. A method of producing an engineered cell, the method comprising: (a) reducing expression of one or more of CACNA1G, HCN4, and SLC8A1; (b) increasing expression of one or more of KCNJ2, TRDN, SRL, HRC, and CASQ2; or (c) a combination thereof, in the cell.

252. The method of claim 251, wherein the method comprises reducing expression of CACNA1G in the cell.

253. The method of claim 251 or claim 252, wherein the method comprises reducing expression of HCN4 and/or SLC8A1 in the cell.

254. The method of any of claims 251-253, wherein the method comprises increasing expression of KCNJ2 in the cell.

255. The method of any of claims 251-254, wherein the method comprises increasing expression of TRDN in the cell.

256. The method of any of claims 251-255, wherein the method comprises increasing expression of SRL in the cell.

257. The method of any of claims 251-256, wherein the method comprises increasing expression of HRC in the cell.

258. The method of any of claims 251-257, wherein the method comprises increasing expression of CASQ2 in the cell.

259. The method of any of claims 251-258, wherein the method comprises: (a) reducing expression of CACNA1G, HCN4, and SLC8A1; and (b) increasing expression of KCNJ2, in the cell.

260. The method of any of claims 251-259, wherein the engineered cell is a pluripotent stem cell (PSC).

261. The method of claim 260, wherein the PSC is an induced pluripotent stem cell (iPSC).

262. The method of claim 260, wherein the PSC is an embryonic stem cell (ESC).

263. The method of any of claims 251-259, wherein the engineered cell is a primary cardiac cell.

264. The method of any of claims 251-259 and 263, wherein the engineered cell is a cardiomyocyte or a precursor thereof.

265. The method of any of claims 251-259, 263, and 264, wherein the engineered cell is a cardiomyocyte.

266. The method of any of claims 251-259 and 263-265, wherein the engineered cell is a primary cardiomyocyte.

267. The method of claim 264 or claim 265, wherein the cardiomyocyte or a precursor thereof has been differentiated from a pluripotent stem cell (PSC) in vitro.

268. The method of claim 267, wherein the in vitro differentiation of the cardiomyocyte or a precursor thereof from a PSC comprises differentiation in suspension culture.

269. The method of any of claims 251-262, wherein the method further comprises differentiating the PSC into a cardiomyocyte.

270. The method of claim 269, wherein differentiation of the cardiomyocyte from the PSC comprises differentiation in suspension culture.

271. The method of any of claims 267-270, wherein the reducing expression and/or the increasing expression is carried out prior to the differentiation.

272. The method of any of claims 267-270, wherein the reducing expression and/or the increasing expression is carried out subsequent to the differentiation.

273. The method of any of claims 267-270, wherein part of the reducing expression and/or the increasing expression is carried out prior to the differentiation; and part of the reducing expression and/or the increasing expression is carried out subsequent to the differentiation.

274. The method of any of claims 267-270, wherein one or more modifications that reduce expression and/or the one or more modifications that increase expression is carried out prior to the differentiation.

275. The method of any of claims 267-270, wherein one or more modifications that reduce expression and/or the one or more modifications that increase expression is carried out subsequent to the differentiation.

276. The method of any of claims 267-270, wherein one or more of the one or more modifications that reduce expression and/or the one or more modifications that increase expression is carried out prior to the differentiation; and one or more of the one or more modifications that reduce expression and/or the one or more modifications that increase expression is carried out subsequent to the differentiation.

277. The method of any of claims 251-276, wherein the engineered cell comprises one or more modifications that:
(a) inactivate or disrupt one or more alleles of:
(i) one or more major histocompatibility complex (MHC) class I molecules or one or more molecules that regulate expression of the one or more MHC class I molecules; and/or
(ii) one or more MHC class II molecules or one or more molecules that regulate expression of the one or more MHC class II molecules; and/or
(b) increase expression of one or more tolerogenic factors in the engineered cell or cardiomyocyte, relative to a cell of the same cell type that does not comprise the one or more modifications, optionally wherein the one or more modifications (i) increase expression of one or more tolerogenic factors; and/or (ii) reduce expression of one or more major histocompatibility complex (MHC) class I molecules and/or MHC class II, relative to a cell of the same cell type that does not comprise the one or more modifications.

278. The method of claim 277, wherein the one or more MHC HLA class I molecules is selected from the group consisting of HLA-A, HLA-B, and HLA-C.

279. The method of claim 277 or claim 278, wherein the one or more molecules that regulate expression of the one or more MHC class I molecules is/are selected from the group consisting of B-2 microglobulin (B2M) gene and/or the transporter 1, ATP binding cassette subfamily B member (TAP1).

280. The method of any of claims 277-279, wherein the one or more molecules that regulate expression of the one or more MHC class I molecules regulate cell surface protein expression of the one or more MHC class I molecules.

281. The method of any of claims 277-280, wherein the one or more modifications in (a) reduce expression of the one or more MHC HLA class I molecules.

282. The method of any of claims 277-281, wherein the one or more modifications in (a)(i) reduce cell surface trafficking of the one or more MHC HLA class I molecules.

283. The method of any of claims 277-282, wherein the one or more modifications in (a) reduce expression of MHC HLA class I molecules HLA-A, HLA-B, and HLA-C.

284. The method of any of claims 277-283, wherein the one or more modifications in (a) reduce protein expression of one or more MHC HLA class I molecules.

285. The method of any of claims 277-284, wherein the one or more molecules that regulate cell surface protein expression of the one or more MHC class I molecules is B2M.

286. The method of any of claims 277-285, wherein the one or more modifications comprise a modification that regulates cell surface protein expression of the one or more MHC class I molecules and the modification inactivates or disrupts one or more alleles of B2M.

287. The method of any of claims 277-286, wherein cell surface trafficking of the one or more MHC class I molecules is reduced in the engineered cell relative to the cell of the same cell type that does not comprise the one or more modifications.

288. The method of any of claims 277-287, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.

289. The method of any of claims 251-288, wherein the engineered cell comprises one or more modifications that reduce cell surface expression of one or more MHC HLA class I molecules.

290. The method of any of claims 251-289, wherein the engineered cell comprises one or more modifications that reduce a function of one or more MHC HLA class I molecules, optionally wherein the function is antigen presentation.

291. The method of any of claims 277-290, wherein the one or more modifications in (a) inactivates or disrupts one or more alleles of B2M, NLRC5, or TAP1.

292. The method of claim 291, wherein the one or more modifications in (a) inactivates or disrupts one or more alleles of B2M.

293. The method of claim 291 or claim 292, wherein the modification that inactivates or disrupts one or more alleles of B2M reduces mRNA expression of the B2M gene.

294. The method of any of claims 291-293, wherein the modification that inactivates or disrupts one or more alleles of B2M reduces protein expression of B2M.

295. The method of any of claims 291-294, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

296. The method of any of claims 291-295, wherein the inactivation or disruption comprises an indel in the CIITA gene.

297. The method of any of claims 291-296, wherein the inactivation or disruption is a frameshift mutation or a deletion of a contiguous stretch of genomic DNA of the CIITA gene.

298. The method of any of claims 277-297, wherein expression of HLA-A, HLA-B, HLA-C, HLA-DP, HLA-DQ, and HLA-DR are reduced in the engineered cell.

299. The method of any of claims 277-298, wherein the one or more modifications in (a) reduce expression of the CIITA gene.

300. The method of any of claims 277-299, wherein the one or more tolerogenic factors comprises CD47.

301. The method of any of claims 277-300, wherein the one or more tolerogenic factors comprise CD47, and wherein the one or more modifications that increases expression of CD47 comprise an exogenous polynucleotide encoding the CD47 protein.

302. The method of any of claims 251-301, wherein the phenotype of the engineered cell comprises B2M^{indel/indel}; CIITA^{indel/indel}; and CD47^{tg}.

303. The method of any of claims 108-142, wherein the reducing in (a) is by one or more gene edits.

304. The engineered cell or cardiomyocyte of any of claims 19-213, wherein the inactivating or disrupting of the one or more alleles is by one or more gene edits.

305. The engineered cell or cardiomyocyte of any of claims 1-213 or the method of any of claims 251-303, wherein the cell comprises a genome editing complex.

306. The engineered cell or cardiomyocyte or the method of claim 304 or claim 305, wherein the one or more gene edits are made by a genome editing complex.

307. The engineered cell or cardiomyocyte or the method of claim 306, wherein the genome editing complex comprises a genome targeting entity and a genome modifying entity.

308. The engineered cell or cardiomyocyte or the method of claim 307, wherein the genome targeting entity localizes the genome editing complex to the one or more alleles that are inactivated or disrupted, optionally wherein the genome targeting entity is a nucleic acid-guided targeting entity.

309. The engineered cell or cardiomyocyte or the method of claim 307 or claim 308, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

310. The engineered cell or cardiomyocyte or the method of any of claims 307-309, wherein the genome targeting entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Casa, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

311. The engineered cell or cardiomyocyte or the method of any of claims 307-309, wherein the genome modifying entity cleaves, deaminates, nicks, polymerizes, interrogates, integrates, cuts, unwinds, breaks, alters, methylates, demethylates, or otherwise destabilizes the target locus.

312. The engineered cell or cardiomyocyte or the method of any of claims 307-311, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

313. The engineered cell or cardiomyocyte or the method of any of claims 307-312, wherein the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

314. The engineered cell or cardiomyocyte or the method of any of claims 307-313, wherein the genome modifying entity is selected from the group consisting of Cas1, Cas1b, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csb1, Csb2, Csb3, Csd1, Csd2, Cas5d, Cse1, Cse2, Cse3, Cse4, Csc1, Csc2, Cas5e, Csf1, Csf2, Csf3, Csf4, Csm1, Csm2, Csm3, Csm4, Csm5, Csm6, Csn1, Csn2, Cst1, Cst2, Cas5t, Csh1, Csh2, Cas5h, Csa1, Csa2, Csa3, Csa4, Csa5, Cas5a, Csx1, Csx3, Csx10, Csx11, Csx14, Csx15, Csx16, Csx17, CsaX, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

315. The engineered cell or cardiomyocyte or the method of any of claims 307-314, wherein the genome targeting entity and the genome modifying entity are different domains of a single polypeptide.

316. The engineered cell or cardiomyocyte or the method of any of claims 307-315, wherein the genome editing entity and genome modifying entity are two different polypeptides that are operably linked together.

317. The engineered cell or cardiomyocyte or the method of any of claims 307-316, wherein the genome editing entity and genome modifying entity are two different polypeptides that are not linked together.

318. The engineered cell or cardiomyocyte or the method of any of claims 307-317, wherein the genome editing complex comprises a guide nucleic acid having a targeting domain that is complementary to at least one target locus, optionally wherein the guide nucleic acid is a guide RNA (gRNA).

319. The engineered cell or cardiomyocyte or the method of any of claims 307-318, wherein the one or more modifications are made by the genome editing complex.

320. The engineered cell or cardiomyocyte or the method of claim 319, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

321. The engineered cell or cardiomyocyte or the method of claim 319 or claim 320, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

322. The engineered cell or cardiomyocyte or the method of any of claims 318-321, wherein the modifications made by the genome editing complex are made using a guide RNA (gRNA) having a targeting domain that is complementary to at least one target site.

323. The engineered cell or cardiomyocyte or the method of claim 304 or claim 305, wherein the genome editing complex is an RNA-guided nuclease.

324. The engineered cell or cardiomyocyte or the method of claim 323, wherein the RNA-guided nuclease comprises a Cas nuclease and a guide RNA (CRISPR-Cas combination).

325. The engineered cell or cardiomyocyte or the method of claim 324, wherein the CRISPR-Cas combination is a ribonucleoprotein (RNP) complex comprising the gRNA and the Cas nuclease.

326. The engineered cell or cardiomyocyte or the method of claim 324 or claim 325, wherein the Cas nuclease is a Type II or Type V Cas protein.

327. The engineered cell or cardiomyocyte or the method of any of claims 324-326, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

328. A cardiac cell therapy comprising a plurality of cardiomyocytes produced by the method of any of claims 251-327.

329. A method of treatment comprising administering the cardiac cell therapy of claim 328 to a subject.

330. A method of treatment comprising administering a cardiac cell therapy comprising a plurality of cardiomyocytes of any of claims 14-99 and 111-250 to a subject.

331. A method of treatment comprising administering a cardiac cell therapy to a subject, wherein the cardiac cell therapy comprises engineered cardiomyocytes comprising one or more modifications that: (a) reduce expression of one or more of CACNA1G, HCN4, and SLC8A1; (b) increase expression of one or more of KCNJ2, TRDN, SRL, HRC, and CASQ2; or (c) a combination thereof, relative to cardiomyocytes that do not comprise the one or more modifications.

332. The method of claim 331, wherein the engineered cardiomyocytes comprise one or more modifications that reduce expression of CACNA1G.

333. The method of claim 331 or claim 332, wherein the engineered cardiomyocytes comprise one or more modifications that reduce expression of HCN4 and/or SLC8A1.

334. The method of any of claims 331-333, wherein the engineered cardiomyocytes comprise one or more modifications that increase expression of KCNJ2.

335. The method of any of claims 331-334, wherein the engineered cardiomyocytes comprise one or more modifications that increase expression of TRDN.

336. The method of any of claims 331-335, wherein the engineered cardiomyocytes comprise one or more modifications that increase expression of SRL.

337. The method of any of claims 331-336, wherein the engineered cardiomyocytes comprise one or more modifications that increase expression of HRC.

338. The method of any of claims 331-337, wherein the engineered cardiomyocytes comprise one or more modifications that increase expression of CASQ2.

339. The method of any of claims 331-338, wherein the engineered cardiomyocytes comprise one or more modifications that (a) reduce expression of CACNA1G, HCN4, and SLC8A1; and (b) increase expression of KCNJ2.

340. The method of any of claims 329-339, wherein the cardiac cell therapy is administered as a suspension of cardiomyocytes or as an engineered tissue graft comprising cardiomyocytes and a matrix.

341. The method of any of claims 329-340, wherein administration of the cardiac cell therapy comprises delivery into a subject's heart tissue, optionally by intravenous injection, intraarterial injection, intracoronary injection, intramuscular injection, intraperitoneal injection, intramyocardial injection, trans-endocardial injection, trans-epicardial injection, and/or infusion.

342. The method of any of claims 329-341, wherein administration of the cardiac cell therapy to the subject results in less engraftment arrhythmia (EA) in the subject, relative to a cardiac cell therapy comprising cardiomyocytes not having the one or more modifications.

343. The method of any of claims 329-342, wherein administration of the cardiac cell therapy to the subject does not cause engraftment arrhythmia (EA) in the subject.

344. The method of any of claims 329-343 or the cardiac cell therapy of claim 328, wherein the cardiac cell therapy comprises between about 5×10⁸ and 1×10¹⁰ engineered cardiomyocytes, inclusive of each.

345. The method of any of claims 329-344 or the cardiac cell therapy of claim 328, wherein the cardiac cell therapy comprises between about 1×10⁹ and about 5×10⁹ engineered cardiomyocytes, inclusive of each.

346. The method of any of claims 329-345 or the cardiac cell therapy of claim 328, wherein the cardiac cell therapy comprises a pharmaceutically acceptable carrier.

347. The method of any of claims 329-346 or the cardiac cell therapy of claim 328, wherein the subject has a heart disease or condition.

348. The method or cardiac cell therapy of claim 347, wherein the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

349. The method or cardiac cell therapy of claim 347 or claim 348, wherein the heart disease or condition is myocardial infarction (MI).

350. The method of any of claims 329-349, further comprising administering one or more immunosuppressive agents to the subject.

351. The method of any of claims 329-350, wherein the subject has been administered one or more immunosuppressive agents.

352. The method of claim 350 or claim 351, wherein the one or more immunosuppressive agents are a small molecule or an antibody.

353. The method of any of claims 350-352, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody.

354. The method of any of claims 350-353, wherein the one or more immunosuppressive agents comprise cyclosporine.

355. The method of any of claims 350-354, wherein the one or more immunosuppressive agents comprise mycophenolate mofetil.

356. The method of any of claims 350-355, wherein the one or more immunosuppressive agents comprise a corticosteroid

357. The method of any of claims 350-356, wherein the one or more immunosuppressive agents comprise cyclophosphamide.

358. The method of any of claims 350-357, wherein the one or more immunosuppressive agents comprise rapamycin.

359. The method of any of claims 350-358, wherein the one or more immunosuppressive agents comprise tacrolimus (FK-506).

360. The method of any of claims 350-359, wherein the one or more immunosuppressive agents comprise anti-thymocyte globulin.

361. The method of any of claims 350-360, wherein the one or more immunosuppressive agents are one or more immunomodulatory agents.

362. The method of claim 361, wherein the one or more immunomodulatory agents are a small molecule or an antibody.

363. The method of claim 352 or claim 362, wherein the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

364. The method of any of claims 350-363, wherein the one or more immunosuppressive agents are or have been administered to the subject prior to administration of the cardiac cell therapy.

365. The method of any of claims 350-364, wherein the one or more immunosuppressive agents are or have been administered to the subject at least 1, 2, 3, 4, 5, 6, 7, 8, 9, 10, 11, 12, 13, or 14 days prior to administration of the cardiac cell therapy.

366. The method of any of claims 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the cardiac cell therapy.

367. The method of any of claims 350-365, wherein the one or more immunosuppressive agents are or have been administered to the subject at least 1, 2, 3, 4, 5, 6, 7, 8, 9, 10, 11, 12, 13, or 14 days after administration of the cardiac cell therapy.

368. The method of any of claims 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the cardiac cell therapy.

369. The method of any of claims 350-365, wherein the one or more immunosuppressive agents are or have been administered to the subject on the same day as the first administration of the cardiac cell therapy.

370. The method of any of claims 350-365, wherein the one or more immunosuppressive agents are or have been administered to the subject after administration of the cardiac cell therapy.

371. The method of any of claims 350-365, wherein the one or more immunosuppressive agents are or have been administered to the subject after administration of a first and/or second administration of the cardiac cell therapy.

372. The method of any of claims 350-365, wherein the one or more immunosuppressive agents are or have been administered to the subject prior to administration of a first and/or second administration of the cardiac cell therapy.

373. The method of any of claims 350-365, wherein the one or more immunosuppressive agents are or have been administered to the subject at least 1, 2, 3, 4, 5, 6, 7, 8, 9, 10, 11, 12, 13, or 14 days prior to administration of a first and/or second administration of the cardiac cell therapy.

374. The method of any of claims 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the cardiac cell therapy.

375. The method of any of claims 350-365, wherein the one or more immunosuppressive agents are or have been administered to the subject at least 1, 2, 3, 4, 5, 6, 7, 8, 9, 10, 11, 12, 13, or 14 days after administration of a first and/or second administration of the cardiac cell therapy.

376. The method of any of claims 350-365, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the cardiac cell therapy.

377. The method of any of claims 350-365, wherein the one or more immunosuppressive agents are administered at a lower dosage compared to the dosage of one or more immunosuppressive agents administered to reduce immune rejection of immunogenic cells that do not comprise the modifications of the cardiac cell therapy.

378. The method of any of claims 329-377, wherein the engineered cardiomyocyte of the plurality of engineered cardiomyocytes is capable of controlled killing of the engineered cardiomyocyte.

379. The method of any of claims 329-378, wherein the engineered cardiomyocyte of the plurality of engineered cardiomyocytes comprises a safety switch.

380. The method of claim 379, wherein the safety switch induces controlled cell death in the presence of a drug or prodrug, or upon activation by a selective exogenous compound.

381. The method of claim 379 or claim 380, wherein the safety switch is a system wherein upon activation, cells downregulate expression of the one or more tolerogenic factors and/or upregulate expression of one or more immune signaling molecules thereby marking the cell for elimination by the host immune system.

382. The method of claim 381, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

383. The method of claim 381 or claim 382, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

384. The method of claim 382 or claim 383, wherein the safety switch is an inducible protein capable of inducing apoptosis of the engineered cardiomyocyte.

385. The method of claim 384, wherein the inducible protein capable of inducing apoptosis of the engineered cardiomyocyte is a caspase protein.

386. The method of claim 385, wherein the caspase protein is caspase 9.

387. The method of claim 379 or claim 380, wherein the safety switch is a suicide gene.

388. The method of claim 387, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

389. The method of claim 387, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

390. The method of any of claims 379-389, wherein the safety switch is activated to induce controlled cell death after the administration of the one or more immunosuppressive agents to the subject.

391. The method of any of claims 379-389, wherein the safety switch is activated to induce controlled cell death prior to the administration of the one or more immunosuppressive agents to the subject.

392. The method of any of claims 379-391, wherein the safety switch is activated to induce controlled cell death after the administration of the cardiac cell therapy to the subject.

393. The method of any of claims 379-392, wherein the safety switch is activated to induce controlled cell death in the event of cytotoxicity or other negative consequences to the subject.

394. The method of any of claims 379-393, comprising administering an agent that allows for depletion of an engineered cardiomyocyte of the plurality of cardiomyocytes.

395. The method of claim 394, wherein the agent that allows for depletion of the engineered cardiomyocyte is an antibody that recognizes a protein expressed on the surface of the engineered cardiomyocyte.

396. The method of claim 395, wherein the antibody is selected from the group consisting of an antibody that recognizes CCR4, CD16, CD19, CD20, CD30, EGFR, GD2, HER1, HER2, MUC1, PSMA, and RQR8.

397. The method of claim 395, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

398. The method of any of claims 329-397, comprising administering an agent that recognizes the one or more tolerogenic factors on the surface of the engineered cardiomyocyte.

399. The method of claim 398, wherein the engineered cardiomyocyte is engineered to express the one or more tolerogenic factors.

400. The method of claim 398 or claim 399, wherein the one or more tolerogenic factors is CD47.

401. The method of any of claims 329-400, further comprising administering one or more additional therapeutic agents to the subject.

402. The method of any of claims 329-401, wherein the subject has been administered one or more additional therapeutic agents.

403. The method of any of claims 329-402, further comprising monitoring the therapeutic efficacy of the method.

404. The method of any of claims 329-403, further comprising monitoring the prophylactic efficacy of the method.

405. The method of any of claims 329-404, wherein the method is repeated until a desired suppression of one or more disease symptoms occurs.

406. The method of any of claims 329-405, wherein the engineered cardiomyocytes comprise one or more modifications that:
(a) inactivate or disrupt one or more alleles of:
(i) one or more major histocompatibility complex (MHC) class I molecules or one or more molecules that regulate expression of the one or more MHC class I molecules; and/or
(ii) one or more MHC class II molecules or one or more molecules that regulate expression of the one or more MHC class II molecules; and/or
(b) increase expression of one or more tolerogenic factors in the engineered cell, relative to a cell of the same cell type that does not comprise the one or more modifications

407. The method of claim 406, wherein the one or more modifications in (a) increase expression of one or more tolerogenic factors, relative to cardiomyocytes that do not comprise the one or more modifications that make the engineered cardiomyocytes hypoimmunogenic.

408. The method of claim 406 or claim 407, wherein the one or more MHC class I molecules is one or more human leukocyte antigen (HLA) class I molecules.

409. The method of claim 408, wherein the one or more MHC HLA class I molecules is selected from the group consisting of HLA-A, HLA-B, and HLA-C.

410. The method of any of claims 406-409, wherein the one or more molecules that regulate expression of the one or more MHC class I molecules is/are selected from the group consisting of B-2 microglobulin (B2M) gene and/or the transporter 1, ATP binding cassette subfamily B member (TAP1).

411. The method of any of claims 406-410, wherein the one or more molecules that regulate expression of the one or more MHC class I molecules regulate cell surface protein expression of the one or more MHC class I molecules.

412. The method of any of claims 406-411, wherein the one or more modifications in (a) reduce expression of the one or more MHC HLA class I molecules.

413. The method of any of claims 406-412, wherein the one or more modifications in (a) reduce cell surface trafficking of the one or more MHC HLA class I molecules.

414. The method of any of claims 406-413, wherein the one or more modifications in (a) reduce expression of MHC HLA class I molecules HLA-A, HLA-B, and HLA-C.

415. The method of any of claims 406-414, wherein the one or more modifications in (a) reduce protein expression of the one or more MHC HLA class I molecules.

416. The method of any of claims 406-415, wherein the one or more molecules that regulate cell surface protein expression of the one or more MHC class I molecules is B2M.

417. The method of any of claims 406-416, wherein the one or more modifications comprise a modification that regulates cell surface protein expression of the one or more MHC class I molecules and the modification inactivates or disrupts one or more alleles of B2M.

418. The method of any of claims 406-417, wherein cell surface trafficking of the one or more MHC class I molecules is reduced in the engineered cell relative to the cell of the same cell type that does not comprise the one or more modifications.

419. The method of claim 415, wherein the one or more modifications that reduce protein expression reduce expression of an HLA-A protein, an HLA-B protein, or HLA-C protein, optionally wherein a gene encoding an HLA-A protein, an HLA-B protein, or an HLA-C protein, respectively, is knocked out.

420. The method of any of claims 406-419, wherein the engineered cardiomyocytes comprise one or more modifications that reduce cell surface expression of one or more MHC HLA class I molecules.

421. The method of any of claims 406-420, wherein the engineered cardiomyocytes comprise one or more modifications that reduce a function of one or more MHC HLA class I molecules, optionally wherein the function is antigen presentation.

422. The method of any of claims 406-421, wherein the one or more modifications in (a) inactivates or disrupts one or more alleles of B2M, NLRC5, or TAP1.

423. The method of claim 422, wherein the one or more modifications in (a) inactivates or disrupts one or more alleles of B2M.

424. The method of claim 423, wherein the modification that inactivates or disrupts one or more alleles of B2M reduces mRNA expression of the B2M gene.

425. The method of any of claims 422-424, wherein the modification that inactivates or disrupts one or more alleles of B2M reduces protein expression of B2M.

426. The method of any of claims 422-425, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

427. The method of any of claims 422-426, wherein the inactivation or disruption comprises an indel in the B2M gene.

428. The method of any of claims 422-427, wherein the inactivation or disruption comprises a frameshift mutation or a deletion of a contiguous stretch of genomic DNA of the B2M gene.

429. The method of any of claims 406-428, wherein the one or more modifications in (a) that reduce expression reduce expression of the B2M gene.

430. The method of any of claims 406-429, wherein the one or more modifications in (a) reduce expression of MHC HLA class I and class II molecules.

431. The method of any of claims 406-430, wherein the one or more modifications in (a) reduce expression of MHC HLA class II molecule HLA-DP, HLA-DQ, or HLA-DR.

432. The method of any of claims 406-431, wherein the one or more modifications in (a) reduce protein expression of one or more MHC class II molecules.

433. The method of claim 432, wherein the one or more modifications that reduce protein expression reduce expression of an HLA-DP protein, an HLA-DQ protein, or an HLA-DR protein, optionally wherein a gene encoding an HLA-DP protein, an HLA-DQ protein, or an HLA-DR protein, respectively, is knocked out.

434. The method of any of claims 329-433, wherein the engineered cardiomyocytes comprise one or more modifications that reduce cell surface expression of one or more MHC HLA class II molecules.

435. The method of any of claims 329-434, wherein the engineered cardiomyocytes comprise one or more modifications that reduce a function of one or more MHC HLA class II molecules, optionally wherein the function is antigen presentation.

436. The method of any of claims 406-435, wherein the one or more modifications in (a) reduce expression of the CIITA gene.

437. The method of any of claims 406-436, wherein the one or more tolerogenic factors comprise CD47.

438. The method of any of claims 406-437, wherein the one or more tolerogenic factors comprise CD47, and wherein the one or more modifications that increases expression of CD47 comprise an exogenous polynucleotide encoding the CD47 protein.

439. The method of any of claims 329-438, wherein the phenotype of the engineered cardiomyocytes comprises B2M^{indel/indel}; CIITA^{indel/indel}; and CD47^{tg}.

440. The method of any of claims 329-439, wherein the cardiomyocytes are autologous to the subject.

441. The method of any of claims 329-439, wherein the cardiomyocytes are allogeneic to the subject.

442. The method of any of claims 329-441, wherein the subject is a human.


SEQUENCE LISTING (35 sequences)

SEQ ID NO: 1 - protein, 305 aa, synthetic construct
  REGION 1..305: note = Human CD47 (without signal sequence)
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 323 aa, synthetic construct
  REGION 1..323: note = Human CD47 (with signal sequence)
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 2377 aa, synthetic construct
  REGION 1..2377: note = Human CaV3.1
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEISKREDASGQLSCIQLPVDSQGGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTDRQLFDTISLLIQGSLEWELKLMDELAGPGGQPSAFPSAPSLGGSDPQIPLAEMEALSLTSEIVSEPSCSLALTDDSLPDDMHTLLLSALESNMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEVSGPSPPLARAYSFWGQSSTQAQQHSRSHSKISKHMTPPAPCPGPEPNWGKGPPETRSSLELDTELSWISGDLLPPGGQEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP

SEQ ID NO: 4 - skipped

SEQ ID NO: 5 - protein, 2353 aa, synthetic construct
  REGION 1..2353: note = Human CaV3.2
MTEGARAADEVRVPLGAPPPGPAALVGASPESPGAPGREAERGSELGVSPSESPAAERGAELGADEEQRVPYPALAATVFFCLGQTTRPRSWCLRLVCNPWFEHVSMLVIMLNCVTLGMFRPCEDVECGSERCNILEAFDAFIFAFFAVEMVIKMVALGLFGQKCYLGDTWNRLDFFIVVAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPGRRELRMPCTLGWEAYTQPQAEGVGAARNACINWNQYYNVCRSGDSNPHNGAINFDNIGYAWIAIFQVITLEGWVDIMYYVMDAHSFYNFIYFILLIIVGSFFMINLCLVVIATQFSETKQRESQLMREQRARHLSNDSTLASFSEPGSCYEELLKYVGHIFRKVKRRSLRLYARWQSRWRKKVDPSAVQGQGPGHRQRRAGRHTASVHHLVYHHHHHHHHHYHFSHGSPRRPGPEPGACDTRLVRAGAPPSPPSPGRGPPDAESVHSIYHADCHIEGPQERARVAHAAATAAASLRLATGLGTMNYPTILPSGVGSGKGSTSPGPKGKWAGGPPGTGGHGPLSLNSPDPYEKIPHVVGEHGLGQAPGHLSGLSVPCPLPSPPAGTLTCELKSCPYCTRALEDPEGELSGSESGDSDGRGVYEFTQDVRHGDRWDPTRPPRATDTPGPGPGSPQRRAQQRAAPGEPGWMGRLWVTFSGKLRRIVDSKYFSRGIMMAILVNTLSMGVEYHEQPEELTNALEISNIVFTSMFALEMLLKLLACGPLGYIRNPYNIFDGIIVVISVWEIVGQADGGLSVLRTFRLLRVLKLVRFLPALRRQLVVLVKTMDNVATFCTLLMLFIFIFSILGMHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRRSTFPSPEAQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQRCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV

SEQ ID NO: 6 - protein, 1203 aa, synthetic construct
  REGION 1..1203: note = Human HCN4
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 7 - protein, 973 aa, synthetic construct
  REGION 1..973: note = Human SLC8A1
MYNMRRLSLSPTFSMGFHLLVTVSLLFSHVDHVIAETEMEGEGNETGECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSTIVGSAAFNMFIIIALCVYVVPDGETRKIKHLRVFFVTAAWSIFAYTWLYIILSVISPGVVEVWEGLLTFFFFPICVVFAWVADRRLLFYKYVYKRYRAGKQRGMIIEHEGDRPSSKTEIEMDGKVVNSHVENFLDGALVLEVDERDQDDEEARREMARILKELKQKHPDKEIEQLIELANYQVLSQQQKSRAFYRIQATRLMTGAGNILKRHAADQARKAVSMHEVNTEVTENDPVSKIFFEQGTYQCLENCGTVALTIIRRGGDLTNTVFVDFRTEDGTANAGSDYEFTEGTVVFKPGDTQKEIRVGIIDDDIFEEDENFLVHLSNVKVSSEASEDGILEANHVSTLACLGSPSTATVTIFDDDHAGIFTFEEPVTHVSESIGIMEVKVLRTSGARGNVIVPYKTIEGTARGGGEDFEDTCGELEFQNDEIVKTISVKVIDDEEYEKNKTFFLEIGEPRLVEMSEKKALLLNELGGFTITGKYLFGQPVFRKVHAREHPILSTVITIADEYDDKQPLTSKEEEERRIAEMGRPILGEHTKLEVIIEESYEFKSTVDKLIKKTNLALVVGTNSWREQFIEAITVSAGEDDDDDECGEEKLPSCFDYVMHFLTVFWKVLFAFVPPTEYWNGWACFIVSILMIGLLTAFIGDLASHFGCTIGLKDSVTAVVFVALGTSVPDTFASKVAATQDQYADASIGNVTGSNAVNVFLGIGVAWSIAAIYHAANGEQFKVSPGTLAFSVTLFTIFAFINVGVLLYRRRPEIGGELGGPRTAKLLTSCLFVLLWLLYIFFSSLEAYCHIKGF

SEQ ID NO: 8 - protein, 427 aa, synthetic construct
  REGION 1..427: note = Human KCNJ2
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 9 - protein, 119 aa, synthetic construct
  REGION 1..119: note = Human B2M
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 10 - protein, 1130 aa, synthetic construct
  REGION 1..1130: note = Human CIITA
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVRPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 11 - protein, 729 aa, synthetic construct
  REGION 1..729: note = Human triadin
MTEITAEGNASTTTTVIDSKNGSVPKSPGKVLKRTVTEDIVTTFSSPAAWLLVIALIITWSAVAIVMFDLVDYKNFSASSIAKIGSDPLKLVRDAMEETTDWIYGFFSLLSDIISSEDEEDDDGDEDTDKGEIDEPPLRKKEIHKDKTEKQEKPERKIQTKVTHKEKEKGKEKVREKEKPEKKATHKEKIEKKEKPETKTLAKEQKKAKTAEKSEEKTKKEVKGGKQEKVKQTAAKVKEVQKTPSKPKEKEDKEKAAVSKHEQKDQYAFCRYMIDIFVHGDLKPGQSPAIPPPLPTEQASRPTPASPALEEKEGEKKKAEKKVTSETKKKEKEDIKKKSEKETAIDVEKKEPGKASETKQGTVKIAAQAAAKKDEKKEDSKKTKKPAEVEQPKGKKQEKKEKHVEPAKSPKKEHSVPSDKQVKAKTERAKEEIGAVSIKKAVPGKKEEKTTKTVEQEIRKEKSGKTSSILKDKEPIKGKEEKVPASLKEKEPETKKDEKMSKAGKEVKPKPPQLQGKKEEKPEPQIKKEAKPAISEKVQIHKQDIVKPEKTVSHGKPEEKVLKQVKAVTIEKTAKPKPTKKAEHREREPPSIKTDKPKPTPKGTSEVTESGKKKTEISEKESKEKADMKHLREEKVSTRKESLQLHNVTKAEKPARVSKDVEDVPASKKAKEGTEDVSPTKQKSPISFFQCVYLDGYNGYGFQFPFTPADRPGESSGQANSPGQKQQGQ

SEQ ID NO: 12 - protein, 473 aa, synthetic construct
  REGION 1..473: note = Human sarcalumenin
MRALVLLGCLLASLLFSGQAEETEDANEEAPLRDRSHIEKTLMLNEDKPSDDYSAVLQRLRKIYHSSIKPLEQSYKYNELRQHEITDGEITSKPMVLFLGPWSVGKSTMINYLLGLENTRYQLYTGAEPTTSEFTVLMHGPKLKTIEGIVMAADSARSFSPLEKFGQNFLEKLIGIEVPHKLLERVTFVDTPGIIENRKQQERGYPFNDVCQWFIDRADLIFVVFDPTKLDVGLELEMLFRQLKGRESQIRIILNKADNLATQMLMRVYGALFWSLAPLINVTEPPRVYVSSFWPQEYKPDTHQELFLQEEISLLEDLNQVIENRLENKIAFIRQHAIRVRIHALLVDRYLQTYKDKMTFFSDGELVFKDIVEDPDKFYIFKTILAKTNVSKFDLPNREAYKDFFGINPISSFKLLSQQCSYMGGCFLEKIERAITQELPGLLGSLGLGKNPGALNCDKTGCSETPKNRYRKH

SEQ ID NO: 13 - protein, 699 aa, synthetic construct
  REGION 1..699: note = Human HRC
MGHHRPWLHASVLWAGVASLLLPPAMTQQLRGDGLGFRNRNNSTGVAGLSEEASAELRHHLHSPRDHPDENKDVSTENGHHFWSHPDREKEDEDVSKEYGHLLPGHRSQDHKVGDEGVSGEEVFAEHGGQARGHRGHGSEDTEDSAEHRHHLPSHRSHSHQDEDEDEVVSSEHHHHILRHGHRGHDGEDDEGEEEEEEEEEEEEASTEYGHQAHRHRGHGSEEDEDVSDGHHHHGPSHRHQGHEEDDDDDDDDDDDDDDDDVSIEYRHQAHRHQGHGIEEDEDVSDGHHHRDPSHRHRSHEEDDNDDDDVSTEYGHQAHRHQDHRKEEVEAVSGEHHHHVPDHRHQGHRDEEEDEDVSTERWHQGPQHVHHGLVDEEEEEEEITVQFGHYVASHQPRGHKSDEEDFQDEYKTEVPHHHHHRVPREEDEEVSAELGHQAPSHRQSHQDEETGHGQRGSIKEMSHHPPGHTVVKDRSHLRKDDSEEEKEKEEDPGSHEEDDESSEQGEKGTHHGSRDQEDEEDEEEGHGLSLNQEEEEEEDKEEEEEEEDEERREERAEVGAPLSPDHSEEEEEEEEGLEEDEPRFTIIPNPLDRREEAGGASSEEESGEDTGPQDAQEYGNYQPGSLCGYCSFCNRCTECESCHCDEENMGEHCDQCQHCQFCYLCPLVCETVCAPGSYVDYFSSSLYQALADMLETPEP

SEQ ID NO: 14 - protein, 399 aa, synthetic construct
  REGION 1..399: note = Human calsequestrin-2
MKRTHLFIVGIYFLSSCRAEEGLNFPTYDGKDRVVSLSEKNFKQVLKKYDLLCLYYHEPVSSDKVTQKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDDNSDEEDNDDSDDDDDE

SEQ ID NO: 15 - protein, 18 aa, synthetic construct
  REGION 1..18: note = T2A
EGRGSLLTCGDVEENPGP

SEQ ID NO: 16 - protein, 24 aa, synthetic construct
  REGION 1..24: note = T2A
LEGGGEGRGSLLTCGDVEENPGPR

SEQ ID NO: 17 - protein, 22 aa, synthetic construct
  REGION 1..22: note = P2A
GSGATNFSLLKQAGDVEENPGP

SEQ ID NO: 18 - protein, 19 aa, synthetic construct
  REGION 1..19: note = P2A
ATNFSLLKQAGDVEENPGP

SEQ ID NO: 19 - protein, 20 aa, synthetic construct
  REGION 1..20: note = E2A
QCTNYALLKLAGDVESNPGP

SEQ ID NO: 20 - protein, 22 aa, synthetic construct
  REGION 1..22: note = F2A
VKQTLNFDLLKLAGDVESNPGP

SEQ ID NO: 21 - protein, 21 aa, synthetic construct
  REGION 1..21: note = T2A
GSGEGRGSLLTCGDVEENPGP

SEQ ID NO: 22 - protein, 23 aa, synthetic construct
  REGION 1..23: note = T2A
AAGSGEGRGSLLTCGDVEENPGP

SEQ ID NO: 23 - RNA, 12 nt, synthetic construct
  misc_feature 1..12: note = crRNA repeat
  source 1..12: mol_type = other RNA
gttttagagcta

SEQ ID NO: 24 - RNA, 63 nt, synthetic construct
  misc_feature 1..63: note = tracrRNA
  source 1..63: mol_type = other RNA
tagcaagttaaaataaggctagtccgttatcaacttgaaaaagtggcaccgagtcggtgcttt

SEQ ID NO: 25 - skipped

SEQ ID NO: 26 - DNA, 20 bp, synthetic construct
  misc_feature 1..20: note = GET000046 guide
  source 1..20: mol_type = other DNA
accccacagtggggccacta

SEQ ID NO: 27 - DNA, 20 bp, synthetic construct
  misc_feature 1..20: note = GET000047 guide
  source 1..20: mol_type = other DNA
tgttggaaggatgaggaaat

SEQ ID NO: 28 - DNA, 20 bp, synthetic construct
  misc_feature 1..20: note = GET000048 guide
  source 1..20: mol_type = other DNA
tcactatgctgccgcccagt

SEQ ID NO: 29 - RNA, 20 nt, synthetic construct
  misc_feature 1..20: note = ABO gRNA
  source 1..20: mol_type = other RNA
tctctccatgtgcagtagga

SEQ ID NO: 30 - RNA, 20 nt, synthetic construct
  misc_feature 1..20: note = FUT1 gRNA
  source 1..20: mol_type = other RNA
ctggatgtcggaggagtacg

SEQ ID NO: 31 - RNA, 20 nt, synthetic construct
  misc_feature 1..20: note = RHD gRNA
  source 1..20: mol_type = other RNA
gtctccggaaactcgaggtg

SEQ ID NO: 32 - RNA, 20 nt, synthetic construct
  misc_feature 1..20: note = F3 (CD142) gRNA
  source 1..20: mol_type = other RNA
acagtgtagacttgattgac

SEQ ID NO: 33 - RNA, 20 nt, synthetic construct
  misc_feature 1..20: note = B2M gRNA
  source 1..20: mol_type = other RNA
cgtgagtaaacctgaatctt

SEQ ID NO: 34 - RNA, 20 nt, synthetic construct
  misc_feature 1..20: note = CIITA gRNA
  source 1..20: mol_type = other RNA
gatattggcataagcctccc

SEQ ID NO: 35 - RNA, 20 nt, synthetic construct
  misc_feature 1..20: note = TRAC gRNA
  source 1..20: mol_type = other RNA
agagtctctcagctggtaca